(19) United States
(12) Patent Application Publication
(10) Pub. No.: US 2025/0011330 A1
(43) Pub. Date: Jan. 9, 2025

(54) Imidazo[1,2- a]pyrazine and Imidazo[1,2- a]pyridine Based Tyrosyl-DNA Phosphodiesterase I (TDP1) Inhibitors

(71) Applicant: The USA, As Represented by the Secretary, Department of Health and Human Services, Bethesda, MD (US)

(72) Inventors: Xuezhi Zhao, Bethesda, MD (US); Wenjie Wang, Bethesda, MD (US); George Themistoclis Lountos, Frederick, MD (US); Evgeny Anatolyevich Kiselev, Bethesda, MD (US); David Spencer Waugh, Bethesda, MD (US); Yves Georges Pommier, Bethesda, MD (US); Terrence Richard Burke, Jr., Bethesda, MD (US)

(21) Appl. No.: 18/262,779

(22) PCT Filed: Jan. 26, 2022
(86) PCT No.: PCT/US2022/013946

§ 371 Date: Jul. 25, 2023

Related U.S. Application Data
(60) Provisional application No. 63/141,634, filed on Jan. 26, 2021.

(51) Int. Cl. C07D 487/04; A61K 31/444; A61K 31/4985; A61P 35/00; C07D 471/04

(52) U.S. Cl. CPC C07D 487/04; A61K 31/444; A61K 31/4985; A61P 35/00; C07D 471/04

(57) ABSTRACT

The present disclosure provides methods, compounds and compositions that are relevant to DNA-repair and DNA-repair proteins such as tyrosyl-DNA phosphodiesterase 1 (TDP1) and type I topoisomerase (TOP1).

### BACKGROUND

Tyrosyl-DNA phosphodiesterase 1 (TDP1) catalyzes the hydrolysis of the phosphodiester bond between a tyrosine residue of Type I topoisomerase (TOP1) and the 3′-phosphate of its DNA substrate. It is a key enzyme involved in repairing stalled TOP1-DNA complexes resulting from administration of TOP1 inhibitors during anticancer therapy. Inhibiting TDP1's ability to repair stalled TOP1-DNA complexes could potentially provide synergistic enhancement of the selectivity and potency of TOP1 inhibitors. Although TDP1 is an attractive new anticancer target, development of bona fide TDP1 inhibitors has proven to be elusive. There are currently no TDP1 inhibitors in clinical trials. Certain groups have reported a variety of TDP1 inhibitors, including those which exhibit potent inhibitory potencies in TDP1 in catalytic assays. Some of these inhibitors may even appear to illicit desired effects in whole cell assays. However, in most cases, convincing rationalization for interactions with TDP1 are not provided and chemical structures frequently point toward promiscuous mechanisms of action. Accordingly, there is a pressing need for the discovery of TDP1 inhibitors with validated binding interactions and mechanisms of actions.

### SUMMARY

The present disclosure provides methods, compounds and compositions that are relevant to DNA-repair and DNA-repair proteins such as tyrosyl-DNA phosphodiesterase 1 (TDP1) and type I topoisomerase (TOP1).

In one aspect the disclosure provides a compound of Formula I

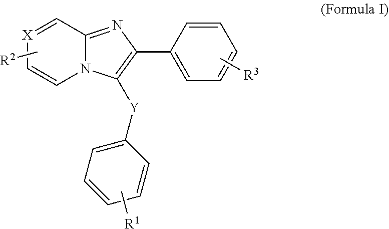

or a pharmaceutically acceptable salt thereof, where R¹ is absent or represents H, halogen, hydroxyl, cyano, nitro, amino, —COOH, —CHO, —CONH₂, —SO₃H, —SO₂F, —OSO₂F, —SO₂(C₁-C₂alkyl), —(C₁-C₂alkyl)CO₂H, —PO₃H, —PO₂(C₁-C₂alkyl), —PO₂NH(C₁-C₂alkyl), —(C₁-C₂alkyl)PO₃H, —(C₁-C₂alkyl)PO₂NH(C₁-C₂alkyl), phenyl, phenoxy, benzyl, benzyloxy, C₁-C₆alkyl, C₁-C₆alkoxy, C₁-C₂haloalkyl, and C₁-C₂haloalkoxy, mono- or di(C₁-C₄alkyl)amino, C₃-C₇cycloalkyl, or 5-7-membered heterocycloalkyl, trifluoromethyl, or trifluoromethoxy; R², and R³ are each independently absent, or represent one or more substituents independently selected from H, halogen, hydroxyl, cyano, nitro, amino, —COOH, —CHO, —CONH₂, —SO₃H, —SO₂F, —OSO₂F, —SO₂(C₁-C₂alkyl), phenyl, phenoxy, benzyl, benzyloxy, C₁-C₆alkyl, C₁-C₆alkoxy, C₁-C₂haloalkyl, and C₁-C₂haloalkoxy, mono- or di(C₁-C₄alkyl)amino, C₃-C₇cycloalkyl, or 5-7-membered heterocycloalkyl, trifluoromethyl, trifluoromethoxy, —(C₁-C₄)ONH₂, an oxime of formula —(C₁-C₄)ON═C—R²⁰, wherein the oxime is formed by the reaction of —(C₁-C₄)ONH₂ and an aldehyde

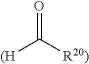

selected from the group consisting of A1-T12 in Table 4; or -L-Rec, wherein L is a linker selected from

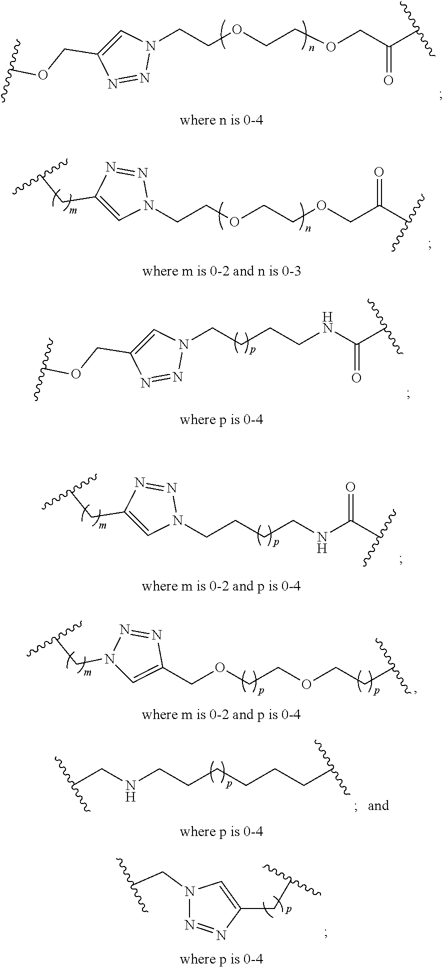

and
Rec is an E3 ubiquitin ligase recruiter. Not all of R¹, R², and R³ are absent and at least one of R¹, R², and R³ is phenyl, —COOH or nitro; X is C or N; and Y is NH, O, or NSO₂F.

In certain embodiments, Rec is a von Hippel-Lindau (VHL) E3 ubiquitin ligase recruiter, a cereblon (CRBN) E3 ubiquitin ligase recruiter, a Inhibitors of Apoptosis Protein (IAPs) E3 ubiquitin ligase recruiter, or a mouse double minute 2 homolog (MDM2) E3 ubiquitin ligase recruiter.

In certain embodiments, Rec is a VHL recruiter according to

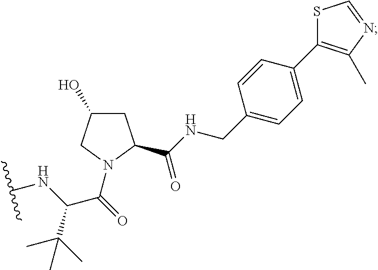 [SMILES: *N[C@H](C(=O)N1C[C@H](O)C[C@H]1C(=O)NCc1ccc(-c2scnc2C)cc1)C(C)(C)C]

or
Rec is a CRBN recruiter according to

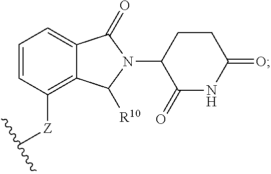

where R¹⁰ is H or carbonyl; and Z is O, NH, or N(C₁-C₃alkyl).

In certain embodiments, the compound or salt of Formula I includes those in which R¹ is absent or one or more substituents independently chosen from hydroxyl, nitro, and —COOH; R² is absent or one or more substituents independently chosen from bromo, —COOH, —SO₂CH₃, and phenyl; and R³ is absent or one or more substituents independently chosen from —COOH, —SO₃H, hydroxyl, nitro, methyl, methoxy, butoxy, phenyl, morpholinyl, trifluoromethyl, and trifluoromethoxy.

In another aspect, the disclosure provides a compound of Formula I.5

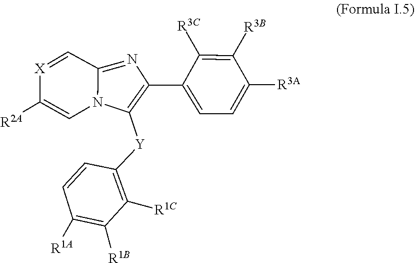

or a pharmaceutically acceptable salt thereof, where R^{1A} is absent or represents H, nitro, —COOH; R^{1B} is absent or represents H, —COOH; R^{1C} is absent or represents H or hydroxyl; R^{2A} is absent or represents H, halogen, —COOH, —SO₂Me, —CH₂ONH₂, an oxime of formula —CH₂ON═C—R²⁰, wherein the oxime is formed by the reaction of —CH₂ONH₂ and an aldehyde

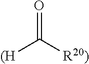

selected from the group consisting of B7, D1, E6, F5, G6, G7, G8, I7, M10, M11, N3, N4, O11, P1, P3, P4, P8, R10, R11, S4, S12, and T12 from Table 4; R^{2B} is absent or represents H, —COOH, phenyl; R^{3A} is absent or represents H, —COOH, hydroxyl, C₁-C₃alkyl, phenyl, benzyloxy, trifluoromethyl, nitro, —SO₃H, —SO₂(C₁-C₂alkyl), morpholinyl, —CH₂ONH₂,

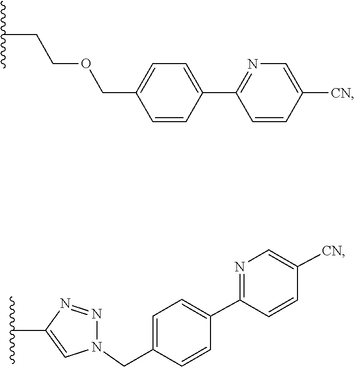 [SMILES: *CCOCc1ccc(-c2ccc(C#N)cn2)cc1 ; *c1cn(Cc2ccc(-c3ccc(C#N)cn3)cc2)nn1]

or an oxime of formula —CH₂ON═C—R²⁰, wherein the oxime is formed by the reaction of —CH₂ONH₂ and an aldehyde

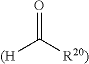

selected from the group consisting of A6, B7, B9, B11, D1, D10, E6, F1, F3, F9, F10, F11, G6, G8, H1, H7, H11, I1, I3, I7, I8, J9, K1, K9, L8, L11, M10, M11, N2, N3, N4, N8, O11, P2, P3, P4, P8, Q9, Q12, R7, R10, R11, S2, S11, T1, T3, and T12 from Table 4; R^{3B} is absent or represents H, hydroxyl; R^{3C} is absent or represents H, hydroxyl; X is C—R^{2B} or N; and Y is NH.

In certain embodiments, the compound or salt of Formula I.5 includes those for which:
X is C—R^{2B}, R^{1A} is —COOH, R^{3C} is hydroxyl, C— and R^{1B}, R^{1C}, R^{2A}, R^{2B}, and R^{3A} are all H (8a);
X is C—R^{2B}, R^{1A} is —COOH, R^{1A} is methyl, and R^{1B}, R^{1C}, R^{2A}, R^{2B}, and R^{3C} are all H (8b);
X is C—R^{2B}, R^{1A} is —COOH, R^{1A} is benzyloxy, and R^{1B}, R^{1C}, R^{2A}, R^{2B}, and R^{3C} are all H (8c);
X is C—R^{2B}, R^{1A} is —COOH, R^{3A} is trifluoromethyl, and R^{1B}, R^{1C}, R^{2A}, R^{2B}, and R^{3C} are all H (8d);
X is C—R^{2B}, R^{1A} is —COOH, R^{3A} is nitro, and R^{1B}, R^{1C}, R^{2A}, R^{2B}, and R^{3C} are all H (8e);
X is C—R^{2B}, R^{3A} is —COOH, and R^{1A}, R^{1B}, R^{1C}, R^{2A}, R^{2B}, and R^{3C} are all H (8i);
X is C—R^{2B}, R^{2A} is —COOH, and R^{1A}, R^{1B}, R^{1C}, R^{2B}, R^{1A}, and R^{3C} are all H (8k);
X is C—R^{2B}, R^{1A} is nitro, R^{2A} is —COOH, and R^{1B}, R^{1C}, R^{2B}, R^{1A}, and R^{3C} are all H (8l);
X is C—R^{2B}, R^{1A} is —COOH, R^{2A} is —COOH, and R^{1B}, R^{1C}, R^{2B}, R^{1A}, and R^{3C} are all H (8m);
X is C—R^{2B}, R^{1A} is —COOH, R^{2A} is —COOH, R^{3A} is phenyl, and R^{1B}, R^{1C}, R^{2B}, and R^{3C} are all H (8n);
X is C—R^{2B}, R^{1A} is —COOH, R^{2A} is Br, and R^{1B}, R^{1C}, R^{2B}, R^{3A}, and R^{3C} are all H (8o);
X is C—R^{2B}, R^{1A} is —COOH, R^{2B} is phenyl, and R^{1B}, R^{1C}, R^{2A}, R^{3A}, and R^{3C} are all H (8p);
X is C—R^{2B}, R^{1A} is —COOH, R^{3A} is —SO₂Me, and R^{1B}, R^{1C}, R^{2A}, R^{2B}, and R^{3C} are all H (8q);
X is C—R^{2B}, R^{1A} is —COOH, R^{2A} is —SO₂Me, R^{3A} is phenyl, and R^{1B}, R^{1C}, R^{2B}, and R^{3C} are all H (8r);
X is C—R^{2B}, R^{1A} is nitro, R^{2A} is —SO₂Me, R^{1B}, R^{1C}, R^{2B}, R^{3A}, and R^{3C} are all H (6u);
X is C—R^{2B}, R^{1A} is —COOH, R^{2B} is —COOH, and R^{1B}, R^{1C}, R^{2A}, R^{3A}, and R^{3C} are all H (8s);
X is C—R^{2B}, R^{1A} is —COOH, R^{1B} is —COOH, and R^{1C}, R^{2A}, R^{2B}, R^{3A}, and R^{3C} are all H (10b);
X is N, R^{1A} is —COOH, R^{3C} is hydroxyl, and R^{1B}, R^{1C}, R^{2A}, R^{3A}, R^{3B}, R^{3D} are all H (M7);
X is N, R^{1B} is —COOH, R^{3A} is methyl, and R^{1A}, R^{1C}, R^{2A}, R^{3A}, R^{3B}, R^{3D} are all H (M8);
X is N, R^{1A} is —COOH, and R^{1B}, R^{1C}, R^{2A}, R^{3A}, R^{3B}, R^{3C}, R^{3D} are all H (7b);
X is N, R^{1A} is —COOH, R^{3B} is hydroxyl, and R^{1B}, R^{1C}, R^{2A}, R^{3A}, R^{3C}, R^{3D} are all H (7d);
X is N, R^{1A} is —COOH, R^{3A} is hydroxyl, and R^{1B}, R^{1C}, R^{2A}, R^{3B}, R^{3C}, R^{3D} are all H (7e);
X is N, R^{1A} is —COOH, R^{1C} is hydroxyl, and R^{1B}, R^{2A}, R^{3A}, R^{3B}, R^{3C}, R^{3D} are all H (7f);
X is N, R^{1B} is —COOH, R^{3C} is hydroxyl, and R^{1A}, R^{1C}, R^{2A}, R^{3A}, R^{3B}, R^{3D} are all H (7m);
X is N, R^{1B} is —COOH, R^{3A} is hydroxyl, and R^{1A}, R^{1C}, R^{2A}, R^{3B}, R^{3C}, R^{3D} are all H (7o);
X is N, R^{1B} is —COOH, R^{3A} is morpholinyl, and R^{1A}, R^{1C}, R^{2A}, R^{3B}, R^{3C}, R^{3D} are all H (7p); or
X is N, R^{1A} and R^{1B} are —COOH, and R^{1C}, R^{2A}, R^{3A}, R^{3B}, R^{3C}, R^{3D} are all H (10a); where for each compound above, Y is NH.

In certain embodiments, the compound or salt of Formula I.5 includes those for which:
X is C—R^{2B}, R^{1A} is —COOH; R^{2B} is phenyl; R^{3A} is —(C₁-C₄)ONH₂, R^{1B}, R^{1C}, R^{2A}, R^{3B}, a R^{3C} are H;
X is C—R^{2B}, R^{1A} is —COOH; R^{2B} is phenyl; R^{3A}, R^{1B}, R^{1C}, R^{2A}, R^{3B}, and R^{3C} are H (664);
X is C—R^{2B}, R^{1A} is —COOH; R^{2B} is phenyl; R^{3A} is —CH₂ONH₂, R^{1B}, R^{1C}, R^{2A}, R^{3B}, and R^{3C} are H (699);
X is C—R^{2B}, R^{1A} is —COOH; R^{2B} is phenyl; R^{1B}, R^{1C}, R^{2A}, R^{3B}, and R^{3C} are H; R^{3A} is an oxime of formula —CH₂ON═C—R²⁰, wherein the oxime is formed by the reaction of —CH₂ONH₂ and an aldehyde

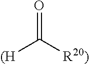

selected from the group consisting of A6, B7 (xz699-B7; 6-B7; or XZ710 (E) isomer; XZ703 (Z) isomer), B9, B11, D1 (XZ699-D1; 6-D1; XZ701 (E) isomer; XZ708 (Z) isomer)), D10, E6 (XZ699-E6; 6-E7; XZ702 (E) isomer; XZ709 (Z) isomer), F1, F3, F9, F10, F11, G6, G8, H1, H7, H11, I1, I3, I7, I8, J9, K1, K9, L8, L11, M10 (XZ699-M10; 6-M10; XZ705 (E) isomer; XZ712 (Z) isomer), M11, N2, N3, N4, N8, O11, P2, P3 (XZ699-P3; 6-P3; XZ704 (E) isomer; XZ711 (Z) isomer), P4, P8, Q9, Q12, R⁷, R¹⁰, R¹¹, S2, S11, T1, T3, or T12 from Table 4;
X is C—R^{2B}, R^{1A} is —COOH; R^{2B} is H or —(C₁-C₄)ONH₂, R^{1B}, R^{1C}, R^{2A}, R^{3A}, R^{3B}, and R^{3C} are H;
X is C—R^{2B}, R^{1A} is —COOH; R^{1B}, R^{1C}, R^{2A}, R^{2B}, R^{3A}, R^{3B}, and R^{3C} are H (XZ615);
X is C—R^{2B}, R^{1A} is —COOH; R^{2A} is —CH₂ONH₂, R^{1B}, R^{1C}, R^{2B}, R^{3A}, R^{3B}, and R^{3C} are H (XZ700);
X is C—R^{2B}, R^{1A} is —COOH; R^{1B}, R^{1C}, R^{2B}, R^{3A}, R^{3B}, and R^{3C} are H; R^{2A} is an oxime of formula —CH₂ON═C—R²⁰, wherein the oxime is formed by the reaction of —CH₂ONH₂ and an aldehyde

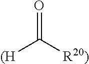

selected from the group consisting of B7 (xz700-B7; 5-B7), D1 (XZ700-D1; 5-D1; XZ706 (E) isomer; XZ717 (Z) isomer)), E6 (XZ700-E6; 5-E6), F5, G6, G7, G8, 17, M10 (XZ700-M10; 5-M10), M11, N3, N4, O11, P1, P3 (XZ700-P3; 5-P3; XZ707 (E) isomer; XZ713 (Z) isomer), P4, P8, R¹⁰, R¹¹, S4, S12, and T12 from Table 4; where for each compound above, Y is NH.

In certain embodiments, the compound or salt of Formula I.5 is one for which Y is NH; X is C—R^{2B}; R is —COOH; R^{1B}, R^{1C}, R^{2A}, R^{3A}, R^{3B}, and R^{3C} are H; and R^{2B} is

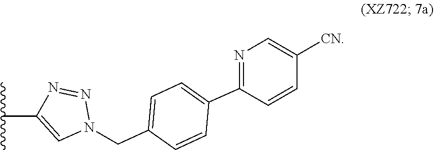 [SMILES: *c1cn(Cc2ccc(-c3ccc(C#N)cn3)cc2)nn1]

In certain embodiments, the compound or salt of Formula I.5 is one for which Y is NH; X is C—R^{2B}; R^{1A} is —COOH; R^{1B}, R^{1C}, R^{2B}, R^{3A}, R^{3B}, and R^{3C} are H; and R^{2A} is

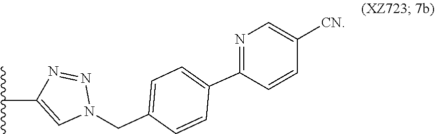 [SMILES: *c1cn(Cc2ccc(-c3ccc(C#N)cn3)cc2)nn1]

In certain embodiments, the compound or salt of Formula I.5 is one for which Y is NH; X is C—R^{2B}; R^{1A} is —COOH; R^{1B}, R^{1C}, R^{2A}, R^{2B}, R^{3B}, and R^{3C} are H; and R^{3A} is —OCH₂—X where X is

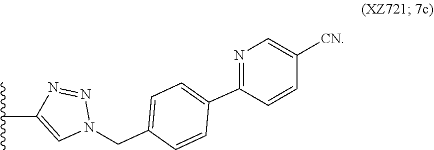 [SMILES: *c1cn(Cc2ccc(-c3ccc(C#N)cn3)cc2)nn1]

In certain embodiments, the compound or salt of Formula I.5 is one for which Y is NH; X is C—R^{2B}; R^{1A} is —COOH; R^{2B} is phenyl; R^{1B}, R^{1C}, R^{2A}, R^{3B}, and R^{3C} are H; and R^{3A} is —OCH₂—X where X is

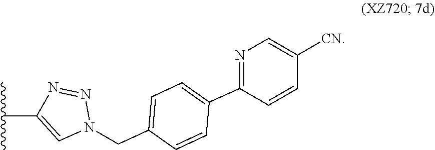 [SMILES: *c1cn(Cc2ccc(-c3ccc(C#N)cn3)cc2)nn1]

In certain embodiments, the compound or salt of Formula I.5 is one for which Y is NH; X is C—R^{2B}; R^{1A} is —COOH; R^{1B}, R^{1C}, R^{2A}, R^{2B}, R^{3B}, and R^{3C} are H; and R^{3A} is

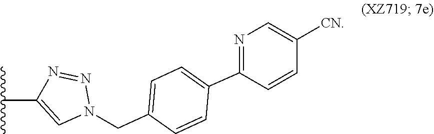 [SMILES: *c1cn(Cc2ccc(-c3ccc(C#N)cn3)cc2)nn1]

In certain embodiments, the compound or salt of Formula I.5 is one for which Y is NH; X is C—R^{2B}; R^{1A} is —COOH; R^{2B} is phenyl; R^{1B}, R^{1C}, R^{2A}, R^{3B}, and R^{3C} are H; and R^{3A}

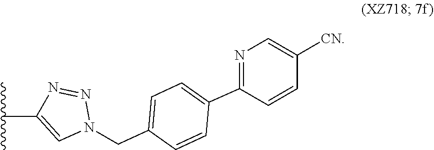 [SMILES: *c1cn(Cc2ccc(-c3ccc(C#N)cn3)cc2)nn1]

In certain embodiments, the compound or salt of Formula I.5 is one for which Y is NH; X is C—R^{2B}; R^{1A} is —COOH; R^{1B}, R^{1C}, R^{2A}, R^{2B}, R^{3B}, and R^{3C} are H; and R^{3A} is

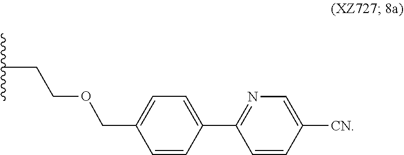 [SMILES: *CCOCc1ccc(-c2ccc(C#N)cn2)cc1]

In certain embodiments, the compound or salt of Formula I.5 is one for which Y is NH; X is C—R^{2B}; R^{1A} is —COOH; R^{2B} is phenyl; R^{1B}, R^{1C}, R^{2A}, R^{3B}, and R^{3C} are H; and R^{3A} is

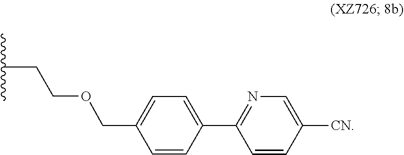 [SMILES: *CCOCc1ccc(-c2ccc(C#N)cn2)cc1]

In certain embodiments, the compound or salt of Formula I.5 is

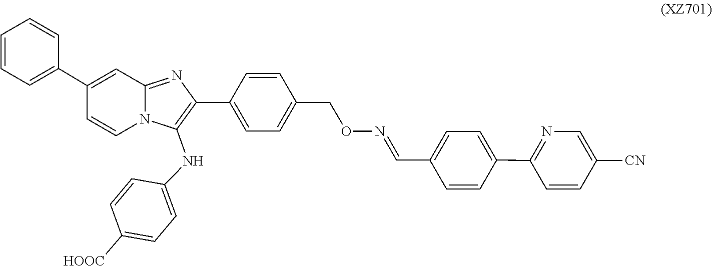 [SMILES: N#Cc1ccc(-c2ccc(/C=N/OCc3ccc(-c4nc5cc(-c6ccccc6)ccn5c4Nc4ccc(C(=O)O)cc4)cc3)cc2)nc1]

In another aspect, the disclosure provides a compound of Formula I-A

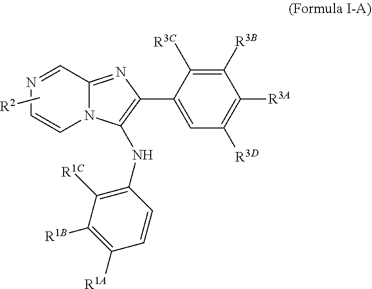

or a pharmaceutically acceptable salt thereof, where one of R^{1A} and R^{1B} is H and the other is —COOH; R^{1C} is H or hydroxyl; R² is 0 to 3 substituents independently chosen from hydroxyl, halogen, C₁-C₂alkyl, C₁-C₂alkoxy; R^{3A} is H, hydroxyl, C₁-C₂alkyl, C₁-C₂alkoxy, C₃-C₇cycloalkyl, or 5-7-membered heterocycloalkyl; and R^{3B}, R^{3C}, and R^{3D} are independently chosen from H, halogen, and hydroxyl.

In certain embodiments, the compound or salt of Formula I-A includes those in which one of RA and RB is H and the other is —COOH; R^{1C} is H or hydroxyl; R² is absent; R^{3A} is H, hydroxyl, or morpholinyl; R^{3B} is H or hydroxyl; R^{3C} is H or hydroxyl; and R^{3D} is H.

In certain embodiments, the compound or salt of Formula I-A includes those for which:
R^{1A} is —COOH, R^{3C} is hydroxyl, and RB, R^{1C}, R^{3A}, R^{3B}, R^{3D} are all H (M7);
R^{1B} is —COOH, R^{3A} is methyl, and R^{1A}, R^{1C}, R^{3A}, R^{3B}, R^{3D} are all H (M8);
R^{1A} is —COOH, and R^{1B}, R^{1C}, R^{3A}, R^{3B}, R^{3C}, R^{3D} are all H (7b);
R^{1A} is —COOH, R^{3B} is hydroxyl, and R^{1B}, R^{1C}, R^{3A}, R^{3C}, R^{3D} are all H (7d);
R^{1A} is —COOH, R^{3A} is hydroxyl, and R^{1B}, R^{1C}, R^{3B}, R^{3C}, R^{3D} are all H (7e);
R^{1A} is —COOH, R^{1C} is hydroxyl, and R^{1B}, R^{3A}, R^{3B}, R^{3C}, R^{3D} are all H (7f);
R^{1B} is —COOH, R^{3C} is hydroxyl, and R^{1A}, R^{1C}, R^{3A}, R^{3B}, R^{3D} are all H (7m);
R^{1B} is —COOH, R^{3A} is hydroxyl, and R^{1A}, R^{1C}, R^{3B}, R^{3C}, R^{3D} are all H (70);
R^{1B} is —COOH, R^{3A} is morpholinyl, and R^{1A}, R^{1C}, R^{3B}, R^{3C}, R^{3D} are all H (7p); or
R^{1A} and R^{1B} are —COOH, and R^{1C}, R^{3A}, R^{3B}, R^{3C}, R^{3D} are all H (10a).

In another aspect, the disclosure provides a compound of Formula I-B:

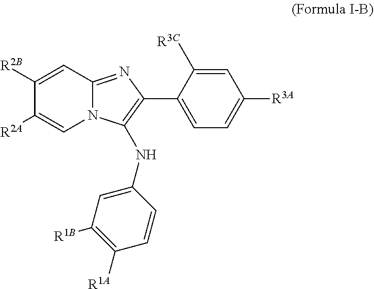

or a pharmaceutically acceptable salt thereof, where R^{1A} and R^{1B} are independently chosen from H, halogen, hydroxyl, cyano, nitro, amino, —COOH, —CHO, —CONH₂, —SO₃H, —SO₂F, C₁-C₆alkyl, C₁-C₆alkoxy, C₁-C₂haloalkyl, and C₁-C₂haloalkoxy; R^{2A} is H, halogen, hydroxyl, cyano, nitro, amino, —COOH, —CHO, —CONH₂, —SO₃H, —SO₂F, 2-SO₂C₁-C₆alkyl, C₁-C₆alkyl, C₁-C₆alkoxy, C₁-C₂haloalkyl, or C₁-C₂haloalkoxy, ethynyl, ethynylbenzene; R^{2B} is H, halogen, hydroxyl, cyano, C₁-C₆alkyl, C₁-C₆alkoxy, mono- or di(C₁-C₄alkyl)amino, phenyl, benzyl, C₁-C₂haloalkyl, or C₁-C₂haloalkoxy, ethynyl, ethynylbenzene; R^{3A} is H, halogen, hydroxyl, cyano, nitro, amino, —COOH, —CHO, —CONH₂, —SO₃H, —SO₂F, phenyl, phenoxy, benzyl, benzyloxy, C₁-C₆alkyl, C₁-C₆alkoxy, mono- or di(C₁-C₄alkyl)amino, C₁-C₂haloalkyl, or C₁-C₂haloalkoxy; and R^{3C} is H, —COOH, halogen, hydroxyl, cyano, C₁-C₆alkyl, C₁-C₆alkoxy, mono- or di(C₁-C₄alkyl)amino, C₁-C₂haloalkyl, or C₁-C₂haloalkoxy; wherein one of R^{1A}, R^{2A}, and R^{3A} is —COOH.

In certain embodiments, the compound or salt of Formula I-B includes those in which R^{1A} and R^{1B} are independently chosen from H, —COOH, and nitro; R^{2A} and R^{2B} are independently chosen from H, halogen, —COOH, —SO₂(C₁-C₂alkyl), and phenyl; and R^{3A} and R^{3C} are independently chosen from H, nitro, hydroxyl, —COOH, —SO₃H, —SO²Me, C₁-C₄alkyl, C₁-C₂alkoxy, phenyl, phenoxy, benzyl, benzyloxy, trifluoromethyl, trifluoromethoxy, and phenyl.

In certain embodiments, the compound or salt of Formula I-B includes those for which:
R^{1A} is —COOH, R^{3C} is hydroxyl, and R^{1B}, R^{2A}, R^{2B}, and R^{3A} are all H (8a);
R^{1A} is —COOH, R^{3A} is methyl, and R^{1B}, R^{2A}, R^{2B}, and R^{3C} are all H (8b);
R^{1A} is —COOH, R^{3A} is benzyloxy, and R^{1B}, R^{2A}, R^{2B}, and R^{3C} are all H (8c);
R^{1A} is —COOH, R^{3A} is trifluoromethyl, and R^{1B}, R^{2A}, R^{2B}, and R^{3C} are all H (8d);
R^{1A} is —COOH, R^{3A} is nitro, and R^{1B}, R^{2A}, R^{2B}, and R^{3C} are all H (8e);
R^{3A} is —COOH, and R^{1A}, R^{1B}, R^{2A}, R^{2B}, and R^{3C} are all H (8i);
R^{2A} is —COOH, and R^{1A}, R^{1B}, R^{2B}, R^{3A}, and R^{3C} are all H (8k);
R^{1A} is nitro, R^{2A} is —COOH, and R^{1B}, R^{2B}, R^{3A}, and R^{3C} are all H (8l);
R^{1A} is —COOH, R^{2A} is —COOH, and R^{1B}, R^{2B}, R^{3A}, and R^{3C} are all H (8m);
R^{1A} is —COOH, R^{2A} is —COOH, R^{3A} is phenyl, and R^{1B}, R^{2B}, and R^{3C} are all H (8n);
R^{1A} is —COOH, R^{2A} is Br, and R^{1B}, R^{2B}, R^{3A}, and R^{3C} are all H (8o);
R^{1A} is —COOH, R^{2B} is phenyl, and R^{1B}, R^{2A}, R^{3A}, and R^{3C} are all H (8p);
R^{1A} is —COOH, R^{3A} is —SO₂Me, and R^{1B}, R^{2A}, R^{2B}, and R^{3C} are all H (8q);
R^{1A} is —COOH, R^{2A} is —SO₂Me, R^{3A} is phenyl, and R^{1B}, R^{2B}, and R^{3C} are all H (8r);
R^{1A} is nitro, R^{2A} is —SO₂Me, R^{1B}, R^{2B}, R^{3A}, and R^{3C} are all H (6u);
R^{1A} is —COOH, R^{2B} is —COOH, and R^{1B}, R^{2A}, R^{3A}, and R^{3C} are all H (8s); or
R^{1A} is —COOH, R^{1B} is —COOH, and R^{2A}, R^{2B}, R^{3A}, and R^{3C} are all H (10b).

In another aspect, the disclosure provides a compound of Formula I-C:

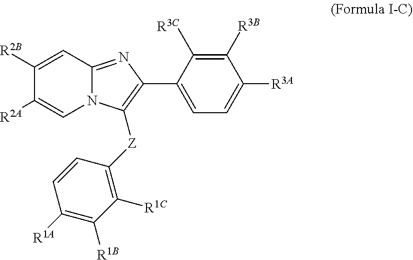

or a pharmaceutically acceptable salt thereof, where R^{1A}, R^{1B} and R^{1C} are each independently H, —COOH, —SO₂F or —OSO₂F; R^{2A} and R^{2B} are each independently H, phenyl, —SO₂F or —OSO₂F; R^{3A}, R^{3B} and R^{3C} are each independently H, —COOH, —SO₂F or —OSO₂F; and Z is O, NH, or N—SO₂F; where at least one of R^{1A}, R^{1B} and R^{1C}, R^{2A} and R^{2B}, R^{3A}, R^{3B}, and R^{3C} is —SO₂F or —OSO₂F.

In certain embodiments, the compound or salt of Formula I-C includes those in which R^{1A}, R^{1B} and R^{1C} are each independently H, —COOH, —SO₂F or —OSO₂F; R^{2A} and R^{2B} are each independently H, phenyl, —SO₂F or —OSO₂F; R^{3A}, R^{3B} and R^{3C} are each independently H, —COOH, —SO₂F or —OSO₂F; Z is NH; and wherein at least one of R^{1A}, R^{1B} and R^{1C}, R^{2A} and R^{2B}, R^{3A}, R^{3B}, and R^{3C} is, —SO₂F or —OSO₂F.

In certain embodiments, the compound or salt of Formula I-C includes those for which:
R^{1A} is —COOH, R^{2B} is phenyl, R^{3A} is —SO₂F, R^{1B}, R^{1C}, R^{2A}, R^{3B}, and R^{3C} are all H, and Z is NH;
R^{1A} is —COOH, R^{2B} is phenyl, R^{3B} Is —SO₂F, R^{1B}, R^{1C}, R^{2A}, R^{3A}, and R^{3C} are all H, and Z is NH;
R^{1A} is —COOH, R^{2B} is phenyl, R^{3C} is —SO₂F, R^{1B}, R^{1C}, R^{2A}, R^{3A}, and R^{3B} are all H, and Z is NH;
R^{1A} is —COOH, R^{2A} is —SO₂F, R^{1B}, R^{1C}, R^{2B}, R^{3A}, R^{3B} and R^{3C} are all H, and Z is NH;
R^{1A} is —COOH, R^{2B} is phenyl, R^{3C} is —SO₂F, R^{1B}, R^{1C}, R^{2A}, R^{3A}, and R^{3B} are all H, and Z is N—SO₂F; or
R^{1A} is —SO₂F, R^{1B}, R^{1C}, R^{2A}, R^{2B}, R^{3A}, R^{3B}, and R^{3C} are all H, and Z is NH.

In another aspect, the disclosure provides a compound of Formula I-D:

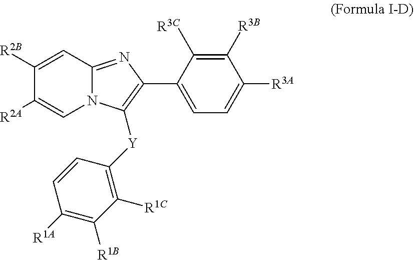

or a pharmaceutically acceptable salt thereof, where R^{1A}, R^{1B} and R^{1C} are each independently H or —COOH; R^{2A}, R^{2B}, R^{3A}, R^{3B} and R^{3C} are each independently H, phenyl or -L-Rec, provided that no more than one of R^{2A}, R^{2B}, R^{3A}, R^{3B} and R^{3C} is -L-Rec; wherein L is a linker that includes:

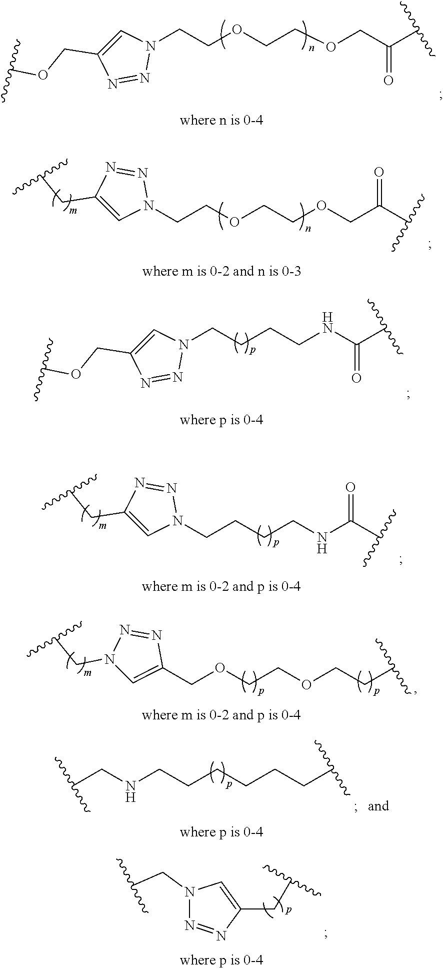

Rec is an E3 ubiquitin ligase recruiter; and
Y is O or NH.

In certain embodiments, the compound or salt of Formula I-D includes those in which Rec is a von Hippel-Lindau (VHL) E3 ubiquitin ligase recruiter, a cereblon (CRBN) E3 ubiquitin ligase recruiter, a Inhibitors of Apoptosis Protein (IAPs) E3 ubiquitin ligase recruiter, or a mouse double minute 2 homolog (MDM2) E3 ubiquitin ligase recruiter.

In certain embodiments, the compound or salt of Formula I-D includes those in which Rec is a VHL recruiter according to

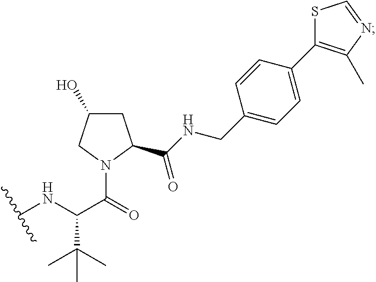 [SMILES: *N[C@H](C(=O)N1C[C@H](O)C[C@H]1C(=O)NCc1ccc(-c2scnc2C)cc1)C(C)(C)C]

or
Rec is a CRBN recruiter according to

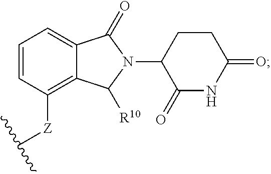

where R¹⁰ is H or carbonyl; and Z is O, NH, or N(C₁-C₃alkyl).

In certain embodiments, Y is NH; R^{1A} is —COOH; R^{2B} is phenyl; R^{1B}, R^{1C}, R^{2A}, R^{3B} and R^{3C} are H; R^{3A} is -L-Rec, wherein L is a linker selected from:

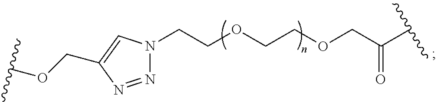

where n is 1-3; and
Rec is a VHL recruiter according to

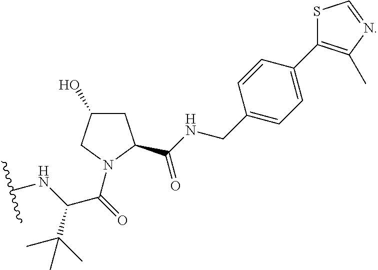 [SMILES: *N[C@H](C(=O)N1C[C@H](O)C[C@H]1C(=O)NCc1ccc(-c2scnc2C)cc1)C(C)(C)C]

In certain embodiments where Y is NH; R^{1A} is —COOH; R^{2B} is phenyl; R^{1B}, R^{1C}, R^{2A}, R^{3B} and R^{3C} are H° R^{3A} is -L-Rec, wherein L is a linker selected from:

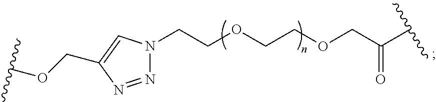

and
Rec is a VHL recruiter according to

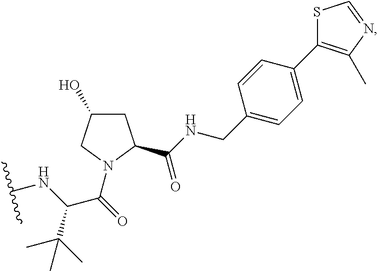 [SMILES: *N[C@H](C(=O)N1C[C@H](O)C[C@H]1C(=O)NCc1ccc(-c2scnc2C)cc1)C(C)(C)C]

where for the linker, n is 1 (XZ679); n is 2 (XZ680); or n is 3 (XZ681).

In certain embodiments, Y is NH; R^{1A} is —COOH; R^{1B}, R^{1C}, R^{2A}, R^{3A}, R^{3B} and R^{3C} are H; R^{2B} -L-Rec, wherein L is a linker selected from:

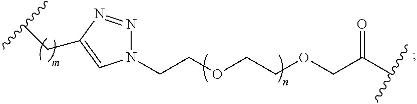

where m is 0-3; and n is 1-3; and
Rec is a VHL recruiter according to

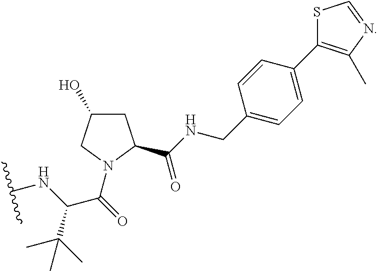 [SMILES: *N[C@H](C(=O)N1C[C@H](O)C[C@H]1C(=O)NCc1ccc(-c2scnc2C)cc1)C(C)(C)C]

In certain embodiments where Y is NH; R^{1A} is —COOH; R^{1B}, R^{1C}, R^{2A}, R^{3A}, R^{3B} and R^{3C} are H; R^{2B} is -L-Rec, wherein L is a linker selected from:

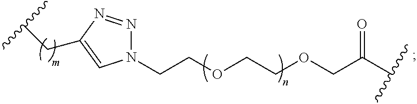

and
Rec is a VHL recruiter according to

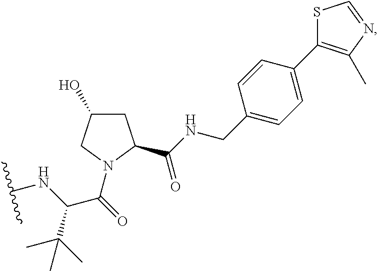 [SMILES: *N[C@H](C(=O)N1C[C@H](O)C[C@H]1C(=O)NCc1ccc(-c2scnc2C)cc1)C(C)(C)C]

where for the linker, m is 0 and n is 1 (XZ682); n is 2 (XZ683); or n is 3 (XZ684).

In certain embodiments, Y is NH; R^{1A} is —COOH; R^{1B}, R^{1C}, R^{2A}, R^{3A}, R^{3B} and R^{3C} are H; R^{2B} is -L-Rec, wherein L is a linker selected from:

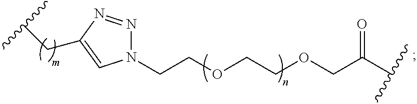

where m is 0-3, and p is 1-3; and
Rec is a CRBN recruiter selected from:

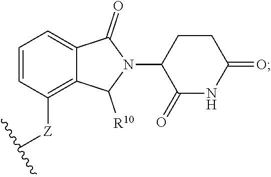

where R¹⁰ is H or carbonyl; and
Z is O or NH, or N(C₁-C₃alkyl).

In certain embodiments where Y is NH; R^{1A} is —COOH; R^{1B}, R^{1C}, R^{2A}, R^{3A}, R^{3B} and R^{3C} are H; R^{2B} is -L-Rec, wherein L is a linker selected from:

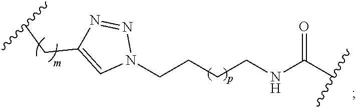

and
Rec is a CRBN recruiter according to:

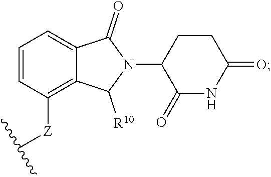

where R¹⁰ is H or carbonyl; and
Z is O or NH, or N(C₁-C₃alkyl);
for the linker, m is 0 and p is 1; and
for the CRBN recruiter, Z is 0 and R¹⁰ is carbonyl (XZ685).

In certain embodiments, Y is NH; R^{1A} is —COOH; R^{2B} is H or phenyl; R^{1B}, R^{1C}, R^{2A}, R^{3B} and R^{3C} are H; R^{3A} is -L-Rec, wherein L is a linker selected from:

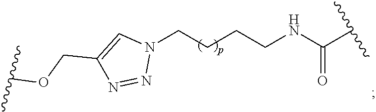

and
Rec is a CRBN recruiter selected from

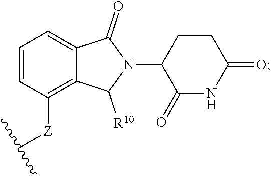

where R¹⁰ is H or carbonyl; and Z is O or NH, or N(C₁-C₃alkyl).

In some embodiments where Y is NH; R^{1A} is —COOH; R^{2B} is H or phenyl;
R^{1B}, R^{1C}, R^{2A}, R^{3B} and R^{3C} are H; R^{3A} is -L-Rec, wherein L is a linker selected from:

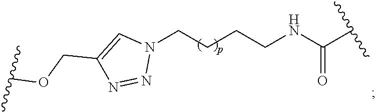

where p is 1-3; and
Rec is a CRBN recruiter according to

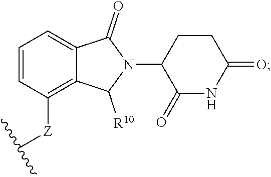

where R¹⁰ is H or carbonyl; and Z is O or NH, or N(C₁-C₃alkyl); for the linker, p is 1; and for the CRBN recruiter, Z is 0 and R¹⁰ is carbonyl; and R^{2B} is H (XZ688) or phenyl (XZ686).

In some embodiments, Y is NH; R^{1A} is —COOH; R^{2B} is H or phenyl; R^{1B}, R^{1C}, R^{2A}, R^{3B} and R^{3C} are H; R^{3A} is -L-Rec, wherein L is a linker selected from:

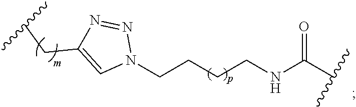

where m is 0-3; p is 1-3; and
Rec is a CRBN recruiter selected from

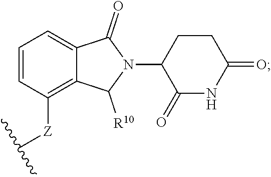

where R¹⁰ is H or carbonyl; and Z is O or NH, or N(C₁-C₃alkyl).

In certain aspects where Y is NH; R^{1A} is —COOH; R^{2B} is H or phenyl; R^{1B}, R^{1C}, R^{2A}, R^{3B} and R^{3C} are H; R^{3A} is -L-Rec, wherein L is a linker selected from:

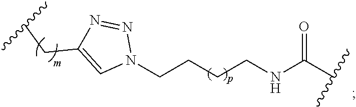

and
Rec is a CRBN recruiter according to

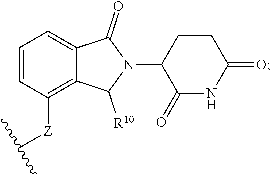

where for the linker, m is 0, p is 1; and for the CRBN recruiter, Z is 0 and R¹⁰ is carbonyl; and R^{2B} is H (XZ689) or phenyl (XZ687).

In certain embodiments, Y is NH; R^{1A} is —COOH; R^{2B} is H or phenyl; R^{1B}, R^{1C}, R^{2A}, R^{3B} and R^{3C} are H; R^{3A} is -L-Rec, wherein L is a linker selected from:

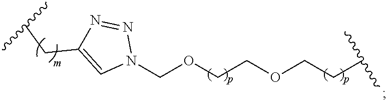

where m is 0-3; p is 1-3; and
Rec is a CRBN recruiter selected from

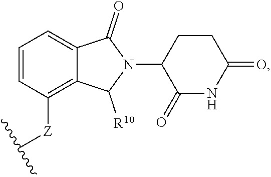

where R¹⁰ is H or carbonyl; and Z is O or NH, or N(C₁-C₃alkyl).

In some embodiments where Y is NH; R^{1A} is —COOH; R^{2B} is H or phenyl; R^{1B}, R^{1C}, R^{2A}, R^{3B} and R^{3C} are H; R^{3A} is -L-Rec, wherein L is a linker selected from:

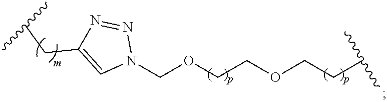

and
Rec is a CRBN recruiter selected from

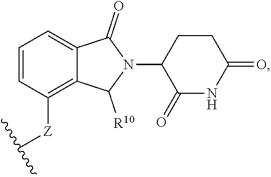

for the linker, m is 1, p is 1; and
for the CRBN recruiter, Z is 0 and R¹⁰ is carbonyl; and
R^{2B} is phenyl (XZ743).

In some embodiments, Y is NH; R^{1A} is —COOH; R^{2B} is H or phenyl; R^{1B}, R^{1C}, R^{2A}, R^{3B} and R^{3C} are H; R^{3A} is -L-Rec, wherein L is a linker selected from:

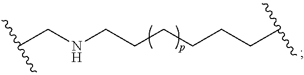

where p is 1-3; and
Rec is a CRBN recruiter selected from

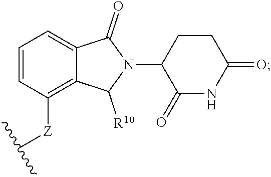

where R¹⁰ is H or carbonyl; and Z is O or NH, or N(C₁-C₃alkyl).

In some embodiments where Y is NH; R^{1A} is —COOH; R^{2B} is H or phenyl; R^{1B}, R^{1C}, R^{2A}, R^{3B} and R^{3C} are H; R^{3A} is -L-Rec, wherein L is a linker selected from:

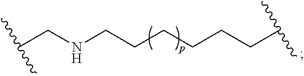

and
Rec is a CRBN recruiter selected from

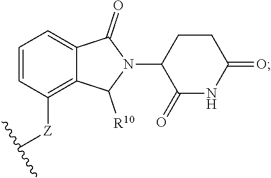

for the linker, p is 1; and for the CRBN recruiter, Z is O and R¹⁰ is H; and R^{2B} is phenyl (XZ742).

In certain embodiments, Y is NH; R^{1A} is —COOH; R^{2B} is H or phenyl; R^{1B}, R^{1C}, R^{2A}, R^{3B} and R^{3C} are H; R^{3A} is -L-Rec, wherein L is a linker selected from:

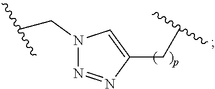

where p is 1-3; and
Rec is a CRBN recruiter selected from

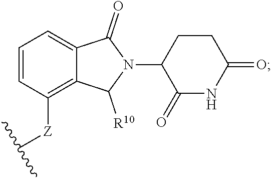

where R¹⁰ is H or carbonyl; and Z is O or NH, or N(C₁-C₃alkyl).

In certain embodiments where Y is NH; R^{1A} is —COOH; R^{2B} is H or phenyl;
R^{1B}, R^{1C}, R^{2A}, R^{3B} and R^{3C} are H; R^{3A} is -L-Rec, wherein L is a linker selected from:

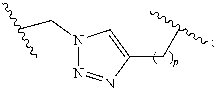

and
Rec is a CRBN recruiter selected from

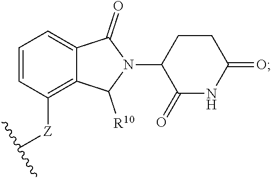

R^{2B} is phenyl; for the linker, p is 1; and for the CRBN recruiter, R¹⁰ is carbonyl, and Z is NH (XZ747) or N—CH₃ (XZ748).

In certain embodiments where Y is NH; R^{1A} is —COOH; R^{2B} is H or phenyl;
R^{1B}, R^{1C}, R^{2A}, R^{3B} and R^{3C} are H; R^{3A} is -L-Rec, wherein L is a linker selected from:

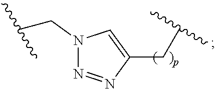

and
Rec is a CRBN recruiter selected from

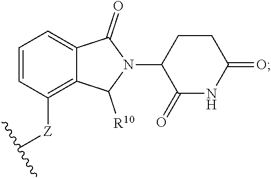

R^{2B} is phenyl;
for the linker, p is 2; and for the CRBN recruiter, R¹⁰ is carbonyl, and Z is NH (XZ749).

In another aspect, the disclosure provides a compound of Formula X, XI, XII, XIII, or XIV:

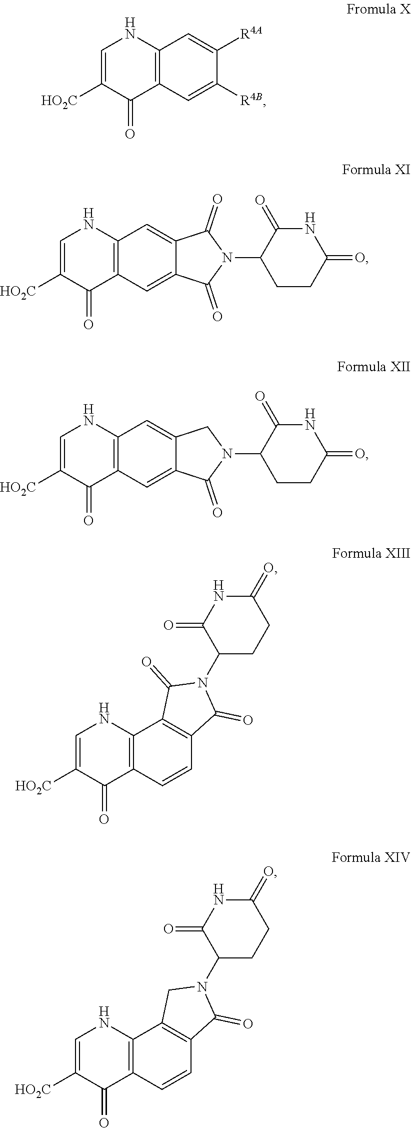

or a pharmaceutically acceptable salt thereof, where for Formula X, Z is O, NH, or N(C₁-C₃alkyl); R¹° is H or carbonyl; and one of R^{4A} or R^{4B} is

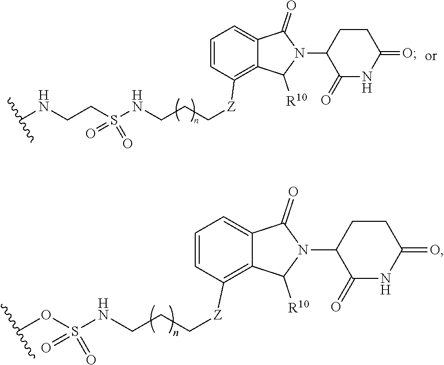

where n is 0-4.

In certain embodiments, for Formula X, n is 1; Z is NH; and R¹⁰ is carbonyl.

In another aspect, the disclosure provides a compound of Formula I-E

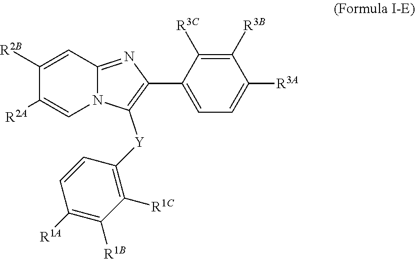

or a pharmaceutically acceptable salt thereof, where R^{1A}, R^{1B} and R^{1C} are H or —COOH; R^{2B} is H or phenyl; R^{3B} and R^{3C} are H; Y is O or NH; and either R^{2A} or R^{3A} is —(C₁-C₄)ONH₂, an oxime of formula —(C₁-C₄)ON═C—R²⁰, wherein the oxime is formed by the reaction of —(C₁-C₄)ONH₂ and an aldehyde

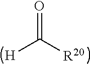

selected from the group consisting of A1-T12 in Table 4.

In certain embodiments, the compound of Formula I-E includes those for which R^{1A} is —COOH; R^{2B} is phenyl; R^{1B}, R^{1C}, R^{2A}, R^{3B}, and R^{3C} are H; and R^{3A} is H or —(C₁-C₄)ONH₂.

In certain embodiments, the compound of Formula I-E includes those for which R^{1A} is —COOH; R^{2B} is phenyl; R^{1B}, R^{1C}, R^{2A}, R^{3B}, and R^{3C} are H; and R^{3A} is H (XZ664) or —(CH₂)ONH₂ (XZ699).

In certain embodiments, the compound of Formula I-E includes those for which R^{1A} is —COOH; R^{2B} is phenyl; R^{1B}, R^{1C}, R^{2A}, R^{3B}, and R^{3C} are H; and R^{3A} is an oxime of formula —(C₁-C₄)ON═C—R²⁰, wherein the oxime is formed by the reaction of —(C₁-C₄)ONH₂ and an aldehyde

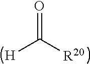

selected from the group consisting of A1-T12 in Table 4.

In another aspect the disclosure provides a pharmaceutical composition comprising one or more compounds or salts of any of the aspects and related embodiments of Formula I, Formula I.5, Formula I-A, Formula I-B, Formula I-C, Formula I-D, Formula I-E, Formula X, Formula XI, Formula XII, Formula XIII, or Formula IV, together with a pharmaceutically acceptable carrier.

In another aspect the disclosure provides a method of treating cancer in a patient including administering to the patient a therapeutically effective amount of one or more compounds, pharmaceutically acceptable salts, or pharmaceutical compositions of any of the aspects and related embodiments of Formula I, Formula I.5, Formula I-A, Formula I-B, Formula I-C, Formula I-D, Formula I-E, Formula X, Formula XI, Formula XII, Formula XIII, or Formula XIV.

In some embodiments, the method of treating cancer in a patient includes administering to the patient a therapeutically effective amount of one or more compounds, pharmaceutically acceptable salts, or pharmaceutical compositions of any of the aspects and related embodiments of Formula I.

In some embodiments, the method of treating cancer in a patient includes administering to the patient a therapeutically effective amount of one or more compounds, pharmaceutically acceptable salts, or pharmaceutical compositions of any of the aspects and related embodiments of Formula I.5.

In some embodiments, the method of treating cancer in a patient includes administering to the patient a therapeutically effective amount of one or more compounds, pharmaceutically acceptable salts, or pharmaceutical compositions of any of the aspects and related embodiments of Formula I-A.

In some embodiments, the method of treating cancer in a patient includes administering to the patient a therapeutically effective amount of one or more compounds, pharmaceutically acceptable salts, or pharmaceutical compositions of any of the aspects and related embodiments of Formula I-B.

In some embodiments, the method of treating cancer in a patient includes administering to the patient a therapeutically effective amount of one or more compounds, pharmaceutically acceptable salts, or pharmaceutical compositions of any of the aspects and related embodiments of Formula I-C.

In some embodiments, the method of treating cancer in a patient includes administering to the patient a therapeutically effective amount of one or more compounds, pharmaceutically acceptable salts, or pharmaceutical compositions of any of the aspects and related embodiments of Formula I-D.

In some embodiments, the method of treating cancer in a patient includes administering to the patient a therapeutically effective amount of one or more compounds, pharmaceutically acceptable salts, or pharmaceutical compositions of any of the aspects and related embodiments of Formula I-E.

In some embodiments, the method of treating cancer in a patient includes administering to the patient a therapeutically effective amount of one or more compounds, pharmaceutically acceptable salts, or pharmaceutical compositions of any of the aspects and related embodiments of Formula X.

In some embodiments, the method of treating cancer in a patient includes administering to the patient a therapeutically effective amount of one or more compounds, pharmaceutically acceptable salts, or pharmaceutical compositions of any of the aspects and related embodiments of Formula XI.

In some embodiments, the method of treating cancer in a patient includes administering to the patient a therapeutically effective amount of one or more compounds, pharmaceutically acceptable salts, or pharmaceutical compositions of any of the aspects and related embodiments of Formula XII.

In some embodiments, the method of treating cancer in a patient includes administering to the patient a therapeutically effective amount of one or more compounds, pharmaceutically acceptable salts, or pharmaceutical compositions of any of the aspects and related embodiments of Formula XIII.

In some embodiments, the method of treating cancer in a patient includes administering to the patient a therapeutically effective amount of one or more compounds, pharmaceutically acceptable salts, or pharmaceutical compositions of any of the aspects and related embodiments of Formula XIV.

In some embodiments, the method of treating cancer in a patient includes administering a topoisomerase I or topoisomerase II inhibitor before, concurrently with or after administering to the patient a therapeutically effective amount of one or more compounds, pharmaceutically acceptable salts, or pharmaceutical compositions of any of the aspects and related embodiments of Formula I, Formula I.5, Formula I-A, Formula I-B, Formula I-C, Formula I-D, Formula I-E, Formula X, Formula XI, Formula XII, Formula XIII, or Formula XIV.

In some embodiments, the cancer being treated in the methods of treating cancer in a patient described herein and above is a cancer expressing TDP1, and in some embodiments, the cancer is ovarian cancer, endometrial cancer, liver cancer, breast cancer, thyroid cancer, prostate cancer, pancreatic cancer, stomach cancer, lung cancer, larynx cancer, colon cancer, esophageal cancer, uterine cancer, cervical cancer, gall bladder cancer, kidney cancer, urinary bladder cancer or malignant lymphoma.

In another aspect the disclosure provides a method of degrading TDP1 in a patient including administering to the patient a therapeutically effective amount of one or more compounds, pharmaceutically acceptable salts, or pharmaceutical compositions of any of the aspects and related embodiments of Formula I, Formula I.5, Formula I-A, Formula I-B, Formula I-C, Formula I-D, Formula I-E, Formula X, Formula XI, Formula XII, Formula XIII, or Formula XIV.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula I.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula I.5.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula I-A.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula I-B.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula I-C.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula I-D.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula I-E.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula X.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula XI.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula XII.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula XIII.

In some embodiments, the method of degrading TDP1 in a patient includes administering to the patient a therapeutically effective amount of one or more compounds, pharmaceutically acceptable salts, or pharmaceutical compositions of any of the aspects and related embodiments of Formula XIV.

In another aspect the disclosure provides a method of inhibiting the repair of a TOP1-DNA covalent complex in a patient including administering to the patient a therapeutically effective amount of one or more compounds, pharmaceutically acceptable salts, or pharmaceutical compositions of any of the aspects and related embodiments of Formula I, Formula I.5, Formula I-A, Formula I-B, Formula I-C, Formula I-D, Formula I-E, Formula X, Formula XI, Formula XII, Formula XIII, or Formula XIV.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula I.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula I.5.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula I-A.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula I-B.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula I-C.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula I-D.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula I-E.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula X.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula XI.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula XII.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula XIII.

In some embodiments, the method of inhibiting the repair of a TOP1-DNA covalent complex in a patient includes administering to the patient a therapeutically effective amount of one or more compounds, pharmaceutically acceptable salts, or pharmaceutical compositions of any of the aspects and related embodiments of Formula XIV.

In another aspect the disclosure provides a method of stabilizing a TOP1-DNA complex in a patient including administering to the patient a therapeutically effective amount of one or more compounds, pharmaceutically acceptable salts, or pharmaceutical compositions of any of the aspects and related embodiments of Formula I, Formula I.5, Formula I-A, Formula I-B, Formula I-C, Formula I-D, Formula I-E, Formula X, Formula XI, Formula XII, Formula XIII, or Formula XIV.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula I.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula I.5.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula I-A.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula I-B.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula I-C.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula I-D.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula I-E.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula X.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula XI.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula XII.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula XIII.

In some embodiments, the method of stabilizing a TOP1-DNA complex in a patient includes administering to the patient a therapeutically effective amount of one or more compounds, pharmaceutically acceptable salts, or pharmaceutical compositions of any of the aspects and related embodiments of Formula XIV.

In another aspect the disclosure provides a method of degrading TOP1 in a patient including administering to the patient a therapeutically effective amount of one or more compounds, pharmaceutically acceptable salts, or pharmaceutical compositions of any of the aspects and related embodiments of Formula I, Formula I.5, Formula I-A, Formula I-B, Formula I-C, Formula I-D, Formula I-E, Formula X, Formula XI, Formula XII, Formula XIII, or Formula XIV.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula I.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula I.5.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula I-A.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula I-B.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula I-C.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula I-D.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula I-E.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula X.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula XI.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula XII.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula XIII.

In some embodiments, the method of degrading TOP1 in a patient includes administering to the patient a therapeutically effective amount of one or more compounds, pharmaceutically acceptable salts, or pharmaceutical compositions of any of the aspects and related embodiments of Formula XIV.

In another aspect the disclosure provides a method of providing a molecular glue to a patient including administering to the patient a therapeutically effective amount of one or more compounds, pharmaceutically acceptable salts, or pharmaceutical compositions of any of the aspects and related embodiments of Formula I, Formula I.5, Formula I-A, Formula I-B, Formula I-C, Formula I-D, Formula I-E, Formula X, Formula XI, Formula XII, Formula XIII, or Formula XIV.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula I.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula I.5.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula I-A.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula I-B.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula I-C.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula I-D.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula I-E.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula X.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula XI.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula XII.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula XIII.

In some embodiments, the method of providing a molecular glue to a patient includes administering to the patient a therapeutically effective amount of one or more compounds, pharmaceutically acceptable salts, or pharmaceutical compositions of any of the aspects and related embodiments of Formula XIV.

### BRIEF DESCRIPTION OF THE DRAWINGS

FIG. 1 shows a Lineweaver-Burk analysis (LB plot) of 7b, 8a, 10a, 10b with TDP1. FIG. 1A: Inhibitory constant of 7b, Ki=0.309±0.122 μM. FIG. 1B: Inhibitory constant of 8a, Ki=12.49±0.49 μM. FIG. 1C: Inhibitory constant of 10a, Ki=109.9±30.9 μM. FIG. 1D: Inhibitory constant of 10b, Ki=17.40±2.80 μM.

FIG. 2 shows the synergistic effect of TDP1 inhibitor 8s with camptothecin (CPT) in human colon cancer cell line HCT116.

FIG. 3 shows the synergistic effect of TDP1 inhibitor 10b with camptothecin (CPT) in human colon cancer cell line HCT116.

FIG. 4 shows the synergistic effect of selective TDP1 inhibitors M7 (FIG. 4A), 7b (FIG. 4B), 6u (FIG. 4C), 8a (FIG. 4D), 8m (FIG. 4E), 8n (FIG. 4F), 8o (FIG. 4G), 8p (FIG. 4H), 8q (FIG. 4I), 8s (FIG. 4J), 10a (FIG. 4K) and 10b (FIG. 4L) with camptothecin (CPT) in human colon cancer cell line HCT116 based on cell viability. Synergy Scores were calculated using SynergyFinder 2.0. See Ianevski, A., et al., Nucleic Acids Res. (2020) 48(W1): W488-W493.

FIG. 5 shows plots of Combination index (CI) vs. Fractional effects (FA). FIG. 5A: for camptotheicin with compounds 8m, 8q, 8o, or 6u. FIG. 5B: for camptothecin with compounds 8a, 6, 10b, 8s, 8m, and 8n. Fractional affect (Fa) and combination index (Ci) were analyzed with CompuSyn software.

FIG. 6 shows lead structures (M7, XZ615, XZ664 and XZ634) from small molecule microarray (SMM) analysis.

FIG. 7 shows a schematic representation of oxime-based strategy design utilized herein to exploit the DNA substrate and TOP1 peptide-binding regions of TDP1. Three stages were involved in the design: Stage 1 was the preparation of oximes (105-X and 6-Y) in parallel in a 96-well plate format starting from aminoxyl-labelled compounds (105 and 106, respectively). Stage 2 included evaluating TDP1 inhibition activity of the formed oximes (105-X and 106-Y) using a gel-based fluorescence assay directly without purification (see, e.g., Example 1). Stage 3 included identifying lead oximes (105-X1 and 106-Y1) based on the related inhibition values. In certain embodiments, inhibition activity was confirmed after purification.

FIG. 8 shows an alternate schematic for the oxime-based strategy design.

FIG. 9 shows TDP1 and TDP2 inhibition values for certain oxime inhibitors.

FIG. 10 shows lead TDP1 selective oxime XZ701 ((E)-6-D1).

FIG. 11 shows TDP1 degradation effect of XZ605 and XZ664 in HEK293 cells (FIG. 11A) and in HCT116 cells (FIG. 11B).

FIG. 12 shows a chart showing TDP1 degradation effect of precursors XZ615 vs XZ664 over time in HEK292 and HCT116 cells. (Here and throughout the instant application, where XZ compound numbers are provided with a “p” and/or with a dash (“-”) the p and/or the dash can be disregarded. For example “XZ-615p” as shown in FIG. 12 is the same compound as XZ-615, which is the same compound as XZ615.)

FIG. 13 shows concentration (FIG. 13A and FIG. 13B) and time (FIG. 13A and FIG. 13C) dependent degradation of TDP1 by XZ664 in HEK293 cells.

FIG. 14 shows gel assay results (FIG. 14B) and TDP1 inhibition and Degradation values (FIG. 14C) for various compounds (FIG. 14A). For FIG. 14C: ⁱThe half maximal inhibitory concentration (IC₅₀) was evaluated by gel-based TDP1 fluorescence assay in vitro. ⁱⁱThe half maximal inhibitory concentration (IC₅₀) was evaluated by gel-based TDP2 fluorescence assay in vitro. ⁱⁱⁱThe fold change (FC) was calculated by the ratio of IC₅₀ TDP2 to TDP1, which reflect the TDP1 selectivity over TDP2. ^{iv}Protein degradation was evaluated by the ratio of TDP1 or TOP1 to Vinculin after 72-hour treatment of HEK193 cell line at a drug concentration of 200 μM.

FIG. 15 shows the degradation effects of other SMM leads.

FIG. 16 shows additional TDP1 degraders.

FIG. 17 shows the degradation effects of XZ726 on TDP1 and TOP1 in HEK293 cells. FIG. 17A: gel assay of XZ726 and XZ679. FIG. 17B: TDP1 degradation as a function of XZ726 concentration and time. FIG. 17C: TOP1 degradation as a function of XZ726 concentration and time.

FIG. 18 shows the degradation effects of XZ726 on TDP1 and TOP1 in HCT116 cells. FIG. 18A: gel assay of XZ726 and XZ679. FIG. 18B: TDP1 degradation as a function of XZ726 concentration and time. FIG. 18C: TOP1 degradation as a function of XZ726 concentration and time.

FIG. 19 shows the synergistic effect of oxime lead XZ701 and derivatives thereof with camptothecin (CPT) in human colon cancer cell line HCT116 based on cell viability. XZ701 (FIG. 19A), XZ708 (FIG. 19B), XZ702 (FIG. 19C), and XZ706 (FIG. 19D).

FIG. 20 shows the synergistic effect oxime lead XZ701 and derivatives thereof with camptothecin (CPT) in human colon cancer cell line HCT116 based on cell viability. XZ701 (FIG. 20A), XZ726 (FIG. 20B), XZ725 (FIG. 20C), and XZ718 (FIG. 20D).

FIG. 21 shows the synergistic effect of triazole derivatives with camptothecin (CPT) in human colon cancer cell line HCT116 based on cell viability. XZ718 (FIG. 21A), XZ719 (FIG. 21B), and XZ720 (FIG. 21C).

FIG. 22: The synergistic effect of TDP1 inhibitor XZ701 with camptothecin (CPT) in human colon cancer cell line HCT116 is shown in FIG. 22A and FIG. 22B.

FIG. 23: The synergistic effect of TDP1 inhibitor XZ702 with camptothecin (CPT) in human colon cancer cell line HCT116 is shown in FIG. 23A and FIG. 23B.

FIG. 24: The synergistic effect of TDP1 inhibitor XZ708 with camptothecin (CPT) in human colon cancer cell line HCT116 is shown in FIG. 24A and FIG. 24B.

FIG. 25: The synergistic effect of TDP1 inhibitor XZ706 with camptothecin (CPT) in human colon cancer cell line HCT116 is shown in FIG. 25A and FIG. 25B.

FIG. 26: The synergistic effect of TDP1 inhibitor XZ726 with camptothecin (CPT) in human colon cancer cell line HCT116 is shown in FIG. 26A and FIG. 26B.

FIG. 27: The synergistic effect of TDP1 inhibitor XZ725 with camptothecin (CPT) in human colon cancer cell line HCT116 is shown in FIG. 27A and FIG. 27B.

FIG. 28: The synergistic effect of TDP1 inhibitor XZ718 with camptothecin (CPT) in human colon cancer cell line HCT116 is shown in FIG. 28A and FIG. 28B.

FIG. 29: The synergistic effect of TDP1 inhibitor XZ719 with camptothecin (CPT) in human colon cancer cell line HCT116 is shown in FIG. 29A and FIG. 29B.

FIG. 30: The synergistic effect of TDP1 inhibitor XZ720 with camptothecin (CPT) in human colon cancer cell line HCT116 is shown in FIG. 30A and FIG. 30B.

FIG. 31 shows the synergistic effect of TDP1 inhibitor XZ722 with camptothecin (CPT).

FIG. 32 shows the results of the gel-based in TDP1 assay for F259-targeting inhibitors XZ690, XZ691, and XZ692.

FIG. 33 shows TDP1 inhibition analogs targeting F259.

FIG. 34 shows a series of covalent/fluorosulfate TDP1 inhibitors.

FIG. 35 shows a primary screen of fluorosulfate inhibitors XZ728 (“28”) through XZ739 for TDP1 and TDP2 inhibition. (TDP assay buffer: 50 mM Tris HCl, pH 7.5, 80 mM KCl, 2 mM EDTA, 1 mM DTT, 40 μg/ml BSA and 0.01% Tween-20. TDP1 (20 mg/mL in 25 mM Tris-HCl pH 7.2, 150 mM NaCl, 2 mM tris(2-carboxyethyl)phosphine) with 2 mL of well solution composed of 0.1 M MOPS/HEPES pH 7.5, 10% (w/v) PEG 8000, 20% (v/v) ethylene glycol, 0.03 M sodium fluoride, 0.03 M sodium bromide, 0.03 M sodium iodide, 0.03 M sodium bromide and sealed over 500 mL well solution. AF647 labelling buffer: Tris-HCl (pH 7.2, 25 mM), NaCl (150 mM), and tris(2-carboxyethyl)phosphine hydrochloride (TCEP, 2 mM).)

FIG. 36 shows a secondary screen of fluorosulfate inhibitors for TDP1 and TDP2 inhibition with a preincubation time of 10 minutes for XZ730 and XZ731 (FIG. 36A) and XZ732 and XZ739 (FIG. 36B). (DNA only: Buffer+DMSO. DMSO only: Enzyme+DMSO. Drug: Enzyme+Drug, 25° C., 10 min+DNA, reaction at 25° C., 15 min.)

FIG. 37 shows a secondary screen of fluorosulfate inhibitors for TDP1 and TDP2 inhibition with a preincubation time of overnight for XZ730 (FIG. 37A, FIG. 37B, and FIG. 37C) and for XZ731 (FIG. 37D, FIG. 37E, and FIG. 37F).

FIG. 38 shows a secondary screen of fluorosulfate inhibitors for TDP1 and TDP2 inhibition with a preincubation time of overnight for XZ732 (FIG. 38A, FIG. 38B, and FIG. 38C) and for XZ739 (FIG. 38D, FIG. 38E, and FIG. 38F).

FIG. 39 shows TDP1 degradation results for various covalent inhibitors.

FIG. 40 shows covalent inhibitors based on phthalic acids.

FIG. 41 shows covalent inhibitors based on quinolones.

FIG. 42 shows certain TDP1 PROTACs design.

FIG. 43 shows PROTACs with a VHL-recruiter.

FIG. 44 shows PROTACs with a CRBN-recruiter.

FIG. 45 shows the gel-based TDP1 assay for PROTACs XZ679 and XZ682 compared to precursors XZ664 and XZ615.

FIG. 46 shows PROTAC XZ687 in a gel-based TDP1 assay compared to precursors XZ664 and XZ615.

FIG. 47 shows TDP1 (FIG. 47A and FIG. 47B) and TOP1 (FIG. 47A and FIG. 47C) degradation by PROTAC XZ679 in HEK293 cells.

FIG. 48 shows TDP1 (FIG. 48A and FIG. 48B) and TOP1 (FIG. 48A and FIG. 48C) degradation by PROTAC XZ679 in HCT116 cells.

FIG. 49 shows the TDP1 degradation effects of PROTACs XZ679 and XZ687 with precursor XZ664 in HEK293 cells at 24 h and 48 h. (RIPA (Radioimmunoprecipitation assay) buffer; RIPA, 10% gel, 12 well, 15 μg (12-well plate, 100 μL RIPA, 30 μL DMSO+1500 mL media); Vinculin is used as a loading control to ensure the same loading for each well.)

FIG. 50 shows TDP1 levels in HEK293 cells exposed to PROTACs XZ679 and XZ687 and precursor XZ664.

FIG. 51 shows certain TDP1 PROTACs with modified linkers.

FIG. 52 shows additional TDP1 PROTACs with modified linkers.

FIG. 53 shows certain TDP1 PROTACs based on quinolone TDP1 binders.

FIG. 54 shows certain TDP1 degraders based on quinolone TDP1 binders.

### DETAILED DESCRIPTION

### Terminology

Certain terms and phrases are defined below and throughout the specification.

Prior to setting forth the invention in detail, it may be helpful to provide definitions of certain terms to be used in this disclosure. Compounds are described using standard nomenclature. Unless defined otherwise, all technical and scientific terms used herein have the same meaning as is commonly understood by one of skill in the art to which this invention belongs. Unless clearly contraindicated by the context each compound name includes the free acid or free base form of the compound as well as all pharmaceutically acceptable salts of the compound.

The term “compounds of Formula I” encompasses all compounds that satisfy Formula I, including any enantiomers, racemates and stereoisomers, as well as all pharmaceutically acceptable salts of such compounds. The phrase “a compound of Formula I” includes all subgeneric groups of Formula I (e.g., Formula I-A, Formula I-B, Formula I-C, Formula I-D, Formula I-E, and the like), and also includes pharmaceutically acceptable salts of a compound of Formula I, unless clearly contraindicated by the context in which this phrase is used.

The term “compound of Formula X, XI, XII, XIII, or XIV” encompasses all compounds that satisfy Formula X, Formula XI, Formula XII, Formula XIII, or Formula XIV, including any enantiomers, racemates and stereoisomers, as well as all pharmaceutically acceptable salts of such compounds. The phrase “a compound of Formula X, XI, XII, XIII, or XIV” includes all subgeneric groups of Formula X, Formula XI, Formula XII, Formula XIII, and Formula XIV, and also includes pharmaceutically acceptable salts of a compound of Formula X, Formula XI, Formula XII, Formula XIII, and Formula XIV, unless clearly contraindicated by the context in which this phrase is used.

A dash (“-”) that is not between two letters or symbols is used to indicate a point of attachment for a substituent.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” or the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. The open-end phrases such as “comprising” include and encompass the close-ended phrases. Comprising may be amended to the more limiting phrases “consisting essentially of” of “consisting of” as needed.

The definition of each expression, e.g., alkyl, m, n, or the like, when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein below. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. This disclosure is not intended to be limited in any manner by the permissible substituents of organic compounds. “H—” is not considered a substituent.

Compounds (and subgeneric groups) of Formula I, Formula X, Formula XI, Formula XII, Formula XIII, and Formula XIV include compounds of the formula having isotopic substitutions at any position. Isotopes include those atoms having the same atomic number but different mass numbers. By way of general example, and without limitation, isotopes of hydrogen include tritium and deuterium and isotopes of carbon include ¹¹C, ¹³C, and ¹⁴C. Compounds (and subgeneric groups) of Formula I, Formula X, Formula XI, Formula XII, Formula XIII, and Formula XIV also require enrichment of deuteration (substitution of a hydrogen atom with deuterium) at identified positions.

The term “cyclic,” as used herein, pertains to compounds and/or groups which have one ring, or two or more rings (e.g., spiro, fused, bridged).

For purposes of this disclosure, the chemical elements are identified in accordance with the Periodic Table of the Elements, CAS version, Handbook of Chemistry and Physics, 67th Ed., 1986-87, inside cover.

The term “heteroatom” as used herein is art-recognized and refers to an atom of any element other than carbon or hydrogen. Illustrative heteroatoms include boron, nitrogen, oxygen, phosphorus, sulfur and selenium.

The term “alkyl” means a branched or unbranched aliphatic radical containing the indicated number of carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl. When alkyl is used as part of another term, e.g. (C₃-C₆cycloalkyl)C₀-C₂alkyl, it has the definition of “alkyl” given in this paragraph and the point of attachment of the group to the moiety it substitutes is through the alkyl portion. C₀alkyl is a single bond.

“Alkylamino” is an alkyl group as defined herein, attached to the group it substitutes through an amino (NH) linker. Di-alkylamino groups are attached to the substituted group via a nitrogen linker and each alkyl group is independently chosen.

“Cycloalkyl” is a saturated carbocyclic group having 3 to 7 ring carbon atoms, preferably 3 to 6 ring carbon atoms, or the indicated number of ring carbon atoms. Examples of cycloalkyl groups include cyclopropyl, cyclobutyl, cyclopentyl, and cyclohexyl.

“Heterocycloalkyl,” is a saturated ring group usually having 4- to 7-ring atoms with 1 or 2 ring atoms independently chosen from N, O, and S: Examples of heterocycloalkyl groups includes azepines, azetidinyl, morpholinyl, pyranyl, oxopiperidinyl, oxopyrrolidinyl, piperazinyl, piperidinyl, pyrrolidinyl, thiomorpholinyl, tetrahydropyranyl and tetrahydrofuranyl.

The term “halo” or “halogen” means —Cl, —Br, —I or —F.

The term “haloalkyl” means an alkyl group, as defined herein, wherein at least one hydrogen is replaced with a halogen, as defined herein. Representative examples of haloalkyl include, but are not limited to, chloromethyl, 2-fluoroethyl, trifluoromethyl, pentafluoroethyl, and 2-chloro-3-fluoropentyl.

The term “hydroxyl” as used herein means an —OH group.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “haloalkoxy” as used herein means an alkoxy group, as defined herein, wherein at least one hydrogen is replaced with a halogen, as defined herein. Representative examples of haloalkoxy include, but are not limited to, chloromethoxy, 2-fluoroethoxy, trifluoromethoxy, and pentafluoroethoxy.

The term “nitro” as used herein means a —NO₂ group.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

A “dosage form” means a unit of administration of an active agent. Examples of dosage forms include tablets, capsules, injections, suspensions, liquids, emulsions, creams, ointments, suppositories, inhalable forms, transdermal forms, and the like.

“Pharmaceutical compositions” are compositions comprising at least one active agent, such as a compound or salt of Formula I, Formula X, Formula XI, Formula XII, Formula XIII, or Formula XIV, and at least one other substance, such as a carrier. Pharmaceutical compositions optionally contain one or more additional active agents. When specified, pharmaceutical compositions meet the U.S. FDA's GMP (good manufacturing practice) standards for human or non-human drugs.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

The term “carrier” applied to pharmaceutical compositions/combinations of the present disclosure refers to a diluent, excipient, or vehicle with which an active compound is provided. To be pharmaceutically acceptable a carrier must be safe, non-toxic and neither biologically nor otherwise undesirable.

A “patient” is a human or non-human animal in need of medical treatment. Medical treatment can include treatment of an existing condition, such as a disease or disorder, prophylactic or preventative treatment, or diagnostic treatment. In certain embodiments disclosed herein “medical treatment” means treatment of a diagnosed cancer or known tumor. In certain embodiments the patient is a human patient.

The term “formed by” refers to a chemical reaction, or the results thereof. For example, for oxime chemistry utilized herein, a hydroxylamine (e.g., —(C₁-C₄)ONH₂) is reacted with an aldehyde from Table 4, where an oxime (—(C₁-C₄)ON═C—R), where R derives from the aldehyde) is “formed by” the reaction. More specifically, the oxime —(C₁-C₄)ON═C— benzyl is formed by a reaction between the hydroxylamine —(C₁-C₄)ONH₂ and aldehyde M6 from Table 4, which is benzaldehyde.

When a compound of Formula I, Formula X, Formula XI, Formula XII, Formula XIII, or Formula XIV is provided with “an additional active agent” the compound of Formula I, Formula X, Formula XI, Formula XII, Formula XIII, or Formula XIV is a first active agent and the additional active agent(s) can be provided simultaneously in a single dosage form, provided concomitantly in separate dosage forms, or provided in separate dosage forms for administration separated by some amount of time that is within the time in which both the compound of Formula I, Formula X, Formula XI, Formula XII, Formula XIII, or Formula XIV and the additional active agent are within the blood stream of a patient. In certain embodiments the compound of Formula I, Formula X, Formula XI, Formula XII, Formula XIII, or Formula XIV and the additional active agent need not be prescribed for a patient by the same medical care worker. In certain embodiments the additional active agent or agents need not require a prescription. Administration of the compound of Formula I, Formula X, Formula XI, Formula XII, Formula XIII, or Formula XIV and the additional active agent can occur via any appropriate route, for example, oral tablets, oral capsules, oral liquids, inhalation, injection, suppositories or topical contact.

“Treatment,” as used herein includes providing a compound of Formula I, Formula X, Formula XI, Formula XII, Formula XIII, or Formula XIV either as the only active agent or together with an additional active agent sufficient to: (a) prevent or decrease the likelihood a disease or a symptom of a disease from occurring in a patient who may be predisposed to the disease but has not yet been diagnosed as having it; (b) inhibiting the disease, i.e. arresting its development; and (c) relieving the disease, i.e., causing a remission of the disease. “Treating” and “treatment” also means providing a therapeutically effective amount of a compound of Formula I, Formula X, Formula XI, Formula XII, Formula XIII, or Formula XIV as the only active agent or together with at least one additional active agent to a patient having a disease or disorder that can be effectively treated with a TDP1 or TOP1 inhibitor, such a cancer, a neurological disease (e.g. Angelman syndrome, autism), or septic shock.

A “therapeutically effective amount” of a pharmaceutical composition/combination of this disclosure means an amount effective, when administered to a patient, to provide a therapeutic benefit such as an amelioration of symptoms, e.g., an amount effective to decrease the symptoms of cancer. For example, a patient having cancer may present detectable levels of certain tumor markers, including CA 125, CEA, CAT9-9, AFP, PSA, and galactosyltransferase. A therapeutically effect amount is thus an amount sufficient to provide a significant reduction in elevated tumor marker levels or an amount sufficient to provide a return of tumor marker levels to the normal range. A therapeutically effective amount is also an amount sufficient to prevent a significant increase in tumor size relative that usually seen in untreated patients having the same cancer, or significantly reduce tumor size or tumor number, or causes tumors to disappear from the patient's body altogether.

A significant increase or reduction in the detectable level of tumor markers, tumor size, or tumor number, is any detectable change that is statistically significant in a standard parametric test of statistical significance such as Student's T-test, where p<0.05.

### Chemical Description

The disclosure provides a method of treating cancer in a patient, comprising administering a therapeutically effective amount of a compound of Formula I, Formula X, Formula XI, Formula XII, Formula XIII, or Formula XIV which may be a TDP1 inhibitor, to a patient in need of such treatment. “Formula I” includes all subformulae thereof (1.5, I-A, I-B). In certain situations, the compounds of Formula I, Formula X, Formula XI, Formula XII, Formula XIII, or Formula XIV may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms, with all isomeric forms of the compounds being included in the present disclosure. In these situations, single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example using a chiral HPLC column.

Where a compound exists in various tautomeric forms, the invention is not limited to any one of the specific tautomers, but rather includes all tautomeric forms.

Compounds and salts of Formula I, Formula X, Formula XI, Formula XII, Formula XIII, or Formula XIV include those in which the variables, e.g. R¹, R², R³ carry any of the definitions set forth herein.

### Pharmaceutical Preparations

Compounds disclosed herein can be administered as the neat chemical, but are preferably administered as a pharmaceutical composition. Accordingly, the disclosure provides pharmaceutical compositions comprising a compound of Formula I, Formula X, Formula XI, Formula XII, Formula XIII, or Formula XIV or pharmaceutically acceptable salt thereof, together with at least one pharmaceutically acceptable carrier. The pharmaceutical composition/combination may contain a compound or salt of Formula I, Formula X, Formula XI, Formula XII, Formula XIII, or Formula XIV as the only active agent, but is preferably contains at least one additional active agent. The additional active agent can be camptothecin, a camptothecin analogue, a poly(ADP-ribose) polymerase (PARP) inhibitor, a cell cycle checkpoint inhibitor targeting ATR (Ataxia Telangiectansia-related kinase), a CHEKI (cell cycle checkpoint kinase) inhibitor, a WEEl inhibitor, a CDK (cyclin dependent kinase) inhibitor or other chemotherapeutic compound. In certain embodiments it is preferred that the additional active agent is compound or salt thereof chosen from camptothecin, irinotecan, and topotecan. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I, Formula X, Formula XI, Formula XII, Formula XIII, or Formula XIV and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition may also include a molar ratio of a compound of TDP1 inhibitor, such as a compound of Formula I, Formula X, Formula XI, Formula XII, Formula XIII, or Formula XIV, and an additional active agent. For example, the pharmaceutical composition may contain a molar ratio of about 0.5:1, about 1:1, about 2:1, about 3:1 or from about 1.5:1 to about 4:1 of the compound of Formula I, Formula X, Formula XI, Formula XII, Formula XIII, or Formula XIV to the additional active agent.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present disclosure.

The pharmaceutical compositions/combinations can be formulated for oral administration. These compositions contain between 0.1 and 99 weight % (wt. %) of a compound of Formula I, Formula X, Formula XI, Formula XII, Formula XIII, or Formula XIV and usually at least about 5 wt. % of a compound of Formula I, Formula X, Formula XI, Formula XII, Formula XIII, or Formula XIV. Some embodiments contain from about 25 wt. % to about 50 wt. % or from about 5 wt. % to about 75 wt. % of the compound of Formula I, Formula X, Formula XI, Formula XII, Formula XIII, or Formula XIV.

### Methods of Treatment

The disclosure provides a method of treating cancer, including effecting tumor regression in vivo, by administering a compound or salt of Formula I, Formula X, Formula XI, Formula XII, Formula XIII, or Formula XIV. The method of treating cancer or effecting tumor regression comprises providing to a patient an effective amount of a compound or salt of the disclosure. In an embodiment the patient is a mammal, and more specifically a human. The disclosure also provides methods of treating non-human patients such as companion animals, e.g. cats, dogs, and livestock animals. An effective amount of a pharmaceutical composition may be an amount sufficient to inhibit the progression of cancer or a cancerous tumor; or cause a regression of a cancer or a cancerous tumor.

An effective amount of a compound or pharmaceutical composition described herein will also provide a sufficient concentration of a compound of the disclosure when administered to a patient. A sufficient concentration is a concentration of the compound in the patient's body necessary to combat the disorder. Such an amount may be ascertained experimentally, for example by assaying blood concentration of the compound, or theoretically, by calculating bioavailability.

Methods of treatment include providing certain dosage amounts of a compound or salt of the disclosure to a patient. Dosage levels of each compound from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 1000 mg of each active compound. In certain embodiments 25 mg to 500 mg, or 25 mg to 200 mg of a compound of the disclosure are provided daily to a patient. Frequency of dosage may also vary depending on the compound used and the particular disease treated.

The disclosure provides a method of using compounds of Formula I, Formula X, Formula XI, Formula XII, Formula XIII, or Formula XIV and salts thereof to treat cancers and effect regression of tumors, including cancerous tumors. In certain embodiments, the patient is suffering from a cell proliferative disorder or disease. The cell proliferative disorder can be cancer, tumor (cancerous or benign), neoplasm, neovascularization, or melanoma. Cancers for treatment include both solid and disseminated cancers. Exemplary solid cancers (tumors) that may be treated by the methods provided herein include e.g. cancers of the lung, prostate, breast, liver, colon, breast, kidney, pancreas, brain, skin including malignant melanoma and Kaposi's sarcoma, testes or ovaries, carcinoma, kidney cancer (renal cell), and sarcoma. Cancers that may be treated with a or salt of the disclosure also include bladder cancer, breast cancer, colon cancer, endometrial cancer, lung cancer, bronchial cancer, melanoma, Non-Hodgkin lymphoma, cancer of the blood, pancreatic cancer, prostate cancer, thyroid cancer, brain or spinal cancer, and leukemia. Exemplary disseminated cancers include leukemias or lymphoma including Hodgkin's disease, multiple myeloma and mantle cell lymphoma (MCL), chronic lymphocytic leukemia (CLL), T-cell leukemia, multiple myeloma, and Burkitt's lymphoma. Particularly included herein are methods of treating cancer by providing a compound or salt of the disclosure to a patient wherein the cancer is a solid tumor or disseminated cancer. TDP1 inhibits, such as the compounds of the disclosure, are particularly useful for treating cancer TDP1 expressing tumors, including cancers in which the tissue of origin is thyroid, breast, liver, endometrium, and ovary. The disclosure includes methods of treating ovarian, endometrial, liver, breast, thyroid, prostate, pancreatic, stomach, lung, larynx, colon, esophageal, uterine and cervical, gall bladder, kidney, and urinary bladder cancer comprising administering a compound of the disclosure to a patient having such a cancer. The disclosure also includes a method of treating malignant lymphoma comprising administering a compound of the disclosure to a patient with malignant lymphoma.

Further included are methods of treating cancer by providing a compound or salt of the disclosure to a patient wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, or colon cancer.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

The compound of Formula I, Formula X, Formula XI, Formula XII, Formula XIII, or Formula XIV or salt thereof may be administered singularly (i.e., sole therapeutic agent of a regime) to treat diseases and conditions such as undesired cell proliferation, cancer, and/or tumor growth or may be administered in combination with another active agent. One or more compounds of the disclosure may be administered in coordination with a regime of one or more other chemotherapeutic agents such as an antineoplastic drug, e.g., an alkylating agent (e.g., mechlorethamine, chlorambucil, cyclophosamide, melphalan, or ifosfamide), an antimetabolite such as a folate antagonist (e.g., methotrexate), a purine antagonist (e.g. 6-mercaptopurine) or a pyrimidine antagonist (e.g., 5-fluorouracil). Other, non-limiting examples of chemotherapeutic agents that might be used in coordination with one or more compounds or salts of the disclosure include taxanes and topoisomerase inhibitors. In addition, other non-limiting examples of active therapeutics include biological agents, such as monoclonal antibodies or IgG chimeric molecules, that achieve their therapeutic effect by specifically binding to a receptor or ligand in a signal transduction pathway associated with cancer (e.g. therapeutic antibodies directed against CD20 (e.g. rituximab) or against VEGF (e.g. bevacizumab)). Compounds of this disclosure can be used in combination with Topoisomerase I inhibitors such as irinotecan, topotecan, camptothecin, and lamellarin D. Compounds of Formula I, Formula X, Formula XI, Formula XII, Formula XIII, or Formula XIV can also be used in combination with Topoisomerase II inhibitors such as etoposide, teniposide, doxorubicin, daunorubicin, mitoxantrone, amsacrine, and aurintricarboxylic acid.

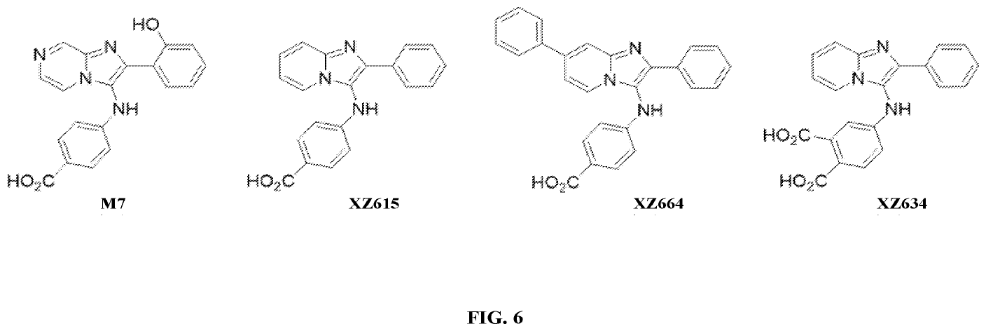

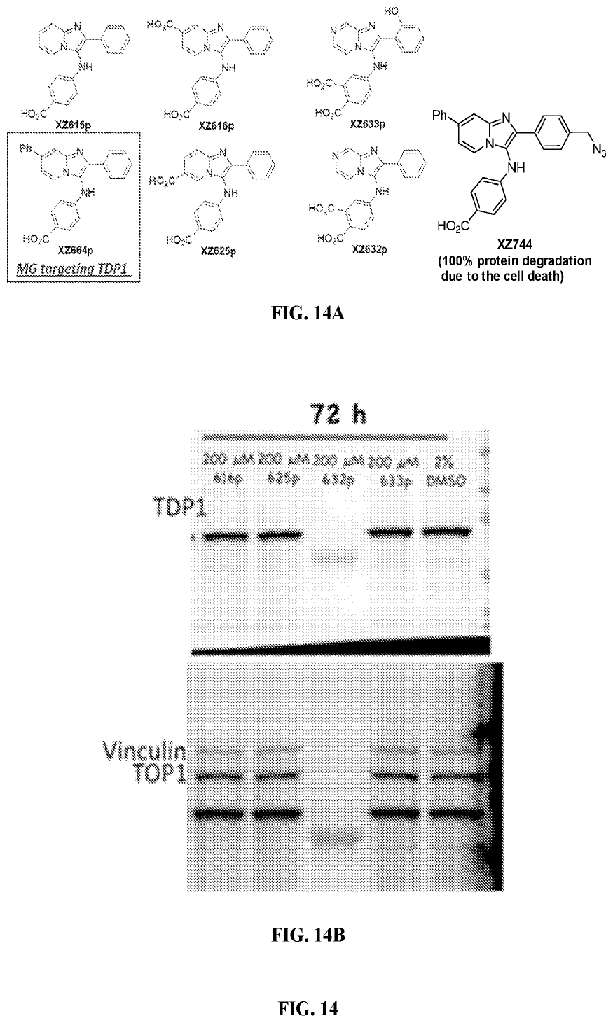

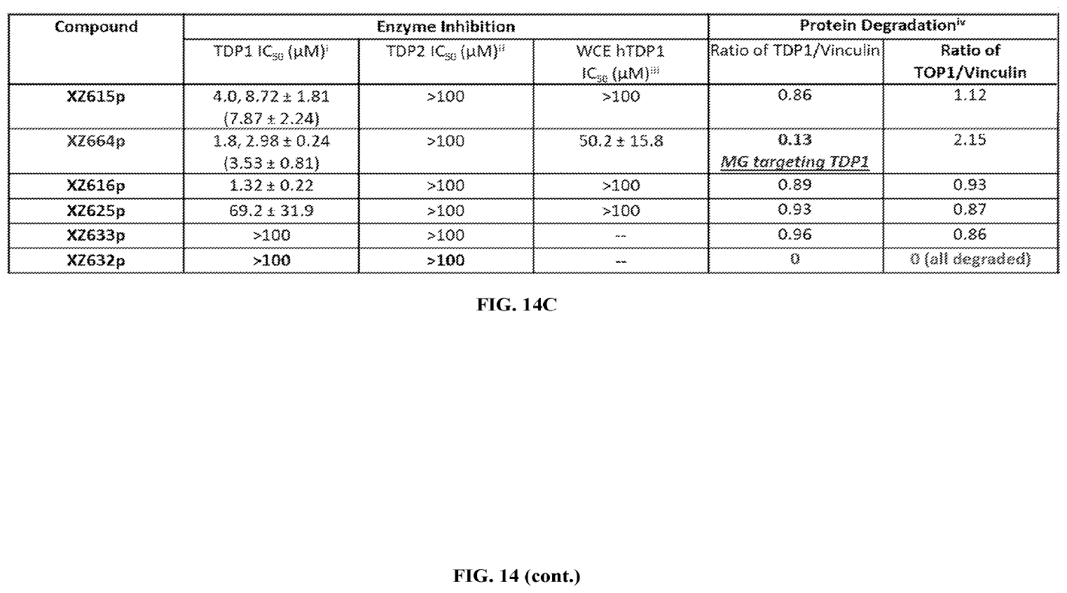

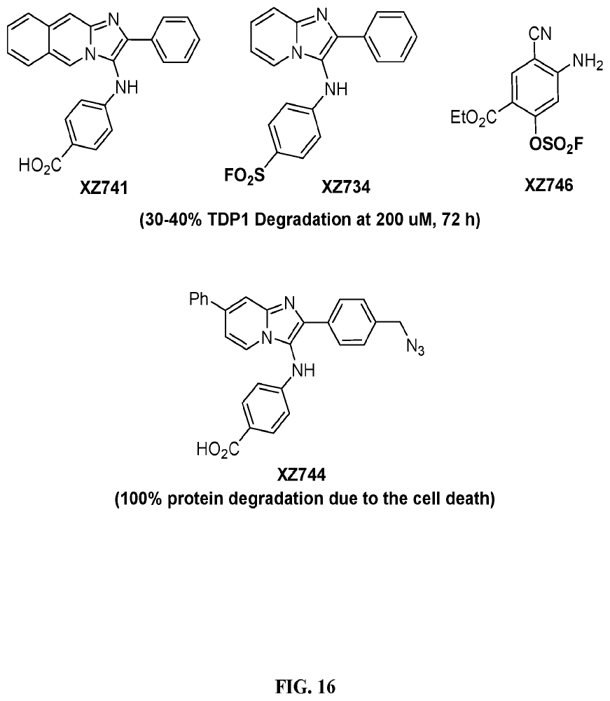

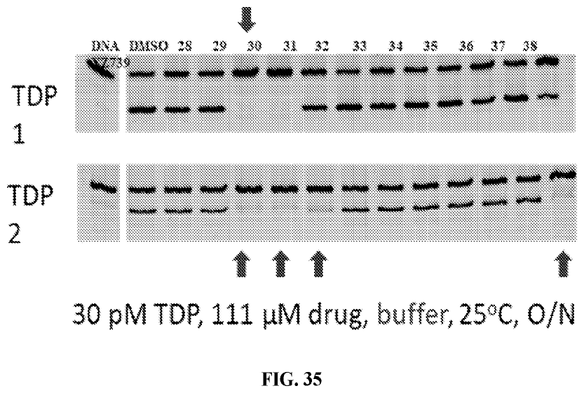

Certain compounds of the disclosure exert their activity through the catalytic site of TDP1, i.e., inhibit or block the catalytic activity of the protein against its natural ligand via an occupancy-driven pharmacology. Inhibitors can be, e.g., competitive (reversable) or suicide inhibitors (irreversible; covalent). Examples of reversible inhibitors of the disclosure include compounds 10b, M7, 8m, 8s, XZ699-D1/H1/T12, XZ699-P3, XZ699-E6, XZ699-B7, XZ700-D1/H1/T12, XZ700-P3, XZ700-E6, XZ700-B7, XZ701, XZ702, XZ703, XZ704, XZ705, XZ706, XZ708, XZ664, XZ615, XZ616, XZ633, XZ635, XZ632, XZ625, XZ744, XZ643, XZ665, XZ640, XZ644, XZ671, XZ7b, XZ718, XZ719, XZ720, XZ721, XZ722, XZ723, XZ724, XZ725, XZ726, XZ727, XZ730, XZ734, XZ735, XZ736, XZ737, XZ741, XZ750, XZ751, XZ752, XZ753, XZ754, and XZ755 (e.g., FIG. 6-10, FIG. 14, FIG. 16). Examples of irreversible, covalent inhibitors of the disclosure include fluorosulfonate (fluorosulfate) compounds such as XZ728, XZ729, XZ730, XZ731, XZ732, XZ733, XZ734, XZ738, XZ739 and XZ503 (e.g., FIG. 35-38).

Certain compounds of the disclosure target TDP1 for proteolysis by proteolysis targeting chimeras (PROTACs). PROTACs induce the degradation of a targeted protein by utilizing innate cellular quality-control machinery. PROTACs are heterobifunctional molecules and comprise two different ligands connected by a linker. One ligand binds to TDP1 and the other ligand (a “recruiter”) recruits E3 ligase or other degradation-promoting protein, which results in a ternary complex between the TDP1, the PROTAC, and the E3 ligase or degradation-promoting protein. Proximity-driven polyubiquitination of TDP1 by the E3 ligase occurs, resulting in subsequent non-natural degradation of TDP1. Thus, in contrast to canonical inhibitors, PROTACs operate by degrading the target protein via an event-driven pharmacology model.

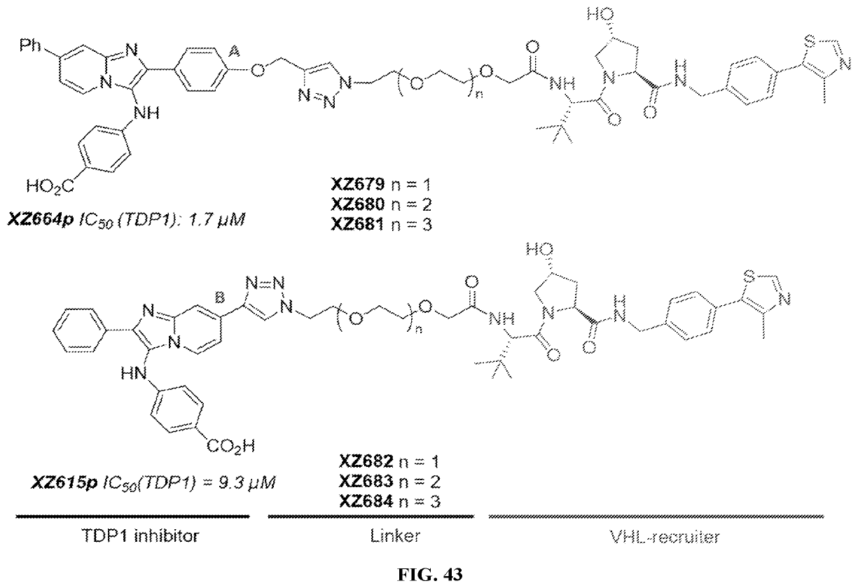

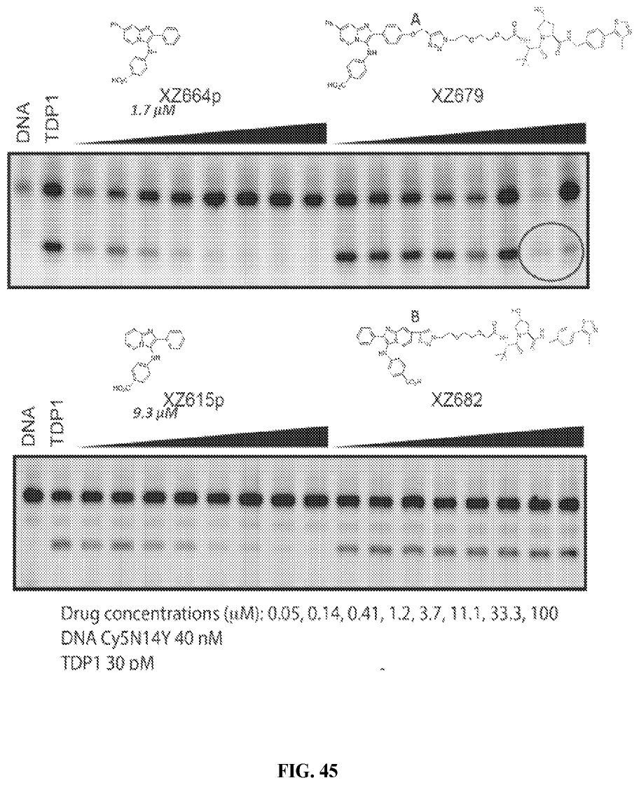

In some embodiments, the E3 recruiter is a von Hippel-Lindau (VHL) recruiter. Example compounds include XZ679, XZ680, XZ681, XZ615, XZ682, XZ683 and XZ684 (e.g., FIG. 43, FIG. 45). In addition to the VHL recruiters described elsewhere herein, VHL recruiters can also include the following.

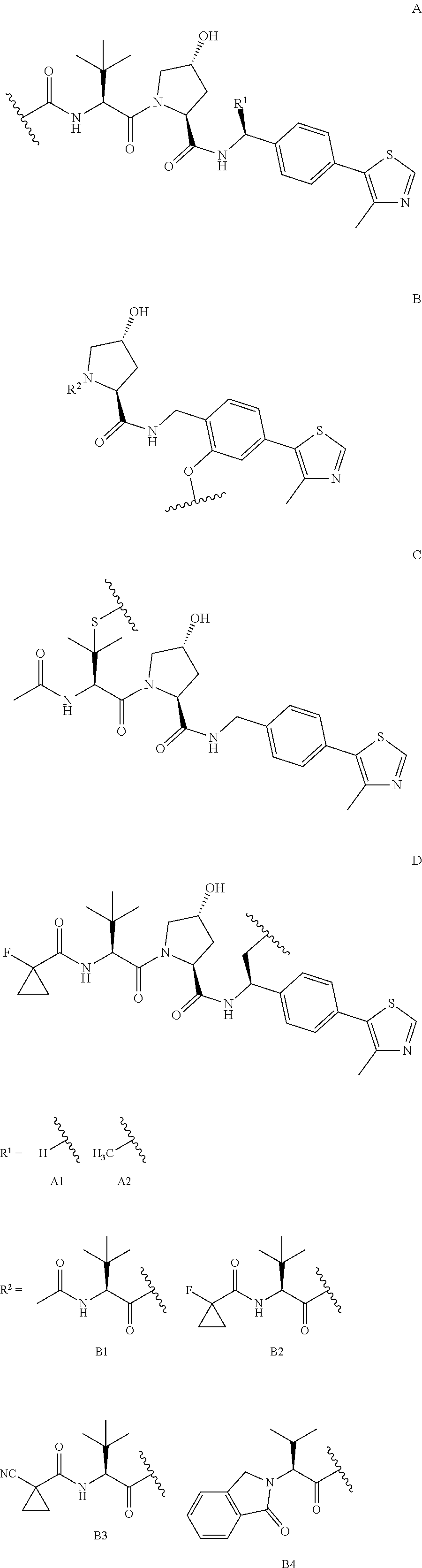

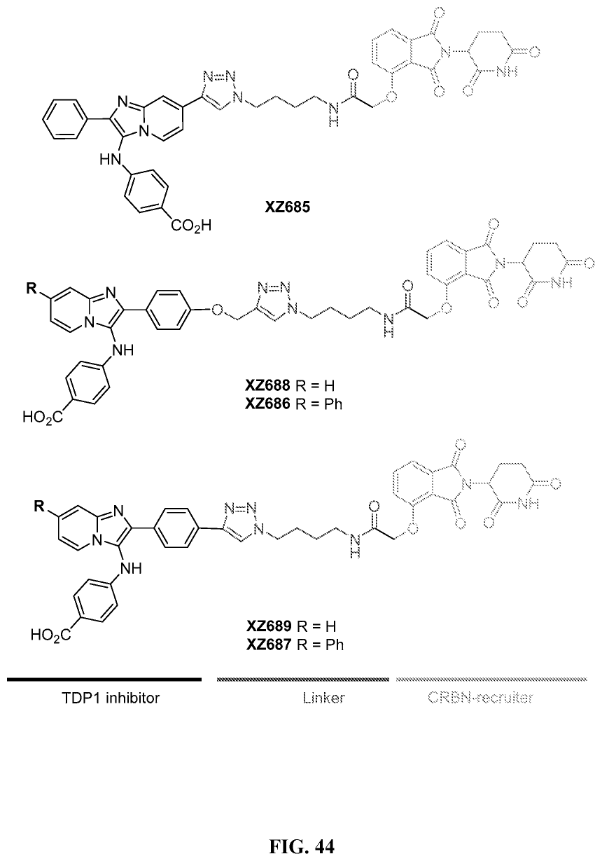

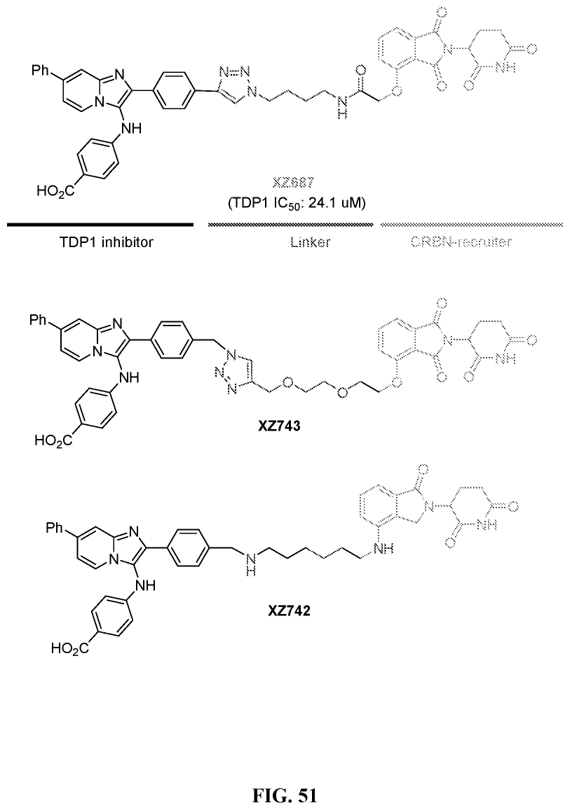

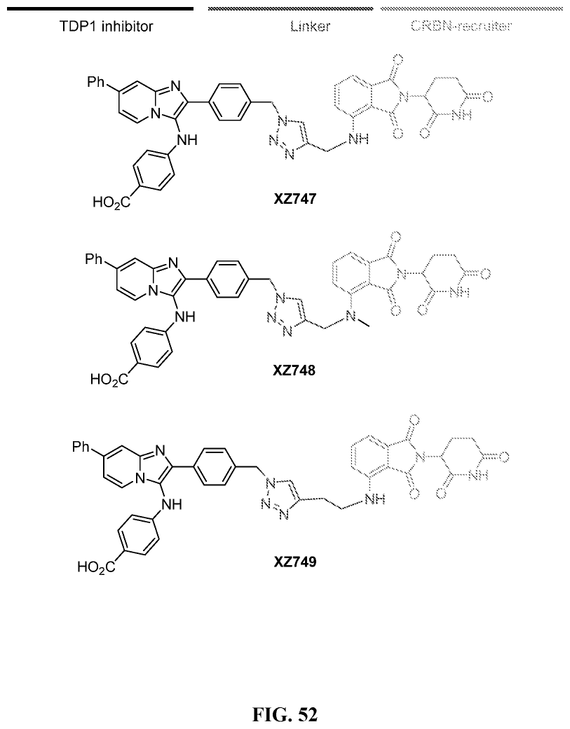

In some embodiments, the E3 recruiter is a cereblon (CRBN) recruiter. Example compounds include XZ685, XZ686, XZ687, XZ681, XZ688, and XZ689 (e.g., FIG. 44, FIG. 51, FIG. 52) In addition to the CRBN recruiters described elsewhere herein, CRBN recruiters can also include the following:

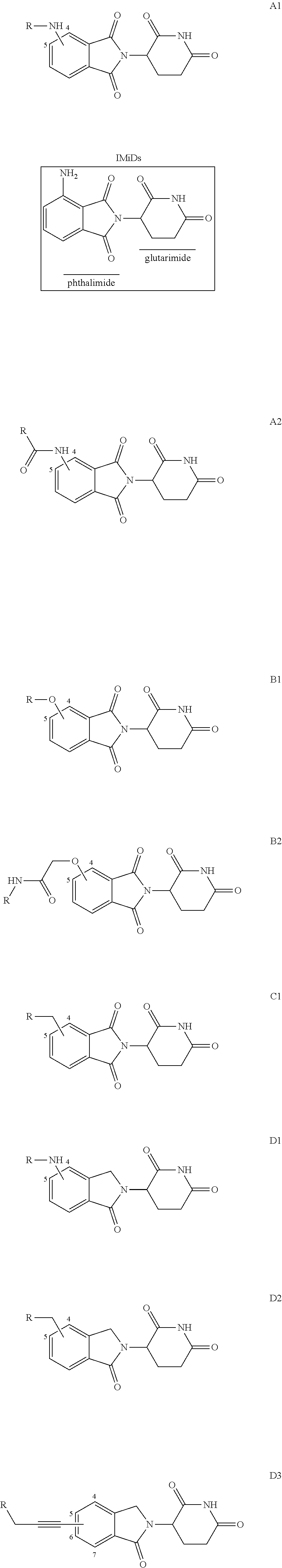

In some embodiments, the E3 recruiter is a (MDM2) recruiter, examples of which include the following:

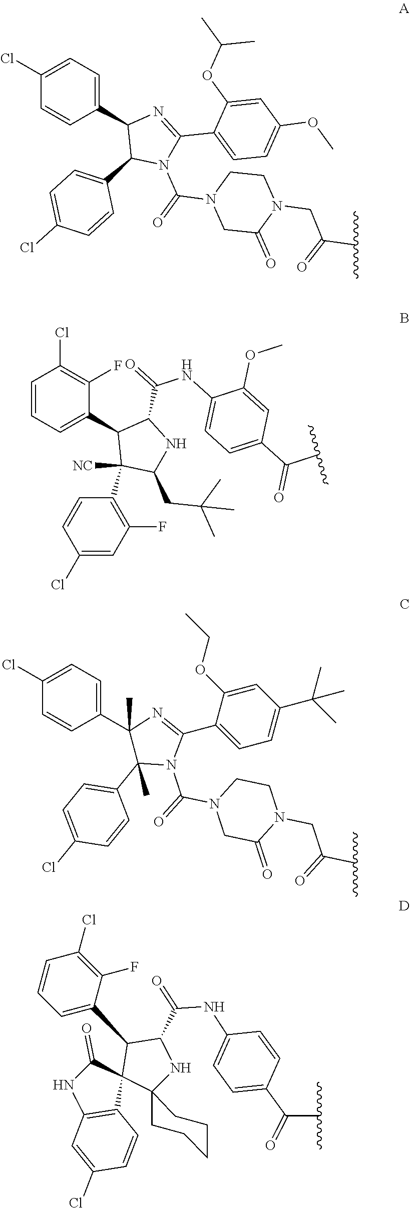 [SMILES: *C(=O)CN1CCN(C(=O)N2C(c3ccc(OC)cc3OC(C)C)=N[C@H](c3ccc(Cl)cc3)[C@@H]2c2ccc(Cl)cc2)CC1=O ; *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; *C(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1=O ; *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, the E3 recruiter is an apoptosis protein (IAP) recruiter, example of which include the following:

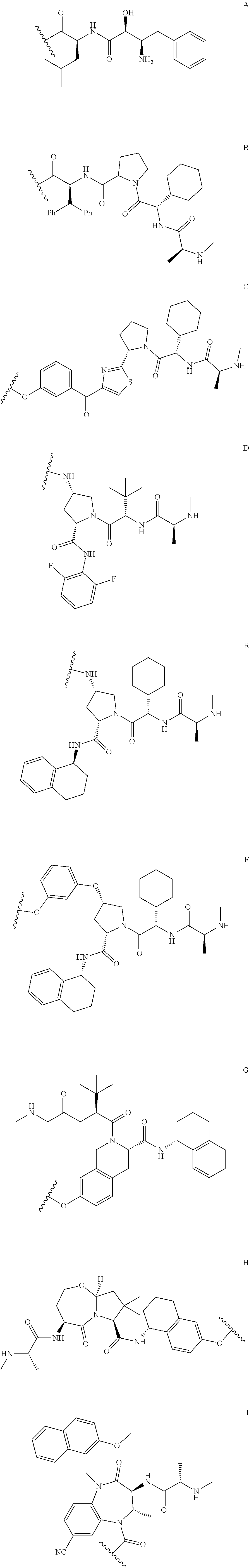

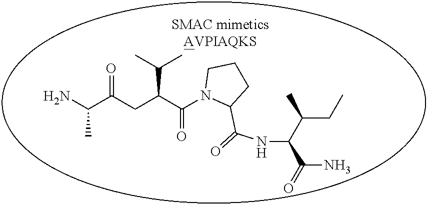 [SMILES: CC[C@H](C)[C@H](NC(=O)C1CCCN1C(=O)[C@@H](CC(=O)[C@H](C)N)C(C)C)C(N)=O]

Linkers contribute to the activity of PROTACs. Linker features, including type, length, attachment position, can affect the formation of E3 ligase: PROTAC: target ternary complex. Consequently, linkers influence the efficient ubiquitination of the target protein, e.g., TDP1, and its ultimate degradation. Commonly used linkers in the development of PROTACs are polyethylene glycols (PEGs), alkyl chains and alkyl/ethers. Certain linkers for use in PROTAC compounds of the disclosure are disclosed elsewhere herein, and certain compounds of the disclosure with equivalent TDP1 target and recruiter moieties but varied linker composition can be seen in, e.g., FIG. 51 and FIG. 52.

Certain compounds of the disclosure exhibit molecular glue or molecular glue-like activity. Molecular glues are monovalent small molecules that enhance protein-protein interactions between a protein of interest (e.g., tyrosyl-DNA phosphodiesterase 1 (TDP1), tyrosyl-DNA phosphodiesterase II (TDP2), topoisomerase I (TopI)) and, for example, E3 ligase, which enables engagement of the protein of interest with the E3 ligase and subsequent polyubiquitylation and degradation of the protein of interest.

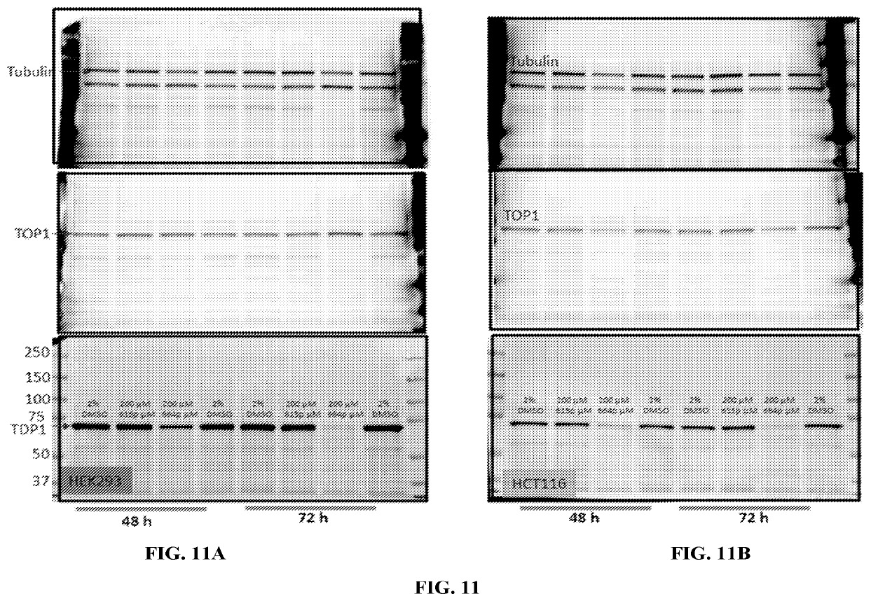

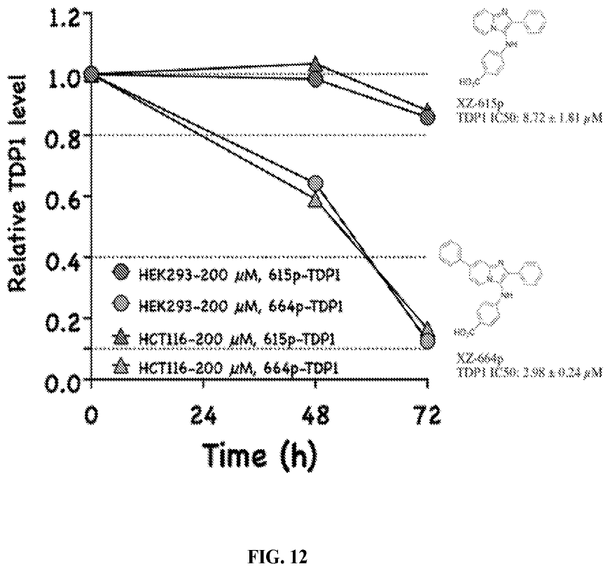

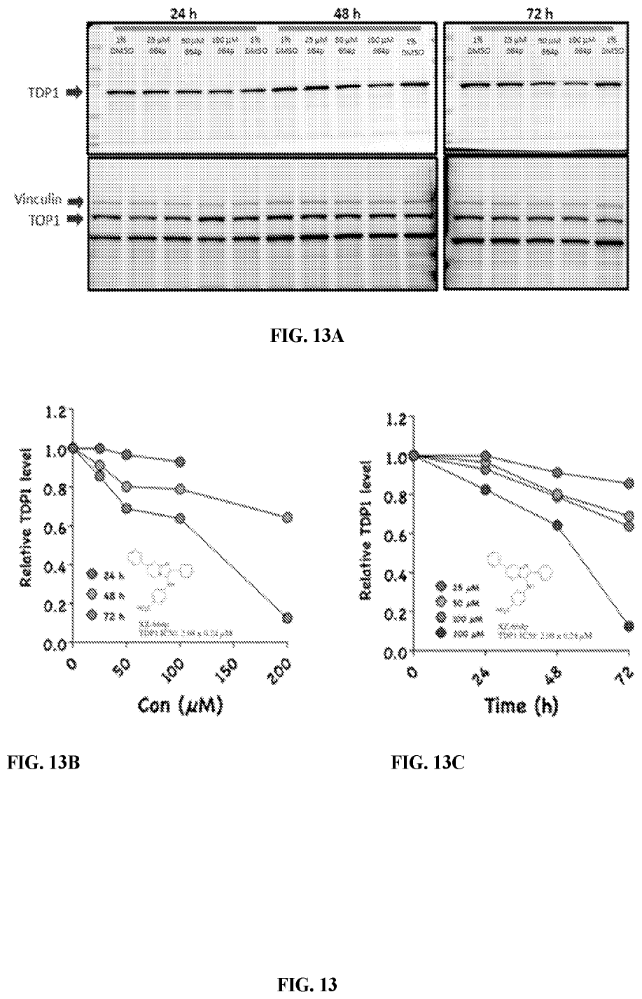

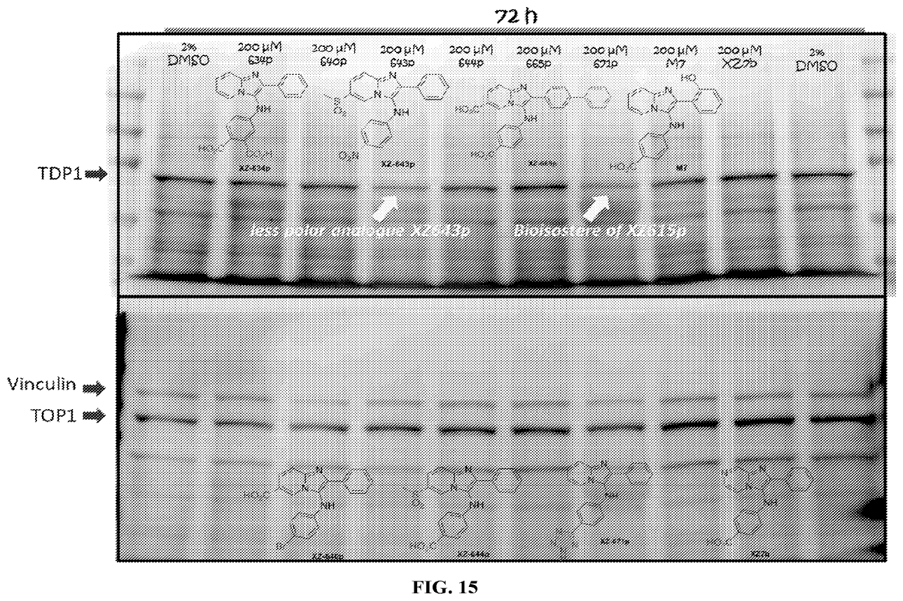

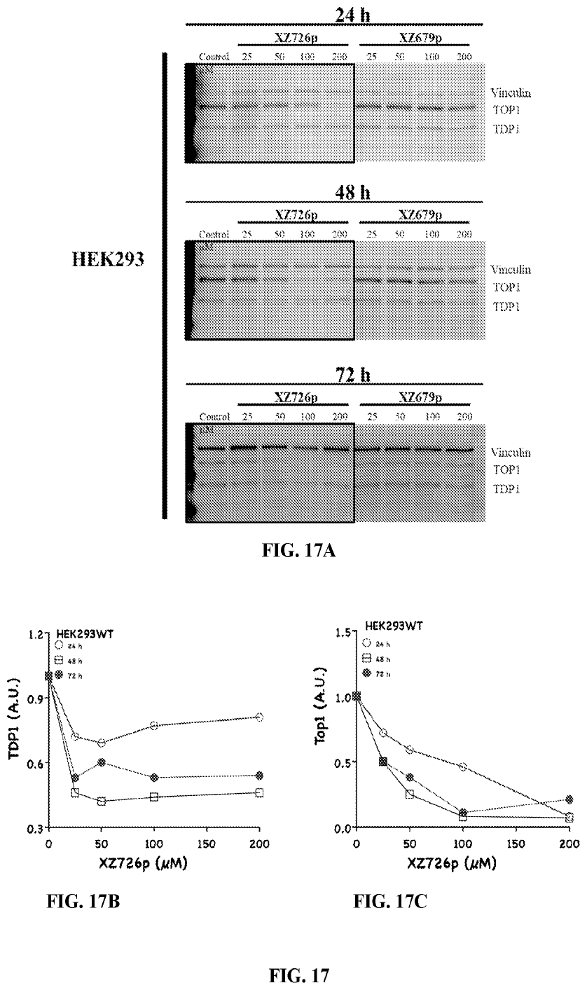

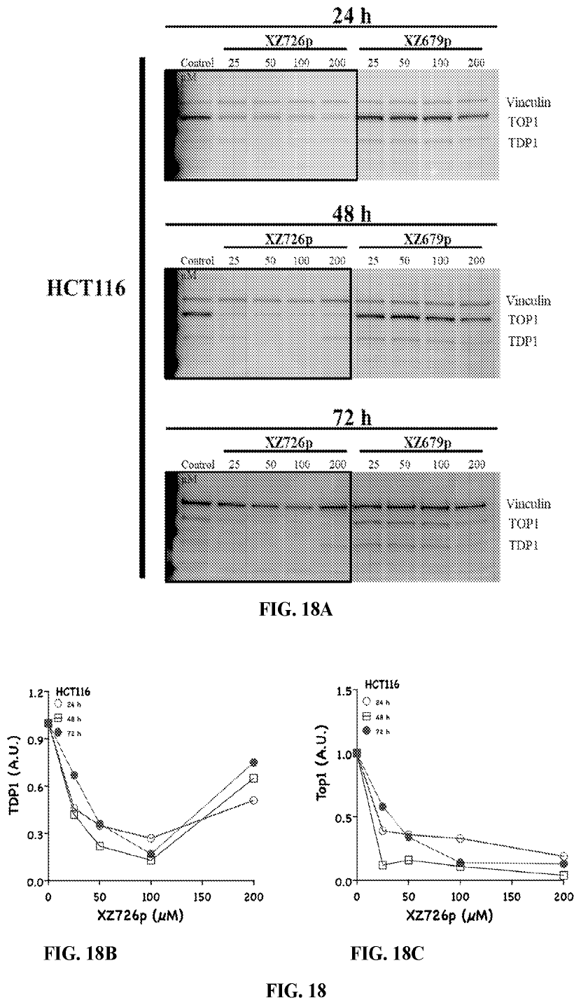

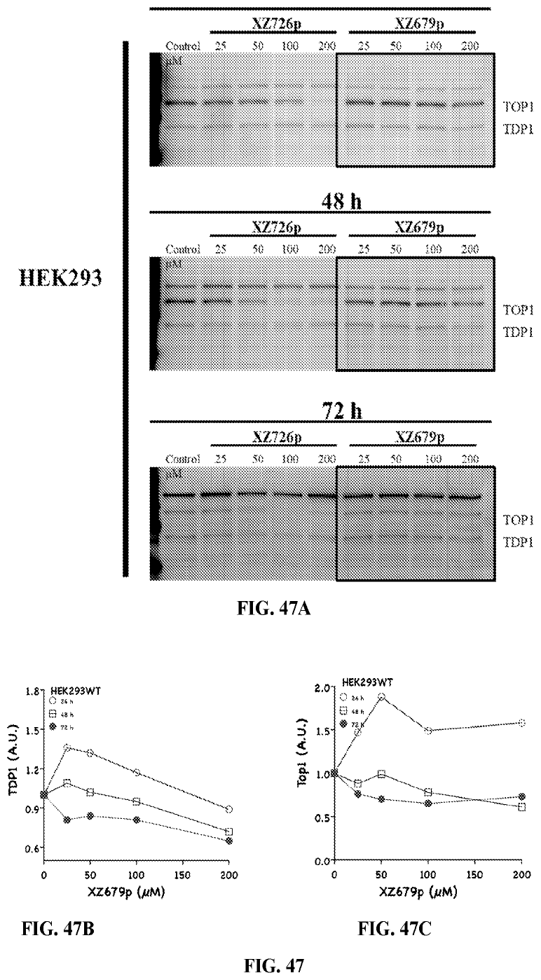

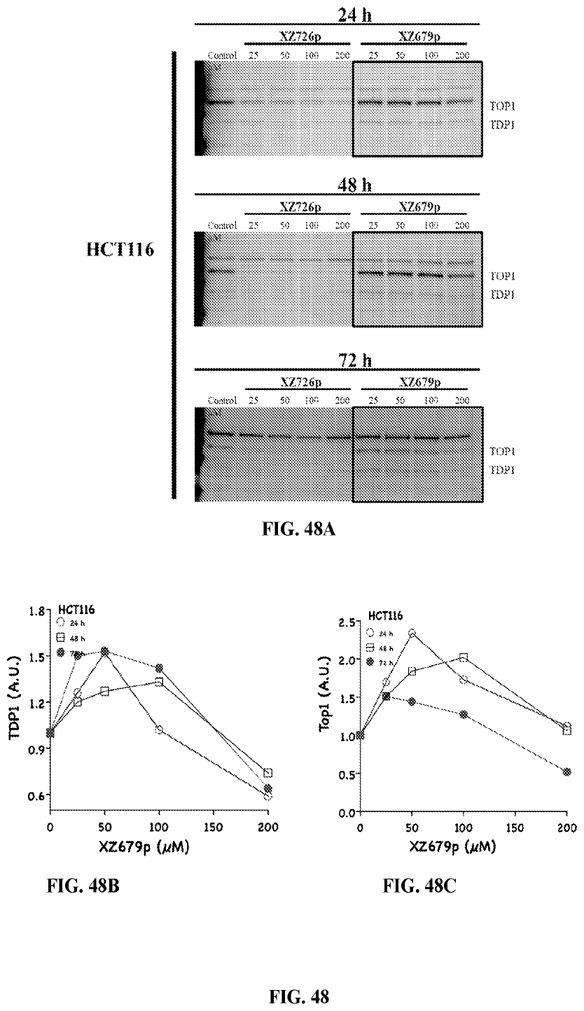

Certain compounds of the disclosure possess TDP1 degrading activity, TDP2 activity, and/or topoisomerase I (TOP1) degrading activity. Examples of compounds that exhibit TDP1 degrading activity include XZ615, XZ664, XZ616, XZ625, XZ643, XZ671, XZ679, XZ730, XZ724, XZ722, XZ701, XZ724, XZ725, XZ726, XZ718, XZ720, XZ730, XZ741, XZ734, and XZ746 (e.g., FIG. 11-15, FIG. 17, FIG. 18, FIG. 47, FIG. 48). Examples of compounds that exhibit TDP2 degrading activity include XZ701, XZ702, XZ703, XZ718, XZ719, XZ720, XZ722, XZ723, XZ727, XZ730, XZ731, XZ732, and XZ739 (e.g., FIG. 9, FIG. 35-38). Examples of compounds that exhibit TOP1 degrading activity include XZ679, XZ616, XZ625, XZ632, XZ701, XZ718, XZ720, XZ722, and XZ726 (e.g., FIG. 11).

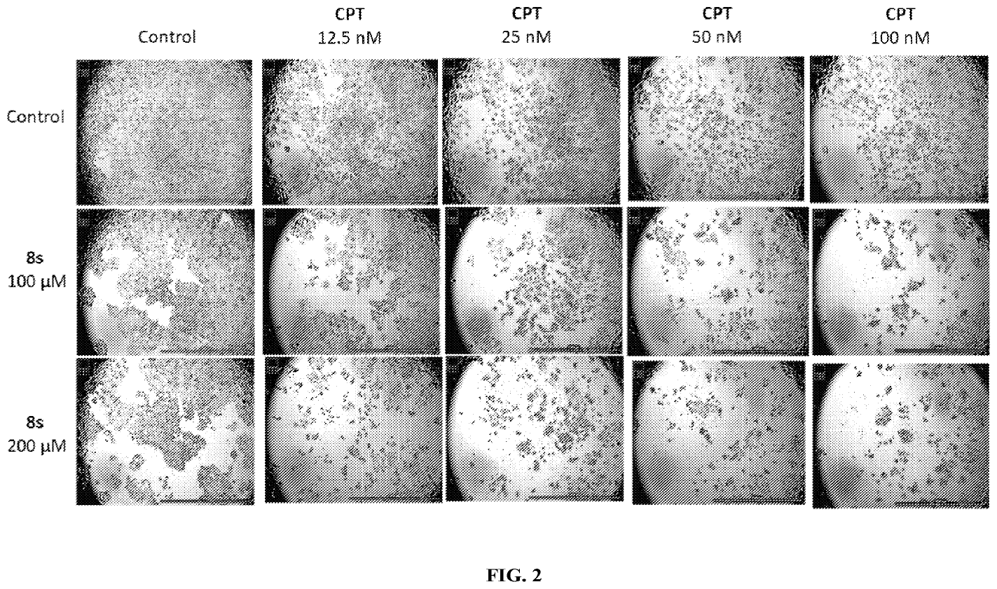

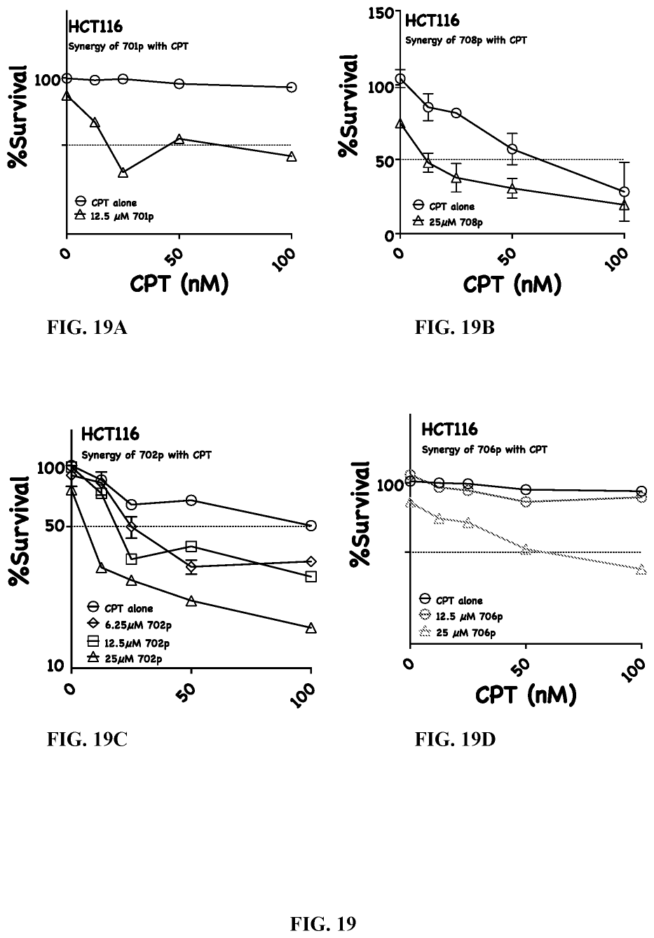

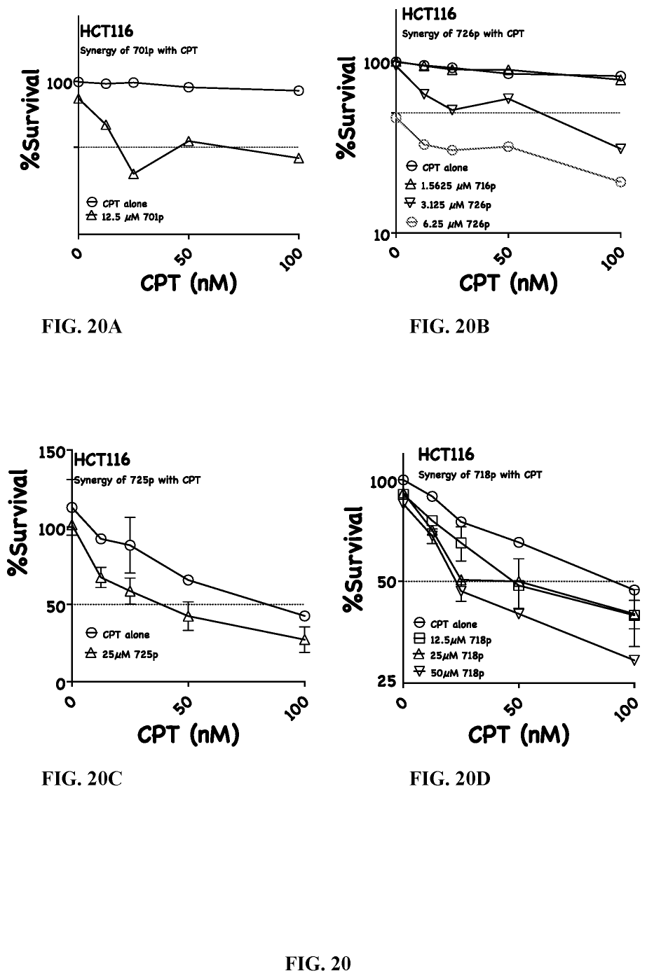

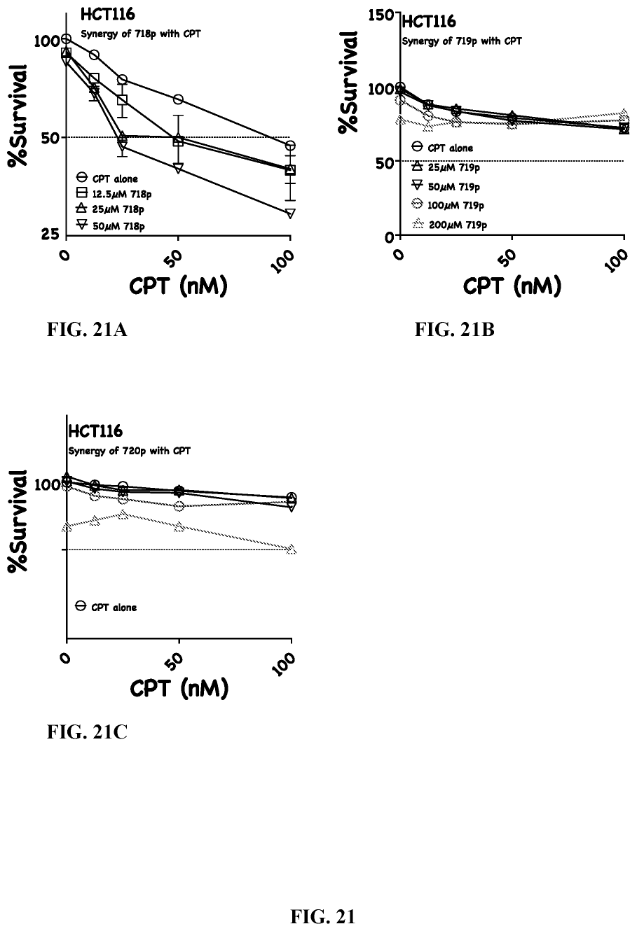

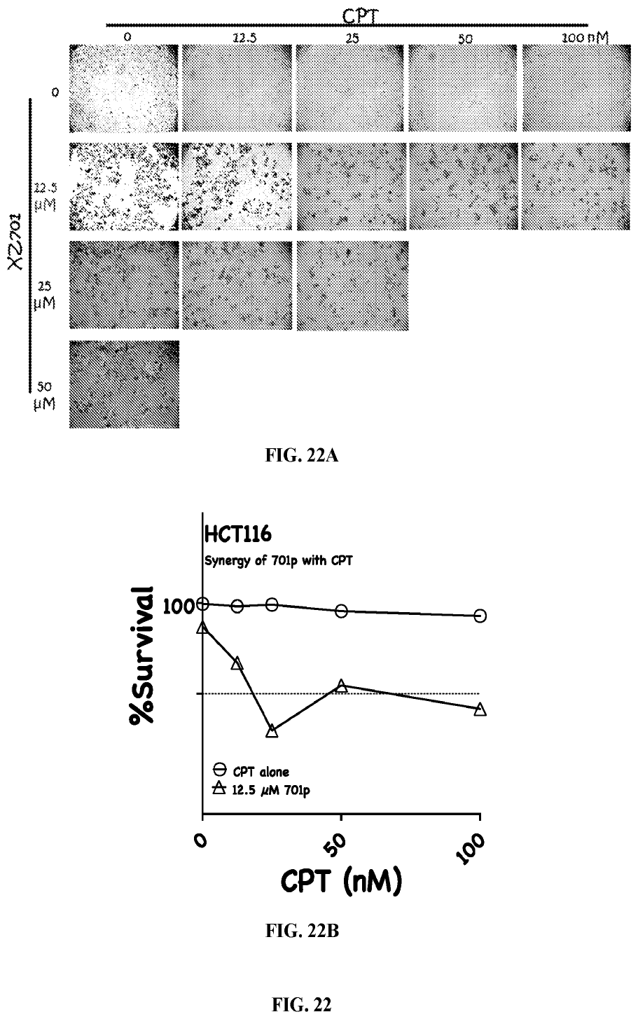

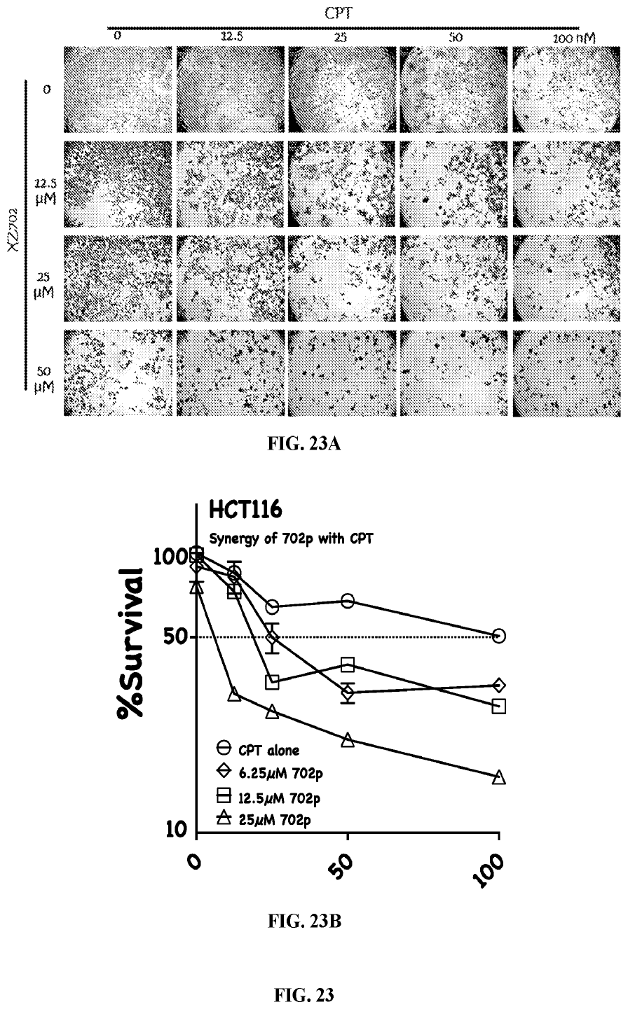

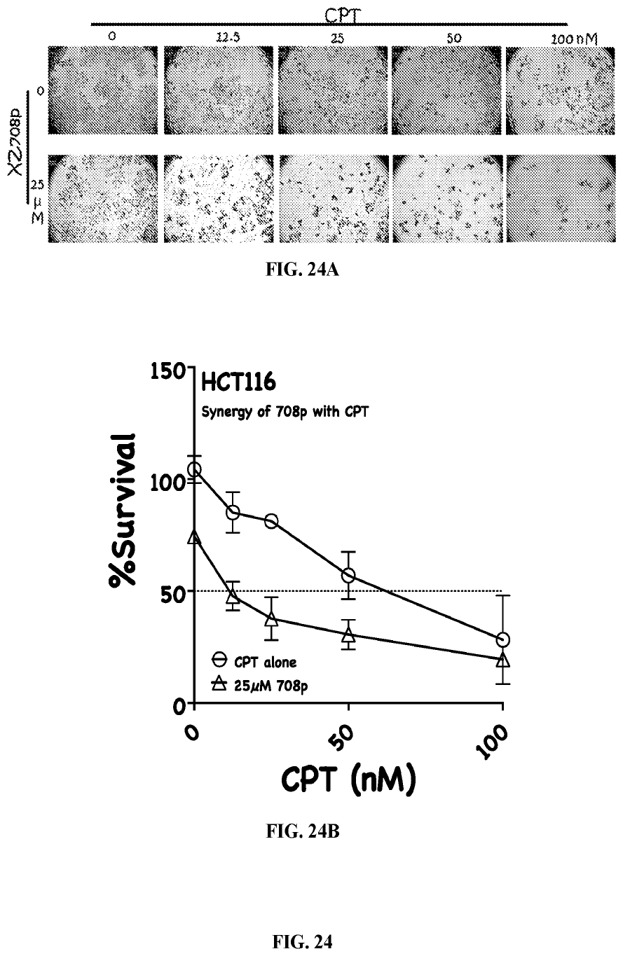

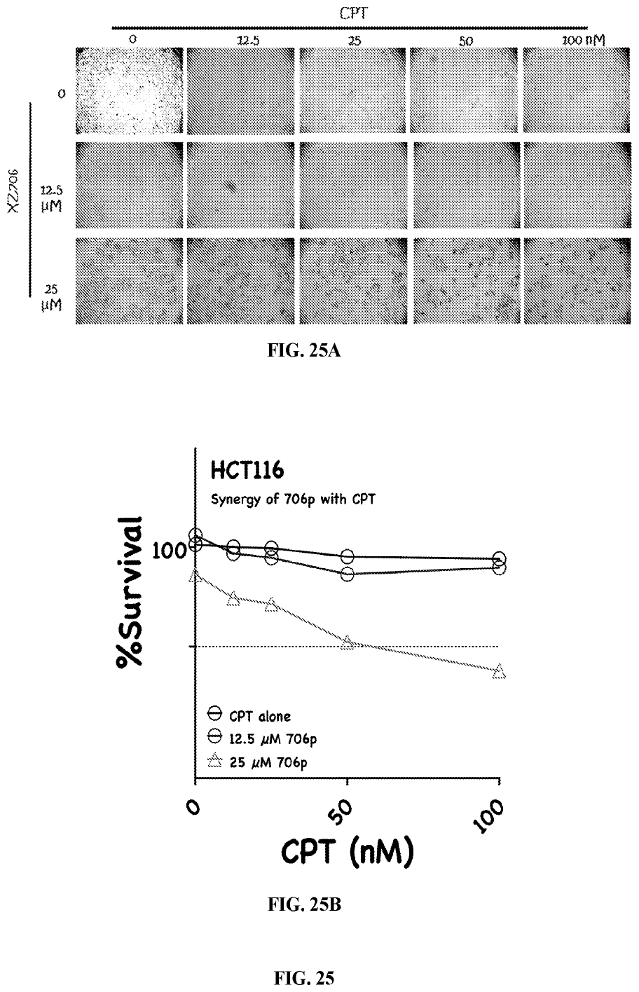

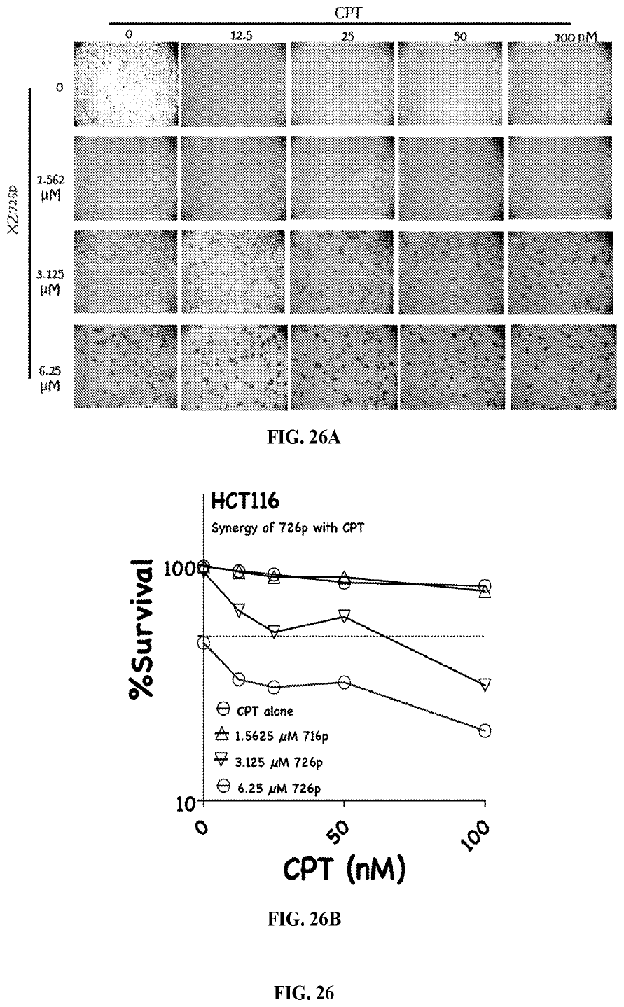

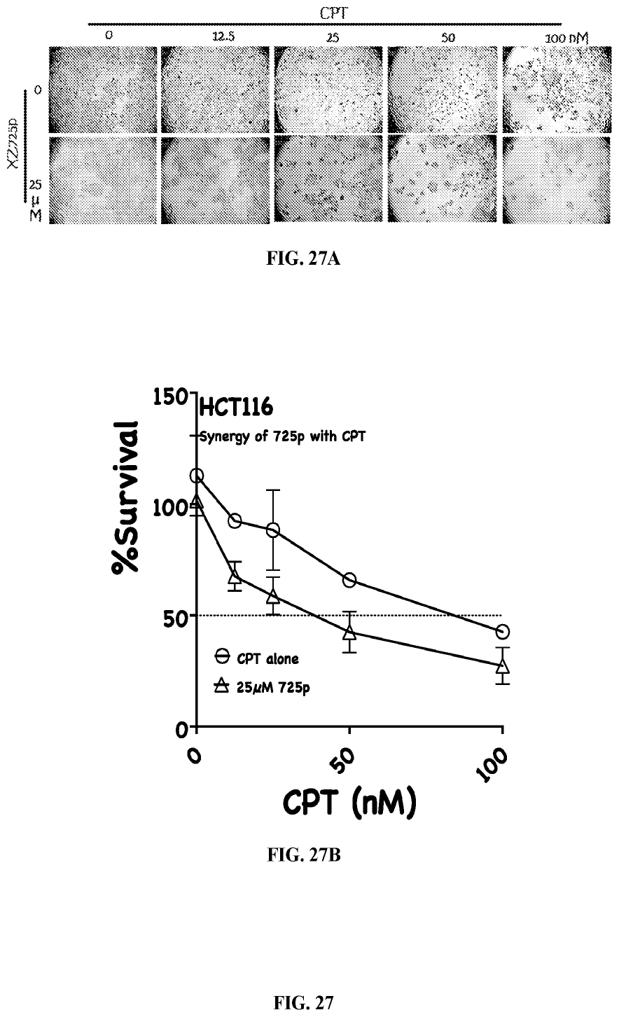

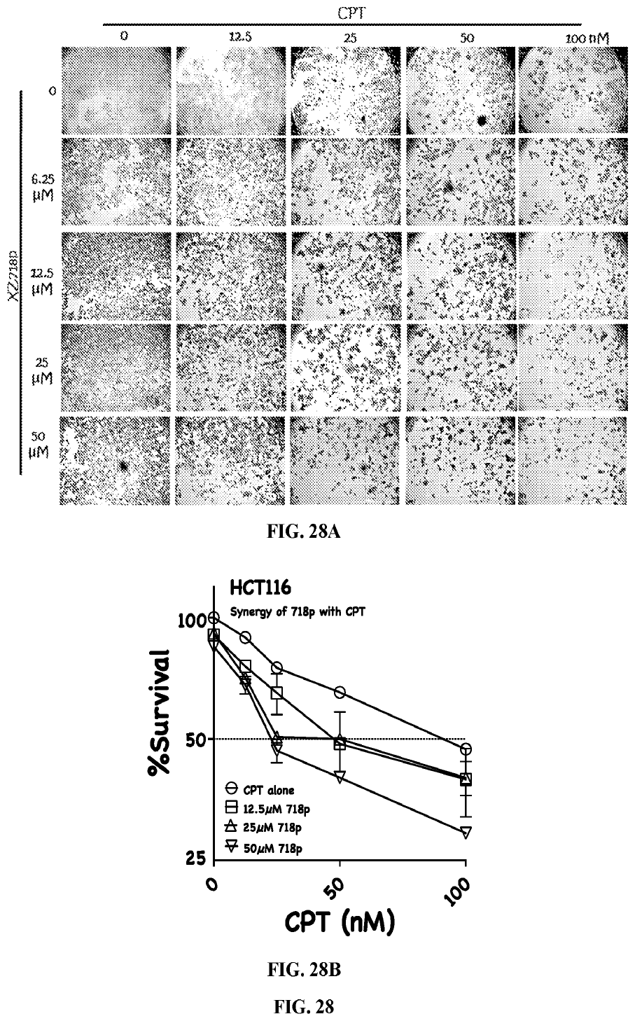

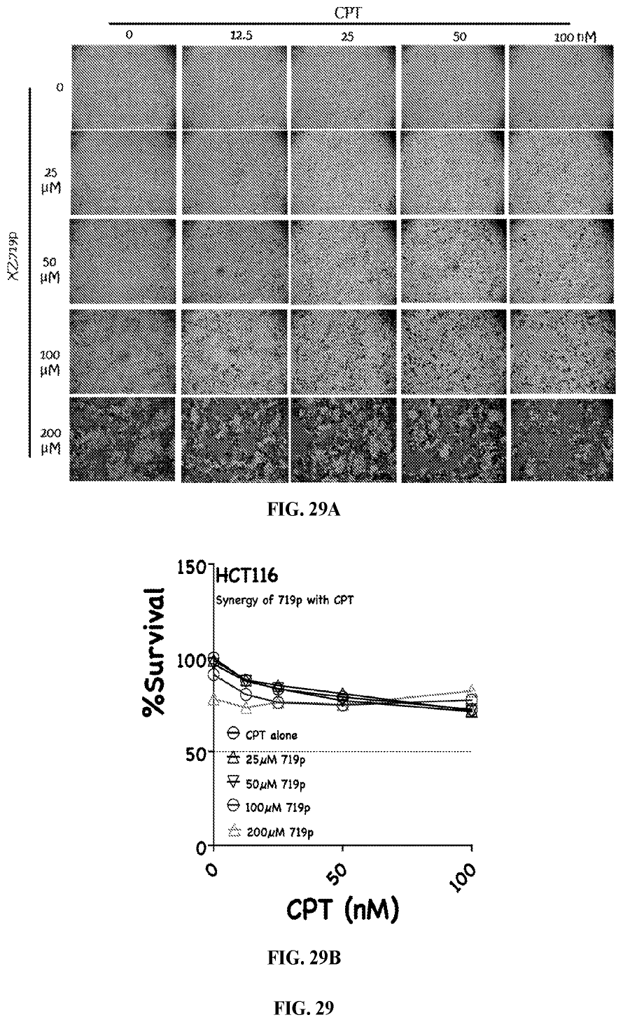

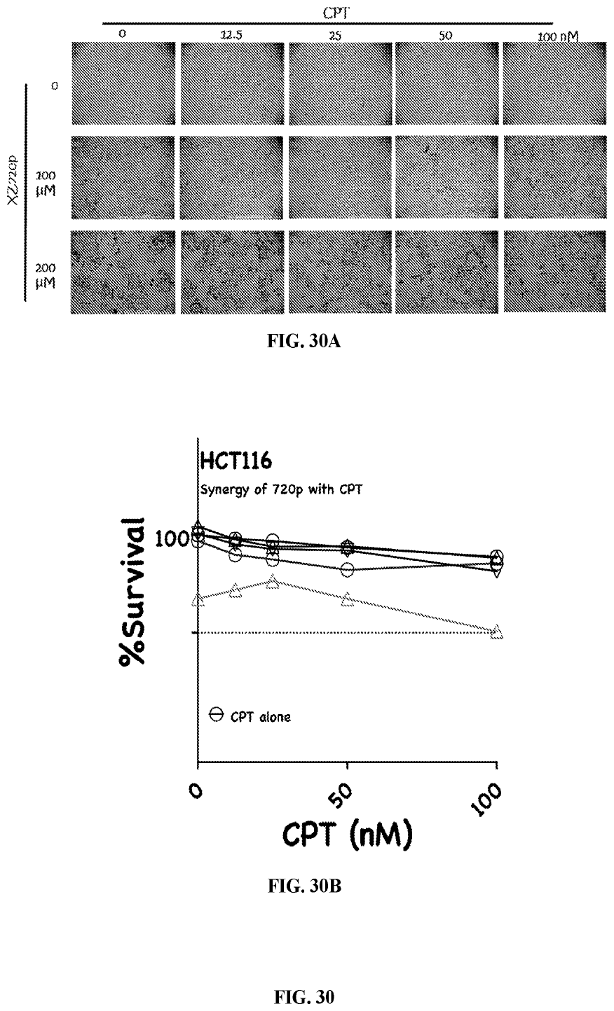

Certain compounds of the disclosure exhibit synergy with TOP1 inhibitors, e.g., camptothecin (CPT). Examples of compounds that exhibit synergy with camptothecin include M7, 8m, 8s, 10b, 8n, 8q, 8o, 6u, XZ701, XZ702, XZ706, XZ708, XZ718, XZ719, XZ720, XZ725, and XZ726 (e.g., FIG. 2-5, FIG. 19-30).

### EXAMPLES

### Synthetic Examples

### SE1: General Procedures

Proton (¹H) and carbon (¹³C) NMR spectra were recorded on a Varian 400 MHz spectrometer or a Varian 500 MHz spectrometer and are reported in ppm relative to TMS and referenced to the solvent in which the spectra were collected. Solvent was removed by rotary evaporation under reduced pressure, and anhydrous solvents were obtained commercially and used without further drying. Purification by silica gel chromatography was performed using Combiflash with EtOAc-hexanes solvent systems. Preparative high pressure liquid chromatography (HPLC) was conducted using a Waters Prep LC4000 system having photodiode array detection and Phenomenex C18 columns (catalogue no. 00G4436-P0-AX, 250 mm×21.2 mm 10 μm particle size, 110 Å pore) at a flow rate of 10 mL/min or 20 mL/min. Binary solvent systems consisting of A=0.1% aqueous TFA and B=0.1% TFA in acetonitrile were employed with gradients as indicated. Products were obtained as amorphous solids following lyophilization. Electrospray ionization-mass spectrometric (ESI-MS) were acquired with an Agilent LC/MSD system equipped with a multimode ion source. Dual ionization mass spectrometric (DUIS-MS) were acquired with a Shimadzu LCMS system equipped with dual ionization source, electrospray ionization (ESI) and atmospheric pressure chemical ionization (APCI). Purities of samples subjected to biological testing were assessed using this system and shown to be >95%. High resolution mass spectrometric (HRMS) were acquired by LC/MS-ESI using LTQ-Orbitrap-XL at 30K resolution.

### SE2: Synthesis of Substituted Isocyanobenzenes (3a-d) (Starting Material)

### Step 1. General Procedure for the Synthesis of Substituted Formamidobenzenes (2)

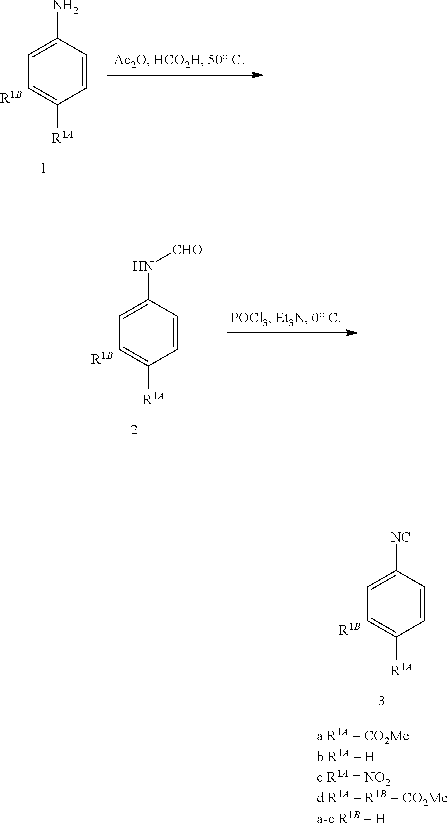

The mixture of formic acid (15 mL, 407 mmol) and acetic anhydride (35 mL, 370 mmol) was stirred (55° C., 2 h) and then cooled to rt. to afford acetic formic anhydride (about 3 equiv.) in situ. The mixture was added dropwise to a solution of substituted aminobenzene (1, 123 mmol) in THF (40 mL) at 0° C. The reaction mixture was stirred (rt, 2 h). The solvent was evaporated and the solid was collected by filtrate and washed by hexanes to afford formamidobenzenes (2).

Methyl 4-formamidobenzoate (2a). Treatment of methyl 4-aminobenzoate (1a) as outlined in Step 1 above provided methyl 4-formamidobenzoate (2a) as white solid (94% yield). ¹H NMR (400 MHz, DMSO-d₆) δ 10.55 (s, 1H), 8.36 (d, J=1.7 Hz, 1H), 7.94 (d, J=8.7 Hz, 2H), 7.72 (d, J=8.6 Hz, 1H), 3.83 (s, 3H). ¹³C NMR (101 MHz, DMSO-d₆) δ 166.20, 160.63, 142.97, 130.87 (2C), 124.82, 119.11 (2C), 52.39. DUIS-MS m/z: 180.0 (MH⁺). ESI-MS m/z: 180.1 (MH⁺).

N-Phenylformamide (2b). Treatment of aniline (1b) as outlined in Step 1 above provided N-phenylformamide (2b) as a white solid (95% yield). ¹H NMR (400 MHz, DMSO-d₆) δ 10.17 (s, 1H), 8.28 (d, J=2.0 Hz, 1H), 7.60 (d, J=7.2 Hz, 2H), 7.31 (t, J=7.9 Hz, 2H), 7.07 (t, J=7.4 Hz, 1H). ¹³C NMR (101 MHz, DMSO-d₆) δ 160.02, 129.84, 129.30 (2C), 124.04, 119.59 (2C). ESI-MS m/z: 122.1 (MH⁺), 144.0 (MNa⁺), 243.1 (M2H⁺).

N-(4-Nitrophenyl)formamide (2c). Treatment of 4-nitroaniline (1c) as outlined in Step 1 above provided N-(4-nitrophenyl)formamide (2c) as a yellow solid (98% yield). ¹H NMR (400 MHz, DMSO-d₆) δ 10.70 (s, 1H), 8.32 (s, 1H), 8.17 (d, J=8.9 Hz, 2H), 7.73 (d, J=8.8 Hz, 2H). ¹³C NMR (101 MHz, DMSO-d₆) δ 161.35, 144.08, 143.16, 125.42 (2C), 119.74 (2C). ESI-MS m/z: 167.0 (MH⁺).

Dimethyl 4-formamidophthalate (2d). Treatment of dimethyl 4-aminophthalate (1d) as outlined in Step 1 above provided dimethyl 4-formamidophthalate (2d) as white solid (94% yield). ¹H NMR (500 MHz, DMSO-d₆) δ 10.66 (s, 1H), 8.37 (s, 1H), 7.95 (s, 1H), 7.78 (s, 2H), 3.82 (s, 3H), 3.80 (s, 3H). ¹³C NMR (126 MHz, DMSO-d₆) δ 168.11, 166.76, 160.88, 141.69, 134.25, 130.93, 125.18, 121.06, 118.62, 53.14, 52.91. ESI-MS m/z: 238.1 (MH⁺).

### Step 2. General Procedure for the Synthesis of Substituted Isocyanobenzenes (3)

To the solution of substituted formamidobenzene (2, 52 mmol) and triethylamine (21 mL, 157 mmol) in THF (50 mL), phosphoryl trichloride (5.9 mL, 63 mmol) was added dropwise at 0° C. for 1 h. The mixture was quenched by Na₂CO₃ (sat. aq.) at 0° C. The mixture was extracted by DCM. The organic phase was washed by brine, dried by Na₂SO₄, filtered and concentrated. The residue was purified by CombiFlash using silica gel chromatography with solid loading. The final substituted isocyanobenzenes (3) were afforded.

### Preparation of Substituted Isocyanobenzenes (3)

### Methyl 4-Isocyanobenzoate (3a)

Treatment of methyl 4-formamidobenzoate (2a) as outlined in Step 2 above provided methyl 4-isocyanobenzoate (3a) as white solid (88% yield). ¹H NMR (400 MHz, CDCl₃) δ 8.05 (d, J=8.6 Hz, 2H), 7.42 (d, J=8.5 Hz, 2H), 3.91 (s, 3H). ¹³C NMR (101 MHz, CDCl₃) δ 167.19, 165.39, 130.90, 130.80 (2C), 126.43 (3C), 52.52.

### Isocyanobenzene (3b)

Treatment of N-phenylformamide (2b) as outlined in Step 2 above provided isocyanobenzene (3b) as a brown oil (80% yield). (Brown oil turn to green and dark green during pump drying.)

### 1-Isocyano-4-Nitrobenzene (3c)

Treatment of N-(4-nitrophenyl)formamide (2c) as outlined in Step 2 above provided 1-isocyano-4-nitrobenzene (3c) as a brown oil (98% yield). ¹H NMR (400 MHz, CDCl₃) δ 8.24 (d, J=8.9 Hz, 2H), 7.52 (d, J=8.8 Hz, 2H). ¹³C NMR (101 MHz, CDCl₃) δ 169.69, 147.52, 127.57 (2C), 125.08 (2C), 119.44.

### Dimethyl 4-Isocyanophthalate (3d)

Treatment of dimethyl 4-formamidophthalate (2d) as outlined in Step 2 above provided dimethyl 4-formamidophthalate (3d) as brown oil (97% yield). ¹H NMR (500 MHz, DMSO-d₆) δ 7.95 (s, 1H), 7.87 (s, 2H), 3.85 (s, 3H), 3.84 (s, 3H). ¹³C NMR (126 MHz, DMSO-d₆) δ 167.55, 166.48, 166.14, 133.47, 132.42, 130.98, 129.93, 128.30, 127.17, 53.47, 53.42. ESI-MS m/z: 220.1 (MH⁺).

### SE3: Synthesis of Substituted Imidazo[1,2-a]pyrazines and Imidazo[1,2-a]pyridines (Compounds 6a-6u)

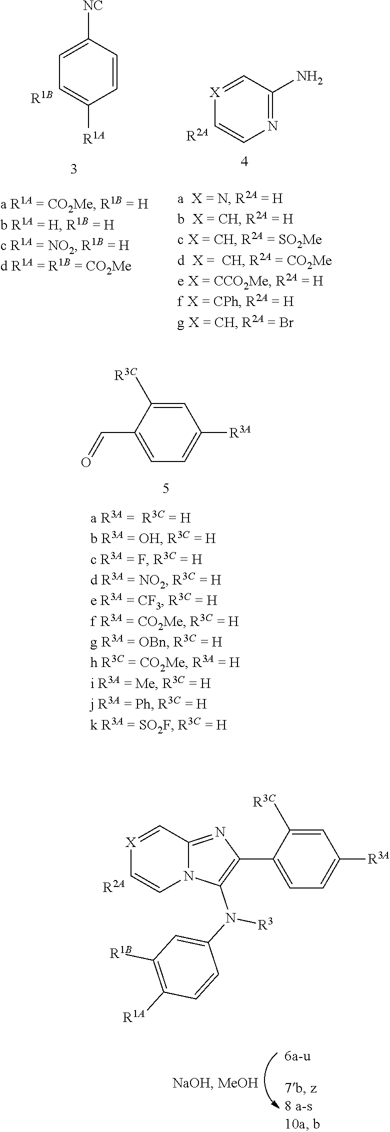

### Synthesis 1. General Procedure for the Synthesis of Imidazo[1,2-a]Pyrazines and Imidazo[1,2-a]Pyridines (6) by Heating

Pyrizan-2-amines (4, 2 mmol), aldehydes (5, 2 mmol), and isonitrile (3, 2 mmol) (freshly prepared according to the literature (Fang. Y., et al., Org. Chem. Front. 2015, 2 (10), 1338-1341, Leifert, D., et al., Chem. Comm. 2016, 52 (35), 5997-6000, Kim, B., et al., J Am. Chem. Soc. 2006, 128 (15), 4970-4971)) were mixed in MeOH (2.0 mL), trimethyl orthoformate (TMOF, 2.0 mL) or dioxane (5.0 mL). The mixture was heated and stirred. The final mixture was purified by HPLC to provide final imidazo[1,2-a]pyrazines (6).

### Synthesis 2. General Procedure for the Synthesis of Imidazo[1,2-a]Pyrazines and Imidazo[1,2-a]Pyridines (6)

Pyrizan-2-amines or pyridine-2-amines (5, 6 mmol), aldehydes (4, 6 mmol), acetic acid (12 mmol) mixed in MeOH (10 mL) (rt, 20 min). Isonitrile (3, 6 mmol) was added. The reaction mixture was stirred (rt, 24 h). The final suspension was filtered and washed by hexanes and water. The solid product was collected and purified by HPLC to provide final imidazo[1,2-a]pyrazines or imidazo[1,2-a]pyridines (6).

### Synthesis 3. General Procedure for the Hydrolysis of Esters (6) to Prepare Acids (7-10)

Esters (6, 2 mmol) were mixed with sodium hydroxide (2 mL, 2N, 4 mmol) in MeOH (2 mL) and THF (2 mL). The mixture was stirred at rt overnight. The reaction mixture was carefully adjusted to pH 3 using HCl (2N) to form the participation. The suspension was filtered and the solid was washed by water and hexanes. The solid was collected. The final acids (7-10) were afforded by HPLC purification.

### Methyl 4-((2-phenylimidazo[1,2-a]pyrazin-3-yl)amino)benzoate (Compound 6a)

Treatment of pyrazin-2-amine (4a), benzaldehyde (5a), and methyl 4-isocyanobenzoate (3a) as outlined in Synthesis 2 above provided methyl 4-((2-phenylimidazo[1,2-a]pyrazin-3-yl)amino)benzoate (6a) as a grey solid (39% yield). ¹H NMR (500 MHz, DMSO-d₆) δ 9.14 (d, J=1.5 Hz, 1H), 9.05 (s, 1H), 8.07 (dd, J=4.6, 1.5 Hz, 1H), 8.03 (dd, J=8.3, 1.3 Hz, 2H), 7.92 (d, J=4.5 Hz, 1H), 7.78 (d, J=9.0 Hz, 2H), 7.44 (t, J=7.6 Hz, 2H), 7.35 (t, J=7.3 Hz, 1H), 6.61 (d, J=8.0 Hz, 2H), 3.76 (s, 3H). ¹³C NMR (101 MHz, DMSO-d₆) δ 166.46, 149.87, 143.58, 140.12, 137.86, 133.02, 131.89 (2C), 129.94, 129.18 (2C), 128.93, 127.19 (2C), 120.34, 119.57, 116.93, 113.31 (2C), 52.03. ESI-MS m/z: 345.1 (MH⁺).

### Ethyl 4-((2-(4-fluorophenyl)imidazo[1,2-a]pyrazin-3-yl)amino)benzoate (Compound 6b)

Treatment of pyrazin-2-amine (4a), 4-fluorobenzaldehyde (5c) and methyl 4-isocyanobenzoate (3a) as outline in Synthesis 1 above (Dioxane, 50° C., 3 h) and purification by HPLC (linear gradient of 20% B to 45% B over 20 min with a flow rate 20 mL/min; retention time=14.1 min) provided methyl 4-((2-(4-fluorophenyl)imidazo[1,2-a]pyrazin-3-yl)amino)benzoate (6b) as a yellow solid (4.5% yield). ¹H NMR (500 MHz, DMSO-d₆) δ 9.19 (d, J=1.5 Hz, 1H), 9.09 (s, 1H), 8.10 (dd, J=4.6, 1.5 Hz, 1H), 8.06 (dd, J=8.7, 5.7 Hz, 2H), 7.94 (d, J=4.6 Hz, 1H), 7.78 (d, J=8.9 Hz, 2H), 7.30 (t, J=8.9 Hz, 2H), 6.62 (d, J=8.1 Hz, 2H), 3.76 (s, 3H). ¹³C NMR (126 MHz, DMSO-d₆) δ 166.44, 162.67 (d, J=246.3 Hz), 149.59, 143.04, 139.59, 137.64, 131.88 (2C), 129.36 (d, J=5.1 Hz), 129.34 (2C), 129.31 (2C, d, J=8.1 Hz), 120.49, 119.74, 117.26, 116.24 (2C, d, J=21.5 Hz), 113.40, 52.04. ESI-MS m/z: 363.1 (MH⁺).

### Methyl 4-((2-phenylimidazo[1,2-a]pyridin-3-yl)amino)benzoate (Compound 6c)

Treatment of pyridin-2-amine (4b), benzaldehyde (5a), and methyl 4-isocyanobenzoate (3a) as outlined in Synthesis 2 above provided 4-((2-phenylimidazo[1,2-a]pyridin-3-yl)amino)benzoate (6c) as a white solid (41% yield). ¹H NMR (500 MHz, DMSO-d₆) δ 8.90 (s, 1H), 8.01 (d, J=6.9 Hz, 2H), 7.97 (d, J=6.9 Hz, 1H), 7.77 (d, J=8.5 Hz, 2H), 7.66 (d, J=9.0 Hz, 1H), 7.40 (t, J=7.7 Hz, 2H), 7.34 (ddd, J=9.1, 6.7, 1.3 Hz, 1H), 7.29 (t, J=7.3 Hz, 1H), 6.94 (td, J=6.7, 1.2 Hz, 1H), 6.58 (brs, 2H), 3.76 (s, 3H). ¹³C NMR (126 MHz, DMSO-d₆) δ 166.50, 150.59, 142.48, 138.07, 133.87, 131.92 (2C), 129.00 (2C), 128.15, 126.88 (3C), 125.86, 123.49, 119.93, 117.97, 117.71, 113.02 (2C), 51.98. ESI-MS m/z: 344.1 (MH⁺).

### Methyl 4-((2-(p-tolyl)imidazo[1,2-a]pyridin-3-yl)amino)benzoate (Compound 6d)

Treatment of pyridin-2-amine (4b), 4-methylbenzaldehyde (5i), and methyl 4-isocyanobenzoate (3a) as outlined in Synthesis 2 above provided methyl 4-((2-(p-tolyl)imidazo[1,2-a]pyridin-3-yl)amino)benzoate (6d) as a yellow solid (47% yield). ¹H NMR (500 MHz, DMSO-d₆) δ 8.86 (s, 1H), 7.96 (d, J=6.7 Hz, 1H), 7.90 (d, J=8.2 Hz, 2H), 7.76 (d, J=9.3 Hz, 2H), 7.63 (d, J=9.1 Hz, 1H), 7.32 (ddd, J=9.1, 6.7, 1.3 Hz, 1H), 7.20 (d, J=7.9 Hz, 2H), 6.93 (td, J=6.7, 1.1 Hz, 1H), 6.56 (brs, 2H), 3.75 (s, 3H), 2.29 (s, 3H). ¹³C NMR (126 MHz, DMSO-d₆) δ 166.50, 150.64, 142.40, 138.22, 137.48, 131.90 (2C), 131.07, 129.57 (2C), 126.81 (2C), 125.71, 123.41, 119.86, 117.58, 117.57, 112.99, 112.90 (2C), 51.97, 21.28. ESI-MS m/z: 358.2 (MH⁺).

### Methyl 4-((2-(4-(benzyloxy)phenyl)imidazo[1,2-a]pyridin-3-yl)amino)benzoate (Compound 6e)

Treatment of pyridin-2-amine (4b), 4-(benzyloxy)benzaldehyde (5g), methyl 4-isocyanobenzoate (3a) as outlined in Synthesis 2 above provided methyl 4-((2-(4-(benzyloxy)phenyl)imidazo[1,2-a]pyridin-3-yl)amino)benzoate (6e) as a white solid (80% yield).

¹H NMR (400 MHz, DMSO-d₆) δ 8.84 (s, 1H), 7.94 (dd, J=7.5, 4.0 Hz, 3H), 7.78 (d, J=8.5 Hz, 2H), 7.63 (d, J=8.9 Hz, 1H), 7.45 (d, J=7.4 Hz, 2H), 7.39 (t, J=7.3 Hz, 2H), 7.34-7.30 (m, 2H), 7.05 (d, J=8.2 Hz, 2H), 6.93 (t, J=6.7 Hz, 1H), 6.57 (s, 2H), 5.12 (s, 2H), 3.77 (s, 3H). ¹³C NMR (126 MHz, DMSO-d₆) δ 166.52, 158.48, 150.69, 142.39, 138.13, 137.46, 131.93 (2C), 128.89 (2C), 128.30 (2C), 128.18 (3C), 126.60, 125.62, 123.36, 119.87, 117.47, 117.00, 115.35 (2C), 112.98 (2C), 112.82, 69.63, 51.98. ESI-MS m/z: 450.2 (MH⁺).

### Methyl 4-((2-(4-(trifluoromethyl)phenyl)imidazo[1,2-a]pyridin-3-yl)amino)benzoate (Compound 6f)

Treatment of pyridin-2-amine (4b), 4-(trifluoromethyl)benzaldehyde (5e), and methyl 4-isocyanobenzoate (3a) as outlined in Synthesis 2 above provided methyl 4-((2-(4-(trifluoromethyl)phenyl)imidazo[1,2-a]pyridin-3-yl)amino)benzoate (6f) as a pale yellow solid (21% yield). ¹H NMR (400 MHz, DMSO-d₆) δ 8.98 (s, 1H), 8.22 (d, J=8.1 Hz, 2H), 8.01 (d, J=6.8 Hz, 1H), 7.79 (d, J=7.7 Hz, 4H), 7.70 (d, J=9.1 Hz, 1H), 7.39 (ddd, J=9.1, 6.8, 1.3 Hz, 1H), 6.98 (td, J=6.8, 1.2 Hz, 1H), 6.61 (s, 2H), 3.76 (s, 3H).

### Methyl 4-((2-(4-hydroxyphenyl)imidazo[1,2-a]pyridin-3-yl)amino)benzoate (Compound 6g)

Treatment of pyridin-2-amine (4b), 4-hydroxybenzaldehyde (5b), and methyl 4-isocyanobenzoate (3a) as outlined in Synthesis 2 above provided methyl 4-((2-(4-hydroxyphenyl)imidazo[1,2-a]pyridin-3-yl)amino)benzoate (6g) as a white solid (50% yield). ¹H NMR (500 MHz, DMSO-d₆) δ 9.56 (s, 1H), 8.81 (s, 1H), 7.91 (d, J=6.8 Hz, 1H), 7.82 (d, J=8.7 Hz, 2H), 7.77 (d, J=8.7 Hz, 2H), 7.60 (d, J=9.0 Hz, 1H), 7.31-7.28 (m, 1H), 6.90 (td, J=6.7, 1.1 Hz, 1H), 6.77 (d, J=8.7 Hz, 2H), 6.55 (s, 2H), 3.76 (s, 3H). ¹³C NMR (126 MHz, DMSO-d₆) δ 166.52, 157.67, 150.76, 142.29, 138.62, 131.90 (2C), 128.30 (2C), 125.40, 124.81, 123.26, 119.78, 117.34, 116.54, 115.77 (2C), 112.95 (2C), 112.66, 51.96. ESI-MS m/z: 360.2 (MH⁺).

### Methyl 4-(3-((4-(methoxycarbonyl)phenyl)amino)imidazo[1,2-a]pyridin-2-yl)benzoate (Compound 6h)

Treatment of pyridin-2-amine (4b), methyl 4-formylbenzoate (5f) and methyl 4-isocyanobenzoate (3a) as outlined in Synthesis 2 above provided methyl 4-(3-((4-(methoxycarbonyl)phenyl)amino)imidazo[1,2-a]pyridin-2-yl)benzoate (6h) as a white solid (28% yield). ¹H NMR (400 MHz, DMSO-d₆) δ 8.98 (s, 1H), 8.16 (d, J=8.1 Hz, 2H), 7.99 (t, J=6.5 Hz, 3H), 7.78 (d, J=8.4 Hz, 2H), 7.69 (d, J=9.1 Hz, 1H), 7.40-7.36 (m, 1H), 6.98 (t, J=6.8 Hz, 1H), 6.61-6.59 (m, 2H), 3.85 (s, 3H), 3.76 (s, 3H). ¹³C NMR (126 MHz, DMSO-d₆) δ 166.47, 166.42, 150.21, 142.70, 138.46, 136.68, 131.92 (2C), 129.93 (2C), 128.82, 126.85 (2C), 126.40, 123.71, 120.15, 119.30, 117.92, 113.37, 113.15 (2C), 52.56, 52.00. ESI-MS m/z: 402.2 (MH⁺).

### Methyl 4-(3-(phenylamino)imidazo[1,2-a]pyridin-2-yl)benzoate (Compound 6i)

Treatment of pyridin-2-amine (4b), methyl 4-formylbenzoate (5f), and isocyanobenzene (3b) as outlined in Synthesis 2 above provided methyl 4-(3-(phenylamino)imidazo[1,2-a]pyridin-2-yl)benzoate (6i) as a white solid (64% yield). ¹H NMR (500 MHz, DMSO-d₆) δ 8.33 (s, 1H), 8.21 (d, J=8.6 Hz, 2H), 7.98-7.96 (m, 3H), 7.66 (d, J=9.0 Hz, 1H), 7.34 (ddd, J=9.1, 6.7, 1.3 Hz, 1H), 7.15 (t, 2H, J=9.0 Hz), 6.94 (td, J=6.7, 1.1 Hz, 1H), 6.74 (t, J=7.3 Hz, 1H), 6.52 (d, J=7.3 Hz, 2H), 3.84 (s, 3H). ¹³C NMR (126 MHz, DMSO-d₆) δ 166.48, 145.67, 142.46, 138.79, 136.53, 130.03 (2C), 129.85 (2C), 128.62, 126.84 (2C), 126.09, 123.74, 120.76, 119.20, 117.84, 113.49, 113.04 (2C), 52.54. ESI-MS m/z: 344.2 (MH⁺).

### Methyl 6-(4-(methoxycarbonyl)phenyl)-5-oxo-5,6-dihydropyrido[2′,1′:2,3]imidazo[4,5-c]isoquinoline-9-carboxylate (Compound 6j)

Treatment of methyl 6-aminonicotinate (4d), methyl 2-formylbenzoate (5h) and methyl 4-isocyanobenzoate (3a) as outlined in Synthesis 1 above (MeOH, Biotage Initiator, microwave irradiation, 85° C., 3 h) and provided methyl 6-(4-(methoxycarbonyl)phenyl)-5-oxo-5,6-dihydropyrido[2′,1′:2,3]imidazo[4,5-c]isoquinoline-9-carboxylate (6j) as a brown solid (5.0% yield). ¹H NMR (400 MHz, CDCl₃) δ 8.56 (d, J=7.4 Hz, 1H), 8.49 (d, J=7.8 Hz, 1H), 8.41 (d, J=8.5 Hz, 2H), 7.92 (ddd, J=8.1, 7.2, 1.3 Hz, 1H), 7.71-7.62 (m, 5H), 7.40 (s, 1H), 4.05 (s, 3H), 3.75 (s, 3H). ¹³C NMR (101 MHz, CDCl₃) δ 165.84, 164.47, 161.34, 143.10, 139.40, 133.87, 131.97, 131.73, 131.64 (2C), 129.69, 129.30 (2C), 127.98, 127.03, 126.45, 124.70, 123.55, 123.26, 122.34, 117.64, 116.06, 52.64, 52.33. ESI-MS m/z: 428.1 (MH⁺), 450.1 (MNa⁺).

### Methyl 2-phenyl-3-(phenylamino)imidazo[1,2-a]pyridine-6-carboxylate (Compound 6k)

Treatment of methyl 6-aminonicotinate (4d), benzaldehyde (5a), and isocyanobenzene (3b) as outlined in Synthesis 2 above provided methyl 2-phenyl-3-(phenylamino)imidazo[1,2-a]pyridine-6-carboxylate (6k) as a pale yellow solid (54% yield). ¹H NMR (500 MHz, DMSO-d₆) δ 8.33 (s, 1H), 8.21 (d, J=8.6 Hz, 2H), 7.98-7.96 (m, 3H), 7.66 (d, J=9.0 Hz, 1H), 7.34 (ddd, J=8.9, 6.7, 1.1 Hz, 1H), 7.14 (t, J=7.9 Hz, 2H), 6.99-6.92 (m, 1H), 6.74 (t, J=7.3 Hz, 1H), 6.52 (d, J=7.2 Hz, 2H), 3.84 (s, 3H). ¹³C NMR (126 MHz, DMSO-d₆) δ 165.22, 145.62, 142.66, 139.55, 133.39, 130.14 (2C), 129.04 (2C), 128.51, 127.06 (2C), 126.77, 124.50, 120.57, 119.43, 117.51, 115.88, 113.51 (2C), 52.90. ESI-MS m/z: 344.1 (MH⁺).

### Methyl 3-((4-nitrophenyl)amino)-2-phenylimidazo[1,2-a]pyridine-6-carboxylate (Compound 61)

Treatment of methyl 6-aminonicotinate (4d), benzaldehyde (5a), and 1-isocyano-4-nitrobenzene (3c) as outlined in Synthesis 2 above and purification by preparative HPLC (linear gradient of 10% B to 40% B over 20 min with a flow rate 20 mL/min; retention time=12.6 min) provided methyl 3-((4-nitrophenyl)amino)-2-phenylimidazo[1,2-a]pyridine-6-carboxylate (61) as a pale brown solid (18% yield). ¹H NMR (500 MHz, DMSO-d₆) δ 9.45 (s, 1H), 8.55 (s, 1H), 8.08 (d, J=8.8 Hz, 2H), 8.00 (dd, J=8.2, 1.2 Hz, 2H), 7.78 (t, J=1.5 Hz, 2H), 7.43 (t, J=7.7 Hz, 2H), 7.35 (t, J=7.4 Hz, 1H), 6.73 (brs, 2H), 3.86 (s, 3H). ESI-MS m/z: 389.1 (MH⁺).

### Methyl 3-((4-(methoxycarbonyl)phenyl)amino)-2-phenylimidazo[1,2-a]pyridine-6-carboxylate (Compound 6m)

Treatment of methyl 6-aminonicotinate (4d), benzaldehyde (5a), and methyl 4-isocyanobenzoate (3a) as outlined in Synthesis 2 above provided methyl 3-((4-(methoxycarbonyl)phenyl)amino)-2-phenylimidazo[1,2-a]pyridine-6-carboxylate (6m) as a green solid (47% yield). ¹H NMR (400 MHz, DMSO-d₆) δ 8.98 (s, 1H), 8.51 (s, 1H), 8.02-8.00 (m, 2H), 7.79-7.7 (m, 4H), 7.41 (t, J=7.6 Hz, 2H), 7.32 (t, J=6.8 Hz, 1H), 6.53 (s, 2H), 3.84 (s, 3H), 3.76 (s, 3H). ESI-MS m/z: 402.2 (MH⁺).

### Methyl 4-((6-bromo-2-phenylimidazo[1,2-a]pyridin-3-yl)amino)benzoate (Compound 6n)

Treatment of 5-bromopyridin-2-amine (4g), benzaldehyde (5a), and methyl 4-isocyanobenzoate (3a) as outlined in Synthesis 2 above provided methyl 4-((6-bromo-2-phenylimidazo[1,2-a]pyridin-3-yl)amino)benzoate (6n) as a pale yellow solid (76% yield). ¹H NMR (500 MHz, DMSO-d₆) δ 8.88 (s, 1H), 8.25-8.21 (m, 1H), 7.98 (d, J=7.0 Hz, 2H), 7.78 (d, J=8.5 Hz, 2H), 7.66 (d, J=9.5 Hz, 1H), 7.46 (dd, J=9.5, 2.0 Hz, 1H), 7.40 (t, J=7.7 Hz, 2H), 7.30 (t, J=7.4 Hz, 1H), 6.60 (brs, 2H), 3.76 (s, 3H). ¹³C NMR (126 MHz, DMSO-d₆) δ 166.48, 150.19, 140.94, 138.81, 133.36, 131.86 (2C), 129.06 (2C), 128.80, 128.44, 126.92 (2C), 123.33, 120.16, 118.97, 118.60, 113.22 (2C), 107.10, 52.00. ESI-MS m/z: 422.1, 424.1 (MH⁺).

### Methyl 4-((2,7-diphenylimidazo[1,2-a]pyridin-3-yl)amino)benzoate (Compound 6o)

Treatment of 4-phenylpyridin-2-amine (4f), benzaldehyde (5a), and methyl 4-isocyanobenzoate (3a) as outlined in Synthesis 2 above provided methyl 4-((2,7-diphenylimidazo[1,2-a]pyridin-3-yl)amino)benzoate (6o) as a white solid (49% yield). ¹H NMR (500 MHz, CDCl₃) δ 8.01-7.97 (m, 2H), 7.95 (d, J=9.0 Hz, 2H), 7.87 (s, 1H), 7.85 (d, J=7.1 Hz, 1H), 7.67 (d, J=7.0 Hz, 2H), 7.51 (t, J=7.5 Hz, 2H), 7.44 (t, J=7.4 Hz, 1H), 7.39 (t, J=7.5 Hz, 2H), 7.34-7.31 (m, 1H), 7.10 (dd, J=7.1, 1.8 Hz, 1H), 6.66 (d, J=8.4 Hz, 2H), 6.08 (brs, 1H), 3.89 (s, 3H). ¹³C NMR (126 MHz, CDCl₃) δ 166.84, 148.88, 143.33, 138.48, 132.97, 132.04 (2C), 130.85, 129.16 (2C), 128.72 (2C), 128.43, 128.21, 127.02 (2C), 126.79 (2C), 126.47, 122.46, 121.83, 116.50, 114.49, 112.86 (2C), 112.53, 51.81. ESI-MS m/z: 420.2 (MH⁺).

### Methyl 4-((6-(methylsulfonyl)-2-phenylimidazo[1,2-a]pyridin-3-yl)amino)benzoate (Compound 6p)

Treatment of 5-(methylsulfonyl)pyridin-2-amine (4c), benzaldehyde (5a), and methyl 4-isocyanobenzoate (3a) as outlined in Synthesis 2 above provided methyl 4-((6-(methylsulfonyl)-2-phenylimidazo[1,2-a]pyridin-3-yl)amino)benzoate (6p) as a green solid (10% yield). ¹H NMR (500 MHz, DMsSO-d₆) δ 9.03 (s, 1H), 8.49 (d, J=2.0 Hz, 1H), 8.02 (d, J=7.3 Hz, 2H), 7.90 (d, J=9.5 Hz, 1H), 7.79 (d, J=8.5 Hz, 2H), 7.75 (dd, J=9.5, 1.9 Hz, 1H), 7.43 (t, J=7.6 Hz, 2H), 7.34 (t, J=7.3 Hz, 1H), 6.67 (brs, 2H), 3.77 (s, 3H), 3.34 (s, 3H). ¹³C NMR (126 MHz, DMSO-d₆) δ 166.46, 149.88, 142.29, 140.03, 132.94, 131.94 (2C), 129.17 (2C), 128.85, 127.54, 127.11 (2C), 125.50, 122.53, 120.46, 119.90, 118.44, 113.36 (2C), 52.04, 44.01.

### Methyl 4-((2-([1,1′-biphenyl]-4-yl)-6-(methylsulfonyl)imidazo[1,2-a]pyridin-3-yl)amino)benzoate (Compound 6q)

Treatment of 5-(methylsulfonyl)pyridin-2-amine (4c), (1,1′-biphenyl)-4-carbaldehyde (5j), and methyl 4-isocyanobenzoate (3a) as outlined in Synthesis 2 above provided methyl 4-((2-([1,1′-biphenyl]-4-yl)-6-(methylsulfonyl)imidazo[1,2-a]pyridin-3-yl)amino)benzoate (6q) as a white solid (27% yield). ¹H NMR (500 MHz, DMSO-d₆) δ 9.08 (s, 1H), 8.50 (dd, J=2.0, 0.9 Hz, 1H), 8.12 (d, J=8.6 Hz, 2H), 7.91 (dd, J=9.4, 0.9 Hz, 1H), 7.81 (d, J=8.6 Hz, 2H), 7.78-7.74 (m, 3H), 7.71 (dd, J=8.3, 1.3 Hz, 2H), 7.46 (t, J=7.7 Hz, 2H), 7.39-7.34 (m, 1H), 6.70 (s, 2H), 3.76 (s, 3H), 3.34 (s, 3H). ¹³C NMR (126 MHz, DMSO-d₆) δ 166.46, 149.88, 142.38, 140.33, 139.85, 139.73, 132.01, 131.97 (2C), 129.43 (2C), 128.12, 127.63 (2C), 127.56, 127.35 (2C), 127.02 (2C), 125.50, 122.60, 120.52, 120.00, 118.42, 113.40 (2C), 52.04, 44.03. ESI-MS m/z: 498.1 (MH⁺).

### Methyl 3-((4-(methoxycarbonyl)phenyl)amino)-2-phenylimidazo[1,2-a]pyridine-7-carboxylate (Compound 6r)

Treatment of methyl 2-aminoisonicotinate (4e), benzaldehyde (5a), and methyl 4-isocyanobenzoate (3a) as outlined in Synthesis 2 above provided methyl 3-((4-(methoxycarbonyl)phenyl)amino)-2-phenylimidazo[1,2-a]pyridine-7-carboxylate (6r) as a green solid (95% yield). ¹H NMR (500 MHz, DMSO-d₆) δ 9.05 (s, 1H), 8.23 (s, 1H), 8.09 (d, J=7.1 Hz, 1H), 8.02 (d, J=7.1 Hz, 2H), 7.78 (d, J=8.5 Hz, 2H), 7.43 (t, J=7.6 Hz, 2H), 7.38 (dd, J=7.0, 1.7 Hz, 1H), 7.34 (t, J=7.2 Hz, 1H), 6.61 (s, 2H), 3.91 (s, 3H), 3.76 (s, 3H). ¹³C NMR (126 MHz, DMSO-d₆) δ 166.45, 165.59, 150.03, 141.23, 140.70, 133.22, 131.93 (2C), 129.13 (2C), 128.72, 127.07 (2C), 126.26, 123.77, 120.24, 119.86, 119.69, 113.21 (2C), 111.70, 53.07, 52.02. ESI-MS m/z: 402.2 (MH⁺).

### Dimethyl 4-((2-phenylimidazo[1,2-a]pyrazin-3-yl)amino)phthalate (Compound 6s)

Treatment of pyrazin-2-amine (4a), benzaldehyde (5a), and dimethyl 4-isocyanophthalate (3d) as outlined in Synthesis 2 above provided dimethyl 4-((2-phenylimidazo[1,2-a]pyrazin-3-yl)amino) (6s) as a white solid (21% yield). ¹H NMR (500 MHz, DMSO-d₆) δ 9.15 (s, 1H), 9.14 (d, J=1.5 Hz, 1H), 8.11 (dd, J=4.6, 1.5 Hz, 1H), 8.03 (d, J=7.1 Hz, 2H), 7.92 (d, J=4.6 Hz, 1H), 7.66 (d, J=8.6 Hz, 1H), 7.45 (t, J=7.6 Hz, 2H), 7.36 (t, J=7.3 Hz, 1H), 6.78 (brs, 1H), 6.62 (brs, 1H), 3.74 (s, 3H), 3.74 (s, 3H). ¹³C NMR (126 MHz, DMSO-d₆) δ 168.86, 166.40, 148.98, 143.61, 140.22, 137.98, 136.44, 132.94, 132.23, 130.00, 129.23 (2C), 128.98, 127.16 (2C), 119.38, 118.98, 116.97, 114.40, 112.85, 52.89, 52.51. ESI-MS m/z: 403.1 (MH⁺).

### N,2-Diphenylimidazo[1,2-a]pyridin-3-amine (Compound 6t)

Treatment of pyridin-2-amine (4b), benzaldehyde (5a), and isocyanobenzene (3b) as outlined in Synthesis 2 above and purification by preparative HPLC (linear gradient of 10% B to 50% B over 20 min with a flow rate 20 mL/min; retention time=11.6 min) provided N,2-diphenylimidazo[1,2-a]pyridin-3-amine as a white solid (61% yield). ¹H NMR (400 MHz, DMSO-d₆) δ 8.54 (s, 1H), 8.27 (d, J=6.8 Hz, 1H), 7.97-7.94 (m, 2H), 7.91 (d, J=9.0 Hz, 1H), 7.79 (t, J=8.0 Hz, 1H), 7.52 (t, J=7.5 Hz, 2H), 7.46-7.42 (m, 1H), 7.32 (t, J=6.8 Hz, 1H), 7.18 (dd, J=8.5, 7.3 Hz, 2H), 6.80 (t, J=7.4 Hz, 1H), 6.66 (d, J=7.4 Hz, 2H). ¹³C NMR (101 MHz, DMSO-d₆) δ 146.05, 142.25, 137.95, 134.20, 130.00 (2C), 128.90 (2C), 127.94, 126.92 (2C), 125.54, 123.53, 119.40, 118.97, 117.63, 113.37 (2C), 112.69. ESI-MS m/z: 286.1 (MH⁺). HRMS calcd. for C₁₉H₁₆N₃(MH⁺), 286.1339; found, 286.1336.

### 6-(Methylsulfonyl)-N-(4-nitrophenyl)-2-phenylimidazo[1,2-a]pyridin-3-amine (Compound 6u)

Treatment of 5-(methylsulfonyl)pyridin-2-amine (4c), benzaldehyde (5a), and 1-isocyano-4-nitrobenzene (3c) as outlined in Synthesis 2 above and purification by preparative HPLC (linear gradient of 10% B to 60% B over 20 min with a flow rate 20 mL/min; retention time=13.7 min) provided 6-(methylsulfonyl)-N-(4-nitrophenyl)-2-phenylimidazo[1,2-a]pyridin-3-amine (6u) as a pale brown solid (18% yield). ¹H NMR (500 MHz, DMSO-d₆) δ 9.48 (s, 1H), 8.54 (s, 1H), 8.09 (d, J=8.9 Hz, 2H), 8.01-7.99 (m, 2H), 7.92 (dd, J=9.5, 0.9 Hz, 1H), 7.77 (dd, J=9.5, 1.9 Hz, 1H), 7.44 (t, J=7.7 Hz, 2H), 7.36 (t, J=7.4 Hz, 1H), 6.75 (brs, 2H), 3.34 (s, 3H). ¹³C NMR (126 MHz, DMSO-d₆) δ 149.74, 140.34, 137.93, 137.64, 130.53, 127.14 (2C), 126.91, 125.66, 124.97 (2C), 124.69 (2C), 123.50, 120.74, 116.96, 116.28, 111.53 (2C), 41.90. ESI-MS m/z: 409.1 (MH⁺). HRMS calcd. for C₂₀H₁₇N₄O₄S (MH⁺), 409.0965; found, 409.0966.

### SE4: Synthesis of Substituted Imidazo[1,2-a]pyrazin-3-amine (Compounds 7′b, s, t, z)

### 4-((2-Phenylimidazo[1,2-a]pyrazin-3-yl)amino)benzoic acid (Compound 7′b)

Treatment of methyl 4-((2-phenylimidazo[1,2-a]pyrazin-3-yl)amino)benzoate (6a) as outlined in Synthesis 3 of SE3 above (70° C., 15 h) provided 4-((2-phenylimidazo[1,2-a]pyrazin-3-yl)amino)benzoic acid as a pale yellow solid (82% yield). ¹H NMR (400 MHz, DMSO-d₆) δ 12.36 (s, 1H), 9.14 (d, J=1.5 Hz, 1H), 8.98 (s, 1H), 8.07-8.04 (m, 3H), 7.92 (d, J=4.6 Hz, 1H), 7.76 (d, J=8.8 Hz, 2H), 7.44 (t, J=7.5 Hz, 2H), 7.38-7.34 (m, 1H), 6.59 (d, J=8.1 Hz, 2H). ¹³C NMR (101 MHz, DMSO-d₆) δ 167.56, 149.51, 143.54, 140.13, 137.83, 133.05, 132.03 (2C), 129.90, 129.18 (2C), 128.91, 127.20 (2C), 121.49, 119.75, 116.95, 113.15 (2C). ESI-MS m/z: 331.1 (MH⁺). HRMS calcd. for C₁₉H₁₅N₄O₂ (MH⁺): 331.1190; found: 331.1180. Commercially obtained 7b: ¹H NMR (500 MHz, DMSO-d₆) δ 12.36 (brs, 1H), 9.13 (d, J=1.5 Hz, 1H), 8.97 (s, 1H), 8.07-8.03 (m, 2H), 7.91 (d, J=4.5 Hz, 1H), 7.75 (d, J=9.0 Hz, 2H), 7.44 (t, J=7.6 Hz, 2H), 7.37-7.34 (m, 1H), 6.58 (d, J=8.2 Hz, 2H). ¹³C NMR (126 MHz, DMSO-d₆) δ 167.67, 149.35, 143.55, 140.09, 137.82, 133.06, 131.99 (2C), 129.90, 129.18 (2C), 128.90, 127.19 (2C), 121.95, 119.80, 116.95, 113.10 (2C). ESI-MS m/z: 331.1 (MH⁺).

### 2-((2-(2-Hydroxyphenyl)imidazo[1,2-a]pyrazin-3-yl)amino)benzoic acid (Compound 7′s)

Treatment of commercially available methyl 2-((2-(2-hydroxyphenyl)imidazo[1,2-a]pyrazin-3-yl)amino)benzoate as outline in Synthesis 3 of SE3 above and purification by HPLC (linear gradient of 20% B to 70% B over 20 min with a flow rate 20 mL/min; retention time=10.0 min) provided 2-((2-(2-hydroxyphenyl)imidazo[1,2-a]pyrazin-3-yl)amino)benzoic acid as a yellow solid (78% yield). ¹H NMR (500 MHz, DMSO-d₆) δ 13.19 (brs, 1H), 9.64 (s, 1H), 9.19 (d, J=1.4 Hz, 1H), 8.07 (dd, J=4.6, 1.5 Hz, 1H), 7.94 (d, J=4.6 Hz, 1H), 7.91 (dd, J=8.0, 1.7 Hz, 1H), 7.72 (dd, J=7.9, 1.7 Hz, 1H), 7.19-7.15 (m, 2H), 6.91 (dd, J=8.2, 1.2 Hz, 1H), 6.79-6.74 (m, 2H), 6.05 (d, J=8.0 Hz, 1H). ¹³C NMR (126 MHz, DMSO-d₆) δ 170.06, 157.05, 147.26, 142.19, 139.16, 135.90, 135.22, 132.41, 130.79, 130.20, 127.92, 119.74, 119.66, 118.90, 117.36, 117.11, 116.68, 113.65 (2C). ESI-MS m/z: 347.1 (MH⁺).

### 3-((2-(4-Carboxyphenyl)imidazo[1,2-a]pyrazin-3-yl)amino)benzoic acid (Compound 7′t)

Treatment of commercially available 4-(3-((3-(ethoxycarbonyl)phenyl)amino)imidazo[1,2-a]pyrazin-2-yl)benzoic acid (7r) as outline in Synthesis 3 of SE3 above and purification by HPLC (linear gradient of 20% B to 70% B over 20 min with a flow rate 20 mL/min; retention time=6.3 min) provided 3-((2-(4-carboxyphenyl)imidazo[1,2-a]pyrazin-3-yl)amino)benzoic acid as a yellow solid (81% yield). ¹H NMR (500 MHz, DMSO-d₆) δ 12.93 (brs, 2H), 9.18 (d, J=1.5 Hz, 1H), 8.75 (s, 1H), 8.19 (d, J=8.5 Hz, 2H), 8.10 (dd, J=4.5, 1.5 Hz, 1H), 7.99 (d, J=8.4 Hz, 2H), 7.93 (d, J=4.6 Hz, 1H), 7.37-7.35 (m, 1H), 7.27 (t, J=7.9 Hz, 1H), 7.16 (s, 1H), 6.73 (d, J=7.4 Hz, 1H). ¹³C NMR (126 MHz, DMSO-d₆) δ 167.69, 167.41, 145.23, 143.76, 138.89, 137.77, 137.19, 132.51, 130.72, 130.34, 130.18 (2C), 129.77, 127.11 (2C), 121.52, 120.57, 117.91, 117.18, 114.41. ESI-MS m/z: 375.1 (MH⁺).

### 4-((2-(4-Fluorophenyl)imidazo[1,2-a]pyrazin-3-yl)amino)benzoic acid (Compound 7′z)

Treatment of methyl 4-((2-(4-fluorophenyl)imidazo[1,2-a]pyrazin-3-yl)amino)benzoate (6b) as outline in Synthesis 3 of SE3 above and purification by HPLC (linear gradient of 20% B to 40% B over 20 min with a flow rate 20 mL/min; retention time=10.4 min) provided 4-((2-(4-fluorophenyl)imidazo[1,2-a]pyrazin-3-yl)amino)benzoic acid as a yellow solid (83% yield). ¹H NMR (500 MHz, DMSO-d₆) δ 9.11 (d, J=1.3 Hz, 1H), 8.94 (s, 1H), 8.03-7.98 (m, 3H), 7.87 (d, J=4.6 Hz, 1H), 7.69 (d, J=8.9 Hz, 2H), 7.23 (t, J=8.9 Hz, 2H), 6.52 (d, J=7.8 Hz, 2H). ¹³C NMR (126 MHz, DMSO-d₆) δ 167.54, 162.65 (d, J=246.1 Hz), 149.25, 143.09, 139.53, 137.65, 132.02 (2C), 129.43 (d, J=3.6 Hz), 129.41, 129.30 (2C, d, J=8.4 Hz), 121.64, 119.85, 116.23 (2C, d, J=21.5 Hz), 117.23, 113.23 (2C). ESI-MS m/z: 349.1 (MH⁺).

### SE5: Preparation of Imidazo[1,2-a]pyrazin-3-amines (Compounds 8a-s)

### 4-((2-Phenylimidazo[1,2-a]pyridin-3-yl)amino)benzoic acid (Compound 8a)

Treatment of methyl 4-((2-phenylimidazo[1,2-a]pyridin-3-yl)amino)benzoate (6c) as outlined in Synthesis 3 of SE3 above (rt, 24 h) and purification by preparative HPLC (linear gradient of 10% B to 50% B over 20 min with a flow rate 20 mL/min; retention time=9.0 min) provided 4-((2-phenylimidazo[1,2-a]pyridin-3-yl)amino)benzoic acid as a dark white solid (88% yield). ¹H NMR (500 MHz, DMSO-d₆) δ 9.39 (s, 1H), 8.42 (d, J=6.7 Hz, 1H), 8.06 (d, J=9.0 Hz, 1H), 7.99 (t, J=6.7 Hz, 3H), 7.77 (d, J=8.7 Hz, 2H), 7.55 (t, J=7.5 Hz, 2H), 7.48 (dt, J=13.8, 7.0 Hz, 2H), 6.82 (d, J=8.2 Hz, 2H). ¹³C NMR (126 MHz, DMSO-d₆) δ 167.49, 148.97, 138.81, 133.72, 131.88 (2C), 130.52, 130.22, 129.70 (2C), 127.44 (2C), 127.16, 125.49, 122.25, 119.78, 117.51, 113.66 (2C), 113.45. ESI-MS m/z: 330.1 (MH⁺). HRMS calcd. for C₂₀H₁₆N₃O₂ (MH⁺), 330.1237; found, 330.1229.

### 4-((2-(p-Tolyl)imidazo[1,2-a]pyridin-3-yl)amino)benzoic acid (Compound 8b)

Treatment of methyl 4-((2-(p-tolyl)imidazo[1,2-a]pyridin-3-yl)amino)benzoate (6d) as outlined in Synthesis 3 of SE3 above (rt, 24 h) and purification by preparative HPLC (linear gradient of 5% B to 30% B over 20 min with a flow rate 20 mL/min; retention time=15.0 min) provided 4-((2-(p-tolyl)imidazo[1,2-a]pyridin-3-yl)amino)benzoic acid as a white solid (88% yield). ¹H NMR (500 MHz, DMSO-d₆) δ 12.35 (brs, 1H), 8.94 (s, 1H), 8.02 (d, J=6.5 Hz, 1H), 7.91 (d, J=8.2 Hz, 2H), 7.75 (d, J=9.1 Hz, 2H), 7.69 (d, J=8.9 Hz, 1H), 7.42-7.38 (m, 1H), 7.22 (d, J=8.0 Hz, 2H), 6.99 (t, J=6.7 Hz, 1H), 6.59 (s, 2H), 2.30 (s, 3H). ¹³C NMR (126 MHz, DMSO-d₆) δ 167.61, 150.15, 141.89, 137.82, 137.19, 132.00 (2C), 130.24, 129.65 (2C), 126.89 (2C), 126.67, 123.69, 121.15, 117.98, 117.01, 113.43, 112.93 (2C), 21.31. ESI-MS m/z: 344.2 (MH⁺). HRMS calcd. for C₂₁H₁₈N₃O₂ (MH⁺), 344.1394; found, 344.1387.

### 4-((2-(4-(Benzyloxy)phenyl)imidazo[1,2-a]pyridin-3-yl)amino)benzoic acid (Compound 8c)

Treatment of methyl 4-((2-(4-(benzyloxy)phenyl)imidazo[1,2-a]pyridin-3-yl)amino)benzoate (6e) as outlined in Synthesis 3 of SE3 above (70° C., 24 h) and purification by preparative HPLC (linear gradient of 10% B to 40% B over 20 min with a flow rate 20 mL/min; retention time=15.8 min) provided 4-((2-(4-(benzyloxy)phenyl)imidazo[1,2-a]pyridin-3-yl)amino)benzoic acid as a white solid (89% yield). ¹H NMR (500 MHz, DMSO-d₆) δ 8.75 (s, 1H), 7.96-7.94 (m, 3H), 7.75 (d, J=9.1 Hz, 2H), 7.62 (d, J=9.0 Hz, 1H), 7.44 (d, J=6.9 Hz, 2H), 7.39 (t, J=7.4 Hz, 2H), 7.34-7.29 (m, 2H), 7.05 (d, J=9.0 Hz, 2H), 6.92 (t, J=6.7 Hz, 1H), 6.54 (s, 2H), 5.12 (s, 2H). ¹³C NMR (126 MHz, DMSO-d₆) δ 167.88, 158.46, 150.01, 142.34, 138.11, 137.48, 131.98, 128.89 (4C), 128.29, 128.21 (2C), 128.18 (4C), 126.69, 125.55, 123.38, 117.44, 117.32, 115.33, 112.74 (2C), 69.63. HRMS calcd. for C₂₇H₂₂N₃O₃ (MH⁺), 436.1656; found, 436.1646.

### 4-((2-(4-(Trifluoromethyl)phenyl)imidazo[1,2-a]pyridin-3-yl)amino)benzoic acid (Compound 8d)

Treatment of methyl 4-((2-(4-(trifluoromethyl)phenyl)imidazo[1,2-a]pyridin-3-yl)amino)benzoate (6f) as outlined in Synthesis 3 of SE3 above (rt, 24 h) and purification by preparative HPLC (linear gradient of 5% B to 30% B over 20 min with a flow rate 20 mL/min; retention time=18.0 min) provided 4-((2-(4-(trifluoromethyl)phenyl)imidazo[1,2-a]pyridin-3-yl)amino)benzoic acid as a brown solid (25% yield). ¹H NMR (400 MHz, DMSO-d₆) δ 12.38 (brs, 1H), 8.93 (s, 1H), 8.23 (d, J=8.2 Hz, 2H), 8.02 (d, J=6.8 Hz, 1H), 7.78 (dd, J=11.4, 8.5 Hz, 4H), 7.71 (d, J=9.0 Hz, 1H), 7.42-7.38 (m, 1H), 7.00 (t, J=6.7 Hz, 1H), 6.59 (d, J=8.3 Hz, 2H). ESI-MS m/z: 398.1 (MH⁺). HRMS calcd. for C₂₁H₁₅F₃N₃O₂ (MH⁺), 398.1111; found, 398.1109.

### 4-((2-(4-Nitrophenyl)imidazo[1,2-a]pyridin-3-yl)amino)benzoic acid (Compound 8e)

Treatment of pyridin-2-amine (4b), 4-nitrobenzaldehyde (5d), and methyl 4-isocyanobenzoate (3a) as outlined in Synthesis 2 of SE3 above provided methyl 4-((2-(4-nitrophenyl)imidazo[1,2-a]pyridin-3-yl)amino)benzoate as a yellow solid (34% yield). ESI-MS m/z: 389.1 (MH⁺). Treatment of methyl 4-((2-(4-nitrophenyl)imidazo[1,2-a]pyridin-3-yl)amino)benzoate as outlined in Synthesis 3 of SE3 above (rt, 24 h) and purification by preparative HPLC (linear gradient of 5% B to 30% B over 20 min with a flow rate 20 mL/min; retention time=15.2 min) provided 4-((2-(4-nitrophenyl)imidazo[1,2-a]pyridin-3-yl)amino)benzoic acid as a brown solid (8.6% yield). ¹H NMR (500 MHz, DMSO-d₆) δ 9.07 (s, 1H), 8.33 (d, J=9.0 Hz, 2H), 8.25 (d, J=8.9 Hz, 2H), 8.12 (d, J=6.8 Hz, 1H), 7.80 (d, J=9.0 Hz, 1H), 7.77 (d, J=8.5 Hz, 2H), 7.55 (t, J=7.9 Hz, 1H), 7.12 (t, J=6.8 Hz, 1H), 6.65 (d, J=8.3 Hz, 2H). ¹³C NMR (126 MHz, DMSO-d₆) δ 167.53, 149.32, 147.15, 142.02, 138.75, 133.93, 132.04 (2C), 128.63, 127.72 (2C), 124.58 (2C), 124.33, 121.78, 120.64, 117.04, 114.61, 113.27 (2C). ESI-MS m/z: 375.1 (MH⁺). HRMS calcd. for C₂₀H₁₅N₄O₄ (MH⁺), 375.1088; found, 375.1082.

### 4-((2-(4-Hydroxyphenyl)imidazo[1,2-a]pyridin-3-yl)amino)benzoic acid (Compound 8f)

Treatment of methyl 4-((2-(4-hydroxyphenyl)imidazo[1,2-a]pyridin-3-yl)amino)benzoate (6g) as outlined in Synthesis 3 of SE3 above (65° C., 24 h) and purification by preparative HPLC (linear gradient of 10% B to 50% B over 20 min with a flow rate 20 mL/min; retention time=8.0 min) provided 4-((2-(4-hydroxyphenyl)imidazo[1,2-a]pyridin-3-yl)amino)benzoic acid as a pale pink solid (58% yield). ¹H NMR (400 MHz, DMSO-d₆) δ 12.33 (s, 1H), 9.63 (s, 1H), 8.80 (s, 1H), 7.97 (d, J=6.6 Hz, 1H), 7.83 (d, J=8.7 Hz, 2H), 7.76 (d, J=9.0 Hz, 2H), 7.64 (d, J=9.0 Hz, 1H), 7.39-7.35 (m, 1H), 6.97 (t, J=6.5 Hz, 1H), 6.80 (d, J=8.7 Hz, 2H), 6.57 (d, J=6.4 Hz, 2H). ¹³C NMR (126 MHz, DMSO-d₆) δ 167.61, 157.89, 150.26, 141.82, 137.71, 132.01 (2C), 128.38 (2C), 126.25, 124.02, 123.50, 121.06, 116.88, 116.84, 115.85 (2C), 113.15, 112.87 (2C). ESI-MS m/z: 346.1 (MH⁺). HRMS calcd. for C₂₀H₁₆N₃O₃ (MH⁺), 346.1186; found, 346.1182.

### 4-((2-(4-Sulfophenyl)imidazo[1,2-a]pyridin-3-yl)amino)benzoic acid (Compound 8g)

To a stirring solution of commercially available 4-formylbenzenesulfonyl chloride (1.1 g, 5.50 mmol) in acetonitrile (25 mL) at rt was added a potassium bifloride (KHF₂, 4.7 g, 61 mmol) in water (25 mL). The resulting biphasic mixture was stirred vigorously (rt, 2 h). The reaction mixture was then diluted with water (20 mL) and ethyl acetate (100 mL) the layers separated. The aqueous phase was extracted with 2×100 mL portions of ethyl acetate, then the combined extracts were washed by brine (80 mL). The organic phase was dried over Na₂SO₄ and filtered. The filtrate was concentrated to afford the 4-formylbenzenesulfonyl fluoride (5k, 780 mg) as brown solid (75% yield). [¹H NMR (400 MHz, DMSO-d₆) δ 10.19 (s, 1H), 8.38 (d, J=8.5 Hz, 2H), 8.26 (d, J=7.7 Hz, 2H). ¹⁹F NMR (376 MHz, DMSO-d₆) δ 65.87. ¹³C NMR (101 MHz, DMSO-d₆) δ 192.81, 141.61, 136.17 (d, J=24.0 Hz) 131.27 (2C), 129.74 (2C).] Treatment of pyridin-2-amine (4b), 4-formylbenzenesulfonyl fluoride (5k), and methyl 4-isocyanobenzoate (3a) as outlined in Synthesis 2 of SE3 above provided methyl 4-((2-(4-(fluorosulfonyl)phenyl)imidazo[1,2-a]pyridin-3-yl)amino)benzoate as white solid (15% yield). [¹H NMR (400 MHz, DMSO-d₆) δ 9.06 (s, 1H), 8.38 (d, J=8.6 Hz, 2H), 8.18 (d, J=8.8 Hz, 2H), 8.02 (d, J=6.8 Hz, 1H), 7.79 (d, J=9.1 Hz, 2H), 7.72 (d, J=9.1 Hz, 1H), 7.41 (ddd, J=9.1, 6.7, 1.3 Hz, 1H), 7.00 (td, J=6.8, 1.1 Hz, 1H), 6.63 (d, J=8.3 Hz, 2H), 3.76 (s, 3H). ¹³C NMR (101 MHz, DMSO-d₆) δ 166.46, 149.87, 142.95, 141.60, 135.36, 131.97 (2C), 130.13 (d, J=23.4 Hz, 1C), 129.43 (2C), 127.92 (2C), 126.95, 123.92, 120.47 (2C), 118.16, 113.75, 113.29 (2C), 52.02. ¹⁹F NMR (376 MHz, DMSO-d₆) δ 66.72. ESI-MS m/z: 426.1 (MH⁺), 448.1 (MNa⁺).] Treatment of methyl 4-((2-(4-(fluorosulfonyl)phenyl)imidazo[1,2-a]pyridin-3-yl)amino)benzoate as outlined in Synthesis 3 of SE3 above (rt, 15 h) and purification by preparative HPLC (linear gradient of 5% B to 20% B over 20 min with a flow rate 20 mL/min; retention time=11.4 min) provided 4-((2-(4-sulfophenyl)imidazo[1,2-a]pyridin-3-yl)amino)benzoic acid as white solid (52% yield). ¹H NMR (400 MHz, DMSO-d₆) δ 9.14 (s, 1H), 8.36 (d, J=6.8 Hz, 1H), 7.98 (d, J=8.9 Hz, 1H), 7.92 (t, J=8.0 Hz, 1H), 7.86 (d, J=8.5 Hz, 2H), 7.77 (d, J=8.9 Hz, 2H), 7.73 (d, J=8.5 Hz, 2H), 7.42 (t, J=6.8 Hz, 1H), 6.78 (d, J=8.3 Hz, 2H). ¹³C NMR (126 MHz, DMSO-d₆) δ 167.48, 150.01, 148.91, 139.12, 133.15, 131.91 (2C), 130.61, 127.59, 126.87 (2C), 126.75 (2C), 125.37, 122.26, 119.94, 117.15, 113.88, 113.63 (2C). ESI-MS m/z: 410.1 (MH⁺). HRMS calcd. for C₂₀H₁₆N₃O₅S (MH⁺), 410.0805; found, 410.0803. HRMS calcd. for C₂₀H₁₅N₃O₅SNa (MNa⁺), 432.0625; found, 432.0622.

### 4-(3-((4-Carboxyphenyl)amino)imidazo[1,2-a]pyridin-2-yl)benzoic acid (Compound 8h)

Treatment of methyl 4-(3-((4-(methoxycarbonyl)phenyl)amino)imidazo[1,2-a]pyridin-2-yl)benzoate (6h) as outlined in Synthesis 3 of SE3 above (65° C., 24 h) and purification by preparative HPLC (linear gradient of 5% B to 20% B over 20 min with a flow rate 20 mL/min; retention time=14.9 min) provided 4-(3-((4-carboxyphenyl)amino)imidazo[1,2-a]pyridin-2-yl)benzoic acid as a yellow solid (70% yield). ¹H NMR (500 MHz, DMSO-d₆) δ 8.92 (s, 1H), 8.10 (d, J=8.1 Hz, 2H), 7.98 (d, J=6.8 Hz, 1H), 7.94 (d, J=8.1 Hz, 2H), 7.75 (d, J=8.4 Hz, 2H), 7.67 (d, J=9.1 Hz, 1H), 7.37-7.34 (m, 1H), 6.96 (t, J=6.7 Hz, 1H), 6.56 (s, 2H). ¹³C NMR (126 MHz, DMSO-d₆) δ 168.05, 167.73, 149.87, 142.60, 137.22, 137.18, 132.26, 132.02 (2C), 129.95 (2C), 126.54 (2C), 126.16, 123.67, 121.65, 119.16, 117.83, 113.19, 112.92 (2C). ESI-MS m/z: 374.1 (MH⁺). HRMS calcd. for C₂₁H₁₆N₃O₄ (MH⁺), 374.1135; found, 374.1126.

### 4-(3-(Phenylamino)imidazo[1,2-a]pyridin-2-yl)benzoic acid (Compound 8i)

Treatment of methyl 4-(3-(phenylamino)imidazo[1,2-a]pyridin-2-yl)benzoate (6i) as outlined in Synthesis 3 of SE3 above (rt, 24 h) and purification by preparative HPLC (linear gradient of 5% B to 30% B over 20 min with a flow rate 20 mL/min; retention time=15.9 min) provided 4-(3-(phenylamino)imidazo[1,2-a]pyridin-2-yl)benzoic acid as a white solid (95% yield). ¹H NMR (500 MHz, DMSO-d₆) δ 13.17 (brs, 1H), 8.82 (s, 1H), 8.33 (d, J=6.8 Hz, 1H), 8.15 (d, J=8.3 Hz, 2H), 8.04 (d, J=8.3 Hz, 2H), 7.99 (d, J=9.0 Hz, 1H), 7.88 (t, J=7.9 Hz, 1H), 7.38 (t, J=6.9 Hz, 1H), 7.18 (t, J=7.7 Hz, 2H), 6.81 (t, J=7.3 Hz, 1H), 6.72 (d, J=7.9 Hz, 2H). ¹³C NMR (126 MHz, DMSO-d₆) δ 167.12, 144.57, 139.45, 132.60, 131.68, 130.32 (3C), 130.05 (2C), 129.91, 127.35 (2C), 125.31, 121.75, 120.11, 116.72, 114.20, 114.12 (2C). ESI-MS m/z: 330.1 (MH⁺). HRMS calcd. for C₂₀H₁₆N₃O₂ (MH⁺), 330.1237; found, 330.1233.

### 2-(2-Carboxyphenyl)-3-((4-carboxyphenyl)amino)imidazo[1,2-a]pyridine-6-carboxylic acid (Compound 8j)

Treatment of methyl 6-(4-(methoxycarbonyl)phenyl)-5-oxo-5,6-dihydropyrido[2′,1′:2,3]imidazo[4,5-c]isoquinoline-9-carboxylate (6j) as outlined in Synthesis 3 of SE3 above (65° C., 18 h) and purification by preparative HPLC (linear gradient of 5% B to 30% B over 20 min with a flow rate 20 mL/min; retention time=14.7 min) provided 4-((2-(4-sulfophenyl)imidazo[1,2-a]pyridin-3-yl)amino)benzoic acid as white solid (52% yield). ¹H NMR (500 MHz, DMSO-d₆) δ 8.86 (s, 1H), 8.59 (s, 1H), 7.89 (d, J=9.5 Hz, 1H), 7.82 (t, J=7.9 Hz, 2H), 7.69 (d, J=8.9 Hz, 2H), 7.60-7.48 (m, 3H), 6.68 (d, J=8.3 Hz, 2H). ¹³C NMR (126 MHz, DMSO-d₆) δ 169.07, 167.51, 165.79, 149.64, 141.18, 138.22, 133.21, 131.73 (2C), 131.51, 131.11, 130.82, 130.13, 129.43, 127.20, 126.92, 121.53, 120.38, 118.30, 116.23, 113.26 (2C). HRMS calcd. for C₂₀H₁₆N₃O₆ (MH⁺), 418.1034; found, 418.1033.

### 2-Phenyl-3-(phenylamino)imidazo[1,2-a]pyridine-6-carboxylic acid (Compound 8k)

Treatment of methyl 2-phenyl-3-(phenylamino)imidazo[1,2-a]pyridine-6-carboxylate (6k) as outlined in Synthesis 3 of SE3 above (rt, 24 h) and purification by preparative HPLC provided 2-phenyl-3-(phenylamino)imidazo[1,2-a]pyridine-6-carboxylic acid as a pale green solid (83% yield). ¹H NMR (500 MHz, DMSO-d₆) δ 13.39 (brs, 1H), 8.52 (s, 1H), 8.38 (s, 1H), 8.06 (d, J=7.3 Hz, 2H), 7.76-7.72 (m, 2H), 7.43 (t, J=7.7 Hz, 2H), 7.33 (t, J=7.3 Hz, 1H), 7.16 (t, J=7.7 Hz, 2H), 6.77 (t, J=7.3 Hz, 1H), 6.58 (d, J=7.9 Hz, 2H). ¹³C NMR (126 MHz, DMSO-d₆) δ 166.13, 145.54, 142.50, 138.82, 133.05, 130.12 (2C), 129.07 (2C), 128.59, 127.06 (2C), 126.86, 125.47, 120.51, 119.46, 117.17, 117.01, 113.55 (2C). ESI-MS m/z: 330.1 (MH⁺). HRMS calcd. for C₂₀H₁₆N₃O₂ (MH⁺), 330.1237; found, 330.1231.

### 3-((4-Nitrophenyl)amino)-2-phenylimidazo[1,2-a]pyridine-6-carboxylic acid (Compound 81)

Treatment of methyl 3-((4-nitrophenyl)amino)-2-phenylimidazo[1,2-a]pyridine-6-carboxylate (61) as outlined in Synthesis 3 of SE3 above (rt, 24 h) and purification by preparative HPLC (linear gradient of 10% B to 50% B over 20 min with a flow rate 20 mL/min; retention time=12.1 min) provided 3-((4-nitrophenyl)amino)-2-phenylimidazo[1,2-a]pyridine-6-carboxylic acid as a brown solid (40% yield). ¹H NMR (400 MHz, DMSO-d₆) δ 13.43 (brs, 1H), 9.44 (s, 1H), 8.51 (t, J=1.4 Hz, 1H), 8.08 (d, J=8.9 Hz, 2H), 8.01-7.98 (m, 2H), 7.76 (s, 2H), 7.43 (t, J=7.5 Hz, 2H), 7.36-7.32 (m, 1H), 6.73 (brs, 2H). ¹³C NMR (126 MHz, DMSO-d₆) δ 166.16, 152.15, 143.18, 139.66, 133.07, 129.21 (2C), 128.77, 127.02 (2C), 126.89 (2C), 126.87, 126.53, 125.55, 118.18, 117.78, 117.36, 112.83 (2C). ESI-MS m/z: 375.1 (MH⁺). HRMS calcd. for C₂₀H₁₅N₄O₄ (MH⁺), 375.1088; found, 375.1083.

### 3-((4-Carboxyphenyl)amino)-2-phenylimidazo[1,2-a]pyridine-6-carboxylic acid (Compound 8m)

Treatment of methyl 3-((4-(methoxycarbonyl)phenyl)amino)-2-phenylimidazo[1,2-a]pyridine-6-carboxylate (6m) as outlined in Synthesis 3 of SE3 above (65° C., 24 h) and purification by preparative HPLC (linear gradient of 5% B to 50% B over 20 min with a flow rate 20 mL/min; retention time=10.1 min) provided 3-((4-carboxyphenyl)amino)-2-phenylimidazo[1,2-a]pyridine-6-carboxylic acid as a dark brown solid (87% yield). ¹H NMR (400 MHz, DMSO-d₆) δ 8.93 (s, 1H), 8.49 (t, J=1.4 Hz, 1H), 8.02-8.00 (m, 2H), 7.79-7.73 (m, 4H), 7.43 (t, J=7.6 Hz, 2H), 7.36-7.31 (m, 1H), 6.63 (d, J=8.0 Hz, 2H). ¹³C NMR (126 MHz, DMSO-d₆) δ 167.53, 166.05, 149.71, 142.71, 138.94, 132.78, 132.10 (2C), 129.17 (2C), 128.78, 127.05 (2C), 126.72, 125.75, 121.58, 119.33, 117.45, 117.10, 113.11 (2C). ESI-MS m/z: 374.2 (MH⁺). HRMS calcd. for C₂₁H₁₆N₃O₄ (MH⁺), 374.1135; found, 374.1128.

### 2-([1,1′-Biphenyl]-4-yl)-3-((4-carboxyphenyl)amino)imidazo[1,2-a]pyridine-6-carboxylic acid (Compound 8n)

Treatment of methyl 6-aminonicotinate (4d), (1,1′-biphenyl)-4-carbaldehyde (5j), and methyl 4-isocyanobenzoate (3a) as outlined in Synthesis 1 of SE3 above (MeOH, Biotage Initiator, microwave irradiation, 85° C., 3 h) provided methyl 2-([1,1′-biphenyl]-4-yl)-3-((4-(methoxycarbonyl)phenyl)amino)imidazo[1,2-a]pyridine-6-carboxylate as a yellow solid (40% yield). ESI-MS m/z: 478.2 (MH⁺). Treatment of methyl 2-([1,1′-biphenyl]-4-yl)-3-((4-(methoxycarbonyl)phenyl)amino)imidazo[1,2-a]pyridine-6-carboxylate as outlined in Synthesis 3 of SE3 above (65° C., 18 h) and purification by preparative HPLC (linear gradient of 10% B to 50% B over 20 min with a flow rate 20 mL/min; retention time=17.1 min) provided 2-([1,1′-biphenyl]-4-yl)-3-((4-carboxyphenyl)amino)imidazo[1,2-a]pyridine-6-carboxylic acid as a brown solid (56% yield). ESI-MS m/z: 450.2 (MH⁺). HRMS calcd. for C₂₇H₂₀N₃O₄ (MH⁺), 450.1448; found, 450.1445.

### 4-((6-Bromo-2-phenylimidazo[1,2-a]pyridin-3-yl)amino)benzoic acid (Compound 8o)

Treatment of methyl 4-((6-bromo-2-phenylimidazo[1,2-a]pyridin-3-yl)amino)benzoate (6n) as outlined in Synthesis 3 of SE3 above (65° C., 18 h) and purification by preparative HPLC (linear gradient of 10% B to 60% B over 20 min with a flow rate 20 mL/min; retention time=11.8 min) provided 4-((6-bromo-2-phenylimidazo[1,2-a]pyridin-3-yl)amino)benzoic acid as light brown solid (60% yield). ¹H NMR (500 MHz, DMSO-d₆) δ 8.86 (s, 1H), 8.30 (s, 1H), 8.01-7.94 (m, 2H), 7.75 (d, J=9.1 Hz, 2H), 7.70 (d, J=9.4 Hz, 1H), 7.54 (d, J=9.3 Hz, 1H), 7.42 (t, J=7.7 Hz, 2H), 7.33 (t, J=7.4 Hz, 1H), 6.60 (d, J=8.7 Hz, 2H). ¹³C NMR (126 MHz, DMSO-d₆) δ 167.60, 149.72, 140.50, 137.75, 132.55, 131.98 (2C), 129.78, 129.16 (2C), 128.74, 127.00 (2C), 123.62, 121.46, 118.98, 118.43, 113.18 (2C), 107.66. HRMS calcd. for C₂₀H₁₅BrN₃O₂ [M(⁷⁹Br)H⁺], 408.0342; found, 408.0338.

### 4-((2,7-Diphenylimidazo[1,2-a]pyridin-3-yl)amino)benzoic acid (Compound 8p; XZ664)

Treatment of methyl 4-((2,7-diphenylimidazo[1,2-a]pyridin-3-yl)amino)benzoate (6o) as outlined in Synthesis 3 of SE3 above (65° C., 18 h) and purification by preparative HPLC (linear gradient of 10% B to 70% B over 20 min with a flow rate 20 mL/min; retention time=13.4 min) provided 4-((2,7-diphenylimidazo[1,2-a]pyridin-3-yl)amino)benzoic acid as a white solid (65% yield). ¹H NMR (500 MHz, DMSO-d₆) δ 9.04 (s, 1H), 8.21 (d, J=7.1 Hz, 1H), 8.04 (d, J=1.6 Hz, 1H), 7.93-7.88 (m, 2H), 7.86-7.80 (m, 2H), 7.71 (d, J=9.0 Hz, 2H), 7.57-7.53 (m, 1H), 7.51 (t, J=7.5 Hz, 2H), 7.47-7.41 (m, 3H), 7.38-7.33 (m, 1H), 6.67 (d, J=8.2 Hz, 2H). ¹³C NMR (126 MHz, DMSO-d₆) δ 167.57, 149.54, 141.64, 140.83, 137.21, 134.24, 132.01 (2C), 130.06, 129.84 (2C), 129.81, 129.61, 129.47 (2C), 127.51 (2C), 127.21 (2C), 124.86, 121.85, 119.08, 114.73, 113.43, 113.40, 111.34. HRMS calcd. for C₂₆H₂₀N₃O₂ (MH⁺), 406.1550; found, 406.1541.

### 4-((6-(Methylsulfonyl)-2-phenylimidazo[1,2-a]pyridin-3-yl)amino)benzoic acid (Compound 8q)

Treatment of methyl 4-((6-(methylsulfonyl)-2-phenylimidazo[1,2-a]pyridin-3-yl)amino)benzoate (6p) as outlined in Synthesis 3 of SE3 above (80° C., 24 h) and purification by preparative HPLC (linear gradient of 10% B to 30% B over 20 min with a flow rate 20 mL/min; retention time=15.7 min) provided 4-((6-(methylsulfonyl)-2-phenylimidazo[1,2-a]pyridin-3-yl)amino)benzoic acid as a brown solid (11% yield). ¹H NMR (500 MHz, DMSO-d₆) δ 8.89 (s, 1H), 8.43-8.39 (m, 1H), 7.95 (d, J=7.1 Hz, 2H), 7.82 (d, J=9.4 Hz, 1H), 7.73-7.65 (m, 3H), 7.36 (t, J=7.7 Hz, 2H), 7.27 (t, J=7.4 Hz, 1H), 6.57 (d, J=8.2 Hz, 2H), 3.26 (s, 3H). ¹³C NMR (126 MHz, DMSO-d₆) δ 167.56, 149.52, 142.21, 139.88, 132.86, 132.06 (2C), 129.18 (2C), 128.87, 127.59, 127.11 (2C), 125.51, 122.64, 121.63, 120.09, 118.36, 113.20 (2C), 44.01. ESI-MS m/z: 408.1 (MH⁺). HRMS calcd. for C₂₁H₁₈N₃O₄S (MH⁺), 408.1013; found, 408.1015.

### 4-((2-([1,1′-Biphenyl]-4-yl)-6-(methylsulfonyl)imidazo[1,2-a]pyridin-3-yl)amino)benzoic acid (Compound 8r)

Treatment of methyl 4-((2-([1,1′-biphenyl]-4-yl)-6-(methylsulfonyl)imidazo[1,2-a]pyridin-3-yl)amino)benzoate (6q) as outlined in Synthesis 3 of SE3 above (65° C., 18 h) and purification by preparative HPLC (linear gradient of 20% B to 60% B over 20 min with a flow rate 20 mL/min; retention time=15.6 min) provided 4-((2-([1,1′-biphenyl]-4-yl)-6-(methylsulfonyl)imidazo[1,2-a]pyridin-3-yl)amino)benzoic acid as a brown solid (40% yield). ¹H NMR (500 MHz, DMSO-d₆) δ 8.94 (s, 1H), 8.46-8.40 (m, 1H), 8.05 (d, J=8.5 Hz, 2H), 7.84 (d, J=9.4 Hz, 1H), 7.75-7.66 (m, 4H), 7.64 (d, J=7.3 Hz, 2H), 7.39 (t, J=7.6 Hz, 2H), 7.30 (t, J=7.3 Hz, 1H), 6.61 (brs, 2H), 3.27 (s, 3H). ¹³C NMR (126 MHz, DMSO-d₆) δ 167.54, 149.51, 142.30, 140.32, 139.84, 139.61, 132.09 (2C), 131.95, 129.42 (2C), 128.12, 127.62 (2C), 127.57, 127.35 (2C), 127.02 (2C), 125.49, 122.65, 121.67, 120.16, 118.36, 113.23 (2C), 44.02. ESI-MS m/z: 484.1 (MH⁺). HRMS calc'd. for C₂₇H₂₂N₃O₄S (MH⁺), 484.1326; found, 484.1321.

### 3-((4-Carboxyphenyl)amino)-2-phenylimidazo[1,2-a]pyridine-7-carboxylic acid (Compound 8s)

Treatment of methyl 3-((4-(methoxycarbonyl)phenyl)amino)-2-phenylimidazo[1,2-a]pyridine-7-carboxylate (6r) as outlined in Synthesis 3 of SE3 above (rt, 24 h) and purification by preparative HPLC (linear gradient of 10% B to 50% B over 20 min with a flow rate 20 mL/min; retention time=9.4 min) provided 3-((4-carboxyphenyl)amino)-2-phenylimidazo[1,2-a]pyridine-7-carboxylic acid as a dark pale yellow solid (95% yield). ¹H NMR (500 MHz, DMSO-d₆) δ 8.97 (s, 1H), 8.17 (s, 1H), 8.04 (t, J=7.3 Hz, 3H), 7.75 (d, J=8.5 Hz, 2H), 7.42 (t, J=7.6 Hz, 2H), 7.37 (dd, J=7.1, 1.6 Hz, 1H), 7.33 (t, J=7.4 Hz, 1H), 6.58 (brs, 2H). ¹³C NMR (126 MHz, DMSO-d₆) δ 167.58, 166.66, 149.77, 141.43, 140.38, 133.39, 132.06 (2C), 129.10 (2C), 128.59, 127.95, 127.05 (2C), 123.50, 121.34, 119.78, 119.50, 113.02 (2C), 112.12. ESI-MS m/z: 374.1 (MH⁺). HRMS calcd. for C₂₁H₁₆N₃O₄ (MH⁺), 374.1135; found, 374.1125.

### SE6: Synthesis of Methyl Benzoates

### General Procedure for the Preparation of Methyl Benzoates (Compounds 12a, b)

Sodium hydride (15 mmol) was suspended in THF (15 mL). Commercial available 2-Phenyl-1H-benzo[d]imidazole (11a, 12 mmol) or 2-phenyl-1H-indole (11b, 12 mmol) and methyl 4-(bromomethyl)benzoate (12 mmol) were added at 0° C. The reaction mixture was stirred (rt, 18 h). The mixture was purified by silica gel column and the methyl benzoates (12a or 12b) were afforded.

### Methyl 4-((2-phenyl-1H-benzo[d]imidazol-1-yl)methyl)benzoate (Compound 12a)

Treatment of commercial available 2-phenyl-1H-benzo[d]imidazole (11a) and methyl 4-(bromomethyl)benzoate as outlined in the general procedure above provided methyl 4-((2-phenyl-1H-benzo[d]imidazol-1-yl)methyl)benzoate (12a) as a white solid (77% yield). ¹H NMR (400 MHz, CDCl₃) δ 8.02 (d, J=8.4 Hz, 2H), 7.90 (d, J=8.0 Hz, 1H), 7.66 (dd, J=8.0, 1.7 Hz, 2H), 7.48-7.42 (m, 3H), 7.36-7.32 (m, 1H), 7.28-7.24 (m, 1H), 7.20-7.17 (m, 3H), 5.50 (s, 2H), 3.91 (s, 3H). ¹³C NMR (101 MHz, CDCl₃) δ 166.54, 154.16, 143.12, 141.44, 135.87, 130.39 (2C), 130.10, 129.86, 129.80, 129.18 (2C), 128.86 (2C), 126.01 (2C), 123.30, 122.96, 120.09, 110.33, 52.23, 48.22.

### Methyl 4-((2-phenyl-1H-indol-1-yl)methyl)benzoate (Compound 12b)

Treatment of commercial available 2-phenyl-1H-indole (11b) and methyl 4-(bromomethyl)benzoate as outlined in the general procedure above provided methyl 4-((2-phenyl-1H-indol-1-yl)methyl)benzoate (12b) as a white solid (47% yield). ¹H NMR (400 MHz, CDCl₃) δ 8.20 (s, 1H), 7.95 (d, J=8.4 Hz, 2H), 7.51 (dd, J=8.4, 1.4 Hz, 2H), 7.45 (t, J=7.2 Hz, 3H), 7.42-7.37 (m, 2H), 7.32 (d, J=8.6 Hz, 2H), 7.24 (ddd, J=8.2, 7.1, 1.2 Hz, 1H), 7.10 (ddd, J=8.0, 7.0, 1.0 Hz, 1H), 4.34 (s, 2H), 3.91 (s, 3H). ¹³C NMR (101 MHz, CDCl₃) δ 167.32, 147.22, 136.08, 135.76, 132.78, 129.83 (2C), 129.32, 128.96 (2C), 128.30 (2C), 127.92, 127.90 (2C), 127.86, 122.54, 119.94, 119.39, 110.98, 110.19, 52.03, 30.66.

### 4-((2-Phenyl-1H-benzo[d]imidazol-1-yl)methyl)benzoic acid (Compound 9a)

Treatment of methyl 4-((2-phenyl-1H-benzo[d]imidazol-1-yl)methyl)benzoate (12a) as outlined in Synthesis 3 of SE3 above and purification by preparative HPLC (linear gradient of 20% B to 80% B over 20 min with a flow rate 20 mL/min; retention time=6.5 min) provided 4-((2-phenyl-1H-benzo[d]imidazol-1-yl)methyl)benzoic acid (9a) as a white solid (97% yield). ¹H NMR (400 MHz, DMSO-d₆) δ 7.91-7.85 (m, 3H), 7.83-7.78 (m, 2H), 7.70-7.59 (m, 4H), 7.52-7.42 (m, 2H), 7.23 (d, J=8.3 Hz, 2H), 5.78 (s, 2H). ¹³C NMR (101 MHz, DMSO-d₆) δ 167.32, 152.48, 141.01, 134.60, 132.02, 130.78, 130.30 (2C), 129.99 (2C), 129.63 (2C), 127.03 (2C), 126.55, 125.15, 117.90, 117.43, 114.98, 112.81, 48.36. ESI-MS m/z: 329.1 (MH⁺). HRMS calcd. for C₂₁H₁₇N₂O₂ (MH⁺), 329.1285; found, 329.1277.

### 4-((2-Phenyl-1H-indol-1-yl)methyl)benzoic acid (Compound 9b)

Treatment of methyl 4-((2-phenyl-1H-indol-1-yl)methyl)benzoate (12b) as outlined in Synthesis 3 of SE3 above and purification by preparative HPLC (linear gradient of 20% B to 80% B over 20 min with a flow rate 20 mL/min; retention time=15.5 min) provided 4-((2-phenyl-1H-indol-1-yl)methyl)benzoic acid (9b) as a white solid (12% yield). ¹H NMR (500 MHz, DMSO-d₆) δ 12.78 (brs, 1H), 11.37 (s, 1H), 7.83 (d, J=9.1 Hz, 2H), 7.59 (d, J=7.5 Hz, 2H), 7.48 (s, 2H), 7.42-7.31 (m, 3H), 7.28 (d, J=8.7 Hz, 2H), 7.18-7.05 (m, 1H), 6.96 (t, J=7.7 Hz, 1H), 4.31 (s, 2H). ¹³C NMR (126 MHz, DMSO-d₆) δ 167.72, 147.43, 136.59, 135.63, 133.07, 129.95 (2C), 129.29 (2C), 129.22, 128.86, 128.57 (2C), 128.07 (2C), 128.00, 122.19, 119.44, 119.13, 111.75, 109.44, 30.48. ESI-MS m/z: 328.1 (MH⁺). HRMS calcd. for C₂₂H₁₈NO₂ (MH⁺), 328.1332; found, 328.1326.

### 4-((2-Phenylimidazo[1,2-a]pyrazin-3-yl)amino)phthalic acid (Compound 10a)

Treatment of dimethyl 4-((2-phenylimidazo[1,2-a]pyrazin-3-yl)amino)phthalate (6s) as outlined in Synthesis 3 of SE3 above (70° C., 24 h) and purification by preparative HPLC (linear gradient of 10% B to 20% B over 20 min with a flow rate 20 mL/min; retention time=16.9 min) provided 4-((2-phenylimidazo[1,2-a]pyrazin-3-yl)amino)phthalic acid as a yellow solid (92% yield). ¹H NMR (400 MHz, DMSO-d₆) δ 12.86 (brs, 2H), 9.15 (d, J=1.5 Hz, 1H), 9.05 (s, 1H), 8.10 (dd, J=4.5, 1.5 Hz, 1H), 8.05 (d, J=7.3 Hz, 2H), 7.93 (d, J=4.5 Hz, 1H), 7.62 (d, J=8.5 Hz, 1H), 7.46 (t, J=7.6 Hz, 2H), 7.37 (t, J=7.3 Hz, 1H), 6.74 (brs, 1H), 6.52 (brs, 1H). ¹³C NMR (126 MHz, DMSO-d₆) δ 170.00, 167.65, 148.28, 143.59, 140.23, 137.94, 137.91, 132.99, 132.22, 129.95, 129.21 (2C), 128.95, 127.17 (2C), 120.90, 119.35, 116.94, 113.61, 112.75. ESI-MS m/z: 375.1 (MH⁺). HRMS calcd. for C₂₀H₁₄N₄O₄ (MH⁺), 375.1088; found, 375.1087.

### 4-((2-Phenylimidazo[1,2-a]pyridin-3-yl)amino)phthalic acid (Compound 10b)

Treatment of pyridin-2-amine (4b), benzaldehyde (5a), and dimethyl 4-isocyanophthalate (3d) as outlined in Synthesis 2 of SE3 above provided dimethyl 4-((2-phenylimidazo[1,2-a]pyridin-3-yl)amino)phthalate as a green solid (3.1% yield). ESI-MS m/z: 402.2 (MH⁺). Treatment of dimethyl 4-((2-phenylimidazo[1,2-a]pyridin-3-yl)amino)phthalate as outlined in Synthesis 3 of SE3 above (rt, 24 h) and purification by preparative HPLC (linear gradient of 5% B to 20% B over 20 min with a flow rate 20 mL/min; retention time=16.5 min) provided 4-((2-phenylimidazo[1,2-a]pyridin-3-yl)amino)phthalic acid as a yellow solid (55% yield, two steps). ¹H NMR (500 MHz, DMSO-d₆) δ 8.85 (s, 1H), 8.02 (d, J=7.0 Hz, 2H), 7.99 (d, J=6.8 Hz, 1H), 7.75-7.69 (m, 1H), 7.66 (d, J=9.0 Hz, 1H), 7.42 (t, J=7.7 Hz, 2H), 7.35 (ddd, J=8.8, 6.7, 1.3 Hz, 1H), 7.30 (t, J=7.4 Hz, 1H), 6.95 (t, J=6.7 Hz, 1H), 6.67 (brs, 1H), 6.51 (brs, 1H). ¹³C NMR (126 MHz, DMSO-d₆) δ 193.73, 169.73, 167.84, 148.73, 142.46, 138.07, 138.05, 135.07, 133.81, 129.96, 129.64, 129.05 (2C), 128.21, 126.87 (2C), 126.00, 123.57, 117.95, 117.67, 113.10. ESI-MS m/z: 374.1 (MH⁺). HRMS calcd. for C₂₁H₁₆N₃O₄ (MH⁺), 374.1135; found, 374.1133.

### SE7: Synthetic Scheme for Aminoxyl-Substituted Analogue 105

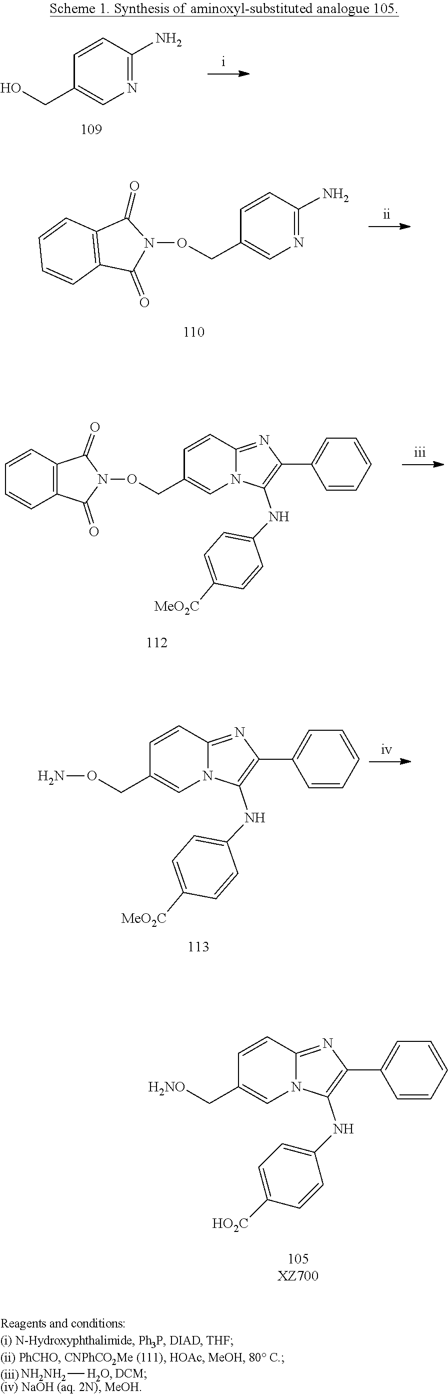

Aminoxyl-substituted analogue 105 was prepared starting from a commercially available (6-aminopyridin-3-yl)methanol 109. (Scheme 1) First, the hydroxyl in 109 was replaced by hydroxyphthalimide to afforded 110 using Mitsunobu Reaction. Using a GBBR multicomponent reactions the phthalimide protected 110 reacted with benzylaldehyde and methyl 4-isocyanobenzoate 111, which was prepared from methyl 4-aminobenzoate as previously reported, to afford imidazo[1,2-a]pyridin-3-amine 112. Deprotection of the phthalimide by hydrazine in 112 and hydrolysis of the formed ester 113 afforded aminoxyl-labelled N,2-diphenylimidazo[1,2-a]pyridine 105 in four steps. After HPLC purification, aminoxyl-labelled N,2-diphenylimidazo[1,2-a]pyridine 105 was used as starting material to prepare the oximes library 105-X targeting TDP1 DNA binding region.

### SE8: Synthetic Scheme for Aminoxyl-Substituted Analogue 106

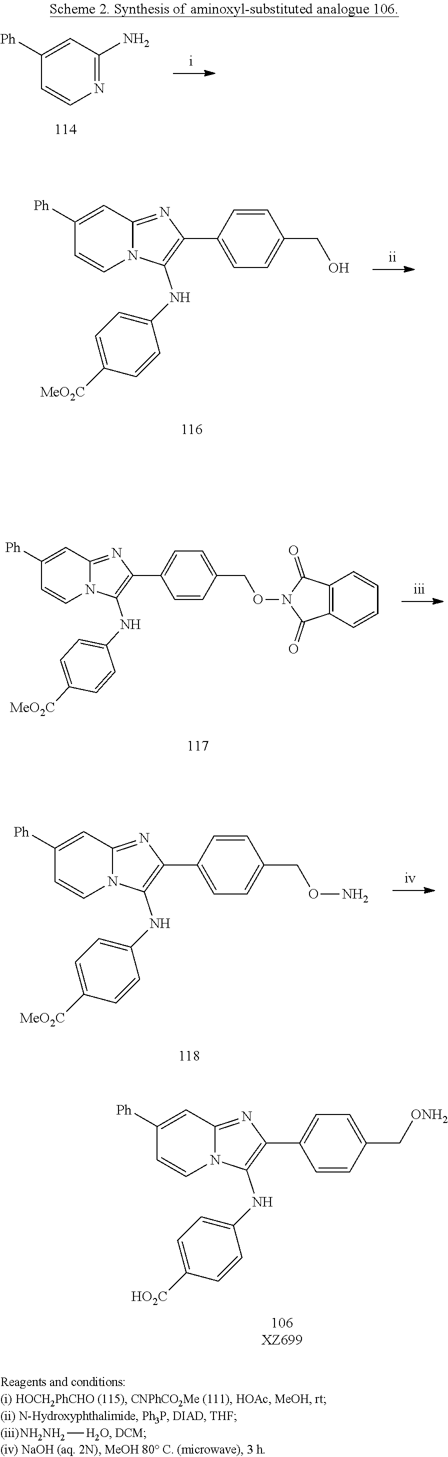

Directly using GBBR multicomponent reaction, hydroxylmethyl-labelled analogue 116 was afforded from a commercially available 4-phenylpyridin-2-amine 114, 4-(hydroxymethyl)benzaldehyde 115 and methyl 4-isocyanobenzoate 111. (Scheme 2) Following by Mitsunobu Reaction with N-hydroxyphthalimide, the hydroxyl in 116 was replaced by hydroxyphthalimide to afforded 117. Deprotection of the phthalimide protected 117 by hydrazine and hydrolysis of the formed ester 118 by microwave-heating afforded aminoxyl-labelled N,2-diphenylimidazo[1,2-a]pyridine 106 in 4 steps successfully. After HPLC purification, aminoxyl-labelled N,2-diphenylimidazo[1,2-a]pyridine 106 was used as starting material to prepare the oximes library 106-Y targeting TOP1 peptide binding region.

### SE9: Synthetic Scheme for triazole analogues 107a-107f

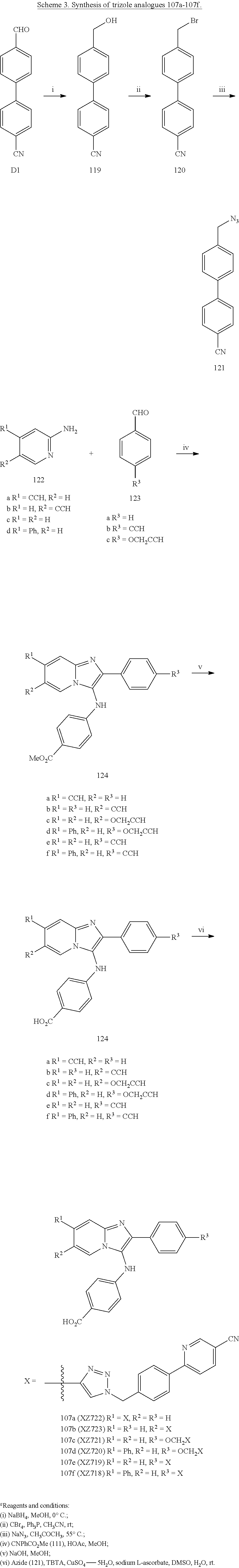

A series of triazole linked analogues 107a-107f have been prepared based on the oxime lead compound oxime (E)-6-D1 using Copper catalysis alkyne-azide [3+2]cycloaddition reaction (CuAAC). (Scheme 3) Azide (121) was prepared from aldehyde D1 by reduction of aldehyde D1 using sodium borohydride, replacement of the hydroxyl in the alcohol 119 using tetrabromide carbon and introduce of azide using sodium azide to the bromide 20. Akyne-labelled 124a-124f were prepared using GBBR reaction of 2-amino-pyridine 122, aldehyde 123 and methyl 4-isocyanobenzoate 111. Deprotection of 124a-124f afford the acids 125a-125f. Cycloaddition of alkynes in 125a-125f with azide 121 using CuAAC, 107a-107f with triazole linker were successfully afforded separately.

### SE10: Synthetic Scheme for TDP1 Inhibitors 108a and 108b

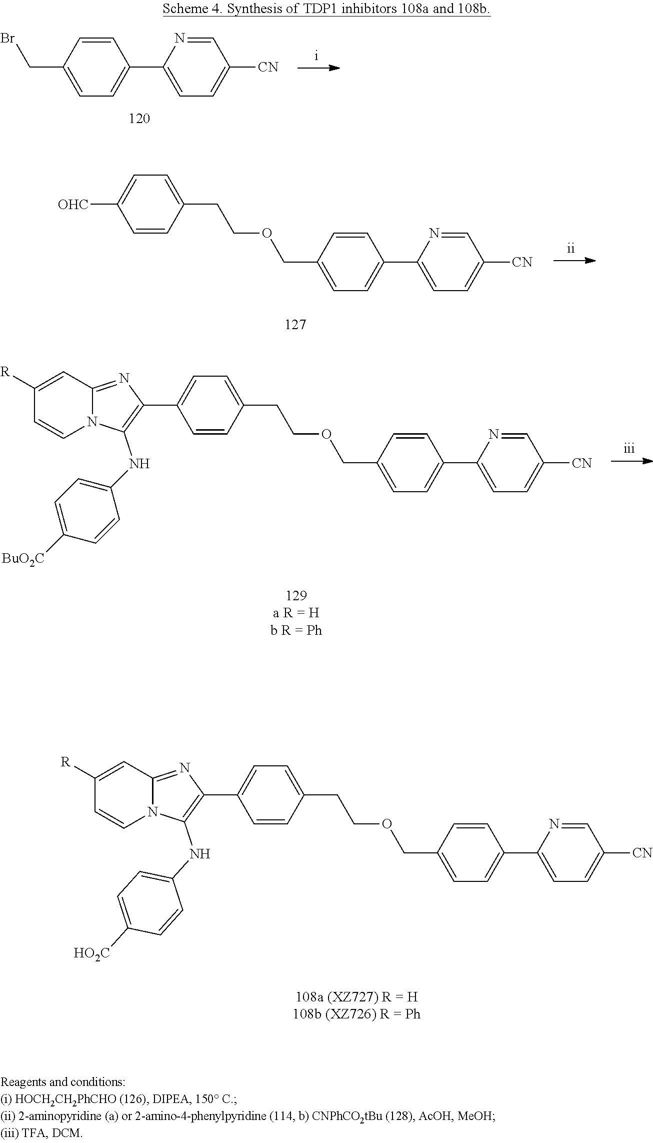

In order to compare with more flexible linkers with oxime (E)-6-D1, we designed and prepared analogues 108a, b with the flexible ether linkers. (Scheme 4) The synthesis of 108a, b were starting from bromide 120. Heating bromide 120 with 2-hydroxylethylbenzylaldehyde 126 and DIEA at 150° C., aldehyde 127 was afforded. Firstly, we coupled aldehyde 127 with 2-amino-4-phenyl-pyridine 114 and methyl 4-isocyanobenzoate 111, and prepared a methyl ester. However, when we deprotected methyl ester using sodium hydroxide and acidified the result mixture by aqueous HCl, there was trace amount of 108b formed. During HPLC purification, we found that the 4-cyane has been hydrolyzed to 4-acid as a major product. To optimize the synthesis procedure, we freshly prepared tert-butyl 4-isocyanobenzoate 128 from tert-butyl 4-aminobenzoate. We coupled aldehyde 127 with 2-amino-4-phenyl-pyridine 114 and tert-butyl 4-isocyanobenzoate 128 using GBBR reaction, and prepared tert-butyl ester 129b. Deprotection of 129b using TFA, 108b was successfully afforded. Using the similar procedure, we prepared 108a from its precursor tert-butyl ester 129a.

### SE11: Mitsunobu Reaction to Prepare Phthalimide Protected Compounds (110 and 117)

Diisopropyl (E)-diazene-1,2-dicarboxylate (DIAD, 7 mmol) was added dropwise to the mixture of alcohols (109 or 116, 6 mmol), 2-hydroxyisoindoline-1,3-dione (6.5 mmol) and triphenylphosphane (7 mmol) in THF (20 mL) at 0° C. The reaction mixture was stirred (rt, 18 h) and concentrated. The residue was stirred with MeOH (5 mL) (rt, 1 h). The suspension was filtered and washed by MeOH (10 mL). The solid was collected to afford the phthalimide protected products (110 or 117) separately.

### SE12: Groebke-Blackburn-Bienayme (GBBR) multicomponent reactions to prepare imidazo[1,2-a]pyrazines and Imidazo[1,2-a]pyridines (112, 116, 124a-124f, 129a, and 129b)

Pyridine-2-amines (110, 114, 123 or 127, 6 mmol), aldehydes (19, 115, 123, or 127, 6 mmol), and acetic acid (12 mmol) were mixed in MeOH (5 mL) and THF (5 mL) (rt, 20 min). Isonitrile (111 or 128, 6 mmol) was added. The reaction solution was stirred (80° C., 4 h/rt, 24 h). The final suspension was filtered and washed by hexanes and water. The solid product was collected to provide final imidazo[1,2-a]pyridines (112, 116, 124a-124f, 129a, or 129b) separately.

### SE13: Deprotection of Phthalimide to Prepare Aminoxyl Compounds (113 and 118)

Phthalimide protected compounds (111 or 116, 1 mmol) was dissolved in DCM (100 mL). Hydrazine hydrate (5 mmol) was added. The suspension was stirred (rt, 5 h). The suspension was filtered and washed by DCM. The filtrate was concentrated. The residue was collected to afford aminooxyl compounds (113 or 118) separately.

### SE14: Deprotection of Methyl Ester to Prepare Carboxylic Acids (105 and 106)

Methyl esters (113 or 117, 1 mmol) was suspended in MeOH (4 mL) in a microwave tube. NaOH (4 mL, aq. 2M) was added. The suspension in the sealed tube was microwave-heated (100° C., 4 h). The reaction mixture was cooled to rt and acidified by HCl (aq. 2N). The formed suspension was filtered and washed by water and hexanes. The solid was collected to afford the carboxylic acids (105 or 106) separately after HPLC purification.

### SE15: Reaction of Aminoxyls and Aldehydes to Prepare (Z)- and (E)-Isomers of Oximes (105-D1, 105-P3, 106-D1, 106-E6, 106-B7, 106-P3, 106-M10)

Aminoxyls (105 or 106, 0.2 mmol) and lead aldehydes (B7, D1, E6, P3 or M10, 0.2 mmol) was mixed in DMSO (1 mL). Acetic acid (1 mmol) was added. The reaction mixture was stirred (rt, 18 h). The formed suspension was filtered and washed by MeOH. The white solid was collected to afforded oximes (105-X or 106-Y), which were purified by HPLC to afforded (Z)- and (E)-isomers of oximes (105-D1, 105-P3, 106-DI, 106-E6, 106-B7, 106-P3, 106-M10) separately.

### SE16: Cu(I)-catalyzed azide-alkyne cycloaddition (CuAAC) 108 to prepare triazole compounds (107a-107f)

Alkynes (125a-125f, 0.1 mmol, 1 mg in 10 μL DMSO), azides (0.1 mmol, 1 mg in 10 μL DMSO) and tris((1-benzyl-1H-1,2,3-triazol-4-yl)methyl)amine (TBTA, 0.04 mmol, 1 mg in 10 μL DMSO) were mixed in a vial with a stirrer bar. Sodium ascorbate (0.1 mmol, 1 mg in 10 μL H₂O) and CuSO₄-5H₂O (0.02 mmol, 1 mg in 10 μL water) were added. The reaction was diluted in DMSO (2 mL). The formed bright yellow solution was stirred at rt overnight under Argon. A yellow suspension was formed. The reaction mixture was dissolved in DMSO and purified by HPLC to afford triazole compounds (107a-107f) separately.

### SE17: Deprotection of Tert-Butyl Protection to Prepare Acids (108a and 108b) Using TFA

tert-Butyl ester (129a or 129b, 0.06 mmol) was mixed with the cocktail of TFA/H₂O/TIS (90/5/5, 0.5 mL). The reaction mixture was stirred (rt, 1.5 h). The final mixture was diluted by MeOH (5 mL) and filtered by a PTFE filter (PHENEX, 0.20 μm pore). The clear solution was purified by preparative HPLC as the describe in general experiments. After lyophilized the correct HPLC fraction, the acids (108a or 108b) were afforded.

### SE18: Synthesis of Oxime Precursors and Oxime TDP1 Inhibitors

### 2-((6-Aminopyridin-3-yl)methoxy)isoindoline-1,3-dione (110)

Treatment of commercially available (6-aminopyridin-3-yl)methanol 109 as outlined in SE11 provided 2-((6-aminopyridin-3-yl)methoxy)isoindoline-1,3-dione (110) as a red solid (54% yield). ¹H NMR (400 MHz, DMSO-d₆) δ 7.94-7.91 (m, 1H), 7.85 (s, 4H), 7.49 (dd, J=8.5, 2.4 Hz, 1H), 6.43 (dd, J=8.5, 0.8 Hz, 1H), 6.14 (s, 2H), 4.97 (s, 2H). ¹³C NMR (101 MHz, DMSO-d₆) δ 163.68, 160.83 (2C), 150.32 (2C), 139.57, 135.25 (2C), 128.95, 123.69 (2C), 117.68, 108.00, 77.57. DUIS-MS: m/z: 519.2 (MH⁺).

### Methyl 4-((6-(((1,3-dioxoisoindolin-2-yl)oxy)methyl)-2-phenylimidazo[1,2-a]pyridin-3-yl)amino)benzoate (112)

Treatment of 2-((6-aminopyridin-3-yl)methoxy)isoindoline-1,3-dione (110), benzaldehyde and methyl 4-isocyanobenzoate (111) as outlined in SE12 (80° C., 4 h) provided methyl 4-((6-(((1,3-dioxoisoindolin-2-yl)oxy)methyl)-2-phenylimidazo[1,2-a]pyridin-3-yl)amino)benzoate (112) as a red solid (38% yield). ¹H NMR (500 MHz, DMSO-d₆) δ 8.89 (s, 1H), 8.24 (s, 1H), 7.98 (d, J=7.7 Hz, 2H), 7.83 (d, J=2.5 Hz, 4H), 7.71 (d, J=9.2 Hz, 1H), 7.67 (d, J=8.5 Hz, 2H), 7.54 (dd, J=9.2, 1.7 Hz, 1H), 7.39 (t, J=7.6 Hz, 2H), 7.30 (t, J=7.4 Hz, 1H), 6.51 (s, 2H), 5.22 (s, 2H), 3.77 (s, 3H). ¹³C NMR (126 MHz, DMSO-d₆) δ 166.47, 163.59 (2C), 150.57, 142.25, 138.52, 135.24 (2C), 133.67, 131.83 (2C), 129.04 (2C), 128.88 (2C), 128.27, 128.09, 126.90 (2C), 124.51, 123.72 (2C), 120.39, 119.87, 118.52, 117.55, 112.90 (2C), 77.02, 51.97. ESI-MS m/z: 519.10 (MH⁺).

### Methyl 4-((6-((aminooxy)methyl)-2-phenylimidazo[1,2-a]pyridin-3-yl)amino)benzoate (113)

Treatment of methyl 4-((6-(((1,3-dioxoisoindolin-2-yl)oxy)methyl)-2-phenylimidazo[1,2-a]pyridin-3-yl)amino)benzoate (112) as outlined in SE13 (rt, 5 h) provided methyl 4-((6-((aminooxy)methyl)-2-phenylimidazo[1,2-a]pyridin-3-yl)amino)benzoate (113) as a yellow solid (95% yield). ¹H NMR (400 MHz, DMSO-d₆) δ 8.91 (s, 1H), 8.00 (d, J=7.7 Hz, 2H), 7.94 (s, 1H), 7.78 (d, J=8.4 Hz, 2H), 7.65 (d, J=9.2 Hz, 1H), 7.40 (t, J=7.5 Hz, 2H), 7.31 (dd, J=12.0, 8.4 Hz, 2H), 6.60 (s, 2H), 6.06 (s, 2H), 4.57 (s, 2H), 3.76 (s, 3H). ¹³C NMR (101 MHz, DMSO-d₆) δ 166.53, 150.66, 142.18, 138.31, 133.88, 131.94 (2C), 129.00 (2C), 128.14, 127.21, 126.85 (2C), 123.50, 122.07, 119.97, 118.15, 117.38, 113.06 (2C), 74.37, 51.98. ESI-MS m/z: 389.10 (MH⁺).

### 4-((6-((Aminooxy)methyl)-2-phenylimidazo[1,2-a]pyridin-3-yl)amino)benzoic acid (105)

Treatment of methyl 4-((6-((aminooxy)methyl)-2-phenylimidazo[1,2-a]pyridin-3-yl)amino)benzoate (113) as outlined in SE14 (pw, 100° C., 4 h) and purification by preparative HPLC (linear gradient of 5% B to 25% B over 20 min with a flow rate 20 mL/min, retention time=16.1 min.) provided 4-((6-((aminooxy)methyl)-2-phenylimidazo[1,2-a]pyridin-3-yl)amino)benzoic acid (5) as a white solid (66% yield). ¹H NMR (500 MHz, DMSO-d₆) δ 8.93 (s, 1H), 8.17 (s, 1H), 7.89 (dd, J=8.4, 1.3 Hz, 2H), 7.77 (d, J=9.3 Hz, 1H), 7.69 (d, J=9.1 Hz, 2H), 7.52-7.48 (m, 1H), 7.38 (t, J=7.7 Hz, 2H), 7.32-7.28 (m, 1H), 6.58 (d, J=8.1 Hz, 2H), 4.94 (s, 2H). ¹³C NMR (126 MHz, DMSO-d₆) δ 167.59, 149.82, 141.08, 136.12, 132.03 (2C), 131.52, 129.31 (2C), 129.10, 127.08 (2C), 124.55, 121.60, 119.21, 118.04, 116.60, 115.69, 113.18 (2C), 73.44. ESI-MS: m/z: 375.10 (MH⁺). HRMS calcd. for C₂₁H₁₉N₄O₃ (MH⁺): 375.1452; found: 375.1437.

### Methyl 4-((2-(4-(hydroxymethyl)phenyl)-7-phenylimidazo[1,2-a]pyridin-3-yl)amino)benzoate (116)

Treatment of commercially available 4-phenylpyridin-2-amine (114), 4-(hydroxymethyl)benzaldehyde (115) and methyl 4-isocyanobenzoate (111) as outlined in SE12 (rt, 18 h) provided methyl 4-((2-(4-(hydroxymethyl)phenyl)-7-phenylimidazo[1,2-a]pyridin-3-yl)amino)benzoate (116) as a white solid (63% yield). ¹H NMR (500 MHz, DMSO-d₆) δ 8.94 (s, 1H), 8.03 (dd, J=7.2, 0.9 Hz, 1H), 7.99 (d, J=8.3 Hz, 2H), 7.97 (d, J=0.9 Hz, 1H), 7.85 (d, J=7.1 Hz, 2H), 7.79 (d, J=9.1 Hz, 2H), 7.53 (dd, J=8.4, 7.0 Hz, 2H), 7.44 (t, J=7.3 Hz, 1H), 7.34 (t, J=8.6 Hz, 3H), 6.62 (brs, 2H), 5.18 (t, J=5.7 Hz, 1H), 4.50 (d, J=5.6 Hz, 2H), 3.76 (s, 3H). ¹³C NMR (126 MHz, DMSO-d₆) δ 166.52, 150.63, 142.87, 142.63, 138.94, 138.31, 137.32, 132.25, 131.94 (2C), 129.61 (2C), 128.80, 127.09 (4C), 126.65 (2C), 123.57, 119.95, 117.75, 113.96, 113.09 (2C), 112.26, 63.14, 51.99. ESI-MS m/z: 450.2 (MH⁺).

### Methyl 4-((2-(4-(((1,3-dioxoisoindolin-2-yl)oxy)methyl)phenyl)-7-phenylimidazo[1,2-a]pyridin-3-yl)amino)benzoate (117)

Treatment of methyl 4-((2-(4-(hydroxymethyl)phenyl)-7-phenylimidazo[1,2-a]pyridin-3-yl)amino)benzoate (116) as outlined in SE11 provided methyl 4-((2-(4-(((1,3-dioxoisoindolin-2-yl)oxy)methyl)phenyl)-7-phenylimidazo[1,2-a]pyridin-3-yl)amino)benzoate (117) as a yellow solid (79% yield). ¹H NMR (500 MHz, DMSO-d₆) δ 8.98 (s, 1H), 8.07 (d, J=8.0 Hz, 2H), 8.04 (d, J=7.1 Hz, 1H), 7.99 (s, 1H), 7.86 (s, 5H), 7.85 (s, 1H), 7.79 (d, J=8.5 Hz, 2H), 7.56 (d, J=8.2 Hz, 2H), 7.53 (t, J=7.6 Hz, 2H), 7.44 (t, J=7.4 Hz, 1H), 7.35 (dd, J=7.1, 1.8 Hz, 1H), 6.63 (s, 2H), 5.17 (s, 2H), 3.77 (s, 3H). ¹³C NMR (126 MHz, DMSO-d₆) δ 166.51, 163.58 (2C), 150.45, 142.98, 138.28, 138.24, 137.55, 135.27 (2C), 134.52, 134.06, 131.96 (2C), 130.41 (2C), 129.62 (2C), 129.00 (2C), 128.85, 127.11 (2C), 126.80 (2C), 123.73 (2C), 123.68, 120.07, 118.33, 114.07, 113.15 (2C), 112.44, 79.45, 52.00. ESI-MS m/z: 595.2 (MH⁺), 617.1 (MNa⁺).

### Methyl 4-((2-(4-((aminooxy)methyl)phenyl)-7-phenylimidazo[1,2-a]pyridin-3-yl)amino)benzoate (118)

Treatment of methyl 4-((2-(4-(((1,3-dioxoisoindolin-2-yl)oxy)methyl)phenyl)-7-phenylimidazo[1,2-a]pyridin-3-yl)amino)benzoate (117) as outlined in SE13 provided methyl 4-((2-(4-((aminooxy)methyl)phenyl)-7-phenylimidazo[1,2-a]pyridin-3-yl)amino)benzoate (118) as a yellow solid (99% yield). ¹H NMR (500 MHz, DMSO-d₆) δ 9.01 (s, 1H), 8.07 (d, J=8.1 Hz, 3H), 8.03 (s, 1H), 7.89 (d, J=7.1 Hz, 2H), 7.84 (d, J=8.9 Hz, 2H), 7.56 (t, J=7.7 Hz, 2H), 7.47 (t, J=7.4 Hz, 1H), 7.40 (d, J=8.2 Hz, 2H), 7.37 (dd, J=7.1, 1.8 Hz, 1H), 6.68 (brs, 2H), 6.11 (brs, 2H), 4.62 (s, 2H), 3.81 (s, 3H). ¹³C NMR (126 MHz, DMSO-d₆) δ 166.53, 150.61, 142.93, 138.79, 138.41, 138.29, 137.40, 133.01, 131.96 (2C), 129.60 (2C), 128.80, 128.63 (2C), 127.09 (2C), 126.71 (2C), 123.59, 120.01, 117.91, 114.02, 113.11 (2C), 112.31, 77.06, 51.99. DUIS-MS: m/z: 465.2 (MH⁺).

### 4-((2-(4-((Aminooxy)methyl)phenyl)-7-phenylimidazo[1,2-a]pyridin-3-yl)amino)benzoic acid (106)

Treatment of methyl 4-((2-(4-((aminooxy)methyl)phenyl)-7-phenylimidazo[1,2-a]pyridin-3-yl)amino)benzoate (118) as outlined in SE14 and purification by preparative HPLC (linear gradient of 10% B to 30% B over 20 min with a flow rate 20 mL/min; retention time=18.0 min) provided 4-((2-(4-((aminooxy)methyl)phenyl)-7-phenylimidazo[1,2-a]pyridin-3-yl)amino)benzoic acid (106) as a light yellow solid (83% yield). ¹H NMR (500 MHz, DMSO-d₆) δ 8.94 (s, 1H), 8.12 (d, J=7.1 Hz, 1H), 7.99 (d, J=6.5 Hz, 2H), 7.98 (s, 1H), 7.80 (d, J=7.2 Hz, 2H), 7.70 (d, J=9.1 Hz, 2H), 7.49 (t, J=7.6 Hz, 2H), 7.45-7.39 (m, 4H), 6.60 (d, J=7.5 Hz, 2H), 4.89 (s, 2H). ¹³C NMR (126 MHz, DMSO-d₆) δ 167.58, 149.73, 141.85, 137.65, 134.43, 132.03 (2C), 130.01 (2C), 129.75 (2C), 129.41, 127.34 (2C), 127.19 (2C), 124.37, 121.60, 120.41, 118.98, 118.06, 115.70, 113.74, 113.23 (2C), 112.59, 76.04. ESI-MS m/z: 451.10 (MH⁺). HRMS calcd. for C₂₇H₂₃N₄O₃ (MH⁺), 451.1765; found: 451.1753.

### (Z)-4-((6-((((4-(5-Cyanopyridin-2-yl)benzylidene)amino)oxy)methyl)-2-phenylimidazo[1,2-a]pyridin-3-yl)amino)benzoic acid [(Z)-105-D1, XZ717] and (E)-4-((6-((((4-(5-cyanopyridin-2-yl)benzylidene)amino)oxy)methyl)-2-phenylimidazo[1,2-a]pyridin-3-yl)amino)benzoic acid [(E)-105-D1, XZ706]

Treatment of 4-((6-((aminooxy)methyl)-2-phenylimidazo[1,2-a]pyridin-3-yl)amino)benzoic acid (105) and commercially available 6-(4-formylphenyl)nicotinonitrile as outlined in SE15 afforded 4-((6-((((4-(5-cyanopyridin-2-yl)benzylidene)amino)oxy)methyl)-2-phenylimidazo[1,2-a]pyridin-3-yl)amino)benzoic acid (105-D1, white solid, 33% yield) as a mixture of (Z) and (E) isomers with a ratio 5:95 based on LC. Purification by preparative HPLC (linear gradient of 30% B to 40% B over 20 min with a flow rate 20 mL/min) provided the title isomers separately.

(Z)-isomer ((Z)-105-D1) at retention time=12.7 min as a white solid. ESI-MS m/z: 565.2 (MH⁺). HRMS calcd. for C₃₄H₂₅N₆O₃ (MH⁺): 565.1983; found: 565.1966.

(E)-isomer ((E)-105-D1) at retention time=15.5 min as a white solid. ¹H NMR (500 MHz, DMSO-d₆) δ 12.25 (s, 1H), 9.04 (d, J=2.1 Hz, 1H), 8.81 (s, 1H), 8.34 (dd, J=8.4, 2.2 Hz, 1H), 8.29 (s, 1H), 8.15 (d, J=8.4 Hz, 1H), 8.13 (d, J=8.4 Hz, 2H), 8.09 (s, 1H), 7.93 (d, J=7.0 Hz, 2H), 7.69 (d, J=8.9 Hz, 2H), 7.63 (d, J=8.4 Hz, 3H), 7.39 (dd, J=9.3, 1.6 Hz, 1H), 7.33 (t, J=7.7 Hz, 2H), 7.23 (t, J=7.4 Hz, 1H), 6.52 (s, 2H), 5.16 (s, 2H). ¹³C NMR (126 MHz, DMSO-d₆) δ 165.52, 156.54, 150.94, 148.14, 147.35, 139.82, 139.40, 136.46, 136.09, 131.95, 131.44, 129.95 (2C), 126.92 (2C), 126.17, 125.96 (2C), 125.78 (2C), 125.41, 124.75 (2C), 120.88 (2C), 119.06, 118.70, 116.42, 115.60, 115.32, 110.77 (2C), 105.99, 71.16. ESI-MS m/z: 565.2 (MH⁺). HRMS calcd. for C₃₄H₂₅N₆O₃ (MH⁺): 565.1983; found: 565.1980.

### (Z)-4-((6-((((4-((6-Methylpyrazin-2-yl)oxy)benzylidene)amino)oxy)methyl)-2-phenylimidazo[1,2-a]pyridin-3-yl)amino)benzoic acid [(Z)-105-P3, XZ713] and (E)-4-((6-((((4-((6-methylpyrazin-2-yl)oxy)benzylidene)amino)oxy)methyl)-2-phenylimidazo[1,2-a]pyridin-3-yl)amino)benzoic acid [(E)-105-P3, XZ707]

Treatment of 4-((6-((aminooxy)methyl)-2-phenylimidazo[1,2-a]pyridin-3-yl)amino)benzoic acid (105) and commercially available 4-((6-methylpyrazin-2-yl)oxy)benzaldehyde as outlined in SE15 afforded 4-((6-((((4-((6-methylpyrazin-2-yl)oxy)benzylidene)amino)oxy)methyl)-2-phenylimidazo[1,2-a]pyridin-3-yl)amino)benzoic acid (105-P3, white solid, 40% yield) as a mixture of (Z)- and (E)-isomers with a ratio 5:95 based on LC. Purification by preparative HPLC (linear gradient of 30% B to 40% B over 20 min with a flow rate 20 mL/min) provided the title isomers separately.

(Z)-isomer ((Z)-105-P3) at retention time=10.6 min as a white solid. ESI-MS m/z: 571.2 (MH⁺). HRMS calcd. for C₃₃H₂₇N₆O₄ (MH⁺): 571.2088; found: 571.2090.

(E)-isomer ((E)-105-P3) at retention time=11.7 min as a white solid. ¹H NMR (500 MHz, DMSO-d₆) δ 8.96 (s, 1H), 8.28 (s, 1H), 8.25 (d, J=10.2 Hz, 3H), 7.86 (d, J=7.2 Hz, 2H), 7.80 (d, J=9.2 Hz, 1H), 7.69 (d, J=8.8 Hz, 3H), 7.53 (d, J=8.7 Hz, 2H), 7.41 (t, J=7.7 Hz, 2H), 7.33 (t, J=7.3 Hz, 1H), 7.15 (d, J=8.7 Hz, 2H), 6.63 (d, J=7.9 Hz, 2H), 5.19 (s, 2H), 2.27 (s, 3H). ¹³C NMR (126 MHz, DMSO-d₆) δ 167.57, 158.75, 155.13, 151.54, 149.67, 149.58, 140.10, 139.24, 132.69, 131.99 (2C), 129.43 (3C), 128.96 (2C), 128.77, 127.13 (2C), 125.64, 123.59, 121.75 (3C), 119.32, 118.03, 115.68, 115.50 (2C), 113.29 (2C), 72.55, 21.05. ESI-MS m/z: 571.2 (MH⁺). HRMS calcd. for C₃₃H₂₇N₆O₄ (MH⁺): 571.2088; found: 571.2089.

### (Z)-4-((2-(4-((((4-(5-Cyanopyridin-2-yl)benzylidene)amino)oxy)methyl)phenyl)-7-phenylimidazo[1,2-a]pyridin-3-yl)amino)benzoic acid [(Z)-106-D1, XZ708] and (E)-4-((2-(4-((((4-(5-Cyanopyridin-2-yl)benzylidene)amino)oxy)methyl)phenyl)-7-phenylimidazo[1,2-a]pyridin-3-yl)amino)benzoic acid [(E)-106-D1, XZ701]

Treatment of 4-((2-(4-((aminooxy)methyl)phenyl)-7-phenylimidazo[1,2-a]pyridin-3-yl)amino)benzoic acid (106) and commercially available 6-(4-formylphenyl)nicotinonitrile as outlined in SE15 afforded 4-((2-(4-((((4-(5-cyanopyridin-2-yl)benzylidene)amino)oxy)methyl)phenyl)-7-phenylimidazo[1,2-a]pyridin-3-yl)amino)benzoic acid (106-D1, white solid, 58% yield) as a mixture of (Z) and (E) isomers with a ratio 4:96 based on LC. Purification by preparative HPLC (linear gradient of 30% B to 50% B over 20 min with a flow rate 20 mL/min) provided the title isomers separately.

(Z)-isomer ((Z)-106-D1) at retention time=17.7 min as a white solid. ¹H NMR (500 MHz, DMSO-d₆) δ 9.13 (dd, J=2.2, 0.9 Hz, 1H), 9.00 (brs, 1H), 8.44 (dd, J=8.3, 2.2 Hz, 1H), 8.30-8.24 (m, 3H), 8.10 (d, J=8.6 Hz, 2H), 8.04 (s, 2H), 8.02 (d, J=8.0 Hz, 3H), 7.88 (d, J=7.4 Hz, 2H), 7.78 (d, J=9.1 Hz, 2H), 7.66 (s, 1H), 7.52 (ddt, J=21.6, 14.6, 7.4 Hz, 6H), 6.68 (brs, 2H), 5.29 (s, 2H). ESI-MS m/z: 641.2 (MH⁺) HRMS calcd. for C₄₀H₂₉N₆O₃ (MH⁺): 641.2296; found: 641.2287.

(E)-isomer ((E)-106-D1) at retention time=19.2 min as a white solid. ¹H NMR (500 MHz, DMSO-d₆) δ 12.37 (brs, 1H), 9.12 (dd, J=2.2, 0.9 Hz, 1H), 9.00 (s, 1H), 8.42 (d, J=8.2 Hz, 1H), 8.41 (s, 1H), 8.25 (dd, J=8.4, 0.9 Hz, 1H), 8.24 (d, J=8.5 Hz, 2H), 8.18 (brs, 1H), 8.04 (s, 1H), 8.01 (d, J=8.2 Hz, 2H), 7.88 (d, J=7.1 Hz, 2H), 7.77 (d, J=8.4 Hz, 4H), 7.59-7.46 (m, 6H), 6.69 (s, 2H), 5.24 (S, 2H). ¹³C NMR (126 MHz, DMSO-d₆) δ 167.82, 158.70, 153.06, 149.24, 142.85, 141.54, 138.50, 138.30, 137.39, 134.28, 133.56, 131.94, 129.60 (3C), 129.01 (2C), 128.79, 128.13 (3C), 127.94 (3C), 127.09 (3C), 126.86 (2C), 123.65, 120.85 (2C), 118.49, 117.72, 114.01, 112.79 (2C), 112.28, 108.12, 75.91. ESI-MS m/z: 641.2 (MH⁺). HRMS calcd. for C₄₀H₂₉N₆O₃ (MH⁺): 641.2296; found: 641.2289.

### (Z)-4-((7-Phenyl-2-(4-((((4-(pyrazin-2-yl)benzylidene)amino)oxy)methyl)phenyl)imidazo[1,2-a]pyridin-3-yl)amino)benzoic acid [(Z)-106-E6, XZ709] and (E)-4-((7-Phenyl-2-(4-((((4-(pyrazin-2-yl)benzylidene)amino)oxy)methyl)phenyl)imidazo[1,2-a]pyridin-3-yl)amino)benzoic acid [(E)-106-E6, XZ702]

Treatment of 4-((2-(4-((aminooxy)methyl)phenyl)-7-phenylimidazo[1,2-a]pyridin-3-yl)amino)benzoic acid (106) and commercially available 4-(pyrazin-2-yl)benzaldehyde as outlined in SE15 afforded 4-((7-phenyl-2-(4-((((4-(pyrazin-2-yl)benzylidene)amino)oxy)methyl)phenyl)imidazo[1,2-a]pyridin-3-yl)amino)benzoic acid (106-E6, white solid, 34% yield) as a mixture of (Z) and (E) isomers with a ratio 4:96 based on LC. Purification by preparative HPLC (linear gradient of 30% B to 45% B over 20 min with a flow rate 20 mL/min) provided the title isomers separately.

(Z)-isomer ((Z)-106-E6) at retention time=14.3 min as a white solid. ¹H NMR (500 MHz, DMSO-d₆) δ 12.34 (brs, 1H), 9.32 (d, J=1.5 Hz, 1H), 8.93 (s, 1H), 8.76 (dd, J=2.5, 1.5 Hz, 1H), 8.66 (d, J=2.5 Hz, 1H), 8.25 (d, J=8.5 Hz, 2H), 8.11 (d, J=8.6 Hz, 2H), 8.05 (d, J=8.3 Hz, 3H), 8.00 (s, 1H), 7.86 (d, J=7.1 Hz, 2H), 7.77 (d, J=9.0 Hz, 2H), 7.65 (s, 1H), 7.54 (t, J=7.7 Hz, 2H), 7.49 (d, J=8.0 Hz, 2H), 7.45 (t, J=7.3 Hz, 1H), 7.39 (d, J=7.2 Hz, 1H), 6.63 (brs, 1H), 6.55 (brs, 1H), 5.27 (s, 2H). ESI-MS m/z: 617.2 (MH⁺). HRMS calcd. for C₃₈H₂₉N₆O₃ (MH⁺): 617.2296; found: 617.2295.

(E)-isomer ((E)-106-E6) at retention time=15.4 min. as a white solid. ¹H NMR (500 MHz, DMSO-d₆) δ 12.38 (brs, 1H), 9.30 (d, J=1.6 Hz, 1H), 9.02 (s, 1H), 8.74 (dd, J=2.5, 1.5 Hz, 1H), 8.65 (d, J=2.5 Hz, 1H), 8.41 (s, 1H), 8.21 (d, J=8.4 Hz, 3H), 8.05 (d, J=1.8 Hz, 1H), 8.01 (d, J=8.2 Hz, 2H), 7.89 (d, J=7.5 Hz, 2H), 7.78 (dd, J=8.8, 2.5 Hz, 4H), 7.60-7.46 (m, 6H), 6.70 (brs, 2H), 5.24 (s, 2H). ESI-MS m/z: 617.2 (MH⁺). HRMS calcd. for C₃₈H₂₉N₆O₃ (MH⁺): 617.2296; found: 617.2301.

### (Z)-4-((2-(4-((((4-(2-Oxopyrrolidin-1-yl)benzylidene)amino)oxy)methyl)phenyl)-7-phenylimidazo[1,2-a]pyridin-3-yl)amino)benzoic acid [(Z)-106-B7, XZ710] and (E)-4-((2-(4-((((4-(2-Oxopyrrolidin-1-yl)benzylidene)amino)oxy)methyl)phenyl)-7-phenylimidazo[1,2-a]pyridin-3-yl)amino)benzoic acid [(E)-106-B7, XZ703]

Treatment of 4-((2-(4-((aminooxy)methyl)phenyl)-7-phenylimidazo[1,2-a]pyridin-3-yl)amino)benzoic acid (106) and commercially available 4-(2-oxopyrrolidin-1-yl)benzaldehyde as outlined in SE15 afforded 4-((2-(4-((((4-(2-oxopyrrolidin-1-yl)benzylidene)amino)oxy)methyl)phenyl)-7-phenylimidazo[1,2-a]pyridin-3-yl)amino)benzoic acid (106-B7, white solid, 28% yield) as a mixture of (Z) and (E) isomers with a ratio 5:95 based on LC. Purification by preparative HPLC (linear gradient of 30% B to 40% B over 20 min with a flow rate 20 mL/min) provided the title isomers separately.

(Z)-isomer ((Z)-106-B7) at retention time=14.2 min as a white solid. ¹H NMR (500 MHz, DMSO-d₆) δ 12.36 (brs, 1H), 8.97 (s, 1H), 8.13 (s, 1H), 8.02 (d, J=8.3 Hz, 3H), 7.96 (d, J=8.9 Hz, 2H), 7.87 (d, J=7.7 Hz, 2H), 7.77 (dd, J=8.8, 6.4 Hz, 3H), 7.72 (d, J=8.8 Hz, 1H), 7.60 (d, J=8.9 Hz, 1H), 7.55 (t, J=7.6 Hz, 2H), 7.50-7.44 (m, 5H), 6.66 (s, 2H), 5.22 (brs, 2H), 3.87-3.84 (m, 2H), 2.56-2.52 (m, 2H), 2.12-2.01 (m, 2H). ESI-MS m/z: 622.2 (MH⁺). HRMS calcd. for C₃₈H₃₂N₅O₄ (MH⁺): 622.2449; found: 622.2444.

(E)-isomer ((E)-106-B7) at retention time=15.3 min as a white solid. ¹H NMR (500 MHz, DMSO-d₆) δ 9.05 (s, 1H), 8.28 (s, 1H), 8.25 (d, J=7.1 Hz, 1H), 8.08 (s, 1H), 7.98 (d, J=8.3 Hz, 2H), 7.90 (d, J=7.2 Hz, 2H), 7.78 (d, J=9.1 Hz, 2H), 7.72 (d, J=8.8 Hz, 2H), 7.58 (dd, J=14.1, 8.4 Hz, 5H), 7.52 (dd, J=13.9, 7.9 Hz, 3H), 6.72 (d, J=7.9 Hz, 2H), 5.19 (s, 2H), 3.84 (t, J=7.0 Hz, 2H), 2.52-2.50 (m, 2H), 2.11-2.02 (m, 2H). ESI-MS m/z: 622.2 (MH⁺). HRMS calcd. for C₃₈H₃₂N₅O₄ (MH⁺): 622.2449; found: 622.2443.

### (Z)-4-((2-(4-((((4-((6-methylpyrazin-2-yl)oxy)benzylidene)amino)oxy)methyl)phenyl)-7-phenylimidazo[1,2-a]pyridin-3-yl)amino)benzoic acid [(Z)-106-P3, XZ711] and (E)-4-((2-(4-((((4-((6-methylpyrazin-2-yl)oxy)benzylidene)amino)oxy)methyl)phenyl)-7-phenylimidazo[1,2-a]pyridin-3-yl)amino)benzoic acid [(E)-106-P3, XZ704]

Treatment of 4-((2-(4-((aminooxy)methyl)phenyl)-7-phenylimidazo[1,2-a]pyridin-3-yl)amino)benzoic acid (106) and commercially available 4-((6-methylpyrazin-2-yl)oxy)benzaldehyde as outlined in SE15 afforded 4-((2-(4-((((4-((6-methylpyrazin-2-yl)oxy)benzylidene)amino)oxy)methyl)phenyl)-7-phenylimidazo[1,2-a]pyridin-3-yl)amino)benzoic acid (106-P3, white solid, 34% yield) as a mixture of (Z) and (E) isomers with a ratio 5:95 based on LC. Purification by preparative HPLC (linear gradient of 30% B to 50% B over 20 min with a flow rate 20 mL/min) provided the title isomers separately.

(Z)-isomer ((Z)-106-P3) at retention time=15.5 min as a white solid. ESI-MS m/z: 647.2 (MH⁺). HRMS calcd. for C₃₉H₃₁N₆O₄ (MH⁺): 647.2401; found: 647.2396.

(E)-isomer ((E)-106-P3) at retention time=16.3 min as a white solid. ¹H NMR (500 MHz, DMSO-d₆) δ 8.99 (s, 1H), 8.28 (s, 1H), 8.27-8.22 (m, 2H), 8.19 (d, J=7.1 Hz, 1H), 8.01 (s, 1H), 7.91 (d, J=8.3 Hz, 2H), 7.83 (d, J=7.1 Hz, 2H), 7.71 (d, J=9.0 Hz, 2H), 7.59 (d, J=8.7 Hz, 2H), 7.54-7.42 (m, 6H), 7.16 (d, J=8.7 Hz, 2H), 6.66 (d, J=8.3 Hz, 2H), 5.13 (s, 2H), 2.27 (s, 3H). ¹³C NMR (126 MHz, DMSO-d₆) δ 167.57, 158.78, 155.05, 151.57, 149.53, 149.22, 141.55, 140.96, 139.23 (2C), 137.26, 134.35, 132.69 (2C), 132.02 (2C), 129.81 (2C), 129.75, 129.16 (2C), 129.00, 128.95 (2C), 127.49 (2C), 127.16 (2C), 124.78, 121.83, 121.77 (2C), 119.08, 114.59, 113.39, 113.35, 111.49, 75.39, 21.05. ESI-MS m/z: 647.2 (MH⁺). HRMS calcd. for C₃₉H₃₁N₆O₄ (MH⁺): 647.2401; found: 647.2396.

### (Z)-4-((2-(4-(((([1,1′-Biphenyl]-4-ylmethylene)amino)oxy)methyl)phenyl)-7-phenylimidazo[1,2-a]pyridin-3-yl)amino)benzoic acid [(Z)-106-M10, XZ712] and (E)-4-((2-(4-(((([1,1′-Biphenyl]-4-ylmethylene)amino)oxy)methyl)phenyl)-7-phenylimidazo[1,2-a]pyridin-3-yl)amino)benzoic acid [(E)-106-M10, XZ705]

Treatment of 4-((2-(4-((aminooxy)methyl)phenyl)-7-phenylimidazo[1,2-a]pyridin-3-yl)amino)benzoic acid (106) and commercially available [1,1′-biphenyl]-4-carbaldehyde as outlined in SE15 afforded 4-((2-(4-(((([1,1′-biphenyl]-4-ylmethylene)amino)oxy)methyl)phenyl)-7-phenylimidazo[1,2-a]pyridin-3-yl)amino)benzoic acid (106-M10, white solid, 20% yield) as a mixture of (Z) and (E) isomers with a ratio 4:96 based on LC. Purification by preparative HPLC (linear gradient of 40% B to 60% B over 20 min with a flow rate 20 mL/min) provided the title isomers separately.

(Z)-isomer ((Z)-106-M10) at retention time=12.7 min as a white solid. ESI-MS m/z: 615.2 (MH⁺). HRMS calcd. for C₄₀H₃₁N₄O₃ (MH⁺): 615.2391; found: 615.2390.

(E)-isomer ((E)-106-M10) at retention time=13.7 min as a white solid. ¹H NMR (500 MHz, DMSO-d₆) δ 8.95 (s, 1H), 8.30 (s, 1H), 8.13 (d, J=7.1 Hz, 1H), 7.98 (s, 1H), 7.93 (d, J=8.3 Hz, 2H), 7.81 (d, J=7.1 Hz, 2H), 7.70 (d, J=9.0 Hz, 2H), 7.68-7.60 (m, 6H), 7.52-7.44 (m, 5H), 7.44-7.39 (m, 3H), 7.32 (t, J=7.4 Hz, 1H), 6.62 (d, J=7.3 Hz, 2H), 5.14 (s, 2H). ¹³C NMR (126 MHz, DMSO-d₆) δ 167.58, 149.72, 149.56, 142.02, 141.51, 139.74, 138.68, 137.55, 132.04 (2C), 131.35, 129.75 (2C), 129.49 (4C), 129.15 (2C), 128.38, 127.98 (2C), 127.53 (2C), 127.38 (2C), 127.14 (4C), 127.08, 124.46, 121.64, 118.85, 113.95, 113.27 (2C), 112.22, 75.53. ESI-MS m/z: 615.2 (MH⁺). HRMS calcd. for C₄₀H₃₁N₄O₃ (MH⁺): 615.2391; found: 615.2387.

### SE19: Synthesis of Triazole TDP1 inhibitors 6-(4-(Hydroxymethyl)phenyl)nicotinonitrile (119)

To a solution of commercially available 6-(4-formylphenyl)nicotinonitrile (D1, 1.02 g, 4.66 mmol) in MeOH (50 mL) and THF (50 mL). Sodium borohydride (176 mg, 4.66 mmol) was added portionwise at 0° C. After 30 min, the reaction mixture was concentrated. The residue was purified by silica gel column chromograph. The fraction was collected and afforded 6-(4-(hydroxymethyl)phenyl)nicotinonitrile (119, 898 mg) as a white solid (92% yield). ¹H NMR (500 MHz, CDCl₃) δ 8.97 (dd, J=2.2, 0.9 Hz, 1H), 8.08 (d, J=8.4 Hz, 2H), 8.04 (dd, J=8.3, 2.2 Hz, 1H), 7.88 (dd, J=8.3, 1.0 Hz, 1H), 7.55 (d, J=8.2 Hz, 2H), 7.29 (s, 1H), 4.82 (s, 2H). ¹³C NMR (126 MHz, CDCl₃) δ 160.15, 152.45, 143.57, 139.94, 136.57, 127.61 (2C), 127.41 (2C), 119.95, 116.99, 107.87, 64.80. DUIS-MS m/z: 211.0 (MH⁺).

### 6-(4-(Bromomethyl)phenyl)nicotinonitrile (120)

To a suspension of 6-(4-(hydroxymethyl)phenyl)nicotinonitrile (119, 346 mg, 1.65 mmol) in acetonitrile (10 mL) was added triphenylphosphane (648 mg, 2.47 mmol). The resulting white suspension was cooled to 0° C. and perbromomethane (819 mg, 2.47 mmol) was added. The formed light brown solution was stirred (rt, 30 min). The reaction mixture was concentrated and purified by silica gel column chromograph to provide 6-(4-(bromomethyl)phenyl)nicotinonitrile (120, 438 mg) as a white solid (97% yield). ¹H NMR (400 MHz, CDCl₃) δ 8.96 (d, J=1.3 Hz, 1H), 8.06-8.02 (m, 3H), 7.87 (dd, J=8.3, 1.0 Hz, 1H), 7.56 (d, J=8.4 Hz, 2H), 4.57 (s, 2H). ¹³C NMR (101 MHz, CDCl₃) δ 159.67, 152.50, 140.34, 139.98, 137.33, 129.77 (2C), 127.83 (2C), 119.99, 116.91, 108.13, 32.59. DUIS-MS m/z: 272.9, 274.9 (MH⁺).

### 6-(4-(Azidomethyl)phenyl)nicotinonitrile (121)

A solution of 6-(4-(bromomethyl)phenyl)nicotinonitrile (120, 286 mg, 1.05 mmol) and sodium azide (272 mg, 4.19 mmol) in acetone (5 mL) and water (1 mL) was heated (55° C., 18 h). The mixture was purified by silica gel column chromograph and 6-(4-(azidomethyl)phenyl)nicotinonitrile (121, 217 mg) was afforded as a white solid (88% yield). ¹H NMR (400 MHz, CDCl₃) δ 8.97 (d, J=2.1 Hz, 1H), 8.10 (d, J=8.3 Hz, 2H), 8.04 (dd, J=8.3, 2.2 Hz, 1H), 7.88 (d, J=8.3 Hz, 1H), 7.50 (d, J=8.0 Hz, 2H), 4.46 (s, 2H). ¹³C NMR (101 MHz, CDCl₃) δ 159.81, 152.50, 139.98, 138.01, 137.30, 128.79 (2C), 127.87 (2C), 120.00, 116.91, 108.11, 54.34. ESI-MS m/z: 236.1 (MH⁺).

### Methyl 4-((7-ethynyl-2-phenylimidazo[1,2-a]pyridin-3-yl)amino)benzoate (124a)

Treatment of 4-ethynylpyridin-2-amine (122a), benzaldehyde (123a) and methyl 4-isocyanobenzoate (111) as outlined in SE12 (rt, 24 h) provided methyl 4-((7-ethynyl-2-phenylimidazo[1,2-a]pyridin-3-yl)amino)benzoate (124a) as a pale yellow solid (95% yield). ¹H NMR (500 MHz, CDCl₃) δ 7.94-7.89 (m, 4H), 7.79 (s, 1H), 7.72 (dd, J=7.0, 1.0 Hz, 1H), 7.37 (t, J=7.4 Hz, 2H), 7.34-7.29 (m, 1H), 6.84 (dd, J=7.0, 1.5 Hz, 1H), 6.59 (d, J=8.4 Hz, 2H), 6.16 (s, 1H), 3.87 (s, 3H), 3.28 (s, 1H). ¹³C NMR (126 MHz, CDCl₃) δ 166.87, 148.59, 142.01, 140.95, 132.51, 132.06 (2C), 128.76 (2C), 128.45, 127.04 (2C), 122.16, 121.91, 121.51, 119.17, 117.58, 115.33, 112.84 (2C), 81.81, 80.39, 51.87. ESI-MS m/z: 368.1 (MH⁺).

### Methyl 4-((6-ethynyl-2-phenylimidazo[1,2-a]pyridin-3-yl)amino)benzoate (124b)

Treatment of 5-ethynylpyridin-2-amine (122b), benzaldehyde (123a) and methyl 4-isocyanobenzoate (111) as outlined in SE12 (rt, 24 h) provided methyl 4-((6-ethynyl-2-phenylimidazo[1,2-a]pyridin-3-yl)amino)benzoate (124b) as a pale brown solid (43% yield). ¹H NMR (400 MHz, CDCl₃) δ 7.95 (d, J=11.5 Hz, 2H), 7.91 (dd, J=6.0, 2.8 Hz, 3H), 7.58 (d, J=9.3 Hz, 1H), 7.39-7.27 (m, 4H), 6.60 (d, J=8.4 Hz, 2H), 5.99 (s, 1H), 3.86 (s, 3H), 3.09 (s, 1H). ¹³C NMR (101 MHz, CDCl₃) δ 166.82, 148.61, 141.94, 140.83, 132.58, 132.10 (2C), 128.76 (2C), 128.44, 128.32, 127.05 (2C), 126.16, 122.08, 117.61, 116.94, 112.85 (2C), 108.25, 79.83, 79.13, 51.84. ESI-MS m/z: 368.1 (MH⁺).

### Methyl 4-((2-(4-(prop-2-yn-1-yloxy)phenyl)imidazo[1,2-a]pyridin-3-yl)amino)benzoate (124c)

Treatment of pyridin-2-amine (122c), 4-(prop-2-yn-1-yloxy)benzaldehyde (123c) and methyl 4-isocyanobenzoate (111) as outlined in SE12 (rt, 24 h) provided methyl 4-((2-(4-(prop-2-yn-1-yloxy)phenyl)imidazo[1,2-a]pyridin-3-yl)amino)benzoate (124c) as a green solid (57% yield). ¹H NMR (500 MHz, CDCl₃) δ 7.91 (t, J=8.6 Hz, 4H), 7.78 (d, J=5.7 Hz, 1H), 7.64 (d, J=9.0 Hz, 1H), 7.28-7.22 (m, 1H), 6.96 (d, J=8.0 Hz, 2H), 6.80 (t, J=6.7 Hz, 1H), 6.61 (d, J=8.1 Hz, 2H), 6.15 (s, 1H), 4.70 (d, J=2.3 Hz, 2H), 3.88 (s, 3H), 2.54 (t, J=2.4 Hz, 1H). ¹³C NMR (126 MHz, CDCl₃) δ 166.88, 157.48, 148.93, 142.79, 139.34, 132.01 (2C), 128.29 (2C), 126.37, 125.41, 122.47, 121.68, 117.50, 115.95, 115.02 (2C), 112.77 (2C), 112.50, 78.40, 75.69, 55.77, 51.82. ESI-MS m/z: 398.2 (MH⁺).

### Methyl 4-((7-phenyl-2-(4-(prop-2-yn-1-yloxy)phenyl)imidazo[1,2-a]pyridin-3-yl)amino)benzoate (124d)

Treatment of 4-phenylpyridin-2-amine (114, 122d), 4-(prop-2-yn-1-yloxy)benzaldehyde (123c) and methyl 4-isocyanobenzoate (111) as outlined in SE12 (rt, 24 h) provided methyl 4-((7-phenyl-2-(4-(prop-2-yn-1-yloxy)phenyl)imidazo[1,2-a]pyridin-3-yl)amino)benzoate (124d) as a white solid (49% yield). ¹H NMR (500 MHz, CDCl₃) δ 7.97-7.89 (m, 4H), 7.80 (d, J=10.8 Hz, 2H), 7.64 (d, J=7.2 Hz, 2H), 7.49 (t, J=7.4 Hz, 2H), 7.42 (t, J=7.3 Hz, 1H), 7.06 (d, J=7.0 Hz, 1H), 6.97 (d, J=8.4 Hz, 2H), 6.64 (d, J=8.2 Hz, 2H), 6.18 (s, 1H), 4.69 (d, J=2.4 Hz, 2H), 3.88 (s, 3H), 2.53 (t, J=2.4 Hz, 1H). ¹³C NMR (126 MHz, CDCl₃) δ 166.87, 157.51, 148.94, 143.21, 140.01, 138.43, 132.03 (2C), 129.15 (2C), 128.41, 128.25 (2C), 126.76 (2C), 126.43, 122.39 (2C), 121.69, 115.80, 115.03 (2C), 114.15, 112.80 (2C), 112.35, 78.39, 75.72, 55.75, 51.82. ESI-MS m/z: 474.1 (MH⁺).

### Methyl 4-((2-(4-ethynylphenyl)imidazo[1,2-a]pyridin-3-yl)amino)benzoate (124e)

Treatment of pyridin-2-amine (122c), 4-ethynylbenzaldehyde (123b) and methyl 4-isocyanobenzoate (111) as outlined in SE12 (rt, 24 h) provided methyl 4-((2-(4-ethynylphenyl)imidazo[1,2-a]pyridin-3-yl)amino)benzoate (124e) as a white solid (35% yield). ¹H NMR (500 MHz, CDCl₃) δ 7.92 (dd, J=8.5, 3.3 Hz, 4H), 7.80 (d, J=6.8 Hz, 1H), 7.65 (d, J=9.1 Hz, 1H), 7.47 (d, J=8.0 Hz, 2H), 7.28 (d, J=8.0 Hz, 1H), 6.82 (t, J=6.7 Hz, 1H), 6.61 (d, J=8.2 Hz, 2H), 6.04 (s, 1H), 3.86 (s, 3H), 3.11 (s, 1H). ¹³C NMR (126 MHz, CDCl₃) δ 166.84, 148.69, 142.95, 138.64, 133.32, 132.33 (2C), 132.00 (2C), 126.62 (2C), 125.70, 122.54, 121.72, 121.51, 117.71, 117.13, 112.74 (2C), 112.70, 83.53, 78.06, 51.81. ESI-MS m/z: 368.1 (MH⁺).

### Methyl 4-((2-(4-ethynylphenyl)-7-phenylimidazo[1,2-a]pyridin-3-yl)amino)benzoate (124f)

Treatment of 4-phenylpyridin-2-amine (114, 122d), 4-ethynylbenzaldehyde (123b) and methyl 4-isocyanobenzoate (111) as outlined in SE12 (rt, 24 h) provided methyl 4-((2-(4-ethynylphenyl)-7-phenylimidazo[1,2-a]pyridin-3-yl)amino)benzoate (124f) as a pale yellow solid (37% yield). ¹H NMR (500 MHz, CDCl₃) δ 7.94 (dd, J=8.8, 1.6 Hz, 4H), 7.84 (d, J=0.8 Hz, 1H), 7.81 (dd, J=7.2, 0.9 Hz, 1H), 7.66 (dd, J=8.3, 1.3 Hz, 2H), 7.51 (t, J=7.6 Hz, 2H), 7.48 (d, J=8.4 Hz, 2H), 7.46-7.41 (m, 1H), 7.10 (dd, J=7.1, 1.8 Hz, 1H), 6.65 (d, J=8.3 Hz, 2H), 6.18 (s, 1H), 3.88 (s, 3H), 3.13 (s, 1H). ¹³C NMR (126 MHz, CDCl₃) δ 166.81, 148.62, 143.41, 139.32, 138.85, 138.34, 133.29, 132.42 (2C), 132.07 (2C), 129.17 (2C), 128.52, 126.80 (2C), 126.67 (2C), 122.47, 121.93, 121.65, 116.93, 114.42, 112.84 (2C), 112.73, 83.56, 78.13, 51.85. ESI-MS m/z: 444.1 (MH⁺). 4-((7-Ethynyl-2-phenylimidazo[1,2-a]pyridin-3-yl)amino)benzoic acid (125a, XZ690)

Treatment of methyl 4-((7-ethynyl-2-phenylimidazo[1,2-a]pyridin-3-yl)amino)benzoate (124a) as outlined in SE14 (rt, 24 h) and purification by preparative HPLC (linear gradient of 20% B to 40% B over 20 min with a flow rate 20 mL/min; retention time=12.3 min) provided 4-((7-ethynyl-2-phenylimidazo[1,2-a]pyridin-3-yl)amino)benzoic acid (125a) as a light yellow solid (70% yield). ¹H NMR (400 MHz, DMSO-d₆) δ 12.35 (s, 1H), 8.90 (s, 1H), 8.02 (d, J=7.2 Hz, 2H), 7.96 (d, J=7.0 Hz, 1H), 7.84 (s, 1H), 7.77 (d, J=8.4 Hz, 2H), 7.41 (t, J=7.6 Hz, 2H), 7.31 (t, J=7.3 Hz, 1H), 6.95 (dd, J=7.0, 1.6 Hz, 1H), 6.58 (d, J=8.2 Hz, 2H), 4.47 (s, 1H). ¹³C NMR (126 MHz, DMSO-d₆) δ 167.59, 149.89, 141.57, 139.47, 133.48, 132.05 (2C), 129.07 (2C), 128.45, 126.94 (2C), 123.66, 121.28, 121.04, 119.18, 118.55, 115.17, 112.97 (2C), 83.95, 82.62. ESI-MS m/z: 354.1 (MH⁺).

### 4-((6-Ethynyl-2-phenylimidazo[1,2-a]pyridin-3-yl)amino)benzoic acid (125b)

Treatment of methyl 4-((6-ethynyl-2-phenylimidazo[1,2-a]pyridin-3-yl)amino)benzoate (124b) as outlined in SE14 (rt, 24 h) provided 4-((6-ethynyl-2-phenylimidazo[1,2-a]pyridin-3-yl)amino)benzoic acid (125b) as a brown solid (48% yield). ¹H NMR (500 MHz, DMSO-d₆) δ 12.34 (s, 1H), 8.83 (s, 1H), 8.16 (s, 1H), 8.01 (d, J=7.7 Hz, 2H), 7.76 (d, J=8.4 Hz, 2H), 7.68 (d, J=9.3 Hz, 1H), 7.41 (t, J=7.6 Hz, 2H), 7.36 (d, J=9.2 Hz, 1H), 7.31 (t, J=7.4 Hz, 1H), 6.58 (s, 2H), 4.31 (s, 1H). ¹³C NMR (126 MHz, DMSO-d₆) δ 167.60, 149.88, 141.31, 138.88, 133.32, 132.02 (2C), 129.08 (2C), 128.49, 128.22, 126.96 (2C), 126.79, 121.34, 118.74, 117.92, 113.07 (2C), 107.79, 82.81, 80.47. ESI-MS m/z: 354.10 (MH⁺).

### 4-((2-(4-(Prop-2-yn-1-yloxy)phenyl)imidazo[1,2-a]pyridin-3-yl)amino)benzoic acid (125c)

Treatment of methyl 4-((2-(4-(prop-2-yn-1-yloxy)phenyl)imidazo[1,2-a]pyridin-3-yl)amino)benzoate (124c) as outlined in SE14 (65° C., 18 h) and purification by preparative HPLC (linear gradient of 10% B to 50% B over 20 min with a flow rate 20 mL/min; retention time=13.9 min) provided 4-((2-(4-(prop-2-yn-1-yloxy)phenyl)imidazo[1,2-a]pyridin-3-yl)amino)benzoic acid (125c) as a white solid (70% yield). ¹H NMR (500 MHz, DMSO-d₆) δ 12.45 (brs, 1H), 9.06 (s, 1H), 8.30 (d, J=6.6 Hz, 1H), 7.92 (d, J=9.0 Hz, 1H), 7.87 (d, J=8.9 Hz, 2H), 7.83 (d, J=7.8 Hz, 1H), 7.77 (d, J=9.1 Hz, 2H), 7.34 (t, J=7.0 Hz, 1H), 7.15 (d, J=8.9 Hz, 2H), 6.73 (d, J=8.3 Hz, 2H), 4.86 (d, J=2.4 Hz, 2H), 3.60 (t, J=2.3 Hz, 1H). ¹³C NMR (126 MHz, DMSO-d₆) δ 165.42, 147.22, 137.32, 129.86 (2C), 126.57 (2C), 122.84, 119.88, 118.41, 116.40, 116.05, 114.34, 113.85 (2C), 113.69, 112.17, 111.33, 111.30 (2C), 77.25, 76.90, 53.88. ESI-MS m/z: 384.1 (MH⁺)

### 4-((7-Phenyl-2-(4-(prop-2-yn-1-yloxy)phenyl)imidazo[1,2-a]pyridin-3-yl)amino)benzoic acid (125d)

Treatment of methyl 4-((7-phenyl-2-(4-(prop-2-yn-1-yloxy)phenyl)imidazo[1,2-a]pyridin-3-yl)amino)benzoate (124d) as outlined in SE14 (rt, 24 h) and purification by preparative HPLC (linear gradient of 20% B to 50% B over 20 min with a flow rate 20 mL/min; retention time=14.8 min) provided 4-((7-phenyl-2-(4-(prop-2-yn-1-yloxy)phenyl)imidazo[1,2-a]pyridin-3-yl)amino)benzoic acid (125d) as a pale yellow solid (57% yield). ¹H NMR (500 MHz, DMSO-d₆) δ 12.36 (brs, 1H), 8.86 (s, 1H), 8.00 (t, J=8.4 Hz, 3H), 7.96 (s, 1H), 7.84 (d, J=7.0 Hz, 2H), 7.78 (d, J=8.5 Hz, 2H), 7.51 (t, J=7.7 Hz, 2H), 7.45-7.40 (m, 1H), 7.31 (dd, J=7.2, 1.8 Hz, 1H), 7.04 (d, J=8.9 Hz, 2H), 6.60 (s, 2H), 4.82 (d, J=2.4 Hz, 2H), 3.57 (t, J=2.3 Hz, 1H). ¹³C NMR (126 MHz, DMSO-d₆) δ 167.65, 157.35, 150.31, 142.81, 138.79, 138.33, 137.17, 132.10 (2C), 129.58 (2C), 128.73, 128.12 (2C), 127.10, 127.05 (2C), 123.49, 121.16, 117.25, 115.41 (2C), 113.83, 112.89 (2C), 112.11, 79.68, 78.77, 55.84. ESI-MS m/z: 460.2 (MH⁺). HRMS calcd. for C₂₉H₂₂N₃O₃ (MH⁺), 460.1656; found, 460.1641.

### 4-((2-(4-Ethynylphenyl)imidazo[1,2-a]pyridin-3-yl)amino)benzoic acid (125e, XZ675p)

Treatment of methyl 4-((2-(4-ethynylphenyl)-7-phenylimidazo[1,2-a]pyridin-3-yl)amino)benzoate (124e) as outlined in SE14 (60° C., 18 h) and purification by preparative HPLC (linear gradient of 10% B to 50% B over 20 min with a flow rate 20 mL/min; retention time=13.5 min) provided 4-((2-(4-ethynylphenyl)imidazo[1,2-a]pyridin-3-yl)amino)benzoic acid (125e) as a white solid (67% yield). ¹H NMR (500 MHz, DMSO-d₆) δ 12.43 (brs, 1H), 9.07 (s, 1H), 8.21 (d, J=6.8 Hz, 1H), 7.96 (d, J=8.5 Hz, 2H), 7.86 (d, J=9.0 Hz, 1H), 7.77 (d, J=9.1 Hz, 2H), 7.69 (t, J=7.9 Hz, 1H), 7.61 (d, J=8.5 Hz, 2H), 7.23 (t, J=6.8 Hz, 1H), 6.69 (d, J=8.2 Hz, 2H), 4.33 (s, 1H). ¹³C NMR (126 MHz, DMSO-d₆) δ 167.55, 149.32, 140.69, 133.09, 132.75 (2C), 132.00 (2C), 130.77, 130.28, 127.19 (2C), 124.66, 122.52, 121.87, 119.59, 115.58 (2C), 113.35 (2C), 83.58, 82.81. ESI-MS m/z: 354.1 (MH⁺).

### 4-((2-(4-Ethynylphenyl)-7-phenylimidazo[1,2-a]pyridin-3-yl)amino)benzoic acid (125f, XZ676p)

Treatment of methyl 4-((2-(4-ethynylphenyl)-7-phenylimidazo[1,2-a]pyridin-3-yl)amino)benzoate (124f) as outlined in SE14 (65° C., 18 h) and purification by preparative HPLC (linear gradient of 20% B to 60% B over 20 min with a flow rate 20 mL/min; retention time=12.9 min) provided 4-((2-(4-ethynylphenyl)-7-phenylimidazo[1,2-a]pyridin-3-yl)amino)benzoic acid (125f) as a brown solid (95% yield). ¹H NMR (500 MHz, DMSO-d₆) δ 9.09 (s, 1H), 8.23 (d, J=7.1 Hz, 1H), 8.09 (dd, J=1.8, 0.9 Hz, 1H), 7.99 (d, J=8.5 Hz, 2H), 7.89 (d, J=7.1 Hz, 2H), 7.78 (d, J=9.1 Hz, 2H), 7.61 (d, J=8.5 Hz, 2H), 7.57 (t, J=7.5 Hz, 3H), 7.53-7.48 (m, 1H), 6.72 (d, J=8.3 Hz, 2H), 4.32 (s, 1H). ¹³C NMR (126 MHz, DMSO-d₆) δ 167.56, 149.39, 141.33, 137.31, 132.73 (2C), 132.38, 132.03 (2C), 131.32, 129.95, 129.79 (2C), 129.69, 127.47 (2C), 127.17 (2C), 124.72, 122.44, 121.85, 119.45, 114.43, 113.39 (2C), 111.85, 83.63, 82.77. ESI-MS m/z: 430.1 (MH⁺).

### 4-((7-(1-(4-(5-Cyanopyridin-2-yl)benzyl)-1H-1,2,3-triazol-4-yl)-2-phenylimidazo[1,2-a]pyridin-3-yl)amino)benzoic acid (107a, XZ722)

Treatment of 4-((7-ethynyl-2-phenylimidazo[1,2-a]pyridin-3-yl)amino)benzoic acid (125a) and 6-(4-(azidomethyl)phenyl)nicotinonitrile (121) as outlined in SE16 and purification by preparative HPLC (linear gradient of 20% B to 50% B over 20 min with a flow rate 20 m/min, retention time=16.0 min) provided 4-((7-(1-(4-(5-cyanopyridin-2-yl)benzyl)-1H-1,2,3-triazol-4-yl)-2-phenylimidazo[1,2-a]pyridin-3-yl)amino)benzoic acid (107a) as a white solid (80% yield). ¹H NMR (500 MHz, DMSO-d₆) δ 12.34 (s, 1H), 9.10 (d, J=2.2 Hz, 1H), 8.88 (d, J=11.1 Hz, 2H), 8.40 (dd, J=8.4, 2.2 Hz, 1H), 8.21 (dd, J=8.4, 4.8 Hz, 3H), 8.11 (s, 1H), 8.04 (dd, J=13.9, 7.5 Hz, 3H), 7.76 (d, J=8.4 Hz, 2H), 7.53 (d, J=8.2 Hz, 2H), 7.49 (dd, J=7.0, 1.5 Hz, 1H), 7.41 (t, J=7.6 Hz, 2H), 7.30 (t, J=7.3 Hz, 1H), 6.60 (s, 2H), 5.80 (s, 2H). ¹³C NMR (126 MHz, DMSO-d₆) δ 158.98, 153.04, 150.17, 145.64, 142.58, 141.49, 138.87, 138.67, 137.28, 133.80, 132.12, 129.04 (2C), 129.02 (2C), 128.24, 128.20 (3C), 128.15, 128.06, 126.91 (2C), 124.01, 123.27, 120.76 (2C), 118.61, 117.71, 112.97 (2C), 112.41, 110.83, 108.04, 53.26. ESI-MS m/z: 589.20 (MH⁺). HRMS calcd. for C₃₅H₂₄N₈O₂ (MH⁺), 589.2095; found, 589.2118.

### 4-((6-(1-(4-(5-Cyanopyridin-2-yl)benzyl)-1H-1,2,3-triazol-4-yl)-2-phenylimidazo[1,2-a]pyridin-3-yl)amino)benzoic acid (107b, XZ723)

Treatment of 4-((6-ethynyl-2-phenylimidazo[1,2-a]pyridin-3-yl)amino)benzoic acid (125b) and 6-(4-(azidomethyl)phenyl)nicotinonitrile (121) as outlined in SE16 and purification by preparative HPLC (linear gradient of 20% B to 50% B over 20 min with a flow rate 20 m/min, retention time=15.1 min) provided 4-((6-(1-(4-(5-cyanopyridin-2-yl)benzyl)-1H-1,2,3-triazol-4-yl)-2-phenylimidazo[1,2-a]pyridin-3-yl)amino)benzoic acid (107b) as a white solid. ¹H NMR (400 MHz, DMSO-d₆) δ 12.38 (brs, 1H), 9.10 (d, J=2.1 Hz, 1H), 8.85 (s, 1H), 8.80 (s, 1H), 8.47 (s, 1H), 8.40 (dd, J=8.3, 2.2 Hz, 1H), 8.19 (d, J=8.3 Hz, 3H), 8.02 (d, J=7.7 Hz, 2H), 7.84-7.70 (m, 4H), 7.49 (d, J=8.0 Hz, 2H), 7.41 (t, J=7.6 Hz, 2H), 7.30 (t, J=7.2 Hz, 1H), 6.61 (s, 2H), 5.75 (s, 2H). ESI-MS m/z: 589.20 (MH⁺). HRMS calcd. for C₃₅H₂₄N₈O₂ (MH⁺), 589.2095; found, 589.2117

### 4-((2-(4-((1-(4-(5-Cyanopyridin-2-yl)benzyl)-1H-1,2,3-triazol-4-yl)methoxy)phenyl)imidazo[1,2-a]pyridin-3-yl)amino)benzoic acid (107c, XZ721)

Treatment of 4-((2-(4-(prop-2-yn-1-yloxy)phenyl)imidazo[1,2-a]pyridin-3-yl)amino)benzoic acid (125c) and 6-(4-(azidomethyl)phenyl)nicotinonitrile (121) as outlined in SE16 and purification by preparative HPLC (linear gradient of 20% B to 50% B over 20 min with a flow rate 20 m/min, retention time=14.2 min) provided 4-((2-(4-((1-(4-(5-cyanopyridin-2-yl)benzyl)-1H-1,2,3-triazol-4-yl)methoxy)phenyl)imidazo[1,2-a]pyridin-3-yl)amino)benzoic acid (107c) as a white solid. ¹H NMR (400 MHz, DMSO-d₆) δ 12.30 (s, 1H), 9.10-9.05 (m, 1H), 8.80 (s, 1H), 8.38 (dd, J=8.4, 2.2 Hz, 1H), 8.34 (s, 1H), 8.21-8.14 (m, 3H), 7.97 (d, J=6.8 Hz, 1H), 7.94 (d, J=8.9 Hz, 2H), 7.74 (d, J=9.0 Hz, 2H), 7.65 (d, J=9.0 Hz, 1H), 7.45 (d, J=8.4 Hz, 2H), 7.36 (s, 1H), 7.08 (d, J=8.9 Hz, 2H), 6.96 (brs, 1H), 6.54 (brs, 2H), 5.71 (s, 2H), 5.17 (s, 2H). ESI-MS m/z: 619.20 (MH⁺). HRMS calcd. for C₃₆H₂₇N₈O₃ (MH⁺), 619.2201; found, 619.2232.

### 4-((2-(4-((1-(4-(5-Cyanopyridin-2-yl)benzyl)-1H-1,2,3-triazol-4-yl)methoxy)phenyl)-7-phenylimidazo[1,2-a]pyridin-3-yl)amino)benzoic acid (107d, XZ720)

Treatment of 4-((7-phenyl-2-(4-(prop-2-yn-1-yloxy)phenyl)imidazo[1,2-a]pyridin-3-yl)amino)benzoic acid (125d) and 6-(4-(azidomethyl)phenyl)nicotinonitrile (121) as outlined in SE16 and purification by preparative HPLC (linear gradient of 30% B to 50% B over 20 min with a flow rate 20 mL/min, retention time=13.9 min) provided 4-((2-(4-((1-(4-(5-cyanopyridin-2-yl)benzyl)-1H-1,2,3-triazol-4-yl)methoxy)phenyl)-7-phenylimidazo[1,2-a]pyridin-3-yl)amino)benzoic acid (107d) as a white solid. ¹H NMR (500 MHz, DMSO-d₆) δ 12.31 (brs, 1H), 9.01 (d, J=2.1 Hz, 1H), 8.91 (s, 1H), 8.32 (dd, J=8.4, 2.2 Hz, 1H), 8.28 (s, 1H), 8.14-8.09 (m, 4H), 7.96 (s, 1H), 7.83 (dd, J=14.8, 7.9 Hz, 4H), 7.70 (d, J=9.1 Hz, 2H), 7.50 (t, J=7.6 Hz, 3H), 7.43 (t, J=7.3 Hz, 1H), 7.39 (d, J=8.5 Hz, 2H), 7.09 (d, J=8.4 Hz, 2H), 6.62 (s, 2H), 5.64 (s, 2H), 5.13 (s, 2H). ESI-MS m/z: 695.2 (MH⁺). HRMS calcd. for C₄₂H₃₁N₈O₃ (MH⁺), 695.2514; found, 695.2520.

### 4-((2-(4-(1-(4-(5-Cyanopyridin-2-yl)benzyl)-1H-1,2,3-triazol-4-yl)phenyl)imidazo[1,2-a]pyridin-3-yl)amino)benzoic acid (107e, XZ719)

Treatment of 4-((2-(4-ethynylphenyl)imidazo[1,2-a]pyridin-3-yl)amino)benzoic acid (125e) and 6-(4-(azidomethyl)phenyl)nicotinonitrile (121) as outlined in SE16 and purification by preparative HPLC (linear gradient of 20% B to 50% B over 20 min with a flow rate 20 mL/min, retention time=14.5 min) provided 4-((2-(4-(1-(4-(5-cyanopyridin-2-yl)benzyl)-1H-1,2,3-triazol-4-yl)phenyl)imidazo[1,2-a]pyridin-3-yl)amino)benzoic acid (107e) as a white solid (33% yield). ¹H NMR (400 MHz, DMSO-d₆) δ 12.32 (s, 1H), 9.11-9.08 (m, 1H), 8.90 (s, 1H), 8.69 (s, 1H), 8.40 (dd, J=8.4, 2.2 Hz, 1H), 8.20 (d, J=8.4 Hz, 3H), 8.08 (d, J=8.5 Hz, 2H), 8.04 (d, J=6.9 Hz, 1H), 7.91 (d, J=8.4 Hz, 2H), 7.76 (d, J=9.1 Hz, 2H), 7.70 (d, J=9.1 Hz, 1H), 7.51 (d, J=8.4 Hz, 2H), 7.45-7.39 (m, 1H), 7.01 (brs, 1H), 6.60 (d, J=8.3 Hz, 2H), 5.75 (s, 2H). ESI-MS m/z: 589.2 (MH⁺). HRMS calcd. for C₃₅H₂₅N₈O₂ (MH⁺), 589.2095; found, 589.2106.

### 4-((2-(4-(1-(4-(5-Cyanopyridin-2-yl)benzyl)-1H-1,2,3-triazol-4-yl)phenyl)-7-phenylimidazo[1,2-a]pyridin-3-yl)amino)benzoic acid (107f, XZ718)

Treatment of 4-((2-(4-ethynylphenyl)-7-phenylimidazo[1,2-a]pyridin-3-yl)amino)benzoic acid (125f) and 6-(4-(azidomethyl)phenyl)nicotinonitrile (121) as outlined in SE16 and purification by preparative HPLC (linear gradient of 30% B to 50% B over 20 min with a flow rate 20 m/min, retention time=13.9 min) provided 4-((2-(4-(1-(4-(5-cyanopyridin-2-yl)benzyl)-1H-1,2,3-triazol-4-yl)phenyl)-7-phenylimidazo[1,2-a]pyridin-3-yl)amino)benzoic acid (107f) as a white solid. ¹H NMR (400 MHz, DMSO-d₆) δ 12.35 (brs, 1H), 9.10 (dd, J=2.2, 0.9 Hz, 1H), 8.98 (s, 1H), 8.70 (s, 1H), 8.41 (dd, J=8.4, 2.2 Hz, 1H), 8.21 (d, J=8.3 Hz, 3H), 8.14 (d, J=7.2 Hz, 1H), 8.09 (d, J=8.6 Hz, 2H), 8.02 (s, 1H), 7.94 (d, J=8.5 Hz, 2H), 7.88 (d, J=7.2 Hz, 2H), 7.78 (d, J=9.1 Hz, 2H), 7.58-7.42 (m, 6H), 6.67 (d, J=8.2 Hz, 2H), 5.76 (s, 2H). ESI-MS m/z: 665.2 (MH⁺). HRMS calcd. for C₄₁H₂₉N₈O₂ (MH⁺), 665.2408; found, 665.2420.

### SE20: Synthesis of Ether Linker Analogs

### 6-(4-((4-Formylphenethoxy)methyl)phenyl)nicotinonitrile (127)

Commercially available 4-(2-hydroxyethyl)benzaldehyde (349 μl, 2.65 mmol), 6-(4-(bromomethyl)phenyl)nicotinonitrile (120, 724 mg, 2.65 mmol) and Hunig's base N, N-Diisopropylethylamine (923 μl, 5.30 mmol) were charged in a flask. The mixture was heated (150° C., 1 h). The reaction suspension turns red brown during heating. The reaction mixture was cooled to rt and purified by CombiFlash. The title compound 6-(4-((4-formylphenethoxy)methyl)phenyl)nicotinonitrile (127, 541 mg) was afforded as a white solid (60% yield). ¹H NMR (400 MHz, CDCl₃) δ 9.98 (s, 1H), 8.91 (dd, J=2.2, 0.9 Hz, 1H), 8.02-7.96 (m, 3H), 7.84-7.79 (m, 3H), 7.41 (dd, J=8.2, 1.7 Hz, 4H), 4.59 (s, 2H), 3.77 (t, J=6.6 Hz, 2H), 3.03 (t, J=6.6 Hz, 2H). ¹³C NMR (101 MHz, CDCl₃) δ 192.01, 160.12, 152.45, 146.57, 141.03, 139.90, 136.64, 134.85, 129.88 (2C), 129.68 (2C), 128.00 (2C), 127.45 (2C), 119.90, 117.03, 107.83, 72.46, 70.58, 36.57. ESI-MS m/z: 343.10 (MH⁺).

### tert-Butyl 4-isocyanobenzoate (128)

The mixture of formic acid (3.2 mL, 85 mmol) and acetic anhydride (7.34 ml, 78 mmol) was heated (55° C., 2 h) and cooled to rt. The mixture was added dropwise to a solution of commercially available tert-butyl 4-aminobenzoate (5 g, 26 mmol) in THF (50 mL) at 0° C. The mixture was stirred (rt, 2 h). The solution was concentrated, and the residue oil was purified by silica gel chromatography. A mixture of tert-butyl 4-formamidobenzoate and (E)-N-(4-(tert-butoxycarbonyl)phenyl)formimidic acid (5.8 g) was afforded as a white solid, which was used in the next reaction directly. [¹H NMR (400 MHz, CDCl₃) δ 8.84 (d, J=11.2 Hz, 1H), 8.61 (d, J=11.1 Hz, 1H), 8.43 (d, J=1.8 Hz, 1H), 7.97 (dd, J=9.6, 8.7 Hz, 4H), 7.84 (s, 1H), 7.62 (d, J=8.7 Hz, 2H), 7.13 (d, J=8.7 Hz, 2H), 1.60 (s, 9H), 1.59 (s, 9H). ¹³C NMR (101 MHz, CDCl₃) δ 165.28, 165.03, 162.13, 159.15, 140.65, 140.49, 131.36 (2C), 130.70 (2C), 128.58, 128.06, 119.00 (2C), 117.15 (2C), 81.32, 81.11, 28.22 (6C). ESI-MS m/z: 166.10 (MH⁺-^{t}Bu), 222.10 (MH⁺).] tert-Butyl 4-formamidobenzoate (5.69 g, 26 mmol) and triethylamine (10.7 mL, 77 mmol) were dissolved in THF (50 mL). Phosphoryl trichloride (POCl₃, 2.9 mL, 31 mmol) was added dropwise at 0° C. The formed yellow suspension was stirred (0° C., 1 h) and quenched by Na₂CO₃ (sat. aq.) at 0° C. The reaction mixture was extracted by DCM, washed by brine and dried by Na₂SO₄. The solution was filtered and concentrated. The residue was purified by silica gel column chromograph. Compound tert-butyl 4-isocyanobenzoate (128, 4.39 g) was afforded as a light green solid (84% yield for two steps). ¹H NMR (400 MHz, CDCl₃) δ 8.03 (d, J=8.6 Hz, 2H), 7.42 (d, J=8.6 Hz, 2H), 1.61 (s, 9H). ¹³C NMR (101 MHz, CDCl₃) δ 166.74, 164.06, 132.82, 130.66 (2C), 129.54, 126.25 (2C), 82.01, 28.11 (3C). ESI-MS m/z: 204.10 (MH⁺).

### tert-Butyl 4-((2-(4-(2-((4-(5-((λ²-azaneylidene)-λ³-methyl)pyridin-2-yl)phenyl)methoxy)ethyl)phenyl)imidazo[1,2-a]pyridin-3-yl)amino)benzoate (129a)

Treatment of pyridin-2-amine (122c), and 6-(4-((4-formylphenethoxy)methyl)phenyl)nicotinonitrile (127), acetic acid and tert-butyl 4-isocyanobenzoate (128) as outlined in SE12 (75° C., 16 h) provided the title compound (129a) as a white solid (21% yield). ¹H NMR (500 MHz, DMSO-d₆) δ 9.01 (d, J=2.2 Hz, 1H), 8.73 (s, 1H), 8.31 (dd, J=8.4, 2.3 Hz, 1H), 8.11 (dd, J=8.4, 0.9 Hz, 1H), 8.06 (d, J=8.3 Hz, 2H), 7.88-7.83 (m, 3H), 7.62 (d, J=8.5 Hz, 2H), 7.57 (d, J=9.0 Hz, 1H), 7.36 (d, J=8.1 Hz, 2H), 7.28-7.23 (m, 1H), 7.21 (d, J=8.0 Hz, 2H), 6.86 (td, J=6.8, 1.2 Hz, 1H), 6.47 (s, 2H), 4.49 (s, 2H), 3.62 (t, J=6.8 Hz, 2H), 2.81 (t, J=6.8 Hz, 2H), 1.40 (s, 9H). ¹³C NMR (126 MHz, DMSO-d₆) δ 165.36, 159.35, 153.00, 150.25, 142.41, 141.74, 141.39, 139.08, 138.16, 136.38, 131.80, 131.74 (2C), 129.53 (2C), 128.26 (2C), 127.63 (2C), 126.80 (2C), 125.72, 123.42, 121.74, 120.54, 117.85, 117.78, 117.63, 112.90 (3C), 107.75, 79.99, 71.70, 71.05, 35.76, 28.36 (3C). ESI-MS m/z: 622.20 (MH⁺).

### 4-((2-(4-(2-((4-(5-((λ²-Azaneylidene)-λ³-methyl)pyridin-2-yl)phenyl)methoxy)ethyl)phenyl)imidazo[1,2-a]pyridin-3-yl)amino)benzoic acid (108a, XZ727)

Treatment of tert-butyl 4-((2-(4-(2-((4-(5-((λ²-azaneylidene)-λ³-methyl)pyridin-2-yl)phenyl)methoxy)ethyl)phenyl)imidazo[1,2-a]pyridin-3-yl)amino)benzoate (129a) as outlined in SE17 (rt, 1.5 h) and purification by preparative HPLC (with eluent solvent B from 25% to 50% within 20 min, flow rate: 20 mL/min, retention time=14.5 min.) provided the title compound (108a) as a white solid (39% yield). ¹H NMR (500 MHz, DMSO-d₆) δ 12.40 (brs, 1H), 9.09 (dd, J=2.2, 0.9 Hz, 1H), 9.05 (s, 1H), 8.39 (dd, J=8.4, 2.2 Hz, 1H), 8.27 (d, J=6.9 Hz, 1H), 8.19 (dd, J=8.4, 0.9 Hz, 1H), 8.13 (d, J=8.3 Hz, 2H), 7.90 (d, J=9.0 Hz, 1H), 7.86 (d, J=8.4 Hz, 2H), 7.77 (d, J=9.0 Hz, 3H), 7.46-7.39 (m, 4H), 7.30 (s, 1H), 6.72 (d, J=8.3 Hz, 2H), 4.57 (s, 2H), 3.71 (t, J=6.7 Hz, 2H), 2.93 (t, J=6.7 Hz, 2H). ¹³C NMR (126 MHz, DMSO-d₆) δ 167.54, 159.32, 153.00, 149.43, 141.68, 141.41, 136.41, 131.98 (2C), 130.05 (2C), 128.27 (4C), 127.63 (4C), 127.10 (2C), 124.85, 121.91, 120.54 (2C), 119.01, 118.19, 117.78, 115.83, 113.38 (2C), 107.77, 71.71, 70.80, 35.73. DUIS-MS m/z: 566.3 (MH⁺); 564.2 (M-H)⁻. ESI-MS m/z: 566.10 (MH⁺).

### tert-Butyl 4-((2-(4-(2-((4-(5-((λ³-azaneylidene)-λ³-methyl)pyridin-2-yl)phenyl)methoxy)ethyl)phenyl)-7-phenylimidazo[1,2-a]pyridin-3-yl)amino)benzoate (129b)

Treatment of commercially available 4-phenylpyridin-2-amine (114), and 6-(4-((4-formylphenethoxy)methyl)phenyl)nicotinonitrile (27) and tert-butyl 4-isocyanobenzoate (128) as outlined in SE12 (75° C., 16 h) provided the title compound (129b) as a white solid (13% yield). ¹H NMR (400 MHz, DMSO-d₆) δ 9.08 (dd, J=2.2, 0.8 Hz, 1H), 8.85 (s, 1H), 8.38 (dd, J=8.3, 2.2 Hz, 1H), 8.18 (dd, J=8.4, 0.9 Hz, 1H), 8.13 (d, J=8.4 Hz, 2H), 8.00 (s, 1H), 7.95 (d, J=8.3 Hz, 3H), 7.84 (d, J=7.1 Hz, 2H), 7.72 (d, J=9.1 Hz, 2H), 7.52 (t, J=7.6 Hz, 2H), 7.44 (d, J=7.8 Hz, 3H), 7.31 (dd, J=7.5, 5.2 Hz, 3H), 6.59 (d, J=7.8 Hz, 2H), 4.57 (s, 2H), 3.70 (t, J=6.8 Hz, 2H), 2.90 (t, J=6.8 Hz, 2H), 1.48 (s, 9H). ¹³C NMR (126 MHz, DMSO-d₆) δ 165.37, 159.34, 152.99, 150.23, 142.84, 141.74, 141.38, 139.17, 138.94, 138.32, 137.25, 136.38, 131.77 (2C), 129.60 (2C), 129.56 (2C), 128.78 (2C), 128.26, 127.63 (2C), 127.07 (2C), 126.79 (2C), 123.52, 121.81, 120.53 (2C), 117.81, 117.77, 113.96, 112.91 (2C), 112.20, 107.75, 80.00, 71.71, 71.05, 35.77, 28.36 (3C). ESI-MS m/z: 698.30 (MH⁺).

### 4-((2-(4-(2-((4-(5-((λ²-Azaneylidene)-λ³-methyl)pyridin-2-yl)phenyl)methoxy)ethyl)phenyl)-7-phenylimidazo[1,2-a]pyridin-3-yl)amino)benzoic acid (108b, XZ726)

Treatment of tert-butyl 4-((2-(4-(2-((4-(5-((λ²-azaneylidene)-λ³-methyl)pyridin-2-yl)phenyl)methoxy)ethyl)phenyl)-7-phenylimidazo[1,2-a]pyridin-3-yl)amino)benzoate (129b) as outlined in SE17 (rt, 1.5 h) and purification by preparative HPLC (with eluent solvent B from 30% to 50% within 20 min, flow rate: 20 mL/min, retention time=16.8 min.) provided the title compound (108b) as a white solid (49% yield). ¹H NMR (500 MHz, DMSO-d₆) δ 12.39 (brs, 1H), 9.08 (dd, J=2.3, 0.9 Hz, 1H), 9.05 (s, 1H), 8.38 (dd, J=8.3, 2.2 Hz, 1H), 8.27 (d, J=7.2 Hz, 1H), 8.18 (dd, J=8.4, 0.9 Hz, 1H), 8.13 (d, J=8.4 Hz, 2H), 8.07 (s, 1H), 7.89 (td, J=6.2, 3.2 Hz, 4H), 7.77 (d, J=9.0 Hz, 2H), 7.62-7.55 (m, 3H), 7.51 (t, J=7.3 Hz, 1H), 7.43 (d, J=8.4 Hz, 2H), 7.40 (d, J=8.1 Hz, 2H), 6.73 (d, J=8.3 Hz, 2H), 4.56 (s, 2H), 3.71 (t, J=6.7 Hz, 2H), 2.92 (t, J=6.7 Hz, 2H). ¹³C NMR (126 MHz, DMSO-d₆) δ 167.57, 159.33, 153.00, 149.59, 141.70, 141.41, 140.86, 137.24, 136.41, 132.01 (2C), 129.98 (2C), 129.82 (2C), 128.27 (4C), 127.63 (4C), 127.51 (2C), 127.07 (2C), 124.79, 121.81, 120.54 (2C), 118.76, 118.19, 117.78, 115.83, 114.71, 113.37 (2C), 111.29, 107.77, 71.71, 70.85, 35.75. ESI-MS m/z: 642.20 (MH⁺).

### SE21: Synthetic Scheme for Fluorosulfates 206

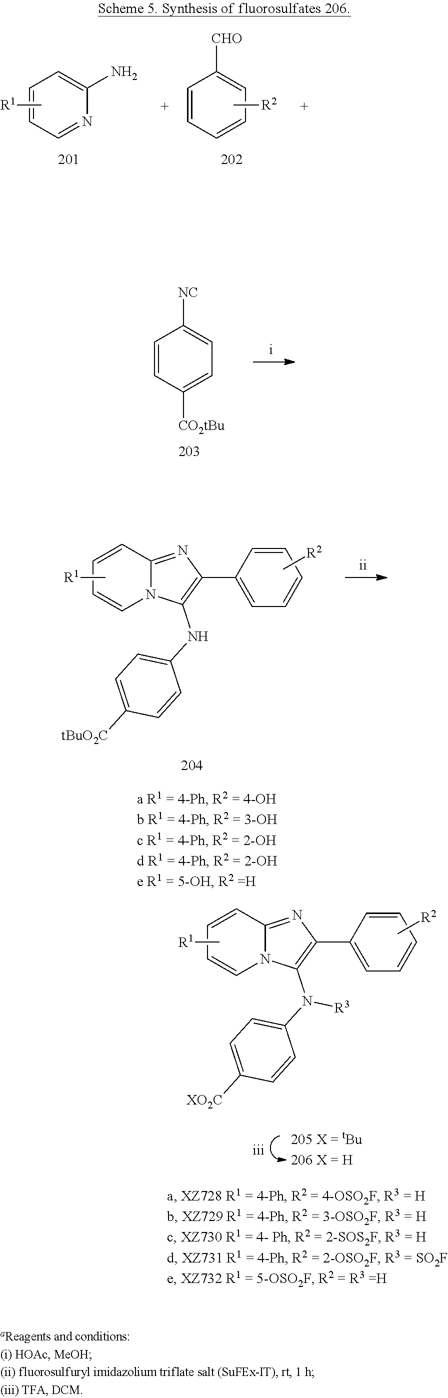

fluorosulfuryl imidazolium triflate salt (SuFEx-IT), rt, 1 h; (iii) TFA, DCM.

### SE22: GBBR Multicomponent Reaction to Prepare Tert-Butyl Esters (204)

Pyridine-2-amines (6 mmol), aldehydes (6 mmol), and acetic acid (12 mmol) were mixed in MeOH (5 mL) and THF (5 mL) (rt, 20 min). Isonitrile (6 mmol) was added. The reaction solution was stirred (80° C., 4 h or rt, 24 h). The final suspension was filtered and washed by hexanes and water. The solid product was collected to provide final tert-butyl esters (204).

### SE23: Synthesis of tert-Butyl 4-((2-(4-hydroxyphenyl)-7-phenylimidazo[1,2-a]pyridin-3-yl)amino)benzoate (204a)

Treatment of 4-phenylpyridin-2-amine, 4-hydroxybenzaldehyde and tert-butyl 4-isocyanobenzoate as outlined in SE22 and purification by CombiFlash, the title compound (204a) was afforded as a white solid (44% yield). ¹H NMR (500 MHz, DMSO-d₆) δ 9.59 (s, 1H), 8.80 (s, 1H), 7.95 (d, J=7.1 Hz, 1H), 7.93 (d, J=1.6 Hz, 1H), 7.84 (t, J=8.7 Hz, 4H), 7.72 (d, J=8.5 Hz, 2H), 7.52 (t, J=7.6 Hz, 2H), 7.43 (t, J=7.4 Hz, 1H), 7.29 (d, J=7.0 Hz, 1H), 6.79 (d, J=8.7 Hz, 2H), 6.58 (brs, 2H), 1.50 (s, 9H). ¹³C NMR (126 MHz, DMSO-d₆) δ 165.39, 157.75, 150.35, 142.69, 139.41, 138.40, 136.91, 131.75 (2C), 129.59 (2C), 128.69, 128.32 (2C), 127.03 (2C), 124.84, 123.34, 121.71, 116.66, 115.81 (2C), 113.71, 112.87 (2C), 111.90, 80.00, 28.38 (3C). ESI-MS m/z: 478.20 (MH⁺).

### SE24: tert-Butyl 4-((2-(3-hydroxyphenyl)-7-phenylimidazo[1,2-a]pyridin-3-yl)amino)benzoate (204b)

Treatment of 4-phenylpyridin-2-amine, 3-hydroxybenzaldehyde and tert-butyl 4-isocyanobenzoate as outlined in SE22 and purification by CombiFlash, the title compound (204b) was afforded as a brown oil (64% yield). ¹H NMR (500 MHz, DMSO-d₆) δ 8.85 (s, 1H), 7.97 (s, 1H), 7.96 (dd, J=4.3, 1.0 Hz, 1H), 7.85 (dd, J=8.4, 1.2 Hz, 2H), 7.73 (d, J=9.2 Hz, 2H), 7.55-7.50 (m, 3H), 7.47-7.42 (m, 2H), 7.32 (dd, J=7.4, 1.6 Hz, 1H), 7.18 (t, J=7.9 Hz, 1H), 6.71 (ddd, J=8.0, 2.5, 1.0 Hz, 1H), 6.59 (brs, 2H), 1.50 (s, 9H). ¹³C NMR (126 MHz, DMSO-d₆) δ 172.51, 170.81, 165.40, 157.91, 150.22, 142.72, 138.97, 138.31, 137.24, 135.12, 131.74, 129.91, 129.60 (2C), 128.78, 127.07 (2C), 123.51, 121.77, 118.00, 117.76, 115.27, 114.01, 113.95, 112.93, 112.20, 80.01, 28.38 (3C). ESI-MS m/z: 478.20 (MH)

### SE25: Synthesis of tert-Butyl 4-((2-(2-hydroxyphenyl)-7-phenylimidazo[1,2-a]pyridin-3-yl)amino)benzoate (204c)

Treatment of 4-phenylpyridin-2-amine, 2-hydroxybenzaldehyde and tert-butyl 4-isocyanobenzoate as outlined in SE22 and purification by CombiFlash, the title compound (204c) was afforded as a white solid (79% yield). ¹H NMR (500 MHz, DMSO-d₆) δ 13.10 (s, 1H), 8.95 (s, 1H), 8.14-8.10 (m, 2H), 7.88 (ddd, J=7.9, 3.1, 1.6 Hz, 3H), 7.74 (d, J=9.1 Hz, 2H), 7.53 (t, J=7.7 Hz, 2H), 7.49-7.43 (m, 2H), 7.20 (ddd, J=8.5, 7.2, 1.7 Hz, 1H), 6.94 (dd, J=8.2, 1.3 Hz, 1H), 6.79 (td, J=7.5, 1.3 Hz, 1H), 6.65 (s, 2H), 1.50 (s, 9H). ¹³C NMR (126 MHz, DMSO-d₆) δ 165.34, 157.63, 149.75, 140.90, 138.63, 137.81, 131.79 (2C), 130.07, 129.64 (2C), 129.12, 127.24 (2C), 126.81, 123.50, 122.19, 119.35, 117.52, 117.28, 116.45, 113.30, 113.13 (2C), 80.08, 28.36 (3C).

### SE26: Synthesis of tert-Butyl 4-((6-hydroxy-2-phenylimidazo[1,2-a]pyridin-3-V)amino)benzoate (204e)

Treatment of 6-aminopyridin-3-ol hydrochloride, benzaldehyde and tert-butyl 4-isocyanobenzoate as outlined in SE22 and purification by CombiFlash, the title compound (204e) was afforded as a pale yellow solid (13% yield). ¹H NMR (500 MHz, DMSO-d₆) δ 10.75 (s, 1H), 9.28 (s, 1H), 7.92 (dd, J=9.0, 3.3 Hz, 3H), 7.74 (d, J=9.0 Hz, 2H), 7.72 (s, 1H), 7.67 (dd, J=9.6, 2.0 Hz, 1H), 7.55 (t, J=7.4 Hz, 2H), 7.51-7.47 (m, 1H), 6.80 (d, J=8.2 Hz, 2H), 1.50 (s, 9H). ¹³C NMR (126 MHz, DMSO-d₆) δ 165.22, 149.74, 148.71, 134.32, 131.66 (2C), 130.48, 130.28, 129.74 (2C), 127.71, 127.25 (3C), 122.98, 119.47, 113.79, 113.60 (2C), 108.53, 80.30, 28.34 (3C). ESI-MS m/z: 402.20 (MH⁺).

### SE27: Synthesis of Fluorosulfonates (205) Using Fluorosulfuryl Imidazolium Triflate Salt (SuFEx-IT)

The tert-butyl esters (204, 0.08 mmol) was dissolved in Acetonitrile (2.0 mL) and DCM (2.0 mL). Triethylamine (0.03 mL, 0.19 mmol) and SuFEx-IT (50 mg, 0.15 mmol) was added at rt. The reaction mixture was stirred (rt, 1 h). The mixture was purified by CombiFlash. The related fraction was collected to afforded fluorosulfonates (205). Angew. Chem. Int. Ed. 2018, 57, 2605.

### SE28: Synthesis of tert-Butyl 4-((2-(4-((fluorosulfonyl)oxy)phenyl)-7-phenylimidazo[1,2-a]pyridin-3-yl)amino)benzoate (205a)

Treatment of tert-butyl 4-((2-(4-hydroxyphenyl)-7-phenylimidazo[1,2-a]pyridin-3-yl)amino)benzoate (204a) as outlined in SE27 and purification by CombiFlash the title compound (205a) was afforded as a colorless solid (35% yield). ¹H NMR (500 MHz, CDCl₃) δ 8.10 (d, J=8.8 Hz, 2H), 7.91 (d, J=8.4 Hz, 2H), 7.87 (d, J=10.3 Hz, 2H), 7.66 (d, J=6.9 Hz, 2H), 7.50 (t, J=7.5 Hz, 2H), 7.43 (t, J=7.3 Hz, 1H), 7.35 (d, J=8.5 Hz, 2H), 7.12 (d, J=7.2 Hz, 1H), 6.64 (d, J=8.3 Hz, 2H), 5.94 (s, 1H), 1.56 (s, 9H). ¹³C NMR (126 MHz, CDCl₃) δ 165.48, 149.56, 148.00, 143.59, 139.12, 138.67, 138.33, 133.84, 131.98 (2C), 129.22 (2C), 128.92 (2C), 128.61, 126.83 (2C), 124.24, 122.56, 121.18 (2C), 117.12, 114.69, 112.96, 112.69 (2C), 80.60, 28.26 (3C). ¹⁹F NMR (376 MHz, CDCl₃) δ 37.87. ESI-MS m/z: 560.10 (MH⁺), 582.10 (MNa⁺).

### SE29: Synthesis of tert-Butyl 4-((2-(3-((fluorosulfonyl)oxy)phenyl)-7-phenylimidazo[1,2-a]pyridin-3-yl)amino)benzoate (205b)

Treatment of tert-butyl 4-((2-(3-hydroxyphenyl)-7-phenylimidazo[1,2-a]pyridin-3-yl)amino)benzoate (204b) as outlined in SE27 and purification by CombiFlash the title compound (205b) was afforded as a colorless oil (63% yield). ¹H NMR (400 MHz, CDCl₃) δ 8.03 (s, 1H), 7.96 (d, J=7.9 Hz, 1H), 7.87 (d, J=8.8 Hz, 2H), 7.82-7.79 (m, 2H), 7.64-7.61 (m, 2H), 7.49 (t, J=7.5 Hz, 2H), 7.44-7.40 (m, 1H), 7.37 (t, J=8.1 Hz, 1H), 7.21 (dd, J=8.1, 2.6 Hz, 1H), 7.07 (dd, J=7.3, 1.5 Hz, 1H), 6.60 (d, J=8.3 Hz, 2H), 6.25 (s, 1H), 1.55 (s, 9H). ¹³C NMR (100 MHz, CDCl₃) δ 165.56, 150.42, 147.92, 143.40, 139.15, 138.19, 137.94, 135.82, 131.88 (2C), 130.52, 129.21 (2C), 128.63, 126.78 (2C), 126.65, 124.04, 122.59, 119.99, 119.18, 117.58, 114.43, 112.92, 112.68 (2C), 80.54, 28.24 (3C). ¹⁹F NMR (376 MHz, CDCl₃) δ 38.14.

### SE30: Synthesis of tert-Butyl 4-((2-(2-((fluorosulfonyl)oxy)phenyl)-7-phenylimidazo[1,2-a]pyridin-3-yl)amino)benzoate (205c) and tert-butyl 4-((fluorosulfonyl)(2-(2-((fluorosulfonyl)oxy)phenyl)-7-phenylimidazo[1,2-a]pyridin-3-yl)amino)benzoate (205d)

Treatment of tert-butyl 4-((2-(2-hydroxyphenyl)-7-phenylimidazo[1,2-a]pyridin-3-yl)amino)benzoate (204c) as outlined in SE27 and purification by CombiFlash the title compounds (205c in 24% yield and 205d in 5% yield) were afforded as a colorless solid. For 205c: ¹H NMR (400 MHz, CDCl₃) δ 7.86-7.80 (m, 4H), 7.75 (dt, J=7.1, 1.0 Hz, 1H), 7.65 (dd, J=8.3, 1.3 Hz, 2H), 7.49 (t, J=7.4 Hz, 2H), 7.45-7.38 (m, 4H), 7.11 (d, J=8.9 Hz, 1H), 6.52 (d, J=8.8 Hz, 2H), 6.21 (s, 1H), 1.54 (s, 9H). ¹⁹F NMR (376 MHz, CDCl₃) δ 41.01. DUIS-MS m/z: 560.4 (MH⁺), 558.1 (M-H)⁻. For 205d: ¹⁹F NMR (376 MHz, CDCl₃) δ 57.24, 41.66. DUIS-MS m/z: 642.1 (MH⁺).

### SE31: Deprotection of Tert-Butyl Group by TFA to Prepare Acids (206)

Compound tert-butyl ester (205, 0.1 mmol) was mixed with TFA/DCM (1/1, 1.0 mL) at rt. The reaction mixture was stirred (rt, 30 min). The solvent was removed and the residue was dissolved in MeOH and purified by preparative HPLC. The related fraction was collected and lyophilized to afford the related acids (206).

### SE32: Synthesis of 4-((2-(4-((Fluorosulfonyl)oxy)phenyl)-7-phenylimidazo[1,2-a]pyridin-3-yl)amino)benzoic acid (XZ728)

Treatment of tert-butyl 4-((2-(4-((fluorosulfonyl)oxy)phenyl)-7-phenylimidazo[1,2-a]pyridin-3-yl)amino)benzoate (205a) as outlined in SE31 and purification by preparative HPLC (CAT #00G-4436-P0-AX) (linear gradient of 20% B to 60% B over 20 min with a flow rate 20 mL/min, retention time=14.4 min) provided the title compound (XZ728) as a white solid (64% yield). DUIS-MS m/z: 504.1 (MH⁺)/502.1 (M-H)⁻.

### SE33: Synthesis of 4-((2-(3-((Fluorosulfonyl)oxy)phenyl)-7-phenylimidazo[1,2-a]pyridin-3-yl)amino)benzoic acid (XZ729)

Treatment of tert-butyl 4-((2-(3-((fluorosulfonyl)oxy)phenyl)-7-phenylimidazo[1,2-a]pyridin-3-yl)amino)benzoate (205b) as outlined in SE31 and purification by preparative HPLC (CAT #00G-4436-P0-AX) (linear gradient of 20% B to 50% B over 20 min with a flow rate 20 mL/min, retention time=17.1 min) provided the title compound (XZ728) as a white solid (36% yield). ¹H NMR (500 MHz, DMSO-d₆) δ 8.94 (s, 1H), 8.09 (d, J=7.1 Hz, 1H), 8.06-8.02 (m, 2H), 7.99 (s, 1H), 7.83-7.79 (m, 2H), 7.71 (d, J=9.1 Hz, 2H), 7.61 (t, J=8.2 Hz, 1H), 7.52 (dd, J=7.6, 2.3 Hz, 1H), 7.48 (t, J=7.7 Hz, 2H), 7.39 (d, J=7.4 Hz, 2H), 6.59 (d, J=8.3 Hz, 2H). ¹³C NMR (126 MHz, DMSO-d₆) δ 167.57, 150.52, 149.55, 142.42, 139.41, 137.77, 135.38, 132.07 (2C), 131.92, 129.70 (2C), 129.28, 127.30 (2C), 127.20, 124.28, 121.70, 121.06, 119.52, 118.86, 117.52, 115.19, 113.53, 113.30, 113.20. ¹⁹F NMR (376 MHz, DMSO-d₆) δ 38.93. ESI-MS m/z: 504.10 (MH⁺).

### SE34: Synthesis of 4-((2-(2-((Fluorosulfonyl)oxy)phenyl)-7-phenylimidazo[1,2-a]pyridin-3-yl)amino)benzoic acid (XZ730)

Treatment of tert-butyl 4-((2-(2-((fluorosulfonyl)oxy)phenyl)-7-phenylimidazo[1,2-a]pyridin-3-yl)amino)benzoate (205c) as outlined in SE31 and purification by preparative HPLC (CAT #00G-4436-P0-AX) (linear gradient of 20% B to 50% B over 20 min with a flow rate 20 mL/min, retention time=14.9 min) provided the title compound (XZ730) as a white solid (45% yield). ¹H NMR (500 MHz, DMSO-d₆) δ 8.87 (s, 1H), 8.05 (d, J=7.1 Hz, 1H), 7.96 (s, 1H), 7.83-7.80 (m, 2H), 7.78-7.75 (m, 1H), 7.65 (t, J=8.4 Hz, 3H), 7.52 (ddd, J=6.9, 4.5, 2.0 Hz, 2H), 7.47 (t, J=7.7 Hz, 2H), 7.43-7.37 (m, 2H), 6.53 (d, J=8.3 Hz, 2H). ¹³C NMR (126 MHz, DMSO-d₆) δ 167.56, 149.56, 147.35, 142.23, 137.75, 131.87 (2C), 131.18, 129.98, 129.95, 129.78, 129.68 (2C), 129.23, 128.11, 127.95, 127.31 (2C), 124.39, 122.85, 121.44, 120.58, 117.59, 115.25, 113.43, 113.18. ¹⁹F NMR (376 MHz, DMSO-d₆) δ 42.58. ESI-MS m/z: 504.10 (MH⁺).

### SE35: Synthesis of 4-((Fluorosulfonyl)(2-(2-((fluorosulfonyl)oxy)phenyl)-7-phenylimidazo[1,2-a]pyridin-3-yl)amino)benzoic acid (206d, XZ731)

Treatment of tert-butyl 4-((fluorosulfonyl)(2-(2-((fluorosulfonyl)oxy)phenyl)-7-phenylimidazo[1,2-a]pyridin-3-yl)amino)benzoate (205d) as outlined in SE31 and purification by preparative HPLC (CAT #00G-4436-P0-AX) (linear gradient of 40% B to 80% B over 20 min with a flow rate 20 mL/min, retention time=16.9 min) provided the title compound (XZ731) as a brown solid (85% yield). ¹H NMR (400 MHz, DMSO-d₆) δ 8.90 (dd, J=7.2, 0.9 Hz, 1H), 8.15 (s, 1H), 8.00 (d, J=8.8 Hz, 2H), 7.96-7.92 (m, 2H), 7.77 (d, J=9.0 Hz, 1H), 7.73 (dd, J=8.3, 1.6 Hz, 1H), 7.66 (dd, J=7.2, 1.8 Hz, 1H), 7.62 (td, J=7.3, 1.8 Hz, 1H), 7.57 (t, J=7.4 Hz, 2H), 7.52-7.47 (m, 1H), 7.42 (dd, J=7.7, 1.6 Hz, 1H), 7.38 (d, J=8.7 Hz, 2H). ¹⁹F NMR (376 MHz, DMSO-d₆) δ 57.67, 42.45. ESI-MS m/z: 586.00 (MH⁺).

### SE36: Synthesis of 4-((6-((Fluorosulfonyl)oxy)-2-phenylimidazo[1,2-a]pyridin-3-VA)amino)benzoic acid (XZ732)

Treatment of tert-butyl 4-((6-hydroxy-2-phenylimidazo[1,2-a]pyridin-3-yl)amino) (204e) as outlined in SE27 afforded tert-butyl 4-((6-((fluorosulfonyl)oxy)-2-phenylimidazo[1,2-a]pyridin-3-yl)amino)benzoate [205e, ESI-MS m/z: 484.10 (MH⁺)], which was used in next step without purification. Treatment of 205e as outlined in SE31 and purification by preparative HPLC (CAT #00G-4436-P0-AX) (linear gradient of 20% B to 50% B over 20 min with a flow rate 20 mL/min, retention time=13.7 min) provided the title compound (XZ732) as a white solid (66% yield). ¹H NMR (500 MHz, DMSO-d₆) δ 8.91 (s, 1H), 8.79 (d, J=2.3 Hz, 1H), 7.94-7.89 (m, 2H), 7.85 (d, J=9.8 Hz, 1H), 7.69 (d, J=9.1 Hz, 2H), 7.66 (dd, J=9.8, 2.3 Hz, 1H), 7.37 (t, J=7.6 Hz, 2H), 7.31-7.26 (m, 1H), 6.55 (d, J=8.3 Hz, 2H). ¹³C NMR (126 MHz, DMSO-d₆) δ 167.60, 149.62, 140.47, 140.20, 138.94, 132.19, 131.94 (2C), 129.24 (2C), 129.03, 127.11 (2C), 121.55, 121.33, 120.53, 119.08, 118.36, 113.23 (2C). ¹⁹F NMR (376 MHz, DMSO-d₆) δ 39.23. DUIS-MS m/z: 428.0 (MH⁺).

### SE37: Synthetic Scheme for sulfonyl fluoride XZ734

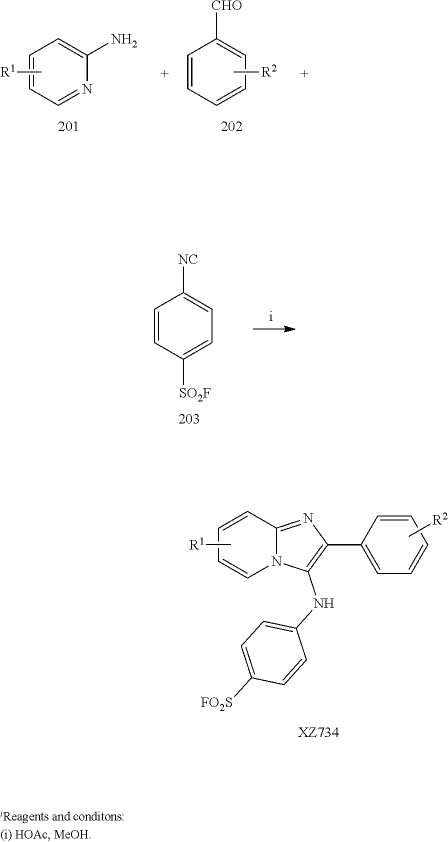

### SE38: Synthesis of 4-((2-Phenylimidazo[1,2-a]pyridin-3-yl)amino)benzenesulfonyl fluoride (XZ734)

Treatment of pyridin-2-amine, benzaldehyde and 4-isocyanobenzenesulfonyl fluoride as outlined in SE22 and purification by preparative HPLC (CAT #00G-4436-P0-AX) (linear gradient of 10% B to 50% B over 20 min with a flow rate 20 mL/min, retention time=13.1 min) provided the title compound (XZ734) as a white solid (15% yield). 1H NMR (500 MHz, DMSO-d₆) δ 9.73 (s, 1H), 8.37 (d, J=6.7 Hz, 1H), 7.96 (d, J=9.0 Hz, 1H), 7.93-7.89 (m, 4H), 7.82 (t, J=8.0 Hz, 1H), 7.54 (t, J=7.7 Hz, 2H), 7.46 (t, J=7.4 Hz, 1H), 7.33 (t, J=6.8 Hz, 1H), 6.97 (brs, 2H). ESI-MS m/z: 368.1 (MH⁺).

### SE39: Synthetic Scheme for Synthesis of PROTACs 308

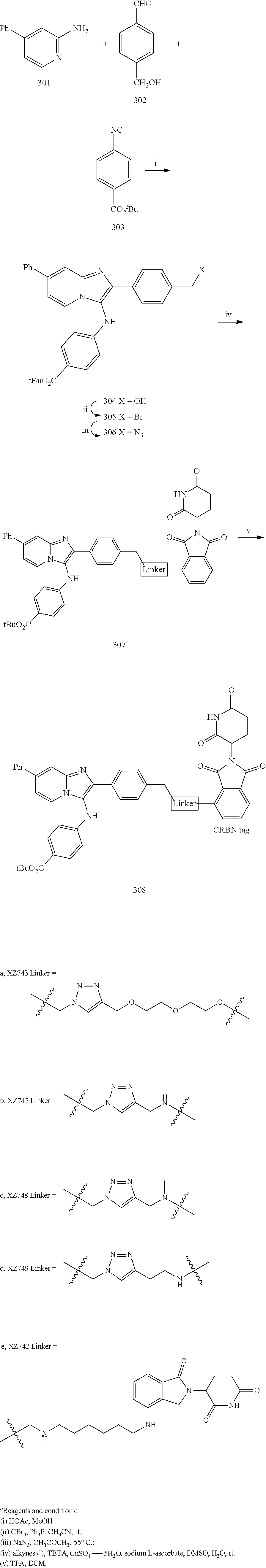

### SE40: Synthetic Scheme for Synthesis of Tdp1 PROTACs 311

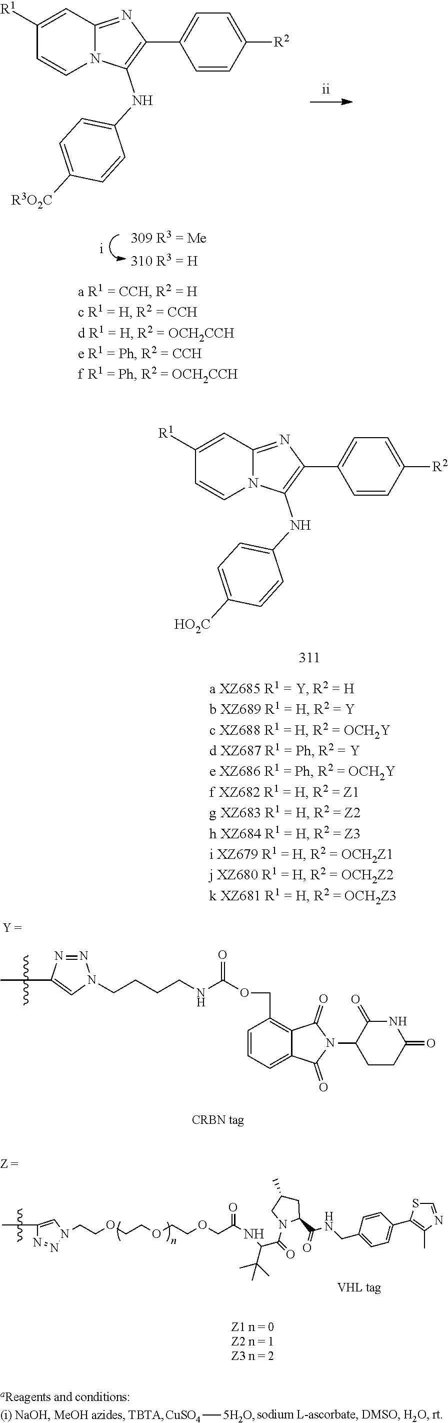

### SE41: GBBR multicomponent reaction to prepare imidazo[1,2-a]pyrazines and imidazo[1,2-a]pyridines (304, 309)

Pyridine-2-amines (6 mmol), aldehydes (6 mmol), and acetic acid (12 mmol) were mixed in MeOH (5 mL) and THF (5 mL) (rt, 20 min). Isonitrile (6 mmol) was added. The reaction solution was stirred (80° C., 4 h or rt, 24 h). The final suspension was filtered and washed by hexanes and water. The solid product was collected to provide final imidazo[1,2-a]pyrazines or imidazo[1,2-a]pyridines (304, 309).

### SE42: Deprotection of methyl ester to prepare carboxylic acids (310)

Methyl esters (309, 0.8 mmol) was suspended in MeOH (4.0 mL) in a tube. NaOH (4 ml, aq. 2M) was added. The suspension in the sealed tube was microwave-heated (100° C., 4 h). The reaction mixture was cooled to rt and acidified by HCl (aq. 2N). The formed suspension was filtered and washed by water and hexanes. The solid was collected to afford the carboxylic acids (310).

### SE43: CuAAC to prepare triazoles PROTACs (308 and 311)

Alkynes (309, 0.1 mmol, 1 mg in 10 μL DMSO), azides (0.1 mmol, 1 mg in 10 μL DMSO) and tris((1-benzyl-1H-1,2,3-triazol-4-yl)methyl)amine (TBTA, 0.04 mmol, 1 mg in 10 μL DMSO) were mixed in a vial with a stirrer bar. Sodium ascorbate (0.1 mmol, 1 mg in 10 μL H₂O) and CuSO₄-5H₂O (0.02 mmol, 1 mg in 10 μL water) were added. The reaction was diluted in DMSO (2 mL). The formed bright yellow solution was stirred at rt overnight under Argon. A yellow suspension was formed. The reaction mixture was dissolved in DMSO and purified by HPLC to afford triazoles (308 and 311).

### SE44: Deprotection of Tert-Butyl Group by TFA to Prepare Acids (308)

Compound tert-butyl ester (7, 0.1 mmol) was mixed with TFA/DCM (1/1, 1.0 mL) at rt. The reaction mixture was stirred (rt, 30 min). The solvent was removed and the residue was dissolved in MeOH and purified by preparative HPLC. The related fraction was collected and lyophilized to afford the related acids (308).

### SE45: Preparation of Pomalidomide with Alkynes 301-303

The commercially available 2-(2,6-dioxopiperidin-3-yl)-4-fluoroisoindoline-1,3-dione (385 mg, 1.40 mmol), N-ethyl-N-isopropylpropan-2-amine (0.73 ml, 4.18 mmol) and alkyne amine (1.53 mmol) in DMSO (5.0 mL) was stirred (130° C., 18 h). The reaction mixture was cooled to rt and purified by preparative HPLC. The related fraction was collected and lyophilized to afford the related alkynes 301-303. (Chem. Sci., 2021, 12, 4519)

### SE46: Synthesis of 2-(2,6-Dioxopiperidin-3-yl)-4-(prop-2-yn-1-ylamino)isoindoline-1,3-dione (alkyne 1)

Treatment of 2-(2,6-dioxopiperidin-3-yl)-4-fluoroisoindoline-1,3-dione and prop-2-yn-1-amine as outlined in SE45 and purification by preparative HPLC (CAT #00G-4436-P0-AX) (linear gradient of 20% B to 65% B over 20 min with a flow rate 20 mL/min, retention time=14.1 min) provided the title compound (alkyne 1) as a white fluffy solid (92% yield). ¹H NMR (500 MHz, CDCl₃) δ 8.25 (s, 1H), 7.57 (dd, J=8.5, 7.2 Hz, 1H), 7.22-7.18 (m, 1H), 7.03 (d, J=8.4 Hz, 1H), 4.93 (dd, J=12.3, 5.4 Hz, 1H), 4.10 (d, J=2.5 Hz, 2H), 2.93-2.87 (m, 1H), 2.85-2.70 (m, 2H), 2.28 (t, J=2.4 Hz, 1H), 2.16-2.10 (m, 1H), 2.01 (s, 1H). ¹³C NMR (126 MHz, CDCl₃) δ 169.24, 167.33, 166.40, 165.56, 143.63, 134.24, 130.48, 115.29, 110.84, 109.44, 77.23, 70.26, 47.01, 30.39, 29.47, 20.82. ESI-MS m/z: 312.10 (MH⁺).

### SE47: Synthesis of 2-(2,6-Dioxopiperidin-3-yl)-4-(methyl(prop-2-yn-1-yl)amino)isoindoline-1,3-dione (alkyne 2)

Treatment of 2-(2,6-dioxopiperidin-3-yl)-4-fluoroisoindoline-1,3-dione and N-methylprop-2-yn-1-amine as outlined in SE45 and purification by silica gel chromatograph provided the title compound (alkyne 2) as a yellow solid (61% yield).

### SE48: Synthesis of 4-(But-3-yn-1-ylamino)-2-(2,6-dioxopiperidin-3-yl)isoindoline-1,3-dione (alkyne 3)

Treatment of 2-(2,6-dioxopiperidin-3-yl)-4-fluoroisoindoline-1,3-dione and but-3-yn-1-amine as outlined in SE45 and purification by silica gel chromatograph provided the title compound (alkyne 3) as a yellow solid (52.1% yield). ¹H NMR (500 MHz, CDCl₃) δ 7.98 (s, 1H), 7.52 (dd, J=8.5, 7.1 Hz, 1H), 7.14 (d, J=7.1 Hz, 1H), 6.93 (d, J=8.5 Hz, 1H), 6.49 (s, 1H), 4.92 (dd, J=12.4, 5.4 Hz, 1H), 3.49 (q, J=5.7, 4.5 Hz, 2H), 2.92-2.69 (m, 4H), 2.55 (td, J=6.9, 2.6 Hz, 2H), 2.16-2.10 (m, 1H), 2.08 (t, J=2.6 Hz, 1H). ¹³C NMR (126 MHz, CDCl₃) δ 170.82, 169.31, 168.15, 167.50, 146.37, 136.20, 132.58, 116.51, 112.03, 110.57, 80.61, 70.77, 48.90, 41.29, 31.42, 22.77, 19.38. ESI-MS m/z: 326.10 (MH⁺).

### SE49: Synthesis of tert-Butyl 4-((2-(4-(hydroxymethyl)phenyl)-7-phenylimidazo[1,2-a]pyridin-3-yl)amino)benzoate (304)

Treatment of 4-phenylpyridin-2-amine (301), 4-(hydroxymethyl)benzaldehyde (302) and tert-butyl 4-isocyanobenzoate (303) as outlined in SE41 (rt, 2 days) provided title compound (304) as a yellow solid (80% yield). ¹H NMR (400 MHz, DMSO-d₆) δ 8.87 (s, 1H), 8.03-7.96 (m, 4H), 7.85 (d, J=7.1 Hz, 2H), 7.72 (d, J=8.8 Hz, 2H), 7.53 (t, J=7.6 Hz, 2H), 7.44 (t, J=7.3 Hz, 1H), 7.34 (dd, J=9.0, 7.6 Hz, 3H), 6.60 (s, 2H), 5.18 (t, J=5.5 Hz, 1H), 4.50 (s, 2H), 1.49 (s, 9H). ¹³C NMR (101 MHz, DMSO-d₆) δ 165.38, 150.20, 142.79, 142.64, 138.83, 138.30, 137.36, 132.22, 131.74 (2C), 129.61 (2C), 128.80, 127.08 (4C), 126.67 (2C), 123.56, 121.84, 117.93, 113.91, 112.94 (2C), 112.26, 80.03, 63.15, 28.38 (3C). DUIS-MS m/z: 492.2 (MH⁺), 490.1 (M-H)⁻.

### SE50: Synthesis of tert-Butyl 4-((2-(4-(bromomethyl)phenyl)-7-phenylimidazo[1,2-a]pyridin-3-yl)amino)benzoate (305)

Compounded tert-butyl 4-((2-(4-(hydroxymethyl)phenyl)-7-phenylimidazo[1,2-a]pyridin-3-yl)amino)benzoate (304, 418 mg, 0.85 mmol) was dissolved in THF (9.0 mL). Triphenylphosphane (335 mg, 1.3 mmol) was added. The formed yellow solution was cooled to 0° C. by ice bath. Perbromomethane (424 mg, 1.28 mmol) was added. The reaction mixture was stirred (rt, 0.5 h). After purification by silica gel chromatography. The title compound (305, 320 mg) was afforded as a yellow solid (67.8% yield). ¹H NMR (500 MHz, CDCl₃) δ 7.90 (d, J=8.3 Hz, 2H), 7.84 (d, J=8.9 Hz, 2H), 7.73 (d, J=7.2 Hz, 2H), 7.57 (dd, J=8.4, 1.4 Hz, 2H), 7.45 (t, J=7.5 Hz, 2H), 7.41-7.36 (m, 1H), 7.30 (d, J=8.4 Hz, 2H), 6.99 (dd, J=6.9, 1.9 Hz, 1H), 6.56 (d, J=8.3 Hz, 2H), 6.50 (s, 1H), 4.43 (s, 2H), 1.54 (s, 9H). ¹³C NMR (126 MHz, CDCl₃) δ 165.65, 148.46, 143.24, 139.34, 138.50, 138.30, 137.36, 133.13, 131.77 (2C), 129.32 (2C), 129.13 (2C), 128.43, 127.18 (2C), 126.70 (2C), 123.50, 122.53, 117.20, 114.14, 112.62 (2C), 112.40, 80.40, 33.45, 28.24 (3C). ESI-MS m/z: 554.10, 556.10 (MH⁺).

### SE51: Synthesis of tert-Butyl 4-((2-(4-(azidomethyl)phenyl)-7-phenylimidazo[1,2-a]pyridin-3-yl)amino)benzoate (306)

Compound tert-butyl 4-((2-(4-(bromomethyl)phenyl)-7-phenylimidazo[1,2-a]pyridin-3-yl)amino)benzoate (305, 360 mg, 0.65 mmol) was dissolved in Acetone (5.0 mL) and Water (1.0 mL). sodium azide (127 mg, 1.95 mmol) was added. The mixture was stirred (55° C., 18 h). The reaction mixture was concentrated and purified by silica gel chromatography. The title compound (306, 143 mg) was afforded as a white solid (42.6% yield). ¹H NMR (500 MHz, CDCl₃) δ 7.96 (d, J=8.3 Hz, 2H), 7.86 (d, J=8.7 Hz, 2H), 7.80-7.76 (m, 2H), 7.61 (dd, J=8.3, 1.3 Hz, 2H), 7.47 (t, J=7.6 Hz, 2H), 7.42-7.38 (m, 1H), 7.30-7.26 (m, 2H), 7.04 (dd, J=7.1, 1.8 Hz, 1H), 6.59 (d, J=8.4 Hz, 2H), 6.27 (s, 1H), 4.31 (s, 2H), 1.55 (s, 9H). ¹³C NMR (126 MHz, CDCl₃) δ 165.63, 148.42, 143.34, 139.61, 138.55, 138.38, 135.12, 133.06, 131.83 (2C), 129.15 (2C), 128.49 (2C), 128.45, 127.32 (2C), 126.75 (2C), 123.69, 122.48, 116.98, 114.33, 112.66 (2C), 112.48, 80.45, 54.55, 28.25 (3C). ESI-MS m/z: 517.20 (MH⁺), 539.20 (MNa⁺).

### SE52: Synthesis of tert-Butyl 4-((2-(4-((4-((2-(2-((2-(2,6-dioxopiperidin-3-yl)-1,3-dioxoisoindolin-4-yl)oxy)ethoxy)ethoxy)methyl)-1H-1,2,3-triazol-1-yl)methyl)phenyl)-7-phenylimidazo[1,2-a]pyridin-3-yl)amino)benzoate (307a)

Treatment of (tert-butyl 4-((2-(4-(azidomethyl)phenyl)-7-phenylimidazo[1,2-a]pyridin-3-yl)amino)benzoate (306) and commercially available 2-(2,6-dioxopiperidin-3-yl)-4-(2-(2-(prop-2-yn-1-yloxy)ethoxy)ethoxy)isoindoline-1,3-dione as outlined in SE43 (rt, 18 h) and purification by preparative HPLC (CAT #00G-4436-P0-AX) (linear gradient of 30% B to 50% B over 20 min with a flow rate 20 mL/min, retention time=18.2 min) provided the title compound (307a) as a white fluffy solid (43.5% yield). ESI-MS m/z: 917.20 (MH⁺), 939.20 (MNa⁺).

### SE53: Synthesis of 4-((2-(4-((4-((2-(2-((2-(2,6-Dioxopiperidin-3-yl)-1,3-dioxoisoindolin-4-yl)oxy)ethoxy)ethoxy)methyl)-1H-1,2,3-triazol-1-yl)methyl)phenyl)-7-phenylimidazo[1,2-a]pyridin-3-yl)amino)benzoic acid (XZ743)

Treatment of tert-butyl 4-((2-(4-((4-((2-(2-((2-(2,6-dioxopiperidin-3-yl)-1,3-dioxoisoindolin-4-yl)oxy)ethoxy)ethoxy)methyl)-1H-1,2,3-triazol-1-yl)methyl)phenyl)-7-phenylimidazo[1,2-a]pyridin-3-yl)amino)benzoate (307a) as outlined in SE44 and purification by preparative HPLC (CAT #00G-4436-P0-AX) (linear gradient of 30% B to 40% B over 20 min with a flow rate 20 mL/min, retention time=8.9 min) provided the title compound (XZ743) as a white fluffy solid (52.3% yield). ESI-MS m/z: 861.20 (MH⁺). HRMS calcd. for C₄₇H₄₁N₈O₉ (MH⁺): 861.2991; found 861.3021. HRMS calcd. for C₄₇H₄₂N₈O₉ [(MH₂)²+]: 431.1532; found 431.1541.

### SE54: Synthesis of tert-Butyl 4-((2-(4-((4-(((2-(2,6-dioxopiperidin-3-yl)-1,3-dioxoisoindolin-4-yl)amino)methyl)-1H-1,2,3-triazol-1-yl)methyl)phenyl)-7-phenylimidazo[1,2-a]pyridin-3-yl)amino)benzoate (307b)

Treatment of (tert-butyl 4-((2-(4-(azidomethyl)phenyl)-7-phenylimidazo[1,2-a]pyridin-3-yl)amino)benzoate (306) and 2-(2,6-Dioxopiperidin-3-yl)-4-(prop-2-yn-1-ylamino)isoindoline-1,3-dione (alkyne 1) as outlined in SE43 (rt, 18 h) and purification by preparative HPLC (CAT #00G-4436-P0-AX) (linear gradient of 20% B to 60% B over 20 min with a flow rate 20 m/min, retention time=18.6 min) provided the title compound (307b) as a yellow fluffy solid (60% yield). ESI-MS m/z: 828.20 (MH⁺).

### SE55: Synthesis of 4-((2-(4-((4-(((2-(2,6-Dioxopiperidin-3-yl)-1,3-dioxoisoindolin-4-yl)amino)methyl)-1H-1,2,3-triazol-1-yl)methyl)phenyl)-7-phenylimidazo[1,2-a]pyridin-3-yl)amino)benzoic acid (XZ747)

Treatment of tert-butyl 4-((2-(4-((4-(((2-(2,6-dioxopiperidin-3-yl)-1,3-dioxoisoindolin-4-yl)amino)methyl)-1H-1,2,3-triazol-1-yl)methyl)phenyl)-7-phenylimidazo[1,2-a]pyridin-3-yl)amino)benzoate (307b) as outlined in SE44 and purification by preparative HPLC (CAT #00G-4436-P0-AX) (linear gradient of 20% B to 60% B over 20 min with a flow rate 20 mL/min, retention time=13.4 min) provided the title compound (XZ747) as a yellow fluffy solid (77% yield). ¹H NMR (500 MHz, DMSO-d₆) δ 11.03 (s, 1H), 8.95 (s, 1H), 8.16 (d, J=7.1 Hz, 1H), 8.02 (s, 1H), 8.00 (s, 1H), 7.86 (d, J=8.1 Hz, 2H), 7.81 (d, J=7.5 Hz, 2H), 7.70 (d, J=9.1 Hz, 2H), 7.53-7.40 (m, 5H), 7.31 (d, J=8.3 Hz, 2H), 7.07 (d, J=8.6 Hz, 1H), 7.04-7.00 (m, 1H), 6.97 (d, J=7.0 Hz, 1H), 6.62 (d, J=8.1 Hz, 2H), 5.52 (s, 2H), 4.99 (dd, J=12.8, 5.4 Hz, 1H), 4.52 (d, J=5.1 Hz, 2H), 2.81 (ddd, J=17.0, 13.8, 5.4 Hz, 1H), 2.58-2.41 (m, 3H), 1.95 (ddd, J=7.5, 5.5, 2.5 Hz, 1H). ESI-MS m/z: 772.20 (MH⁺).

### SE56: Synthesis of tert-Butyl 4-((2-(4-((4-(((2-(2,6-dioxopiperidin-3-yl)-1,3-dioxoisoindolin-4-yl)(methyl)amino)methyl)-1H-1,2,3-triazol-1-yl)methyl)phenyl)-7-phenylimidazo[1,2-a]pyridin-3-yl)amino)benzoate (307c)

Treatment of (tert-butyl 4-((2-(4-(azidomethyl)phenyl)-7-phenylimidazo[1,2-a]pyridin-3-yl)amino)benzoate (306) and 2-(2,6-Dioxopiperidin-3-yl)-4-(methyl(prop-2-yn-1-yl)amino)isoindoline-1,3-dione (alkyne 2) as outlined in SE43 (rt, 18 h) and purification by preparative HPLC (CAT #00G-4436-P0-AX) (linear gradient of 20% B to 70% B over 20 min with a flow rate 20 m/min, retention time=16.2 min) provided the title compound (307c) as a yellow solid (47% yield). ESI-MS m/z: 842.20 (MH⁺).

### SE57: Synthesis of 4-((2-(4-((4-(((2-(2,6-Dioxopiperidin-3-yl)-1,3-dioxoisoindolin-4-yl)(methyl)amino)methyl)-1H-1,2,3-triazol-1-yl)methyl)phenyl)-7-phenylimidazo[1,2-a]pyridin-3-yl)amino)benzoic acid (XZ748)

Treatment of tert-butyl 4-((2-(4-((4-(((2-(2,6-dioxopiperidin-3-yl)-1,3-dioxoisoindolin-4-yl)(methyl)amino)methyl)-1H-1,2,3-triazol-1-yl)methyl)phenyl)-7-phenylimidazo[1,2-a]pyridin-3-yl)amino)benzoate (307c) as outlined in SE44 and purification by preparative HPLC (CAT #00G-4436-P0-AX) (linear gradient of 20% B to 60% B over 20 min with a flow rate 20 mL/min, retention time=12.9 min) provided the title compound (XZ748) as a yellow fluffy solid (79% yield). ¹H NMR (500 MHz, DMSO-d₆) δ 11.10 (s, 1H), 9.06 (s, 1H), 8.29 (d, J=7.1 Hz, 1H), 8.08 (d, J=14.6 Hz, 2H), 7.91 (t, J=7.7 Hz, 4H), 7.78 (d, J=8.5 Hz, 2H), 7.63 (d, J=7.1 Hz, 1H), 7.61-7.56 (m, 3H), 7.52 (t, J=7.4 Hz, 1H), 7.32 (d, J=8.1 Hz, 2H), 7.27 (dd, J=13.6, 7.8 Hz, 2H), 6.73 (d, J=8.2 Hz, 2H), 5.61 (s, 2H), 5.11 (dd, J=12.9, 5.4 Hz, 1H), 4.76 (s, 2H), 2.96 (s, 3H), 2.89 (ddd, J=17.5, 14.1, 5.4 Hz, 1H), 2.63-2.53 (m, 2H), 2.03 (ddd, J=10.8, 5.6, 3.2 Hz, 1H). ESI-MS m/z: 786.20 (MH⁺).

### SE58: Synthesis of tert-Butyl 4-((2-(4-((4-(2-((2-(2,6-dioxopiperidin-3-yl)-1,3-dioxoisoindolin-4-yl)amino)ethyl)-1H-1,2,3-triazol-1-yl)methyl)phenyl)-7-phenylimidazo[1,2-a]pyridin-3-yl)amino)benzoate (307d)

Treatment of (tert-butyl 4-((2-(4-(azidomethyl)phenyl)-7-phenylimidazo[1,2-a]pyridin-3-yl)amino)benzoate (306) and (4-(but-3-yn-1-ylamino)-2-(2,6-dioxopiperidin-3-yl)isoindoline-1,3-dione (alkyne 3) as outlined in SE43 (rt, 18 h) and purification by preparative HPLC (CAT #00G-4436-P0-AX) (linear gradient of 30% B to 60% B over 20 min with a flow rate 20 m/min, retention time=16.4 min) provided the title compound (307d) as a yellow solid (49.6% yield). ESI-MS m/z: 842.20 (MH⁺).

### SE59: Synthesis of 4-((2-(4-((4-(2-((2-(2,6-Dioxopiperidin-3-yl)-1,3-dioxoisoindolin-4-yl)amino)ethyl)-1H-1,2,3-triazol-1-yl)methyl)phenyl)-7-phenylimidazo[1,2-a]pyridin-3-V)amino)benzoic acid (XZ749)

Treatment of tert-butyl 4-((2-(4-((4-(2-((2-(2,6-dioxopiperidin-3-yl)-1,3-dioxoisoindolin-4-yl)amino)ethyl)-1H-1,2,3-triazol-1-yl)methyl)phenyl)-7-phenylimidazo[1,2-a]pyridin-3-yl)amino)benzoate (307d) as outlined in SE44 and purification by preparative HPLC (CAT #00G-4436-P0-AX) (linear gradient of 20% B to 60% B over 20 min with a flow rate 20 mL/min, retention time=13.4 min) provided the title compound (XZ749) as a yellow fluffy solid (91% yield).

### SE60: Synthesis of tert-Butyl 4-((2-(4-(((6-((2-(2,6-dioxopiperidin-3-yl)-1,3-dioxoisoindolin-4-yl)amino)hexyl)amino)methyl)phenyl)-7-phenylimidazo[1,2-a]pyridin-3-yl)amino)benzoate (307e)

Compound tert-butyl 4-((2-(4-(bromomethyl)phenyl)-7-phenylimidazo[1,2-a]pyridin-3-yl)amino)benzoate (305, 122 mg, 0.20 mmol) and commercially available 2-(2,6-dioxopiperidin-3-yl)-4-((6-((2,2,2-trifluoroacetyl)-14-azaneyl)hexyl)amino)isoindoline-1,3-dione (93 mg, 0.20 mmol) were mixed with potassium carbonate (82 mg, 0.59 mmol) in DMF (1.0 mL). The reaction was stirred (50° C., 18 h). The reaction mixture was cooled to rt and quenched by water. The formed yellow suspension was filtered and washed by methanol. The title compound (307e, 122 mg) was afforded as a yellow solid (72.8%). DUIS-MS m/z: 424.0 (MH₂²⁺), 846.4 (MH⁺).

### SE61: Synthesis of 4-((2-(4-(((6-((2-(2,6-Dioxopiperidin-3-yl)-1,3-dioxoisoindolin-4-yl)amino)hexyl)amino)methyl)phenyl)-7-phenylimidazo[1,2-a]pyridin-3-yl)amino)benzoic acid (XZ742)

Treatment of tert-butyl 4-((2-(4-(((6-((2-(2,6-dioxopiperidin-3-yl)-1,3-dioxoisoindolin-4-yl)amino)hexyl)amino)methyl)phenyl)-7-phenylimidazo[1,2-a]pyridin-3-yl)amino)benzoate (307e) as outlined in SE44 and purification by preparative HPLC (CAT #00G-4436-P0-AX) (linear gradient of 20% B to 35% B over 20 min with a flow rate 20 mL/min, retention time=16.9 min) provided the title compound (XZ742) as a yellow fluffy solid (27% yield). ESI-MS m/z: 395.70 (MH₂²⁺), 790.20 (MH⁺). HRMS calcd. for C₄₆H₄₄N₇O₆ (MH⁺): 790.3348; found 790.3374. HRMS calcd. for C₄₆H₄₅N₇O₆ [(MH₂)²+]: 395.6710; found 395.6718.

### SE62: Synthesis of Methyl 4-((7-ethynyl-2-(4-ethynylphenyl)imidazo[1,2-a]pyridin-3-yl)amino)benzoate (3092)

Treatment of 4-ethynylpyridin-2-amine, 4-ethynylbenzaldehyde and methyl 4-isocyanobenzoate as outlined in SE41 (rt, 3 days) provided methyl 4-((7-ethynyl-2-(4-ethynylphenyl)imidazo[1,2-a]pyridin-3-yl)amino)benzoate (309g) as a pale yellow solid (62.1% yield).

### SE63: Synthesis of Methyl 4-((6-ethynyl-2-(4-ethynylphenyl)imidazo[1,2-a]pyridin-3-yl)amino)benzoate (309h)

Treatment of 5-ethynylpyridin-2-amine, 4-ethynylbenzaldehyde and methyl 4-isocyanobenzoate as outlined in SE41 (rt, 3 days) provided methyl 4-((6-ethynyl-2-(4-ethynylphenyl)imidazo[1,2-a]pyridin-3-yl)amino)benzoate (309h) as a brown solid (74.8% yield).

### SE64: Synthesis of 4-((7-Ethynyl-2-(4-ethynylphenyl)imidazo[1,2-a]pyridin-3-yl)amino)benzoic acid (310g)

Treatment of methyl 4-((7-ethynyl-2-(4-ethynylphenyl)imidazo[1,2-a]pyridin-3-yl)amino)benzoate (309g) as outlined in SE42 (rt, 24 h) and purification by preparative HPLC (linear gradient of 20% B to 50% B over 20 min with a flow rate 20 mL/min; retention time=12.2 min) provided 4-((7-ethynyl-2-(4-ethynylphenyl)imidazo[1,2-a]pyridin-3-yl)amino)benzoic acid (310g) as a light yellow solid (34.7% yield). ESI-MS m/z: 378.10 (MH⁺). HRMS calcd C₂₄H₁₆N₃O₂ (MH⁺), 378.1237; found 378.1231.

### SE65: Synthesis of 4-((6-Ethynyl-2-(4-ethynylphenyl)imidazo[1,2-a]pyridin-3-yl)amino)benzoic acid (310h)

Treatment of methyl 4-((6-ethynyl-2-(4-ethynylphenyl)imidazo[1,2-a]pyridin-3-yl)amino)benzoate (309h) as outlined in SE42 (rt, 24 h) and purification by preparative HPLC (linear gradient of 20% B to 50% B over 20 min with a flow rate 20 mL/min; retention time=12.1 min) provided 4-((6-ethynyl-2-(4-ethynylphenyl)imidazo[1,2-a]pyridin-3-yl)amino)benzoic acid (310h) as a light yellow solid (39.1% yield). ESI-MS m/z: 378.10 (MH⁺). HRMS calcd C₂₄H₁₆N₃O₂ (MH⁺), 378.1237; found 378.1230.

### SE66: Synthesis of 4-((7-(1-(4-(2-((2-(2,6-Dioxopiperidin-3-yl)-1,3-dioxoisoindolin-4-yl)oxy)acetamido)butyl)-1H-1,2,3-triazol-4-yl)-2-phenylimidazo[1,2-a]pyridin-3-V)amino)benzoic acid (XZ685)

Treatment of 4-((7-ethynyl-2-phenylimidazo[1,2-a]pyridin-3-yl)amino)benzoic acid (325a) and commercially available N-(4-azidobutyl)-2-((2-(2,6-dioxopiperidin-3-yl)-1,3-dioxoisoindolin-4-yl)oxy)acetamide as outlined in SE43 and purification by preparative HPLC (Cat #00F-4436-U0-AX) (linear gradient of 20% B to 40% B over 20 min with a flow rate 20 mL/min, retention time=14.3 min) provided 4-((7-(1-(4-(2-((2-(2,6-dioxopiperidin-3-yl)-1,3-dioxoisoindolin-4-yl)oxy)acetamido)butyl)-1H-1,2,3-triazol-4-yl)-2-phenylimidazo[1,2-a]pyridin-3-yl)amino)benzoic acid (XZ685) as a white solid (60.1% yield). ESI-MS m/z: 782.2 (MH⁺). HRMS calcd C₄₁H₃₆N₉O₈ (MH⁺), 782.2681; found, 782.2677.

### SE67: Synthesis of 4-((2-(4-(1-(4-(2-((2-(2,6-Dioxopiperidin-3-yl)-1,3-dioxoisoindolin-4-yl)oxy)acetamido)butyl)-1H-1,2,3-triazol-4-yl)phenyl)imidazo[1,2-a]pyridin-3-V)amino)benzoic acid (XZ689)

Treatment of 4-((2-(4-ethynylphenyl)imidazo[1,2-a]pyridin-3-yl)amino)benzoic acid (325e) and commercially available N-(4-azidobutyl)-2-((2-(2,6-dioxopiperidin-3-yl)-1,3-dioxoisoindolin-4-yl)oxy)acetamide as outlined in SE43 and purification by preparative HPLC (CAT #00F-4436-U0-AX) (linear gradient of 20% B to 25% B over 20 min with a flow rate 20 mL/min, retention time=12.5 min) provided 4-((2-(4-((1-(4-(2-((2-(2,6-dioxopiperidin-3-yl)-1,3-dioxoisoindolin-4-yl)oxy)acetamido)butyl)-1H-1,2,3-triazol-4-yl)methoxy)phenyl)imidazo[1,2-a]pyridin-3-yl)amino)benzoic acid 4-((2-(4-(1-(4-(2-((2-(2,6-dioxopiperidin-3-yl)-1,3-dioxoisoindolin-4-yl)oxy)acetamido)butyl)-1H-1,2,3-triazol-4-yl)phenyl)imidazo[1,2-a]pyridin-3-yl)amino)benzoic acid (XZ689) as a white fluffy solid (46.6% yield). ESI-MS m/z: 782.2 (MH⁺). HRMS calcd C₄₁H₃₆N₉O₈ (MH⁺), 782.2681; found, 782.2684. HRMS calcd C₄₁H₃₇N₉O₉ (MH₂)²⁺, 391.6377; found, 391.6376.

### SE68: Synthesis of 4-((2-(4-((1-(4-(2-((2-(2,6-Dioxopiperidin-3-yl)-1,3-dioxoisoindolin-4-yl)oxy)acetamido)butyl)-1H-1,2,3-triazol-4-yl)methoxy)phenyl)imidazo[1,2-a]pyridin-3-yl)amino)benzoic acid (XZ688)

Treatment of 4-((2-(4-(prop-2-yn-1-yloxy)phenyl)imidazo[1,2-a]pyridin-3-yl)amino)benzoic acid (325c) and commercially available N-(4-azidobutyl)-2-((2-(2,6-dioxopiperidin-3-yl)-1,3-dioxoisoindolin-4-yl)oxy)acetamide as outlined in SE43 and purification by preparative HPLC (CAT #00F-4436-U0-AX) (linear gradient of 30% B to 35% B over 20 min with a flow rate 20 mL/min, retention time=13.2 min) provided 4-((2-(4-((1-(4-(2-((2-(2,6-dioxopiperidin-3-yl)-1,3-dioxoisoindolin-4-yl)oxy)acetamido)butyl)-1H-1,2,3-triazol-4-yl)methoxy)phenyl)imidazo[1,2-a]pyridin-3-yl)amino)benzoic acid (XZ688) as a white fluffy solid (54.7% yield). ESI-MS m/z: 812.2 (MH⁺). HRMS calcd C₄₂H₃₈N₉O₉ (MH⁺), 812.2787; found, 812.2762. HRMS calcd C₄₂H₃₉N₉O₉ (MH₂)²⁺, 406.6430; found, 406.6415.

### SE69: Synthesis of 4-((2-(4-(1-(4-(2-((2-(2,6-Dioxopiperidin-3-yl)-1,3-dioxoisoindolin-4-yl)oxy)acetamido)butyl)-1H-1,2,3-triazol-4-yl)phenyl)-7-phenylimidazo[1,2-a]pyridin-3-yl)amino)benzoic acid (XZ687)

Treatment of (4-((2-(4-ethynylphenyl)-7-phenylimidazo[1,2-a]pyridin-3-yl)amino)benzoic acid (325f) and commercially available N-(4-azidobutyl)-2-((2-(2,6-dioxopiperidin-3-yl)-1,3-dioxoisoindolin-4-yl)oxy)acetamide as outlined in SE43 and purification by preparative HPLC (CAT #00F-4436-U0-AX) (linear gradient of 30% B to 35% B over 20 min with a flow rate 20 mL/min, retention time=13.3 min) provided 4-((2-(4-(1-(4-(2-((2-(2,6-dioxopiperidin-3-yl)-1,3-dioxoisoindolin-4-yl)oxy)acetamido)butyl)-1H-1,2,3-triazol-4-yl)phenyl)-7-phenylimidazo[1,2-a]pyridin-3-yl)amino)benzoic acid (XZ687) as a white fluffy solid (33.2% yield). ESI-MS m/z: 858.3 (MH⁺). HRMS calcd C₄₇H₄₀N₉O₈ (MH⁺), 858.2994; found, 858.2990. HRMS calcd C₄₇H₄₁N₉O₈ (MH₂)²⁺, 429.6534; found, 429.6529.

### SE70: Synthesis of 4-((2-(4-((1-(4-(2-((2-(2,6-Dioxopiperidin-3-yl)-1,3-dioxoisoindolin-4-yl)oxy)acetamido)butyl)-1H-1,2,3-triazol-4-yl)methoxy)phenyl)-7-phenylimidazo[1,2-a]pyridin-3-yl)amino)benzoic acid (XZ686)

Treatment of 4-((7-phenyl-2-(4-(prop-2-yn-1-yloxy)phenyl)imidazo[1,2-a]pyridin-3-yl)amino)benzoic acid (325d) and commercially available N-(4-azidobutyl)-2-((2-(2,6-dioxopiperidin-3-yl)-1,3-dioxoisoindolin-4-yl)oxy)acetamide as outlined in SE43 and purification by preparative HPLC (CAT #00F-4436-U0-AX) (linear gradient of 30% B to 35% B over 20 min with a flow rate 20 mL/min, retention time=12.6 min) provided 4-((2-(4-((1-(4-(2-((2-(2,6-dioxopiperidin-3-yl)-1,3-dioxoisoindolin-4-yl)oxy)acetamido)butyl)-1H-1,2,3-triazol-4-yl)methoxy)phenyl)-7-phenylimidazo[1,2-a]pyridin-3-yl)amino)benzoic acid (XZ686) as a white fluffy solid (55.3% yield). ESI-MS m/z: 888.1 (MH⁺). HRMS calcd C₄₈H₄₂N₉O₉ (MH⁺), 888.3100; found, 888.3076. HRMS calcd C₄₈H₄₃N₉O₉ (MH₂)²⁺, 444.6586; found, 444.6572.

### SE71: Synthesis of 4-((7-(1-(2-(2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)-1H-1,2,3-triazol-4-yl)-2-phenylimidazo[1,2-a]pyridin-3-yl)amino)benzoic acid (XZ682)

Treatment of 4-((7-ethynyl-2-phenylimidazo[1,2-a]pyridin-3-yl)amino)benzoic acid (325a) and commercially available (2S,4R)-1-((S)-2-(2-(2-(2-azidoethoxy)ethoxy)acetamido)-3,3-dimethylbutanoyl)-4-hydroxy-N-(4-(4-methylthiazol-5-yl)benzyl)pyrrolidine-2-carboxamide as outlined in SE43 and purification by preparative HPLC (CAT #00F-4436-U0-AX) (linear gradient of 25% B to 30% B over 20 min with a flow rate 20 mL/min, retention time=15.6 min) provided 4-((7-(1-(2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)-1H-1,2,3-triazol-4-yl)-2-phenylimidazo[1,2-a]pyridin-3-yl)amino)benzoic acid (XZ682) as a white fluffy solid (33.6% yield). MS m/z: 955.2 (MH⁺). HRMS calcd C₅₀H₅₅N₁₀O₈S (MH⁺), 955.3920; found, 955.3918. HRMS calcd C₅₀H₅₆N₁₀O₈S (MH₂)²⁺, 478.1996; found, 478.1990.

### SE72: Synthesis of 4-((7-(1-((S)-13-((2S,4R)-4-Hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl)-1H-1,2,3-triazol-4-yl)-2-phenylimidazo[1,2-a]pyridin-3-yl)amino)benzoic acid (XZ683)

Treatment of 4-((7-ethynyl-2-phenylimidazo[1,2-a]pyridin-3-yl)amino)benzoic acid (325a) and commercially available (2S,4R)-1-((S)-14-azido-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide as outlined in SE43 and purification by preparative HPLC (CAT #00F-4436-U0-AX) (linear gradient of 20% B to 35% B over 20 min with a flow rate 20 mL/min, retention time=18.8 min) provided 4-((7-(1-((S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl)-1H-1,2,3-triazol-4-yl)-2-phenylimidazo[1,2-a]pyridin-3-yl)amino)benzoic acid (XZ683) as a white fluffy solid (58.2% yield). ESI-MS m/z: 999.3 (MH⁺). HRMS calcd C₅₂H₅₉N₁₀O₉S (MH⁺), 999.4182; found, 999.4167. HRMS calcd C₅₂H₆₀N₁₀O₉S (MH₂)²⁺, 500.2127; found, 500.2114.

### SE73: Synthesis of 4-((7-(1-((S)-16-((2S,4R)-4-Hydroxy-2-((4-(4-methylthiazol-5-yl)benzyl)carbamoyl)pyrrolidine-1-carbonyl)-17,17-dimethyl-14-oxo-3,6,9,12-tetraoxa-15-azaoctadecyl)-1H-1,2,3-triazol-4-yl)-2-phenylimidazo[1,2-a]pyridin-3-yl)amino)benzoic acid (XZ684)

Treatment of 4-((7-ethynyl-2-phenylimidazo[1,2-a]pyridin-3-yl)amino)benzoic acid (325a) and commercially available (2S,4R)-1-((S)-17-azido-2-(tert-butyl)-4-oxo-6,9,12,15-tetraoxa-3-azaheptadecanoyl)-4-hydroxy-N-(4-(4-methylthiazol-5-yl)benzyl)pyrrolidine-2-carboxamide as outlined in SE43 and purification by preparative HPLC (Cat #00F-4436-U0-AX) (linear gradient of 20% B to 35% B over 20 min with a flow rate 20 mL/min, retention time=19.2 min) provided 4-((7-(1-((S)-16-((2S,4R)-4-hydroxy-2-((4-(4-methylthiazol-5-yl)benzyl)carbamoyl)pyrrolidine-1-carbonyl)-17,17-dimethyl-14-oxo-3,6,9,12-tetraoxa-15-azaoctadecyl)-1H-1,2,3-triazol-4-yl)-2-phenylimidazo[1,2-a]pyridin-3-yl)amino)benzoic acid (XZ684) as a white fluffy solid (61.1% yield). ESI-MS m/z: 1143.2 (MH⁺). HRMS calcd C₅₄H₆₃N₁₀O₁₀S (MH⁺), 1043.4444; found, 1043.4425. HRMS calcd C₅₄H₆₄N₁₀O₁₀S (MH₂)²⁺, 522.2258; found, 522.2243.

### SE74: Synthesis of 4-((2-(4-((1-(2-(2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)-1H-1,2,3-triazol-4-yl)methoxy)phenyl)-7-phenylimidazo[1,2-a]pyridin-3-yl)amino)benzoic acid (XZ679)

Treatment of 4-((7-phenyl-2-(4-(prop-2-yn-1-yloxy)phenyl)imidazo[1,2-a]pyridin-3-yl)amino)benzoic acid (325d) and commercially available (2S,4R)-1-((S)-2-(2-(2-(2-azidoethoxy)ethoxy)acetamido)-3,3-dimethylbutanoyl)-4-hydroxy-N-(4-(4-methylthiazol-5-yl)benzyl)pyrrolidine-2-carboxamide as outlined in SE43 and purification by preparative HPLC (CAT #00F-4436-U0-AX) (linear gradient of 30% B to 35% B over 20 min with a flow rate 20 m/min, retention time=13.7 min) provided 4-((2-(4-((1-(2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)-1H-1,2,3-triazol-4-yl)methoxy)phenyl)-7-phenylimidazo[1,2-a]pyridin-3-yl)amino)benzoic acid (XZ679) as a white fluffy solid (43.6% yield). ESI-MS m/z: 1061.2 (MH⁺), 531.3 (MH₂)²⁺. HRMS calcd C₅₇H₆₁N₁₀O₉S (MH⁺), 1061.4338; found, 1061.4331. HRMS calcd C₅₇H₆₂N₁₀O₉S (MH₂)²⁺, 531.2205; found, 531.2196.

### SE75: Synthesis of 4-((2-(4-((1-((S)-13-((2S,4R)-4-Hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl)-1H-1,2,3-triazol-4-yl)methoxy)phenyl)-7-phenylimidazo[1,2-a]pyridin-3-V)amino)benzoic acid (XZ680)

Treatment of 4-((7-phenyl-2-(4-(prop-2-yn-1-yloxy)phenyl)imidazo[1,2-a]pyridin-3-yl)amino)benzoic acid (325d) and commercially available (2S,4R)-1-((S)-14-azido-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide as outlined in SE43 and purification by preparative HPLC (CAT #00F-4436-U0-AX) (linear gradient of 20% B to 50% B over 20 min with a flow rate 20 mL/min, retention time=15.3 min) provided 4-((2-(4-((1-((S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl)-1H-1,2,3-triazol-4-yl)methoxy)phenyl)-7-phenylimidazo[1,2-a]pyridin-3-yl)amino)benzoic acid (XZ680) as a white fluffy solid (41.6% yield). ESI-MS m/z: 1105.2 (MH⁺). HRMS calcd C₅₉H₆₅N₁₀O₁₀S (MH⁺), 1105.4600; found, 1105.2327. HRMS calcd C₅₉H₆₆N₁₀O₁₀S (MH₂)²⁺, 553.2337; found, 553.2327.

### SE76: Synthesis of 4-((2-(4-((1-((S)-16-((2S,4R)-4-Hydroxy-2-((4-(4-methylthiazol-5-yl)benzyl)carbamoyl)pyrrolidine-1-carbonyl)-17,17-dimethyl-14-oxo-3,6,9,12-tetraoxa-15-azaoctadecyl)-1H-1,2,3-triazol-4-yl)methoxy)phenyl)-7-phenylimidazo[1,2-a]pyridin-3-yl)amino)benzoic acid (XZ681)

Treatment of 4-((7-phenyl-2-(4-(prop-2-yn-1-yloxy)phenyl)imidazo[1,2-a]pyridin-3-yl)amino)benzoic acid (325d) and commercially available (2S,4R)-1-((S)-17-azido-2-(tert-butyl)-4-oxo-6,9,12,15-tetraoxa-3-azaheptadecanoyl)-4-hydroxy-N-(4-(4-methylthiazol-5-yl)benzyl)pyrrolidine-2-carboxamide as outlined in SE43 and purification by preparative HPLC (CAT #00F-4436-U0-AX) (linear gradient of 30% B to 35% B over 20 min with a flow rate 20 m/min, retention time=13.9 min) provided 4-((2-(4-((1-((S)-16-((2S,4R)-4-hydroxy-2-((4-(4-methylthiazol-5-yl)benzyl)carbamoyl)pyrrolidine-1-carbonyl)-17,17-dimethyl-14-oxo-3,6,9,12-tetraoxa-15-azaoctadecyl)-1H-1,2,3-triazol-4-yl)methoxy)phenyl)-7-phenylimidazo[1,2-a]pyridin-3-yl)amino)benzoic acid (XZ681) as a white fluffy solid (53.2% yield). ESI-MS m/z: 1149.3 (MH⁺). HRMS calcd C₆₁H₆₉N₁₀O₁₁S (MH⁺), 1149.4863; found, 1149.4847. HRMS calcd C₆₁H₇₀N₁₀O₁₁S (MH₂)²⁺, 575.2468; found, 575.2457.

### Examples

### Example 1: TDP1 Gel-based in vitro assay—imidazo[1,2-a]pyridin-3-amines and analogs thereof

TDP1 Gel-based in vitro assay was carried out as previously described Lountos, G. T., et al. (Nucleic Acids Res. (2019) 47(19) 10134-10150.) (5′-Cy5-labeled DNA substrate (1 nM; N14Y; 5′-GATCTAAAAGACTT-pY-3′) was incubated with 10 μM recombinant TDP1 in the absence or presence of inhibitor (at concentrations ranging from 20 nM to 10 mM) for 15 min at room temperature in a buffer containing 50 mM Tris HCl, pH 7.5, 80 mM KCl, 2 mM EDTA, 1 mM DTT, 40 μg/ml BSA and 0.01% Tween-20. Reactions were terminated by addition of 1 volume of gel loading buffer [99.5% (v/v) formamide, 5 mM EDTA, 0.01% (w/v) xylene cyanol, and 0.01% (w/v) bromophenol blue]. Samples were subjected to a 16% denaturing PAGE and gels were exposed after drying to a PhosphorImager screen (GE Healthcare). Gel images were scanned using a Typhoon FLA 9500 scanner (GE Healthcare) and densitometric analyses were performed using the ImageQuant software (GE Healthcare). The IC50 of TDP1 inhibitors was calculated by comparing the percentage of cleavage product (5′Cy5-GATCTAAAAGACTT-p-3′) to DMSO control. Results for evaluation of imidazo[1,2-a]pyridin-3-amines are shown in Table 1. Results for evaluation of analogues based imidazo[1,2-a]pyrazin-3-amine core are shown in Table 2.

TABLE 1

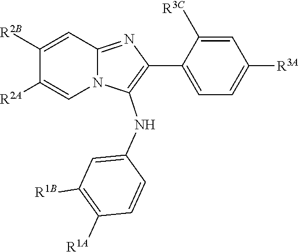

| Cmp. | | | | | | | IC₅₀ |
|---|---|---|---|---|---|---|---|
| No. | R^{1A} | R^{1B} | R^{2A} | R^{2B} | R^{3A} | R^{3C} | (μM) |
| 6t | H | H | H | H | H | H | >100 |
| 6u | NO₂ | H | SO₂Me | H | H | H | 16.1 |
| 8a | COOH | H | H | H | H | OH | 9.3 |
| 8b | COOH | H | H | H | CH₃ | H | 14.8 |
| 8c | COOH | H | H | H | OBn | H | 12.7 |
| 8d | COOH | H | H | H | CF₃ | H | 4.2 |
| 8e | COOH | H | H | H | NO₂ | H | 3.2 |
| 8f | COOH | H | H | H | OH | H | >100 |
| 8g | COOH | H | H | H | SO₃H | H | >100 |
| 8h | COOH | H | H | H | COOH | H | >100 |
| 8i | H | H | H | H | COOH | H | 40.1 |
| 8j | COOH | H | COOH | H | H | COOH | >100 |
| 8k | H | H | COOH | H | H | H | 2.4 |
| 8l | NO₂ | H | COOH | H | H | H | 22.0 |
| 8m | COOH | H | COOH | H | H | H | 0.9 |
| 8n | COOH | H | COOH | H | Ph | H | 1.5 |
| 8o | COOH | H | Br | H | H | H | 1.6 |
| 8p | COOH | H | H | Ph | H | H | 1.8 |
| 8q | COOH | H | H | H | SO₂Me | H | 1.8 |
| 8r | COOH | H | SO₂Me | H | Ph | H | 9.95 |
| 8s | COOH | H | H | COOH | H | H | 4.0 |
| 10b | COOH | COOH | H | H | H | H | 11.0 |

TABLE 2

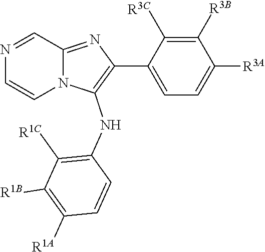

| Cmp. | | | | | | | IC₅₀ |
|---|---|---|---|---|---|---|---|
| No. | R^{1A} | R^{1B} | R^{1C} | R^{3A} | R^{3B} | R^{3C} | (μM) |
| M7 | COOH | H | H | H | H | OH | 3.0 |
| M8 | H | COOH | H | Me | H | H | 233 |
| 7a | CO₂Et | H | H | H | H | OH | >1000 |
| 7b | COOH | H | H | H | H | H | 1.5 |
| 7c | CONH₂ | H | H | OMe | H | H | >1000 |
| 7d | COOH | H | H | H | OH | H | 127 |
| 7e | COOH | H | H | OH | H | H | 20 |
| 7f | COOH | H | OH | H | H | H | 18 |
| 7g | H | H | OMe | COOH | H | H | 2300 |
| 7h | CONH₂ | H | H | Me | H | H | >1000 |
| 7i | CONHMe | H | H | H | H | H | >1000 |
| 7j | H | CO₂Me | H | H | H | OH | >1000 |
| 7k | H | H | CO₂Me | H | H | OH | >1000 |
| 71 | H | COOH | H | H | H | H | >1000 |
| 7m | H | COOH | H | H | H | OH | 370 |
| 7n | H | COOH | H | H | OH | H | 2050 |
| 7o | H | COOH | H | OH | H | H | 216 |
| 7p | H | COOH | H | N(CH₂CH)₂O | H | H | 942 |
| 7q | H | COOH | H | NMe₂ | H | H | 2900 |
| 7r | H | CO₂Et | H | COOH | H | H | >1000 |
| 7′s | H | H | COOH | OH | H | H | >1000 |
| 7′t | H | COOH | H | COOH | H | H | >1000 |
| 7v | COOH | H | H | NMe₂ | H | H | >1000 |
| 7w | COOH | H | H | Cl | H | H | >1000 |
| 7x | COOH | H | H | H | Cl | H | >1000 |
| 7y | COOH | H | H | H | Br | H | >1000 |
| 7′z | COOH | H | H | F | H | H | >1000 |
| 10a | COOH | COOH | H | H | H | H | 45.5 |

### Example 2: Lineweaver Burke Plot Analysis

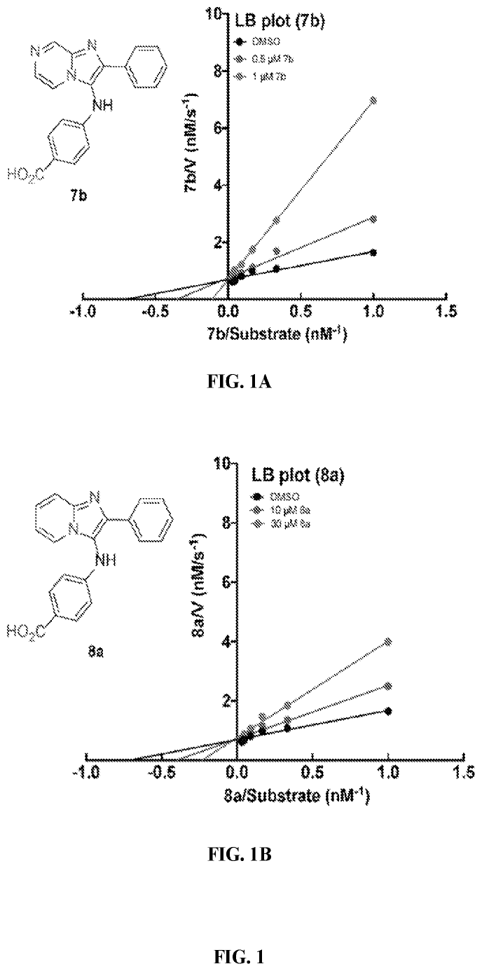

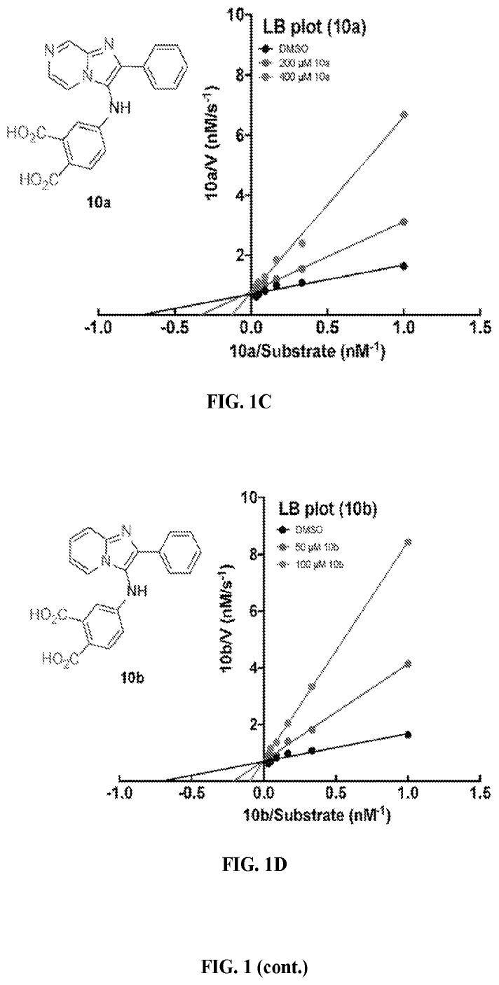

To determine the kinetic parameters for the inhibition of TDP1 by 7b, 8a, 10a, and 10b, 400 nM of recombinant human TDP1 enzyme was incubated with 40 nM of labeled DNA substrate (CY5N14Y) with 0, 0.08, 0.2, 0.4, 0.8, or 1.2 μM of unlabeled DNA substrate (N14Y) in the presence or absence of the inhibitors in a final volume of 10 μL in 1×LMP 1 reaction buffer (50 mM Tris-HCl, pH 7.5, 80 mM KCl, 2 mM EDTA, 1 mM DTT, 40 μg/mL BSA, 0.01% Tween 20). The reactions were carried out at room temperature for 0.25, 0.5, 1, 1.5, 3, 6, or 10 min and terminated by adding 1 volume of 2× stop buffer (99.5% formamide, 10 mM EDTA). Samples were then analyzed with a 20% DNA sequencing gel and exposed to a PhosphorImager screen for further analysis by Typhoon FLA 9500 (GE Healthcare). The Linearweaver-Burk plots (FIG. 1) were generated based on the substrate concentrations and corresponding reaction velocities.

### Example 3: IV Synergistic Effect of TDP1 Inhibitors with Camptothecin (CPT) in Human Colon Cancer Cell Line HCT116

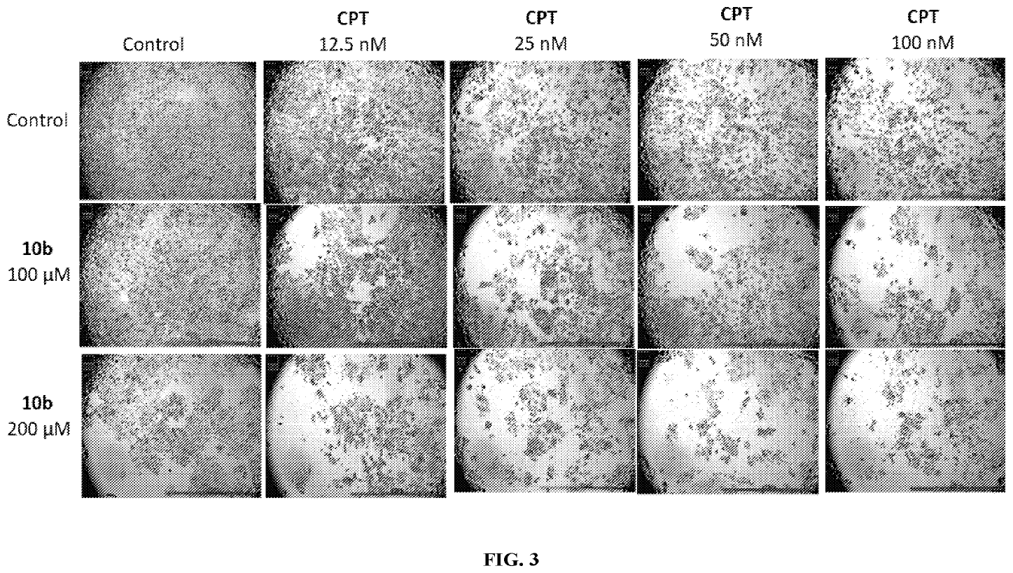

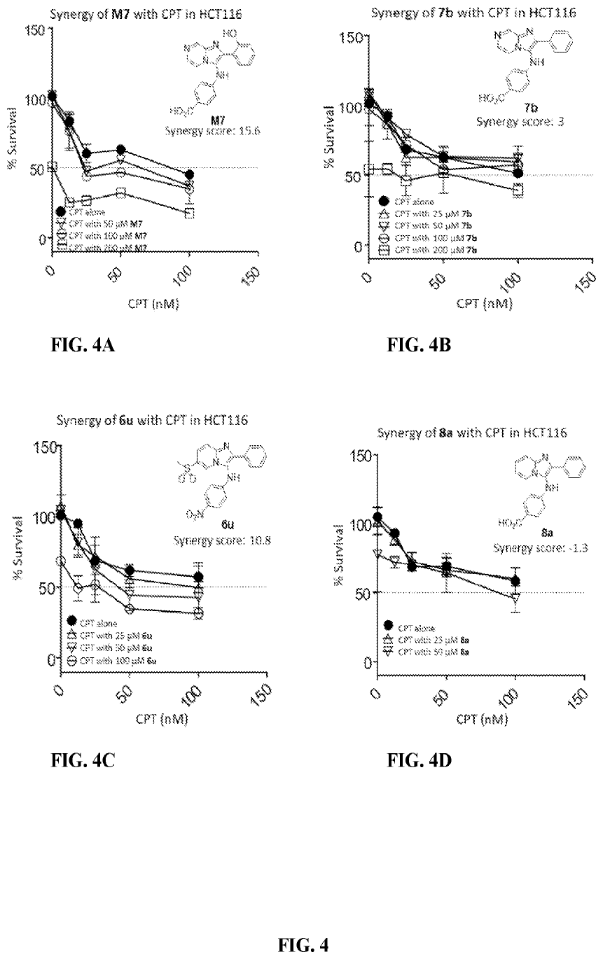

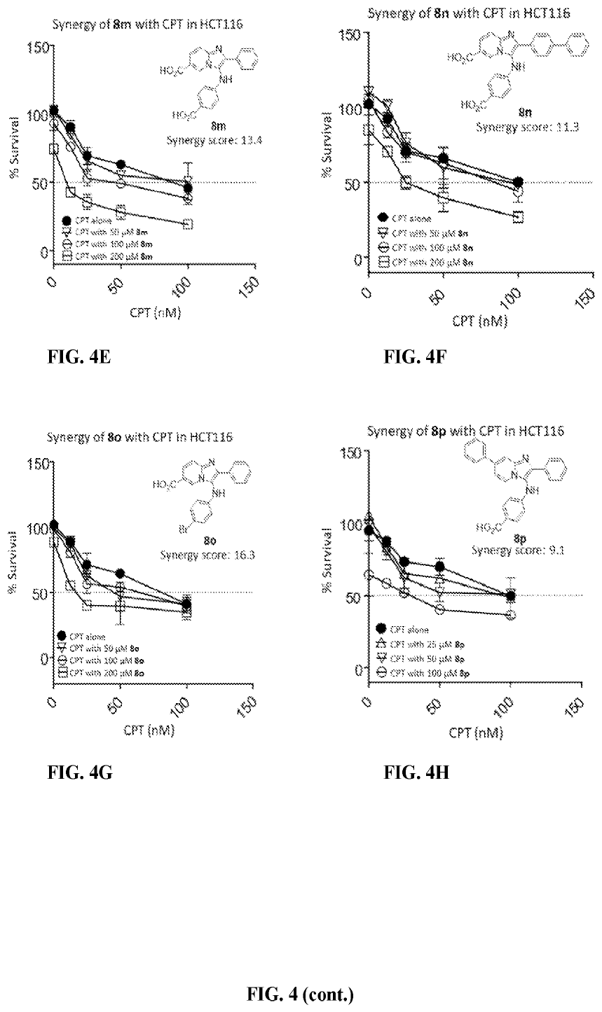

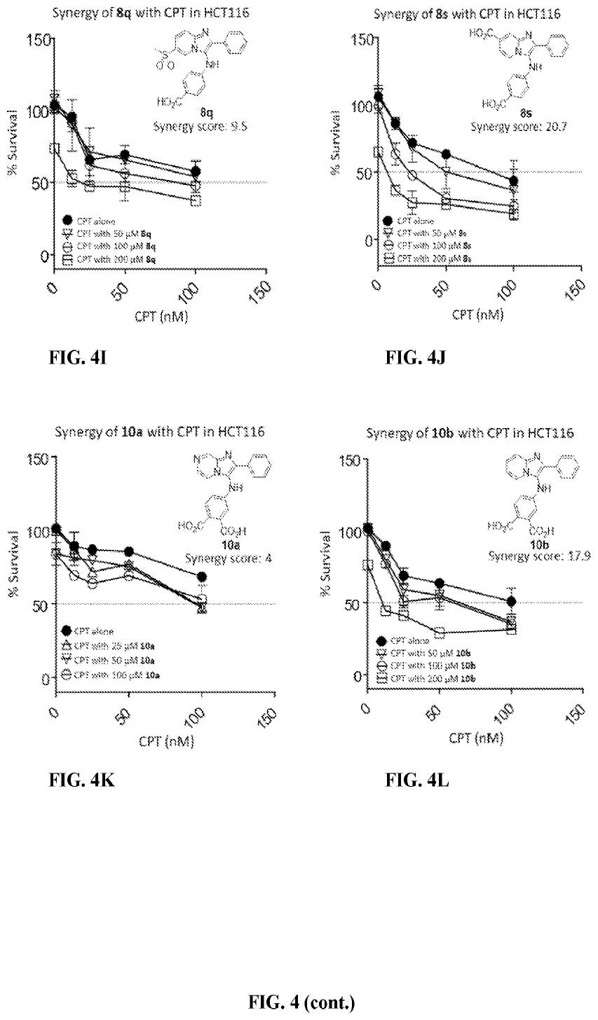

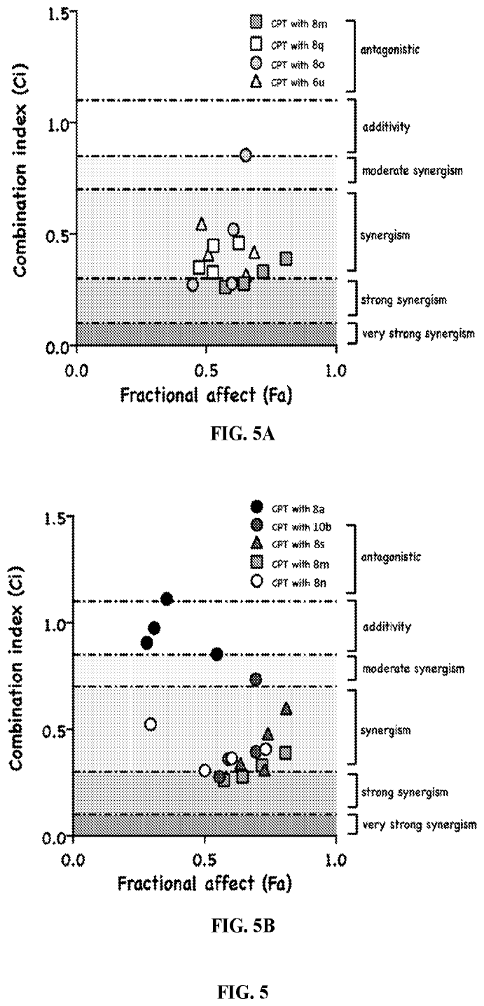

The synergistic effects of the TDP1 inhibitors with CPT were tested in human colon cancer cell line HCT116 based on cell viability (Table 3, FIGS. 2-5). Cells were first seeded in a 384-well black-clear plate until 30% confluency and then incubated with a serial dilution of CPT at the range of 0-100 nM in the present or the absence of TDP1 inhibitors for 72 h at 37° C. Viable cell numbers were counted from the brightfield images taken by Biotek Cytation 5 (FIGS. 2-3). Fractional affect (Fa) and combination index (Ci) were analyzed with CompuSyn software. Synergistic scores of each TDP1 inhibitors were calculated and collected based on the data analysis from SynergyFinder (FIG. 4). Fractional affect (Fa) and combination index (Ci) were analyzed with CompuSyn software (FIG. 5).

TABLE 3
The TDP1 selectivity of lead compounds over TDP2 using gel-based assays in vitro

| | | | | WCE | | |
|---|---|---|---|---|---|---|
| | TDP1 IC₅₀ | TDP2 IC₅₀ | TDP1 | hTDP1 IC₅₀ | Ki | Synergy |
| Compound | (μM)ⁱ | (μM)ⁱⁱ | Selectivityⁱⁱⁱ | (μM) ^{iv} | (μM)^{v} | Score ^{vii} |
| M7 | 19 ± 5.9 | >100 | 5 | >100 | — | 15.6 |
| | (3.0)^{vi} | | | | | |
| M8 | >100 | >100 | — | — | — | — |
| | (233)^{vi} | | | | | |
| 7b | 0.71 ± 0.03 | >100 | >140 | 28.7 ± 8.7 | 0.309 ± 0.122 | 3 |
| | (1.54)^{vi} | | | | | |
| 8a | 8.72 ± 1.81 | >100 | >11 | >100 | 12.49 ± 0.49 | −1.3 |
| | (4.0)^{vi} | | | | | |
| 8n | 2.94 ± 0.47 | >100 | >34 | >100 | — | 11.3 |
| | (1.5)^{vi} | | | | | |
| 8p | 2.98 ± 0.24 | >100 | >33 | 50.2 ± 15.8 | — | 9.1 |
| | (1.8)^{vi} | | | | | |
| 8q | 3.03 ± 1.19 | 80 | 26 | 38.4 ± 9.95 | — | 9.5 |
| | (1.8)^{vi} | | | | | |
| 8s | 1.32 ± 0.22 | >100 | >75 | >100 | — | 20.7 |
| | (2.9)^{vi} | | | | | |
| 10a | >100 | >100 | — | — | 109.9 ± 30.9 | 4 |
| | (45.5)^{vi} | | | | | |
| 10b | 15.9 ± 1.21 | >100 | >6 | >100 | 17.40 ± 2.80 | 17.9 |
| | (11)^{vi} | | | | | |

ⁱThe half maximal inhibitory concentration (IC₅₀) based on gel based TDP1 fluorescence assay.
ⁱⁱThe half maximal inhibitory concentration (IC₅₀) based on gel based TDP2 fluorescence assay.
ⁱⁱⁱTDP1 selectivity based on the ratio of IC₅₀ values of TDP1/TDP2.
^{iv} The half maximal inhibitory concentration (IC₅₀) based on gel based whole cell extract (WCE) human TDP1 fluorescence assay. (Marchand, C., et al., “Biochemical Assays for the Discovery of TDP1 Inhibitors,” Mol. Chancer Ther. (2014) 13(8): 2116-2126)
^{v}Constant of inhibition Ki (FIG. 1).
^{vi}Separated data from previous experiments.
^{vii} Synergy Scores of TDP1 inhibitors with camptothecin (CPT) were calculated based on SynergyFinder 2.0 to reflect the synergistic effect (FIG. 4).

### Example 4: Aldehydes Used in the Oxime-Based TDP1 Inhibitor Series

Table 4 describes aldehydes used in the oxime-based TDP1 inhibitor series with IUPAC names and Simplified Molecular-Input Line-Entry System (SMILES) strings.

TABLE 4

| Aldehyde | IUPAC Name | SMILES |
|---|---|---|
| A1 | 3,4-dimethyl-benzaldehyde | Cc1ccc(C═O)cc1C |
| A2 | 1-tosyl-1H-pyrrole-2-carbaldehyde | Cc1ccc(cc1)S(n1cccc1C═O)(═O)═O |
| A3 | 2-(4-fluoro-phenyl)-benzaldehyde | C(c1ccccc1c1ccc(cc1)F)═O |
| A4 | methyl 5-chloro-4-formyl-1- | Cn1c(c(C═O)c(C(═O)OC)n1)[Cl] |
| | methyl-1H-pyrazole-3-carboxylate | |
| A5 | ethyl 4-(2-formyl-phenyl)- | CCOC(N1CCN(CC1)c1ccccc1C═O)═O |
| | piperazine-1-carboxylate | |
| A6 | 4-(1H-1,2,3,4-tetraazol-5-yl)- | C(c1ccc(cc1)c1nnn[nH]1)═O |
| | benzaldehyde | |
| A7 | 4-(2-chloro-phenyl)-tetrahydro-2H- | C1COCCC1(C═O)c1ccccc1[Cl] |
| | pyran-4-carbaldehyde | |
| A8 | 2-fluoro-6-phenoxy-benzaldehyde | C(c1c(cccc1F)Oc1ccccc1)═O |
| A9 | phenyl-methyl 2-formyl-piperidine- | C1CCN(C(C1)C═O)C(═O)OCc1ccccc1 |
| | 1-carboxylate | |
| A10 | 6-cyclohexyloxy-pyridine-3- | C1CCC(CC1)Oc1ccc(C═O)cn1 |
| | carbaldehyde | |
| A11 | 7-thia-9-aza-bicyclo[4.3.0]nona- | C(c1nc2ccccc2s1)═O |
| | 1,3,5,8-tetraene-8-carbaldehyde | |
| A12 | 5-(4-methoxy-phenyl)-thiophene-2- | COc1ccc(cc1)c1ccc(C═O)s1 |
| | carbaldehyde | |
| B1 | 7-thia-bicyclo[4.3.0]nona-1,3,5,8- | C(c1cc2ccccc2s1)═O |
| | tetraene-8-carbaldehyde | |
| B2 | 4-(1H-imidazol-1-yl)-benzaldehyde | C(c1ccc(cc1)n1ccnc1)═O |
| B3 | 2-butyl-1H-imidazole-4- | CCCCc1nc(C═O)c[nH]1 |
| | carbaldehyde | |
| B4 | 4-bromo-2H-pyrazole-3- | C(c1c(cn[nH]1)[Br])═O |
| | carbaldehyde | |
| B5 | methyl 2-formyl-benzoate | COC(c1ccccc1C═O)═O |
| B6 | 2-amino-3,6-dimethoxy- | COc1ccc(c(c1C═O)N)OC |
| | benzaldehyde | |
| B7 | 4-(2-oxo-pyrrolidin-1-yl)- | C1CC(N(C1)c1ccc(C═O)cc1)═O |
| | benzaldehyde | |
| B8 | 7-allyl-8-methyl-7-aza- | Cc1c(C═O)c2ccccc2n1CC═C |
| | bicyclo[4.3.0]nona-1,3,5,8- | |
| | tetraene-9-carbaldehyde | |
| B9 | 4-(3-formyl-2,5-dimethyl-1H- | Cc1cc(C═O)c(C)n1c1ccc(cc1)C(O)═O |
| | pyrrol-1-yl)-benzoic acid | |
| B10 | 7-allyl-7-aza-bicyclo[4.3.0]nona- | C═CCn1cc(C═O)c2ccccc12 |
| | 1,3,5,8-tetraene-9-carbaldehyde | |
| B11 | 2-(9-formyl-7-aza- | C(C#N)n1cc(C═O)c2ccccc12 |
| | bicyclo[4.3.0]nona-1,3,5,8-tetraen- | |
| | 7-yl)-ethanenitrile | |
| B12 | 3-(4-formyl-5-methyl-1H-pyrazol- | Cc1c(C═O)cnn1CCC(O)═O |
| | 1-yl)-propanoic acid | |
| C1 | 5,8-dimethyl-1,7-diaza- | Cc1cccn2c(C═O)c(C)nc12 |
| | bicyclo[4.3.0]nona-2,4,6,8- | |
| | tetraene-9-carbaldehyde | |
| C2 | 3-benzyl-3H-imidazole-4- | C(c1ccccc1)n1cncc1C═O |
| | carbaldehyde | |
| C3 | 3-(pyrimidin-2-yloxy)- | C(c1cccc(c1)Oc1ncccn1)═O |
| | benzaldehyde | |
| C4 | 3-dimethylamino-2-p-tolyl-prop-2- | Cc1ccc(cc1)C(═CN(C)C)C═O |
| | enal | |
| C5 | 1,3-dimethyl-5-(3-(trifluoro- | Cc1c(C═O)c(n(C)n1)Oc1cccc(c1)C(F)(F)F |
| | methyl)-phenoxy)-1H-pyrazole-4- | |
| | carbaldehyde | |
| C6 | 4-dimethylamino-2-(2-formyl-1H- | CN(C)c1ccnc(c1C#N)n1cccc1C═O |
| | pyrrol-1-yl)-pyridine-3-carbonitrile | |
| C7 | 5-chloro-2-phenyl-1H-imidazole-4- | C(c1c([nH]c(c2ccccc2)n1)[Cl])═O |
| | carbaldehyde | |
| C8 | 8-(2-(furan-3-yl)-vinyl)-3-hydroxy- | C(═Cc1c(C═O)c2cc(ccc2o1)O)c1ccoc1 |
| | 7-oxa-bicyclo[4.3.0]nona-1,3,5,8- | |
| | tetraene-9-carbaldehyde | |
| C9 | 4-(4-(trifluoro-methyl)-pyridin-2- | C(c1ccc(cc1)c1cc(ccn1)C(F)(F)F)═O |
| | yl)-benzaldehyde | |
| C10 | hexanal | CCCCCC═O |
| C11 | 2-methyl-2-phenylsulfanyl- | CC(C)(C═O)Sc1ccccc1 |
| | propanal | |
| C12 | 7-chloro-bicyclo[4.4.0]deca- | C1Cc2ccccc2C(═C1C═O)[Cl] |
| | 1,3,5,7-tetraene-8-carbaldehyde | |
| D1 | 6-(4-formyl-phenyl)-pyridine-3- | C(c1ccc(cc1)c1ccc(C#N)cn1)═O |
| | carbonitrile | |
| D2 | 4-(thiomorpholin-4-yl)- | C1CSCCN1c1ccc(C═O)cc1 |
| | benzaldehyde | |
| D3 | 4-((2-chloro-thiazol-5-yl)- | C(c1cnc(s1)[Cl])Oc1ccc(C═O)cc1 |
| | methoxy)-benzaldehyde | |
| D4 | 3-chloro-8-methyl-2-aza- | CC1C═CC2C(C═1)C═C(C═O)C(═N2)[Cl] |
| | bicyclo[4.4.0]deca-2,4,7,9-tetraene- | |
| | 4-carbaldehyde | |
| D5 | 7-(3-fluoro-phenyl)-4-thia-1,6- | C(c1c(c2cccc(c2)F)nc2n1ccs2)═O |
| | diaza-bicyclo[3.3.0]octa-2,5,7- | |
| | triene-8-carbaldehyde | |
| D6 | 1-(4-(thiophen-2-yl)-pyrimidin-2- | C(c1cccn1c1nccc(c2cccs2)n1)═O |
| | yl)-1H-pyrrole-2-carbaldehyde | |
| D7 | 2-(4-chloro-phenoxy)-pyridine-3- | C(c1cccnc1Oc1ccc(cc1)[Cl])═O |
| | carbaldehyde | |
| D8 | 5-chloro-1-methyl-3- | Cn1c(c(C═O)c(CSc2ccccc2)n1)[Cl] |
| | (phenylsulfanyl-methyl)-1H- | |
| | pyrazole-4-carbaldehyde | |
| D9 | 8-ethylsulfanyl-7-methyl-7-aza- | CCSc1c(C═O)c2ccccc2n1C |
| | bicyclo[4.3.0]nona-1,3,5,8- | |
| | tetraene-9-carbaldehyde | |
| D10 | 10-formyl-7,9-dioxo-8-oxa-2-aza- | C(C1C(═O)OC(c2cccnc12)═O)═O |
| | bicyclo[4.4.0]deca-1(6),2,4-triene | |
| D11 | 1-phenyl-1H-1,2,3-triazole-4- | C(c1cn(c2ccccc2)nn1)═O |
| | carbaldehyde | |
| D12ⁱⁱ | N/A | N/A |
| E1 | 2-(2-cyano-phenyl)-2-(5-formyl-1- | Cn1c(C═O)ccc1C(C#N)c1ccccc1C#N |
| | methyl-1H-pyrrol-2-yl)- | |
| | ethanenitrile | |
| E2 | 3-methylsulfanyl-2-aza- | CSc1c(C═O)cc2ccccc2n1 |
| | bicyclo[4.4.0]deca-1,3,5,7,9- | |
| | pentaene-4-carbaldehyde | |
| E3ⁱⁱ | N/A | N/A |
| E4ⁱⁱ | N/A | N/A |
| E5 | 1-((2-chloro-thiazol-5-yl)-methyl)- | C(c1cnc(s1)[Cl])n1cccc1C═O |
| | 1H-pyrrole-2-carbaldehyde | |
| E6 | 4-(pyrazin-2-yl)-benzaldehyde | C(c1ccc(cc1)c1cnccn1)═O |
| E7 | 2-(3-chloro-5-(trifluoro-methyl)- | CN(C)C═C(C═O)c1c(cc(cn1)C(F)(F)F)[Cl] |
| | pyridin-2-yl)-3-dimethylamino- | |
| | prop-2-enal | |
| E8 | 5-methoxy-1,3-dimethyl-1H- | Cc1c(C═O)c(n(C)n1)OC |
| | pyrazole-4-carbaldehyde | |
| E9 | 2-(methyl-(phenyl-methyl)-amino)- | CN(Cc1ccccc1)c1ncc(C═O)s1 |
| | thiazole-5-carbaldehyde | |
| E10 | methyl 6-formyl-pyridine-3- | COC(c1ccc(C═O)nc1)═O |
| | carboxylate | |
| E11 | 2-(2-methoxy-4-nitro-phenyl)- | COc1cc(ccc1CC═O)[N+]([O−])═O |
| | acetaldehyde | |
| E12 | 1-(4-chloro-benzyl)-1H-1,2,3- | C(c1ccc(cc1)[Cl])n1cc(C═O)nn1 |
| | triazole-4-carbaldehyde | |
| F1 | 5-chloro-6-hydroxy-pyridine-3- | C(c1cc(c(nc1)O)[Cl])═O |
| | carbaldehyde | |
| F2 | 1-(3,4-dichloro-benzyl)-6-oxo-1,6- | C(c1ccc(c(c1)[Cl])[Cl])N1C═C(C═CC1═O)C═O |
| | dihydro-pyridine-3-carbaldehyde | |
| F3 | 4-(4-(hydroxy-methyl)-piperidin-1- | C1CN(CCC1CO)c1ccc(C═O)cc1 |
| | yl)-benzaldehyde | |
| F4 | 5-bromo-7-oxa-bicyclo[4.3.0]nona- | C1COc2c1cc(C═O)cc2[Br] |
| | 1(6),2,4-triene-3-carbaldehyde | |
| F5 | 4-((8-thia-7,9-diaza- | C(c1ccc2c(c1)nsn2)Oc1ccc(C═O)cc1 |
| | bicyclo[4.3.0]nona-1(9),2,4,6- | |
| | tetraen-3-yl)-methoxy)- | |
| | benzaldehyde | |
| F6 | 4-(1H-pyrazol-1-yl)-benzaldehyde | C(c1ccc(cc1)n1cccn1)═O |
| F7 | 2-(1,1-dimethyl-ethylsulfonyl)- | CC(C)(C)S(c1ccccc1C═O)(═O)═O |
| | benzaldehyde | |
| F8 | 2-formyl-bicyclo[4.4.0]deca- | CC(═O)Oc1ccc2ccccc2c1C═O |
| | 1,3,5,7,9-pentaen-3-yl ethanoate | |
| F9 | 10-formyl-bicyclo[4.4.0]deca- | C(c1cccc2cccc(C(O)═O)c12)═O |
| | 1(6),2,4,7,9-pentaene-2-carboxylic | |
| | acid | |
| F10 | ethyl 4-chloro-5-formyl-3-methyl- | CCOC(c1c(C)c2C(═C(CCc2o1)C═O)[Cl])═O |
| | 6,7-dihydrobenzofuran-2- | |
| | carboxylate | |
| F11 | 5-formyl-2-hydroxy-4- | C(c1cnc(c(C#N)c1Nc1ccccc1)O)═O |
| | phenylamino-pyridine-3- | |
| | carbonitrile | |
| F12 | 4-formylphenyl | C1Oc2ccc(cc2O1)C(═O)Oc1ccc(C═O)cc1 |
| | benzo[d][1,3]dioxole-5-carboxylate | |
| G1 | 2-formyl-3-methyl-1-oxo-1,2- | CC1C(C═O)C(n2c3ccccc3nc2C═1C#N)═O |
| | dihydrobenzo[4,5]imidazo[1,2- | |
| | a]pyridine-4-carbonitrile | |
| G2 | 5-formyl-4-(4-methylpyridin-1- | Cc1cc[n+](cc1)c1c(C═O)sc(n1)[O−] |
| | ium-1-yl)thiazol-2-olate | |
| G3 | 4-(4-chloro-phenylsulfonyl)- | C(c1ccc(cc1)S(c1ccc(cc1)[Cl])(═O)═O)═O |
| | benzaldehyde | |
| G4 | 7-methoxy-8-phenyl-7-aza- | COn1c(c2ccccc2)c(C═O)c2ccccc12 |
| | bicyclo[4.3.0]nona-1,3,5,8- | |
| | tetraene-9-carbaldehyde | |
| G5 | 3-(5-chloro-pyridin-2-ylamino)-2- | C(═C(C═O)c1nc2ccccc2o1)Nc1ccc(cn1)[Cl] |
| | (7-oxa-9-aza-bicyclo[4.3.0]nona- | |
| | 1,3,5,8-tetraen-8-yl)-acrylaldehyde | |
| G6 | 9-methyl-4-nitro-7,9-diaza- | Cn1c2ccc(cc2nc1C═O)[N+]([O−])═O |
| | bicyclo[4.3.0]nona-1,3,5,7- | |
| | tetraene-8-carbaldehyde | |
| G7 | 9-methyl-2-phenyl-9H- | Cn1c2ccccc2n2c(C═O)c(c3ccccc3)nc12 |
| | benzo[d]imidazo[1,2-a]imidazole- | |
| | 3-carbaldehyde | |
| G8 | 5-(9-methyl-7,9-diaza- | Cn1c2ccccc2nc1c1ccc(C═O)s1 |
| | bicyclo[4.3.0]nona-1,3,5,7-tetraen- | |
| | 8-yl)-thiophene-2-carbaldehyde | |
| G9 | 4-hydroxy-6-methyl-2-(piperidin-1- | Cc1c(C═O)c(nc(n1)N1CCCCC1)O |
| | yl)-pyrimidine-5-carbaldehyde | |
| G10 | 2,5-dimethyl-1-(3-nitro-4- | Cc1cc(C═O)c(C)n1c1ccc(c(c1)[N+]([O−])═O)N1CCCC1 |
| | (pyrrolidin-1-yl)-phenyl)-1H- | |
| | pyrrole-3-carbaldehyde | |
| G11 | 4-allyloxy-3-iodo-benzaldehyde | C═CCOc1ccc(C═O)cc1I |
| G12 | 1-(2-methoxy-4-nitro-phenyl)-2,5- | Cc1cc(C═O)c(C)n1c1ccc(cc1OC)[N+]([O−])═O |
| | dimethyl-1H-pyrrole-3- | |
| | carbaldehyde | |
| H1 (D1)ⁱ | 6-(4-formyl-phenyl)-pyridine-3- | C(c1ccc(cc1)c1ccc(C#N)cn1)═O |
| | carbonitrile | |
| H2 | 3-(2-hydroxy-3-nitro-phenyl)-2- | CC(═Cc1cccc(c1O)[N+]([O−])═O)C═O |
| | methyl-prop-2-enal | |
| H3 (D12)ⁱ | 2-((5-chloro-1,2,3-thiadiazol-4-yl)- | C(c1c(snn1)[Cl])Oc1ccccc1C═O |
| | methoxy)-benzaldehyde | |
| H4 | 2-(9-formyl-8-methyl-7-aza- | Cc1c(C═O)c2ccccc2n1CC(O)═O |
| | bicyclo[4.3.0]nona-1,3,5,8-tetraen- | |
| | 7-yl)-acetic acid | |
| H5 | 2,5-dimethyl-1-(pyridin-4-yl)-1H- | Cc1cc(C═O)c(C)n1c1ccncc1 |
| | pyrrole-3-carbaldehyde | |
| H6 | 4-formyl-2-iodo-6-methoxy-phenyl | CC(═O)Oc1c(cc(C═O)cc1I)OC |
| | ethanoate | |
| H7 | 2-dimethylamino-5-formyl-6-oxo- | CN(C)C1═NC(═C(C═O)C(═O)O1)c1ccccc1 |
| | 4-phenyl-6H-1,3-oxazine | |
| H8 | 2-methoxy-4-(pyrrolidin-1-yl)- | COc1cc(ccc1C═O)N1CCCC1 |
| | benzaldehyde | |
| H9 | 5-dimethylamino-7-methoxy- | CN(C)c1ccc(C═O)c2cccc(c12)OC |
| | bicyclo[4.4.0]deca-1(6),2,4,7,9- | |
| | pentaene-2-carbaldehyde | |
| H10 | 1-methyl-5-(3-nitro-phenyl)-1H- | Cn1c(C═O)ccc1c1cccc(c1)[N+]([O−])═O |
| | pyrrole-2-carbaldehyde | |
| H11 | 7,9-dimethyl-4-nitro-8-oxo-7,9- | CN1C(N(C)c2cc(c(C═O)cc12)[N+]([O−])═O)═O |
| | diaza-bicyclo[4.3.0]nona-1,3,5- | |
| | triene-3-carbaldehyde | |
| H12 | 10-ethoxy-2-aza- | CCOc1cccc2ccc(C═O)nc12 |
| | bicyclo[4.4.0]deca-1(6),2,4,7,9- | |
| | pentaene-3-carbaldehyde | |
| I1 | 7-methyl-4-thia-1,6-diaza- | Cc1c(C═O)n2ccsc2n1 |
| | bicyclo[3.3.0]octa-2,5,7-triene-8- | |
| | carbaldehyde | |
| I2 | 3-(1-benzyl-1H-1,2,3-triazol-4-yl)- | C(c1ccccc1)n1cc(C═CC═O)nn1 |
| | acrylaldehyde | |
| I3 | 5-formyl-2,3- | Cc1[n+](C)c2cc(C═O)ccc2s1.[I−] |
| | dimethylbenzo[d]thiazol-3-ium | |
| | iodide | |
| I4 | 1-cyclopentyl-2,5-dimethyl-1H- | Cc1cc(C═O)c(C)n1C1CCCC1 |
| | pyrrole-3-carbaldehyde | |
| I5 | 5-chloro-1-(2-chloro-phenyl)-3- | Cc1c(C═O)c(n(c2ccccc2[Cl])n1)[Cl] |
| | methyl-1H-pyrazole-4- | |
| | carbaldehyde | |
| I6 | 3-(5-methyl-furan-2-yl)-1-phenyl- | Cc1ccc(c2c(C═O)cn(c3ccccc3)n2)o1 |
| | 1H-pyrazole-4-carbaldehyde | |
| I7 | 1-((acetoxyimino)methyl)-2- | CC(═O)ON═Cc1c(C)c(C═O)n2ccccc12 |
| | methylindolizine-3-carbaldehyde | |
| I8 | 5-oxo-1,2,4,5- | C1CN2C(═C1C═O)NC(c1ccccc12)═O |
| | tetrahydropyrrolo[1,2- | |
| | a]quinazoline-3-carbaldehyde | |
| I9 | 9-(4-methyl-benzyl)-10-oxo-1,8- | Cc1ccc(C═C2C(N3CCCCC(C═O)═C3N2)═O)cc1 |
| | diaza-bicyclo[5.3.0]dec-6-ene-6- | |
| | carbaldehyde | |
| I10 | 3-((4-chloro-phenyl)- | C(c1cccc(c1)NC(c1ccc(cc1)[Cl])═O)═O |
| | formylamino)-benzaldehyde | |
| I11 | 3-(7-formyl-8-methyl-1-aza- | Cc1c(C═O)c2ccccn2c1CCC#N |
| | bicyclo[4.3.0]nona-2,4,6,8-tetraen- | |
| | 9-yl)-propanenitrile | |
| I12 | 2-(5-formyl-2-methoxy-phenoxy)- | COc1ccc(C═O)cc1OCC#N |
| | ethanenitrile | |
| J1 | bicyclo[2.2.1]hept-2-ene-5- | Cc1c(C(═O)OC)c2c(C═O)c(c(cc2o1)[Br])O |
| | carbaldehyde | |
| J2 | 9H-fluorene-2-carbaldehyde | C1c2ccccc2c2ccc(C═O)cc12 |
| J3 | 4-methyl-3-nitro-benzaldehyde | Cc1ccc(C═O)cc1[N+]([O−])═O |
| J4 | 5-methoxy-7-oxa- | COc1cccc2cc(C═O)oc12 |
| | bicyclo[4.3.0]nona-1,3,5,8- | |
| | tetraene-8-carbaldehyde | |
| J5 | 3-(9-formyl-7-aza- | C(Cn1cc(C═O)c2ccccc12)C#N |
| | bicyclo[4.3.0]nona-1,3,5,8-tetraen- | |
| | 7-yl)-propanenitrile | |
| J6 | 4-(phenyl-methoxy)-benzaldehyde | C(c1ccccc1)Oc1ccc(C═O)cc1 |
| J7 | 3-amino-8-fluoro-5-oxo-2-oxa- | C(C1═C(N)Oc2ccc(cc2C1═O)F)═O |
| | bicyclo[4.4.0]deca-1(10),3,6,8- | |
| | tetraene-4-carbaldehyde | |
| J8 | 1-((2,5-dioxa-bicyclo[4.4.0]deca- | Cc1cc(C═O)c(C)n1CC1COc2ccccc2O1 |
| | 1(10),6,8-trien-3-yl)-methyl)-2,5- | |
| | dimethyl-1H-pyrrole-3- | |
| | carbaldehyde | |
| J9 | 2-(acetyl-phenyl-amino)-thiazole- | CC(N(c1ccccc1)c1nc(C═O)cs1)═O |
| | 4-carbaldehyde | |
| J10 | 2-acetylamino-5-bromo-thiazole-4- | CC(Nc1nc(C═O)c(s1)[Br])═O |
| | carbaldehyde | |
| J11 | 5-(7-thia-9-aza-bicyclo[4.3.0]nona- | C(c1ccc(c2nc3ccccc3s2)o1)═O |
| | 1,3,5,8-tetraen-8-yl)-furan-2- | |
| | carbaldehyde | |
| J12 | 8-(pyrrolidin-1-yl)-1,7-diaza- | C1CCN(C1)c1c(C═O)n2ccccc2n1 |
| | bicyclo[4.3.0]nona-2,4,6,8- | |
| | tetraene-9-carbaldehyde | |
| K1 | 5-(piperidin-1-yl)-furan-2- | C1CCN(CC1)c1ccc(C═O)o1 |
| | carbaldehyde | |
| K2 | 2,5-dimethyl-1-(5-methyl-isoxazol- | Cc1cc(C═O)c(C)n1c1cc(C)on1 |
| | 3-yl)-1H-pyrrole-3-carbaldehyde | |
| K3 | 4-chloro-2-(4-methoxy- | COc1ccc(cc1)Nc1nc(c(C═O)s1)[Cl] |
| | phenylamino)-thiazole-5- | |
| | carbaldehyde | |
| K4 | 9-chloro-7-methyl-7-aza- | Cn1c(C═O)c(c2ccccc12)[Cl] |
| | bicyclo[4.3.0]nona-1,3,5,8- | |
| | tetraene-8-carbaldehyde | |
| K5 | 8-nitro-2,4-dioxa- | C1c2cc(cc(C═O)c2OCO1)[N+]([O−])═O |
| | bicyclo[4.4.0]deca-1(10),6,8-triene- | |
| | 10-carbaldehyde | |
| K6 | 4-(1H-1,2,4-triazol-1-yl)- | C(c1ccc(cc1)n1cncn1)═O |
| | benzaldehyde | |
| K7 | 5-nitro-2-(4-phenyl-piperazin-1- | C1CN(CCN1c1ccccc1)c1ccc(cc1C═O)[N+]([O−])═O |
| | yl)-benzaldehyde | |
| K8 | 3-(2,2,2-trifluoro-acetylamino)- | C(c1cccc(c1)NC(C(F)(F)F)═O)═O |
| | benzaldehyde | |
| K9 | 2-((1,5,7-triaza-bicyclo[4.3.0]nona- | C(c1cn2cccnc2n1)Oc1ccccc1C═O |
| | 2,4,6,8-tetraen-8-yl)-methoxy)- | |
| | benzaldehyde | |
| K10 | 3-(2,6-dioxa-bicyclo[5.4.0]undeca- | Cn1cc(C═O)c(c2ccc3c(c2)OCCCO3)n1 |
| | 1(11),7,9-trien-9-yl)-1-methyl-1H- | |
| | pyrazole-4-carbaldehyde | |
| K11 | 8-fluoro-2,4-dioxa- | C1c2cc(cc(C═O)c2OCO1)F |
| | bicyclo[4.4.0]deca-1(10),6,8-triene- | |
| | 10-carbaldehyde | |
| K12 | 4-chloro-2-dimethylamino- | CN(C)c1nc(c(C═O)s1)[Cl] |
| | thiazole-5-carbaldehyde | |
| L1 | 1-(2-chloro-benzyl)-1H-pyrazole-4- | C(c1ccccc1[Cl])n1cc(C═O)cn1 |
| | carbaldehyde | |
| L2 | 3-(3-(3-bromo-phenyl)-4-formyl- | C(Cn1cc(C═O)c(c2cccc(c2)[Br])n1)C#N |
| | 1H-pyrazol-1-yl)-propanenitrile | |
| L3 | 7-thia-bicyclo[4.3.0]nona-1,3,5,8- | C(c1csc2ccccc12)═O |
| | tetraene-9-carbaldehyde | |
| L4 | 9-nitro-2,5-dioxa- | C1COc2cc(c(C═O)cc2O1)[N+]([O−])═O |
| | bicyclo[4.4.0]deca-1(10),6,8-triene- | |
| | 8-carbaldehyde | |
| L5 | 2-(7,9-diaza-bicyclo[4.3.0]nona- | CS(C(C═O)c1nc2ccccc2[nH]1)(═O)═O |
| | 1,3,5,7-tetraen-8-yl)-2- | |
| | methylsulfonyl-acetaldehyde | |
| L6 | 4-(5-formylfuran-2-yl)-N,N- | CN(C)S(c1ccc(cc1)c1ccc(C═O)o1)(═O)═O |
| | dimethylbenzenesulfonamide | |
| L7 | 3-fluoro-4-(1H-1,2,4-triazol-1-yl)- | C(c1ccc(c(c1)F)n1cncn1)═O |
| | benzaldehyde | |
| L8 | 3,5,7-trimethyl-9-thia-2,4-diaza- | Cc1c2c(C)nc(C)nc2sc1C═O |
| | bicyclo[4.3.0]nona-1(6),2,4,7- | |
| | tetraene-8-carbaldehyde | |
| L9 | 8-methyl-2-(trifluoro-methyl)- | Cc1c(C═O)c2nccc(C(F)(F)F)n2n1 |
| | 1,5,9-triaza-bicyclo[4.3.0]nona- | |
| | 2,4,6,8-tetraene-7-carbaldehyde | |
| L10 | 3-chloro-7,9-dimethyl-2,8,9-triaza- | Cc1c2cc(C═O)c(nc2n(C)n1)[Cl] |
| | bicyclo[4.3.0]nona-1,3,5,7- | |
| | tetraene-4-carbaldehyde | |
| L11 | 4-methoxy-7-methyl-2-oxo-1,5- | CC1═CC═CN2C1═NC(═C(C═O)C2═O)OC |
| | diaza-bicyclo[4.4.0]deca-3,5,7,9- | |
| | tetraene-3-carbaldehyde | |
| L12 | 5-chloro-1-(2-chloro-benzyl)-3- | Cc1c(C═O)c(n(Cc2ccccc2[Cl])n1)[Cl] |
| | methyl-1H-pyrazole-4- | |
| | carbaldehyde | |
| M1 | 5-chloro-2-((pyridin-3-yl)- | C(c1cccnc1)Oc1ccc(cc1C═O)[Cl] |
| | methoxy)-benzaldehyde | |
| M2 | 4-(2-methyl-morpholin-4-yl)-3- | CC1CN(CCO1)c1ccc(C═O)cc1[N+]([O−])═O |
| | nitro-benzaldehyde | |
| M3ⁱⁱ | N/A | N/A |
| M4ⁱⁱ | N/A | N/A |
| M5 | 4-dimethylamino-benzaldehyde | CN(C)c1ccc(C═O)cc1 |
| M6 | benzaldehyde | C(c1ccccc1)═O |
| M7 | 7,9-dioxa-bicyclo[4.3.0]nona- | C1Oc2cccc(C═O)c2O1 |
| | 1(6),2,4-triene-2-carbaldehyde | |
| M8 | 2-aza-bicyclo[4.4.0]deca- | C(c1cc2ccccc2nc1)═O |
| | 1(10),2,4,6,8-pentaene-4- | |
| | carbaldehyde | |
| M9 | 4,10-dimethoxy- | COc1ccc2c(ccc(C═O)c2c1)OC |
| | bicyclo[4.4.0]deca-1,3,5,7,9- | |
| | pentaene-7-carbaldehyde | |
| M10 | 4-phenyl-benzaldehyde | C(c1ccc(cc1)c1ccccc1)═O |
| M11 | 4-(2-oxo-2-(2-oxo-imidazolidin-1- | C1CN(C(COc2ccc(C═O)cc2)═O)C(N1)═O |
| | yl)-ethoxy)-benzaldehyde | |
| M12 | 8-methoxy-bicyclo[4.4.0]deca- | COc1ccc2ccccc2c1C═O |
| | 1,3,5,7,9-pentaene-7-carbaldehyde | |
| N1 | 8-hydroxy-3-nitro- | C(c1c(ccc2cc(ccc12)[N+]([O−])═O)O)═O |
| | bicyclo[4.4.0]deca-1,3,5,7,9- | |
| | pentaene-7-carbaldehyde | |
| N2 | 2-(7-oxa-9-aza-bicyclo[4.3.0]nona- | C(C(C#N)c1nc2ccccc2o1)═O |
| | 1,3,5,8-tetraen-8-yl)-3-oxo- | |
| | propanenitrile | |
| N3 | 8-(2-dimethylamino-vinyl)-7- | CN(C)C═Cc1c(C═O)c2cc(ccc2n1C)[N+]([O−])═O |
| | methyl-3-nitro-7-aza- | |
| | bicyclo[4.3.0]nona-1,3,5,8- | |
| | tetraene-9-carbaldehyde | |
| N4 | 4-((2,5-dioxo-imidazolidin-4- | C(═C1C(NC(N1)═O)═O)c1ccc(C═O)cc1 |
| | ylidene)-methyl)-benzaldehyde | |
| N5 | 10-hydroxy-2-aza- | C(c1ccc(c2c1cccn2)O)═O |
| | bicyclo[4.4.0]deca-1(10),2,4,6,8- | |
| | pentaene-7-carbaldehyde | |
| N6 | 4-fluoro-3-methoxy-benzaldehyde | COc1cc(C═O)ccc1F |
| N7 | 2-fluoro-5-(trifluoro-methyl)- | C(c1cc(ccc1F)C(F)(F)F)═O |
| | benzaldehyde | |
| N8 | 3-hydroxy-4-iodo-benzaldehyde | C(c1ccc(c(c1)O)I)═O |
| N9 | 9-thia-2-aza-bicyclo[4.3.0]nona- | C(c1cc2cccnc2s1)═O |
| | 1(6),2,4,7-tetraene-8-carbaldehyde | |
| N10 | 2,4-dimethyl-thiazole-5- | Cc1c(C═O)sc(C)n1 |
| | carbaldehyde | |
| N11 | 5-(thiophen-2-yl)-thiophene-2- | C(c1ccc(c2cccs2)s1)═O |
| | carbaldehyde | |
| N12 | 1,3-dimethyl-5-(morpholin-4-yl)- | Cc1c(C═O)c(N2CCOCC2)n(C)n1 |
| | 1H-pyrazole-4-carbaldehyde | |
| O1 | 3-(1H-pyrrol-1-yl)-benzaldehyde | C(c1cccc(c1)n1cccc1)═O |
| O2 | 3-(1H-pyrrol-1-yl)-thiophene-2- | C(c1c(ccs1)n1cccc1)═O |
| | carbaldehyde | |
| O3 | 1,1-dimethyl-ethyl 4-(5-formyl-4- | Cc1c(C═O)sc(C2CCN(CC2)C(═O)OC(C)(C)C)n1 |
| | methyl-thiazol-2-yl)-piperidine-1- | |
| | carboxylate | |
| O4 | 1,1-dimethyl-ethyl 4-formyl-3,5- | Cc1c(C═O)c(C)n(C(═O)OC(C)(C)C)n1 |
| | dimethyl-1H-pyrazole-1- | |
| | carboxylate | |
| O5 | 3-(morpholin-4-yl)-benzaldehyde | C1COCCN1c1cccc(C═O)c1 |
| O6 | 6-(piperidin-1-yl)-pyridine-2- | C1CCN(CC1)c1cccc(C═O)n1 |
| | carbaldehyde | |
| O7 | 7-methyl-7-aza-bicyclo[4.3.0]nona- | Cn1ccc2ccc(C═O)cc12 |
| | 1,3,5,8-tetraene-4-carbaldehyde | |
| O8 | 4-((piperidin-1-yl)-methyl)- | C1CCN(CC1)Cc1ccc(C═O)cc1 |
| | benzaldehyde | |
| O9 | 1-methyl-5-phenyl-1H-pyrazole-3- | Cn1c(cc(C═O)n1)c1ccccc1 |
| | carbaldehyde | |
| O10 | 2-(3-chloro-phenyl)-thiazole-4- | C(c1csc(c2cccc(c2)[Cl])n1)═O |
| | carbaldehyde | |
| O11 | 4-(3-methyl-1,2,4-oxadiazol-5-yl)- | Cc1nc(c2ccc(C═O)cc2)on1 |
| | benzaldehyde | |
| O12 | 6-(thiophen-2-yl)-pyridine-3- | C(c1ccc(c2cccs2)nc1)═O |
| | carbaldehyde | |
| P1 | 1-(6-methyl-pyrazin-2-yl)- | Cc1cncc(n1)N1CCC(CC1)C═O |
| | piperidine-4-carbaldehyde | |
| P2 | 5-methyl-2-oxa-5,7-diaza- | CN1CCOc2cc(C═O)cnc12 |
| | bicyclo[4.4.0]deca-1(10),6,8-triene- | |
| | 9-carbaldehyde | |
| P3 | 4-(6-methyl-pyrazin-2-yloxy)- | Cc1cncc(n1)Oc1ccc(C═O)cc1 |
| | benzaldehyde | |
| P4 | 3-phenyl-isoxazole-5-carbaldehyde | C(c1cc(c2ccccc2)no1)═O |
| P5 (G8)ⁱ | 5-(9-methyl-7,9-diaza- | Cn1c2ccccc2nc1c1ccc(C═O)s1 |
| | bicyclo[4.3.0]nona-1,3,5,7-tetraen- | |
| | 8-yl)-thiophene-2-carbaldehyde | |
| P6 | 3-dimethylamino-2-(2-nitro- | CN(C)C═C(C═O)c1ccccc1[N+]([O−])═O |
| | phenyl)-acrylaldehyde | |
| P7 | 4,6-dimethyl-2,8-dithia- | Cc1csc2c1c(C)c(C═O)s2 |
| | bicyclo[3.3.0]octa-1(5),3,6-triene- | |
| | 3-carbaldehyde | |
| P8 | 3-phenoxy-thiophene-2- | C(c1c(ccs1)Oc1ccccc1)═O |
| | carbaldehyde | |
| P9 | 5-((7,9-dioxo-8-aza- | C(c1ccc(C═O)o1)N1C(c2ccccc2C1═O)═O |
| | bicyclo[4.3.0]nona-1,3,5-trien-8- | |
| | yl)-methyl)-furan-2-carbaldehyde | |
| P10 | 3-(2-formyl-1H-pyrrol-1-yl)- | C(c1cccn1c1cccc(C#N)c1)═O |
| | benzonitrile | |
| P11 | 2-fluoro-5-formyl-benzoic acid | C(c1ccc(c(c1)C(O)═O)F)═O |
| P12 | 6-amino-1,3-dimethyl-2,4-dioxo- | CN1C(═C(C═O)C(N(C)C1═O)═O)N |
| | 1,2,3,4-tetrahydro-pyrimidine-5- | |
| | carbaldehyde | |
| Q1 | 3-chloro-thiophene-2-carbaldehyde | C(c1c(ccs1)[Cl])═O |
| Q2 | 6-formyl-2-methylsulfanyl- | CSc1c(C#N)ccc(C═O)n1 |
| | pyridine-3-carbonitrile | |
| Q3 | 2-chloro-4-methylsulfonyl- | CS(c1ccc(C═O)c(c1)[Cl])(═O)═O |
| | benzaldehyde | |
| Q4 (G6)ⁱ | 9-methyl-4-nitro-7,9-diaza- | C1c2ccccc2c2ccc(C═O)cc12 |
| | bicyclo[4.3.0]nona-1,3,5,7- | |
| | tetraene-8-carbaldehyde | |
| Q5 | 2,5-dihydroxy-benzaldehyde | C(c1cc(ccc1O)O)═O |
| Q6 | 2-fluoro-5-formyl-benzonitrile | C(c1ccc(c(C#N)c1)F)═O |
| Q7 | 3-vinyl-benzaldehyde | C═Cc1cccc(C═O)c1 |
| Q8 | 2-fluoro-6-methoxy-benzaldehyde | COc1cccc(c1C═O)F |
| Q9 | 5,8-dichloro-4-formyl-3-oxo-2-oxa- | C(C1═C(c2cc(ccc2OC1═O)[Cl])[Cl])═O |
| | bicyclo[4.4.0]deca-1(10),4,6,8- | |
| | tetraene | |
| Q10 | 3,5-dichloro-pyridine-4- | C(c1c(cncc1[Cl])[Cl])═O |
| | carbaldehyde | |
| Q11 | 3-(7-methyl-7-aza- | [H][C@](C)(CC═O)c1cn(C)c2ccccc12 |
| | bicyclo[4.3.0]nona-1,3,5,8-tetraen- | |
| | 9-yl)-butanal | |
| Q12 | 5-(4-fluoro-phenyl)-isoxazole-3- | C(c1cc(c2ccc(cc2)F)on1)═O |
| | carbaldehyde | |
| R1 | methyl 4-(6-formyl-pyridin-2-yl)- | COC(c1ccc(cc1)c1cccc(C═O)n1)═O |
| | benzoate | |
| R2 | 2-methoxy-4-nitro-benzaldehyde | COc1cc(ccc1C═O)[N+]([O−])═O |
| R3 | 4-bromo-2-methoxy-benzaldehyde | COc1cc(ccc1C═O)[Br] |
| R4 | 4,5-dimethyl-thiophene-2- | Cc1cc(C═O)sc1C |
| | carbaldehyde | |
| R5 | 6-bromo-2,3-dimethoxy- | COc1ccc(c(C═O)c1OC)[Br] |
| | benzaldehyde | |
| R6 | 2-hydroxy-5-methoxy-3- | COc1cc(CN2CCOCC2)c(c(C═O)c1)O |
| | ((morpholin-4-yl)-methyl)- | |
| | benzaldehyde | |
| R7 | 8-chloro-4-formyl-3,5-dioxo-2-oxa- | C(C1C(c2cc(ccc2OC1═O)[Cl])═O)═O |
| | bicyclo[4.4.0]deca-1(10),6,8-triene | |
| R8 | 2-chloro-6-phenyl-pyridine-4- | C(c1cc(c2ccccc2)nc(c1)[Cl])═O |
| | carbaldehyde | |
| R9 | 5-(4-chloro-phenylsulfonyl)-furan- | C(c1ccc(o1)S(c1ccc(cc1)[Cl])(═O)═O)═O |
| | 2-carbaldehyde | |
| R10 (I7)ⁱ | 1-((acetoxyimino)methyl)-2- | CC(═O)ON═Cc1c(C)c(C═O)n2ccccc12 |
| | methylindolizine-3-carbaldehyde | |
| R11 | 5-(thiocyanato-methyl)-furan-2- | C(c1ccc(C═O)o1)SC#N |
| | carbaldehyde | |
| R12 | Phenanthrene-9-carboxaldehyde | C(c1cc2ccccc2c2ccccc12)═O |
| S1 | methyl 3-formyl-2-hydroxy- | COC(c1cccc(C═O)c1O)═O |
| | benzoate | |
| S2 | 4,5-dibromo-furan-2-carbaldehyde | C(c1cc(c(o1)[Br])[Br])═O |
| S3 | 3-iodo-4,5-dimethoxy- | COc1cc(C═O)cc(c1OC)I |
| | benzaldehyde | |
| S4 | 2-aza-bicyclo[4.4.0]deca- | C(c1cccc2cccnc12)═O |
| | 1(6),2,4,7,9-pentaene-10- | |
| | carbaldehyde | |
| S5 | 5-chloro-2-hydroxy-3-methoxy- | COc1cc(cc(C═O)c1O)[Cl] |
| | benzaldehyde | |
| S6 | 4-((1,1-dioxidobenzo[d]isothiazol- | C(c1ccc(cc1)OC1c2ccccc2S(N═1)(═O)═O)═O |
| | 3-yl)oxy)benzaldehyde | |
| S7 | N-(3- | CS(Nc1cccc(C═O)c1)(═O)═O |
| | formylphenyl)methanesulfonamide | |
| S8 | methyl 4-formyl-benzoate | COC(c1ccc(C═O)cc1)═O |
| S9 | 2-phenyl-acetaldehyde | C(C═O)c1ccccc1 |
| S10 | 4-methoxy-3-((1H-pyrazol-1-yl)- | COc1ccc(C═O)cc1Cn1cccn1 |
| | methyl)-benzaldehyde | |
| S11 | 4-ethynyl-benzaldehyde | C#Cc1ccc(C═O)cc1 |
| S12 | -ethyl 2-chloro-3-formyl-9-methyl- | CCOC(c1c(C)c2C(═C(CCc2[nH]1)C═O)[Cl])═O |
| | 7-aza-bicyclo[4.3.0]nona-1(6),2,8- | |
| | triene-8-carboxylate | |
| T1 | 8-(2-formyl-1H-pyrrol-1-yl)-7-thia- | C1CCc2c(C1)c(C(O)═O)c(n1cccc1C═O)s2 |
| | bicyclo[4.3.0]nona-1(6),8-diene-9- | |
| | carboxylic acid | |
| T2 | 7-aza-bicyclo[4.3.0]nona-1,3,5,8- | C(c1cccc2c1cc[nH]2)═O |
| | tetraene-2-carbaldehyde | |
| T3 | 5-formyl-4-hydroxy- | C(c1c(c(cc2ccccc12)C(O)═O)O)═O |
| | bicyclo[4.4.0]deca-1,3,5,7,9- | |
| | pentaene-3-carboxylic acid | |
| T4 | 4-(prop-2-ynoxy)-benzaldehyde | C#CCOc1ccc(C═O)cc1 |
| T5 | 4-chloro-2-methyl-2H-pyrazole-3- | Cn1c(C═O)c(cn1)[Cl] |
| | carbaldehyde | |
| T6 | 7-aza-bicyclo[4.3.0]nona-1,3,5,8- | C(c1ccc2c(cc[nH]2)c1)═O |
| | tetraene-3-carbaldehyde | |
| T7 | methyl 2-formyl-benzoate | COC(c1ccccc1C═O)═O |
| T8 | 5-((2,3,5,6-tetrafluoro-phenoxy)- | C(c1ccc(C═O)o1)Oc1c(c(cc(c1F)F)F)F |
| | methyl)-furan-2-carbaldehyde | |
| T9 | 2-(4-bromo-2-formyl-phenoxy)- | C(C#N)Oc1ccc(cc1C═O)[Br] |
| | ethanenitrile | |
| T10 | 1-(2-hydroxy-phenyl)-ethanone | CC(c1ccccc1O)═O |
| T11 | 1-(4-hydroxy-phenyl)-ethanone | CC(c1ccc(cc1)O)═O |
| T12 (H1, | 6-(4-formyl-phenyl)-pyridine-3- | C(c1ccc(cc1)c1ccc(C#N)cn1)═O |
| D1)ⁱ | carbonitrile | |

ⁱRepeat aldehydes for re-evaluation.
ⁱⁱBlank spaces for either blank DMSO or aminoxyl parent compounds as reference.

### Example 5: Development of TDP1 Inhibitors Via an Oxime-Based Optimization Strategy and Molecular Glue Degrader Discovery

By screening more than 600 low molecular weight fragments for their ability to co-crystallize with TDP1, we have solved several crystal structures of TDP1-bound compounds. Unfortunately, most of the identified compounds show very poor IC50 values (mM level) in TDP1 assays in vitro and a majority of the fragments represent variations on common hydroxyquinoline carboxylic acids or phthalic acids, which bind within the catalytic site in highly similar fashions. Recently, we have used Alexa Fluor 647 (AF647)-tagged TDP1(148-608) fluorescence probe to conduct a small molecule microarrays (SMMs) screen against 21,000 drug-like small molecules and identified 101 (M7) having a N,2-diphenylimidazo[1,2-a]pyrazin-3-amine nucleus as a new TDP1-binding motif (FIG. 6). Using Groebke-Blackbumin-Bienayme (GBBR) multicomponent one-pot reactions employing readily available building blocks of aldehyde, pyrazin-2-amine or pyridin-2-amine and isocyanide, we have conducted structure-activity relationship (SAR) studies, which further extended this class of compounds to N,2-diphenylimidazo[1,2-a]pyridin-3-amines such as 102 (XZ615) and 103 (XZ664) (FIG. 6). By introducing a phthalic acid motif, we have been able to confirm that 104 (XZ634) (FIG. 6) is binding at the catalytic site of TDP1, where they mimic interactions of the phosphate joining the DNA-TOP1 substrates and binds to TDP1 in a tri-dentate form at the catalytic site and extends to both of the open pockets that encompass the DNA and peptide substrate binding sites.

Progress in developing TDP1 inhibitors has been slow. This is due in part to a lack of high-quality crystal structures of lead small molecule inhibitor bound to the TDP1 catalytic site. Several crystal structures have been reported with vanadate or tungstate phosphate mimics bound at the TDP1 catalytic site as well as with DNA or substrate surrogates. As a member of phospholipase D (PLD) superfamily, TDP1 has two conserved “His-Lys-Asn” (HKN) motifs at the active site, which are responsible for sequential nucleophilic attacks on the DNA phosphodiester bond. TOP1-DNAcc represents a physiological bi-substrate for TDP1 having both protein and DNA components joined by a phosphoryl linkage. Crystal structures have been solved that approximate critical reaction intermediates associated with cleavage of the DNA strand. These structures consist of a three-component construct, in which a single stranded substrate DNA and a tyrosyl-containing peptide are situated in regions of TDP1 normally occupied by polydeoxynucleotide and TOP1 substrates. A vanadate moiety binds in place of the cognate phosphate ester, where it mimics the geometry of the TDP1-bound phosphoryl transition state complex. These define the probable binding regions for the major components of the substrate. There is a deep catalytic cleft, whose bottom is formed by the two HKN motifs covalently linked to the vanadate phosphoryl mimetic. Extending from one side of the active site is a long, positively charged cleft, where the single-stranded DNA binds. In the opposite direction the TOP-derived peptide binds in a more open pocket. These interactions may serve as guides for designing inhibitors. Yet, although the HKN phosphoryl-binding pocket is well defined, to date inhibitor development has been extremely challenging and this maybe a reflection of the open, extended nature of the DNA and peptide-binding regions.

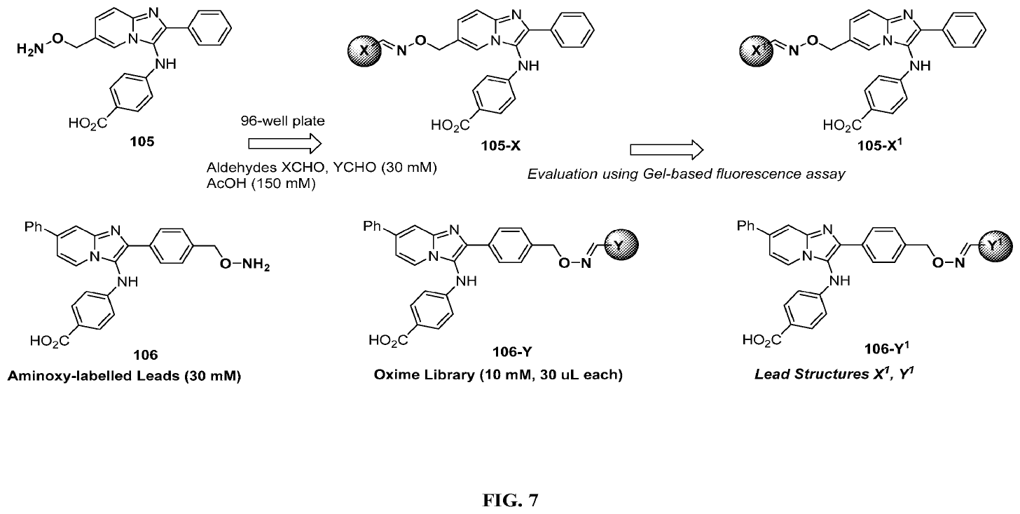

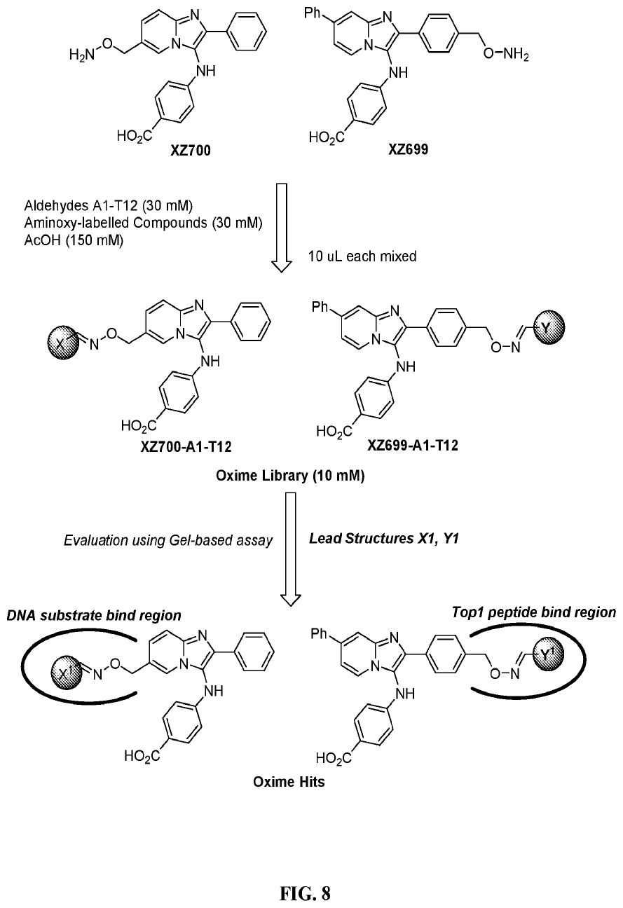

We designed an oxime-based optimization strategy and screen diversity of oximes (about 500 different structures, Table 4, Example 4) based on the SMM leads imidazo[1,2-a]pyridin-3-amines (102 and 103) as well as the X-ray cocrystal structure of 104 in order to extend the binding surface to the DNA and TOP1 peptide binding areas in TDP1 to optimize their activities. See FIG. 7 and FIG. 8. Some of the lead compounds serve as molecular glue degraders of TDP1 in cells.

Although the actual HKN phosphoryl-binding pocket is well defined, to date development of TDP1 inhibitors has been extremely challenging. The reasons may be due in part to the open, extended nature of the DNA and peptide-binding regions. Based on the co-crystal structure, the N,2-diphenylimidazo[1,2-a]pyridin-3-amine analogues bind at the TDP1 catalytic site between the open regions of the DNA substrate and peptide-binding pockets. According to the co-crystal structure, 104 binds HKN motifs at the catalytic site of TDP1. Pyridine in 104 points to a long, positively charged cleft, where the single-stranded DNA binds, and phenyl in 104 points to a more open pocket in the opposite direction which is the TOP-derived peptide binds. To efficiently explore the DNA substrate and TOP1 peptide-binding regions, we proposed to employ oxime strategy to extend the binding surface of the lead compounds and optimize ligand binding affinity. We designed the aminoxyl-labelled N,2-diphenylimidazo[1,2-a]pyridines 105 and 106, which can be prepared using the GBBR multi-components synthetic protocol. (FIG. 7) Aminoxyl in 105 points to the open region of DNA substrate binding site, and aminoxyl in 106 points to the open region of TOP1 peptide binding site. Importantly, both binding sites could be easily explored using a library of aldehydes (˜250 different aldehydes, X—CHO and Y—CHO, Table 4). Oximes can be prepared in parallel in a 96-well plate format starting from aminoxyl-labelled compounds (105 and 106) and a library of about 250 aldehydes with acetic acid in DMSO. The TDP1 inhibition of the formed oximes (105-X and 106-Y) could be evaluated by using gel-based fluorescence assay directly without purification. The lead oximes (105-X¹ and 106-Y¹) could be identified based on the related inhibition values. These oximes would extend the originally binding surface to both DNA and TOP1 binding sites. We further developed some analogues based one the oxime lead structure by replacing the oxime linker with a triazole linkers or ether linkers. Using this approach, we have found lower micromolar to nanomolar TDP1 inhibitors. Some of the lead compounds serve as molecular glue degraders of TDP1 in micromolar level in cells, and some display a synergistic effect with camptothecin (CPT) in human colon cancer cell line HCT116.

### Degradation Assay

The degradation effect of the TDP1 inhibitors on TDP1 in human colon cancer cell line HCT116 and embryonic kidney cell line HEK293 were studied. Cells were first seeded in a 6-well plate until 60-80% confluency and then incubated with a serial dilution of TDP1 inhibitors at the range of 25-200 μM for 24-72 h at 37° C. Drug treated cells were collected and washed twice by DPBS followed by lysing in 1×RIPA buffer (50 mM Tris HCl, pH 7.4, 150 mM NaCl, 1 mM EDTA, 1 mM DTT, 0.1% sodium deoxycholate, 0.1% SDS, and 1% TritonX-100) supplemented with 1× protease inhibitor cocktail (Thermo). Samples were then sonicated at 30% for 10 s and centrifuged for 15 min at 15000 rpm at 4 Celsius. Supernatant were collected and the concentration of total protein were measured by BCA Protein Assay Kit according to its protocols. Four micrograms of total protein for each sample were loaded and TDP1 level were conducted by western blotting.

### Results

The oximes were evaluated by gel-based TDP1 fluorescence assay in a concentration of 100 μM in DMSO. The fluorescence of DMSO blank vial was set as 0 and the fluorescence for the reference without TDP1 was set as 100%. Preparation: A mixture of aminooxy-containing 105 or 106 (10 μL, 30 mM in DMSO), aldehydes M1-T12 (10 μL, 30 mM in DMSO) and acetic acid (10 μL, 150 mM in DMSO) were agitated at room temperature overnight. Oximes 5-M1-T12 or 6-M1-T12 (30 uL, 10 mM in DMSO) were afforded.

Two set of oximes 105-X and 106-Y (XZ700-M1-T12, XZ699-M1-T12) (20 uL, 10 mM in DMSO) were prepared about 240 each starting from aminooxy-containing 105 (XZ700) or 106 (XZ699) based on the following method. A mixture of aminooxy-containing 105 or 106 (10 μL, 30 mM in DMSO) with about 240 aldehydes M1-T12 (10 μL, 30 mM in DMSO) (Table 4) separately in the present of acetic acid (10 μL, 150 mM in DMSO) were agitated at room temperature overnight in three 96-well plates in parallel. The formed oximes 105-M1 to 105-T12 and 106-M1 to 106-T12 (30 uL, 10 mM in DMSO) were diluted to 100 μM in DMSO and evaluated by gel-based TDP1 fluorescence assay. The fluorescence of the gel band of DMSO blank vial was set as 0 and the fluorescence for the reference gel band without TDP1 was set as 100%. As shown in Table 5 and Table 6, 19 oximes in 105-X series library show good inhibition (>90%) and 47 oximes in 106-Y series library show good inhibition (>90%). Among them, with same aldehyde structures D1 and P3, both oximes 105-series (105-D1, 105-P3) and 106-series (106-D1, 106-P3) show great (>90%) TDP1 inhibition. Most interestingly, aldehyde having 2-phenylpyridine retained good TDP1 inhibition in repeat test (D1, H1 and T12) in both 105-series and 106-series. With similar structures, 106-B7 and 106-E6 shows good inhibition. However, 105-M10 and 106-M10 shows poor inhibition. To confirm the inhibitions, we have further evaluated the purified lead oximes by HPLC. (Table 7; FIG. 9)

TABLE 7
Inhibitory potencies of HPLC-purified oxime leads determined
in gel based TDP binding fluorescence assays.

| COMPOUND | TDP1 IC₅₀ (μM)ⁱ | TDP2 IC₅₀ (μM)ⁱⁱ | FCⁱⁱⁱ |
|---|---|---|---|
| XZ615 | 7.87 ± 2.24 | >100 | >13 |
| XZ717 | 50.3 ± 10.7 | >100 | >2 |
| XZ706 | 17.4 ± 3.2 | 62.1 ± 10.1 | 4 |
| XZ713 | >100 | >100 | — |
| XZ707 | >100 | >100 | — |
| XZ664 | 3.53 ± 0.81 | >100 | >28 |
| XZ708 | 5.65 ± 3.29 | >100 | >18 |
| XZ701 | 0.38 ± 0.06 | 28.1 ± 8.9 | 74 |
| XZ709 | 65.3 ± 17.8 | >100 | >2 |
| XZ702 | 3.1 ± 0.5 | 8.8 ± 2.9 | 3 |
| XZ710 | 7.67 ± 2.3 | >100 | >13 |
| XZ703 | 10.2 ± 0.33 | 10.9 ± 1.1 | 1 |
| XZ711 | 54.2 ± 18.7 | >100 | >2 |
| XZ704 | 76.3 ± 41.9 | >100 | >1 |
| XZ712 | 63.1 ± 27.5 | >100 | >2 |
| XZ705 | 67.7 ± 17.6 | >100 | >2 |

ⁱThe half maximal inhibitory concentration (IC₅₀) have been evaluated by gel-based TDP1 fluorescence assay in vitro.
ⁱⁱThe half maximal inhibitory concentration (IC₅₀) have been evaluated by gel-based TDP2 fluorescence assay in vitro.
ⁱⁱⁱThe fold change (FC) have been calculated by the ratio of IC₅₀ TDP2 to TDP1, which reflect the TDP1 selectivity over TDP2.

When we prepared the lead oximes and purified by HPLC, we found two products with same molecular weight but different retention time. The major product and the minor product always have a ratio around 95:5 according to 254 nm UV signals. Based on the thermodynamic stability of the oxime double bond, we assigned the major product would be (E)-oxime isomer and the minor one should be (Z)-oxime isomer. We evaluated the inhibitory potencies of both lead oxime isomers using TDP1 and TDP2 fluorescence binding assays in vitro. (Table 7) SMM lead compounds 102 and 103 were included as reference. As shown in Table 7, both 102 and 103 show single digital micromolar inhibition selectivity against TDP1 but not TDP2 in vitro. Most of the lead oximes retain the TDP1 selectivity. For 105-series oximes, the major (E)-105-D1 (TDP1 IC₅₀=17.4±3.2 μM) shows better inhibition than the minor (Z)-105-DI (TDP1 IC₅₀=50.3±10.7 μM) against TDP1. However, it shows about 2-fold loss potency than the originally 102 (TDP1 IC₅₀=7.87±2.24 μM). Slight change the structure to 105-P3 (TDP1 IC₅₀>100 μM) leads to lose TDP1 inhibition. For oximes 106-D1 and 106-E1 among 106-series oximes, (E)-isomers show 10-fold better TDP1 inhibition than (Z)-isomers. For oximes 106-B7, 106-P3 and 106-M10 among 106-series oximes, (Z)-isomers show slightly better TDP1 inhibition than (E)-isomers. Several oximes including (E)-106-D1, (Z)-106-D1, (E)-106-E6 and (Z)-106-B7 show single digital TDP1 inhibition. Oxime (E)-106-B7 retains inhibitory potencies (IC₅₀˜10 μM) against both TDP1 and TDP2. Oxime (E)-106-E6 show single digital inhibitory potencies against both TDP1 (TDP1 IC₅₀=3.1±0.5 μM) and TDP2 (TDP2 IC₅₀=8.8±2.9 μM). Most promisingly, inhibitory potencies against TDP1 of the lead oxime (E)-106-D1 shows nanomolar inhibition (TDP1 IC₅₀=0.38±0.06 μM) and good selectivity (74-fold) against TDP1 than against TDP2. The TDP1 inhibition of (E)-106-D1 is 10-fold more than the SMM lead 3 (TDP1 IC₅₀=3.53±0.81 μM). The oxime (E)-106-D1 has extend the TDP1 binding surface to both DNA and peptide-binding regions and shows nanomolar TDP1 inhibitory potencies and good selectivity over TDP2.

Molecular glue degraders induce protein-protein interactions and lead to protein degradation. Phenylpyridine moiety have been conferred to contribute to the glue activity in cyclin-dependent kinase (CDK) inhibitor as glue degrader that depletes cyclin K. Herein our nanomolar oxime lead (E)-106-D1 have 4-(phenyl)nicotinonitrile might contribute the TDP1 degradation too. Therefore, we prepared a series of analogues with different linkers such as triazoles in XZ718-XZ723 and ethers in XZ726 and XZ727 other than oxime XZ701. To compare the SAR, we prepared both analogues with or without phenyl as XZ664 or XZ615. We found that these analogues show micromolar TDP1 inhibition in gel-based fluorescence assay in vitro. (Table 8) Some of them show TDP1 selectivity over TDP2. In 200 uM concentration, XZ664 shows TDP1 degradation in cells but XZ615 does not. (Table 8 and FIGS. 11-16).

TABLE 8
Inhibitory potencies of lead compounds with different linkers
determined in gel based TDP binding fluorescence assays.

| Compound | TDP1 IC₅₀ (μM)ⁱ | TDP2 IC₅₀ (μM)ⁱⁱ | FCⁱⁱⁱ |
|---|---|---|---|
| 102 (XZ615) | 7.87 ± 2.24 | >100 | |
| 103 (XZ664) | 2.98 ± 0.24 | >100 | |
| | (3.53 ± 0.81) | | |
| (E)-106-D1 | 0.38 ± 0.06 | 28.1 ± 8.9 | |
| (XZ701) | | | |
| 107a (XZ722) | 5.95 ± 1.25 | 18 ± 1.2 | 3 |
| 107b (XZ723) | 66.4 ± 3.7 | 69.1 ± 11.8 | 1 |
| 107c (XZ721) | 60 ± 17 | >100 | >1 |
| 107d (XZ720) | 23.5 ± 3.2 | 32.9 ± 2.7 | 1 |
| 107e (XZ719) | 21.6 ± 5.0 | 62.4 ± 18.6 | 3 |
| 107f (XZ718) | 3.1 ± 0.2 | 25.9 ± 0.4 | 8 |
| 108a (XZ727) | 25.1 ± 1.25 | 70.5 ± 13.5 | 3 |
| 108b (XZ726) | 2.75 ± 0.25 | >100 | >36 |
| XZ725 | 25.6 ± 4.05 | >100 | |
| XZ730 | 17.8 | 39.5 | |
| | (25° C., 24 h) | (25° C., 24 h) | |
| XZ724 | >100 | >100 | |

ⁱThe half maximal inhibitory concentration (IC₅₀) have been evaluated by gel-based TDP1 fluorescence assay in vitro.
ⁱⁱThe half maximal inhibitory concentration (IC₅₀) have been evaluated by gel-based TDP2 fluorescence assay in vitro.
ⁱⁱⁱThe fold change (FC) have been calculated by the ratio of IC₅₀ TDP2 to TDP1, which reflect the TDP1 selectivity over TDP2.

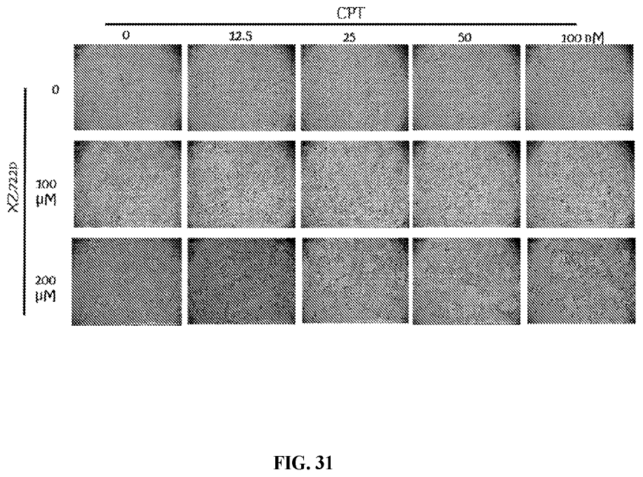

The synergistic effect with camptothecin (CPT) of various oxime lead compounds and related compounds is shown in FIGS. 19-31. Assays were conducted as described in Example 3.

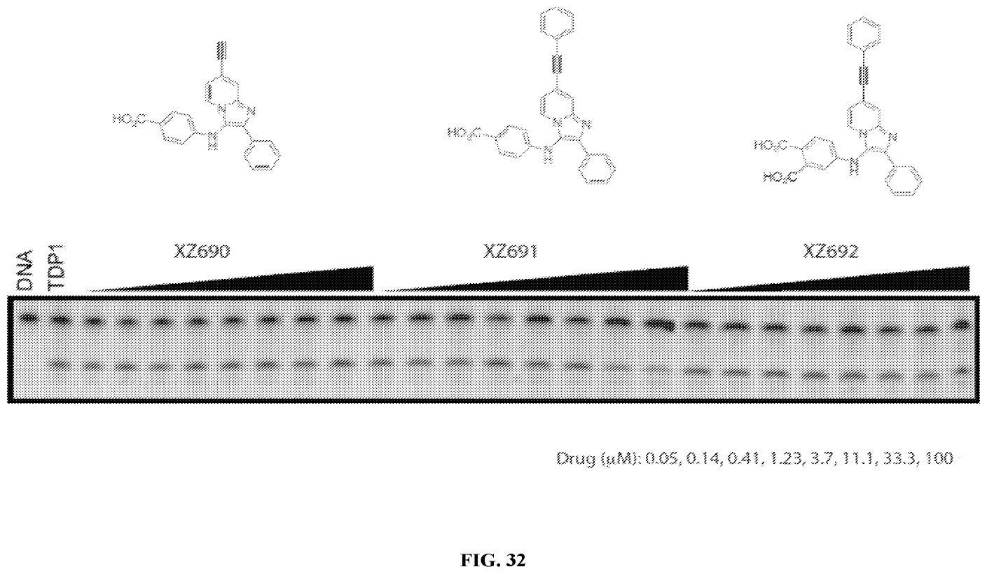

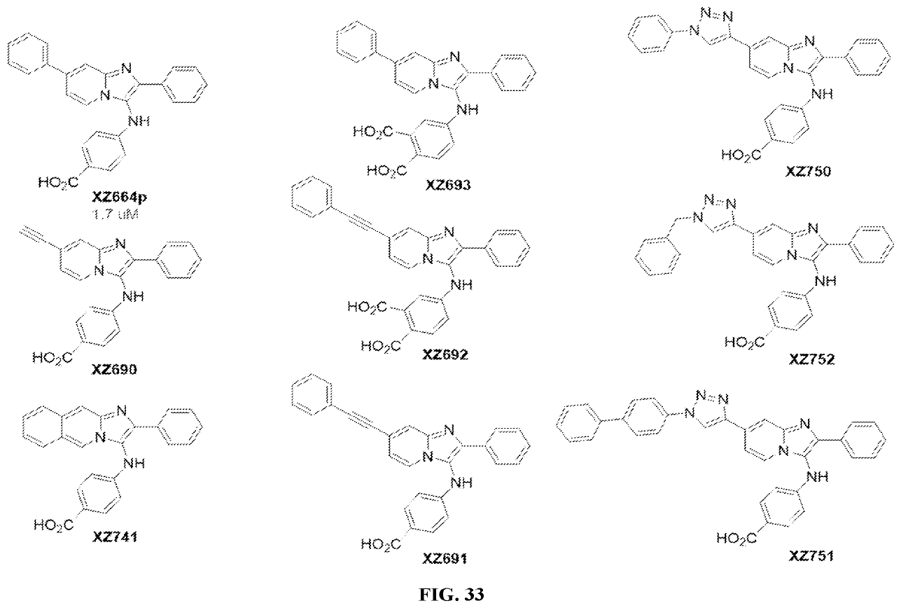

TDP1 residue phenylalanine 259 (F259) is highly conserved across species and critical for activity. Thus, we developed a series of inhibitory analogs targeting F259, as shown in FIGS. 32-33.

### Conclusions

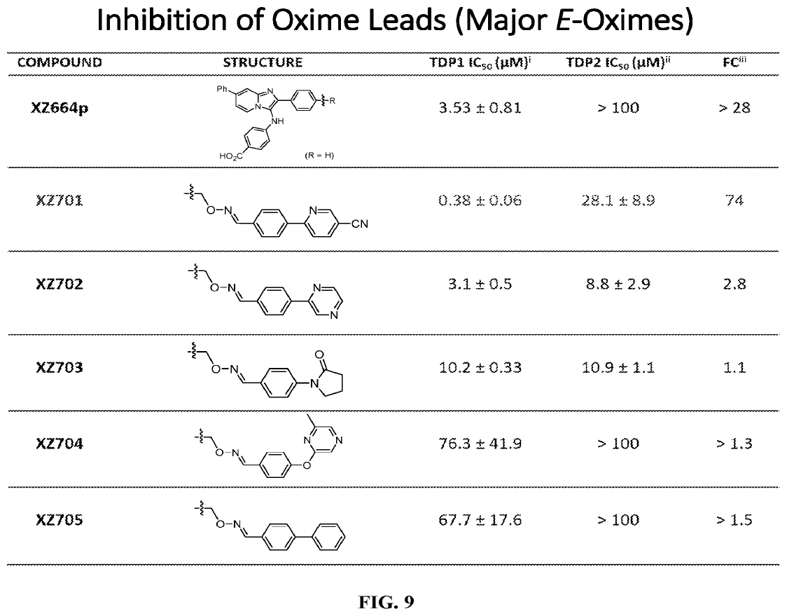

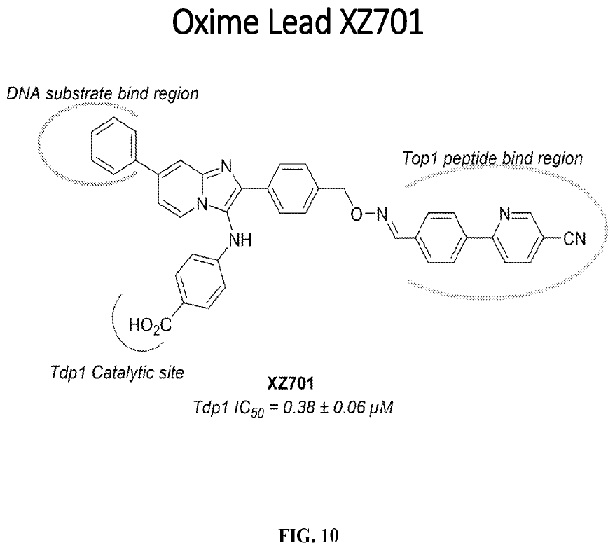

Based on our previously reported X-ray co-crystal structure of TDP1 bound to small molecule 104, we have designed and prepared the aminoxyl-labelled N,2-diphenylimidazo[1,2-a]pyridines (105 and 106), using the GBBR multi-components one-pot synthetic protocol. Aminoxyls in 105 and 106 point to the open region of DNA substrate binding site and TOP1 peptide binding site separately. Importantly, both binding sites can be approached parallelly using oxime-based strategy with a library including about 250 aldehydes. We have employed this approach to optimize ligand design and increase ligand affinity. The formed oximes extend the originally binding surface to both DNA and TOP1 binding sites. Inhibitory potencies against TDP1 of the lead oxime (E)-106-D1 (XZ701) (FIG. 10) shows nanomolar inhibition and good selectivity (74-fold) against TDP1 than against TDP2. Structural interactions of the promising analogs have been analyzed using MolSoft ICM modeling software to elucidate molecular interactions with TDP1. The potent inhibitors can specifically degrade the TDP1 as molecular glue degraders and may serve as a new genre of anticancer chemotherapeutics.

### Example 6: Inhibition of TDP1 and TDP2 by Fluorosulfates and Sulfonyl Fluorides

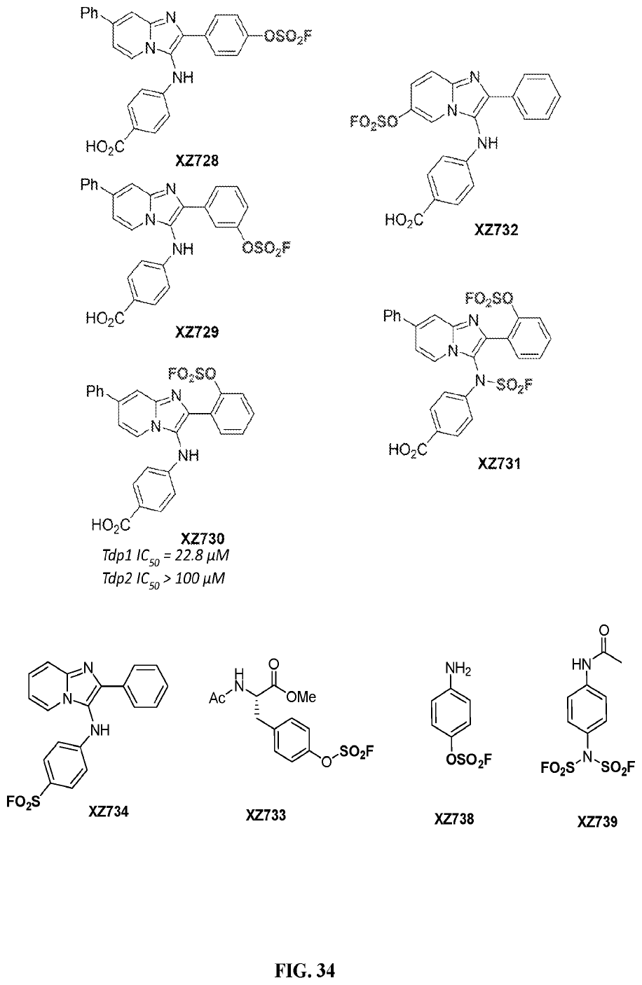

We designed series of fluorosulfate and sulfonyl fluoride covalent inhibitor compounds via sulfur-fluoride exchange click chemistry; inhibition studies were conducted using a gel-based fluorescence assay in vitro as described above. Structures are shown in FIG. 34. Results are presented in Table 9.

TABLE 9

| Compound | TDP1 IC₅₀ (μM) | TDP2 IC₅₀ (μM) |
|---|---|---|
| XZ728 | >100 | >100 |
| XZ729 | >100 | >100 |
| XZ730 | 17.8 | 39.5 |
| XZ731 | 22.7 | 45.5 |
| XZ732 | >100 | 65.8 |
| XZ734 | >100 | >100 |

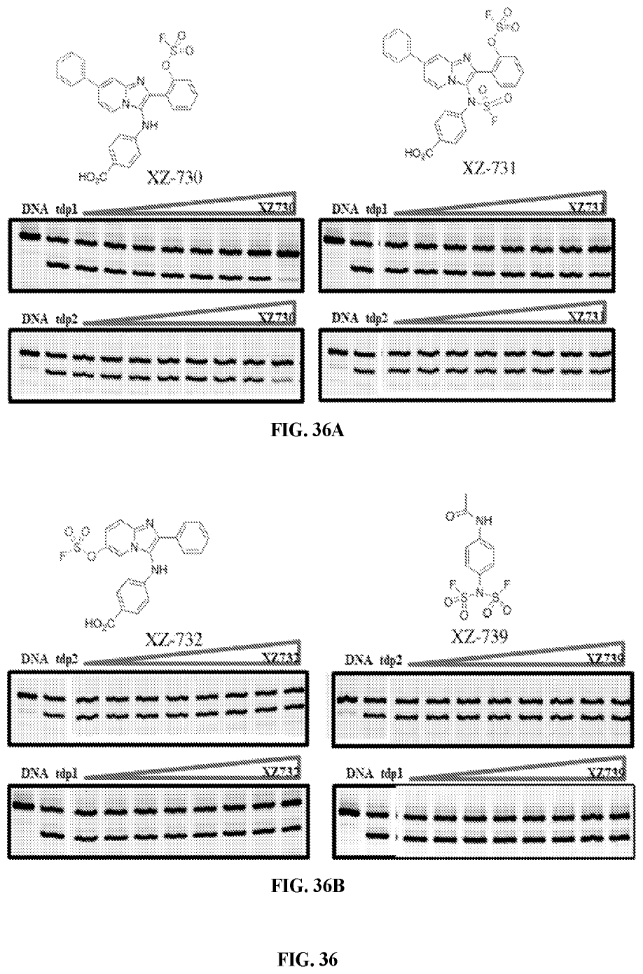

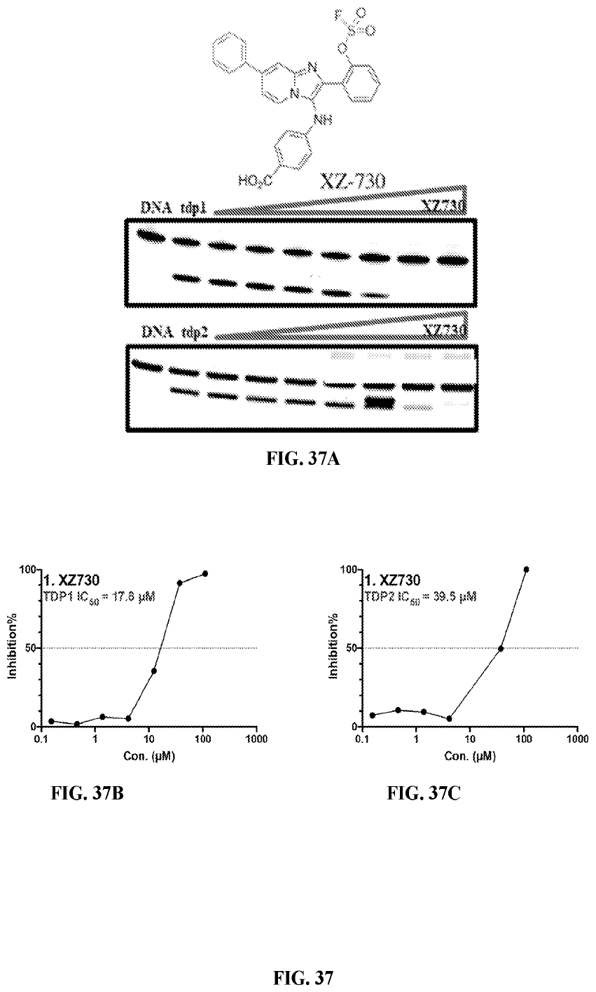

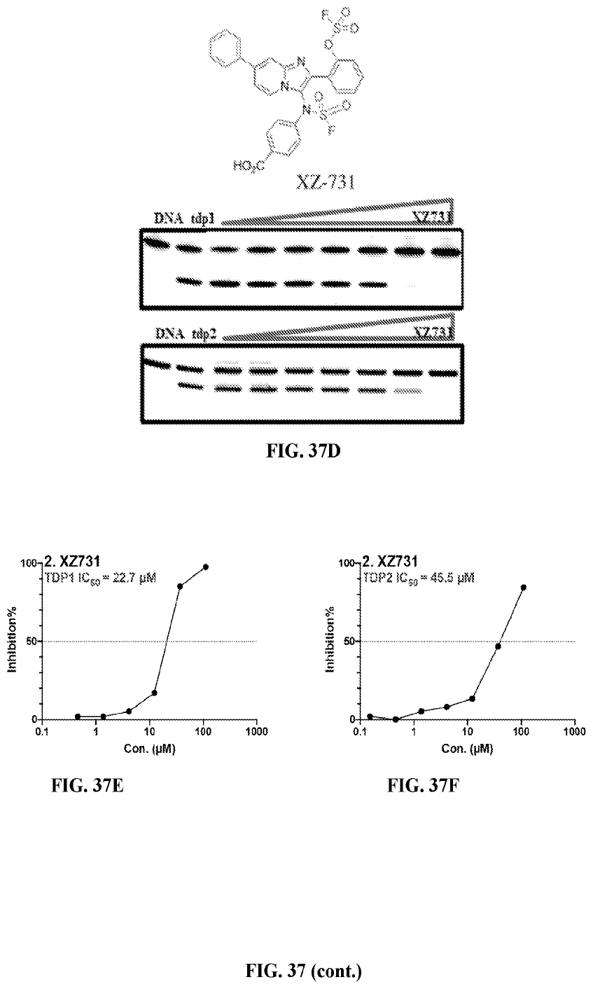

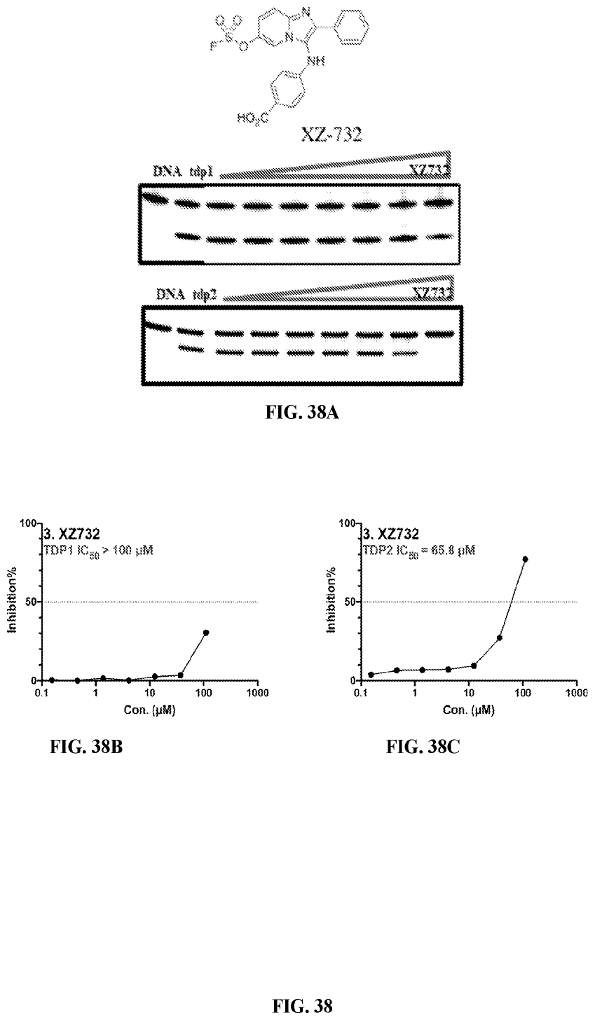

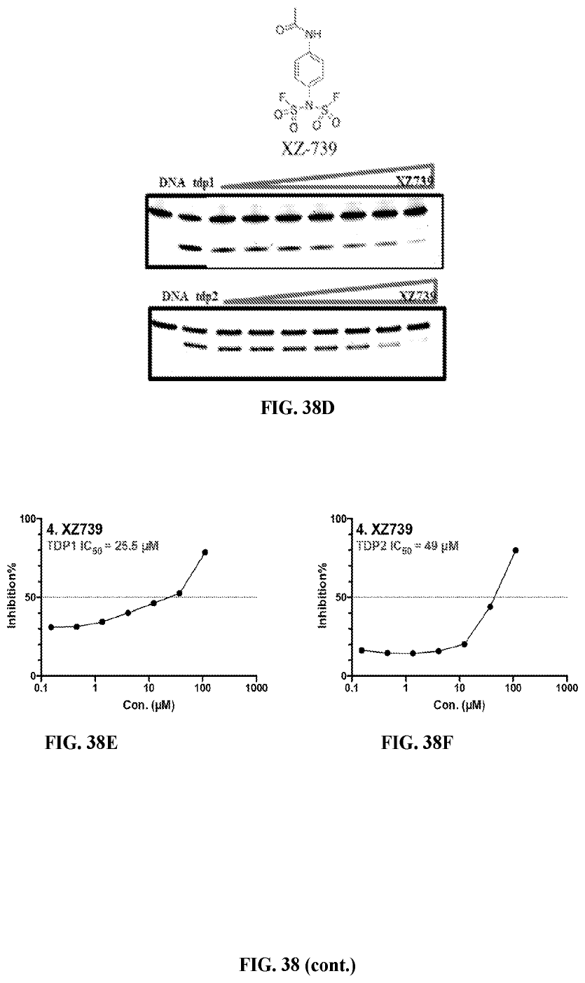

A primary screen of fluorosulfate compounds revealed inhibition by XZ730 and XZ731 at 111 μM after preincubation with TDP1 at room temperature overnight, and inhibition by XZ730, XZ731, XZ732, and XZ739 at 111 μM after preincubation with TDP2 at room temperature overnight. (FIG. 35). A secondary screen of XZ730 and XZ731 revealed showed no inhibition of TDP1 at room temperature after 10 minutes except for XZ730. (FIG. 36), however with preincubation with TDP overnight at room temperature, μM inhibition was observed for XZ730 and XZ731, with 2-fold selectivity comparted with TDP2 (FIG. 37) and μM inhibition was also observed for XZ739 (FIG. 38).

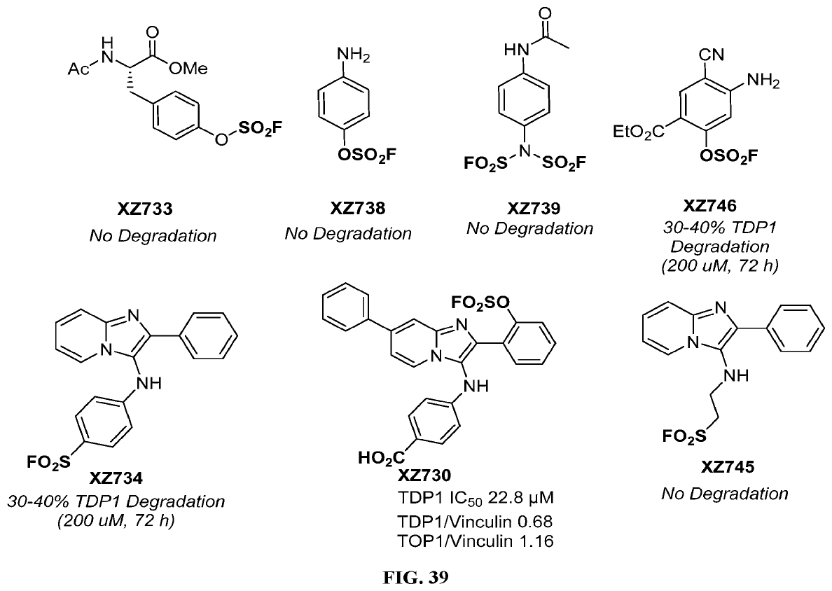

XZ746, XZ734, and XZ730 were found to degrade TDP1 as shown in FIG. 39.

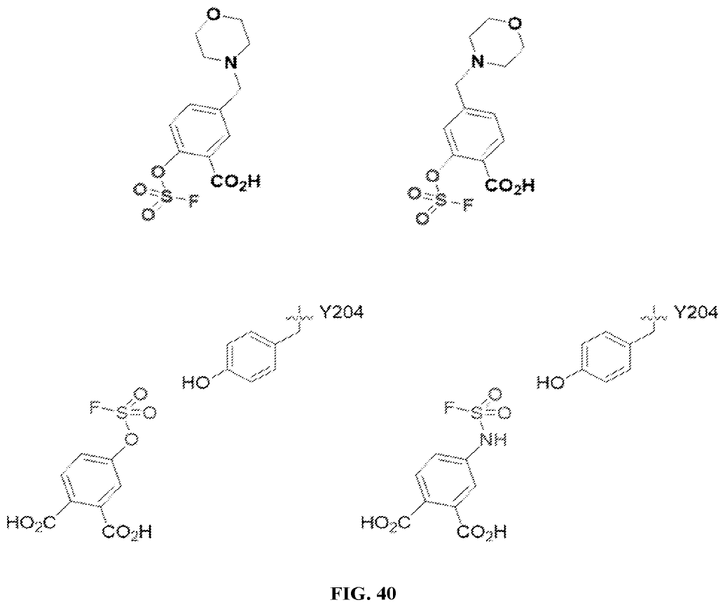

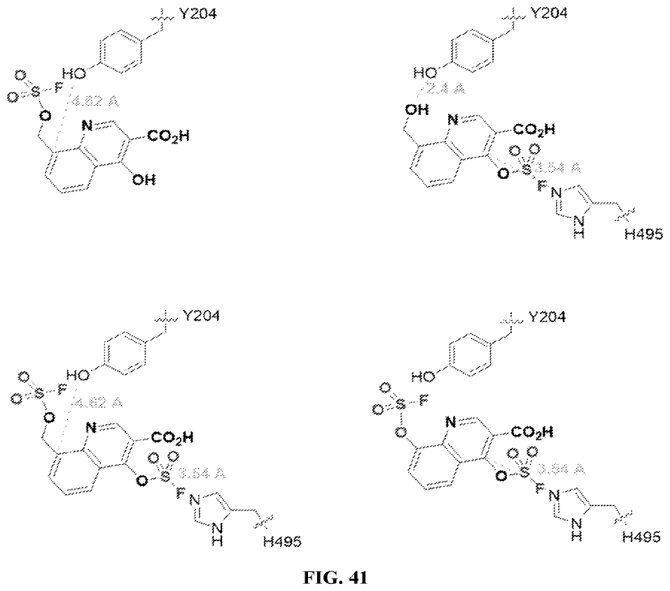

Additional covalent inhibitor compounds were designed based on phthalic acids (FIG. 40) and quinolones (FIG. 41).

### Example 7: PROTAC TDP1 Inhibitors

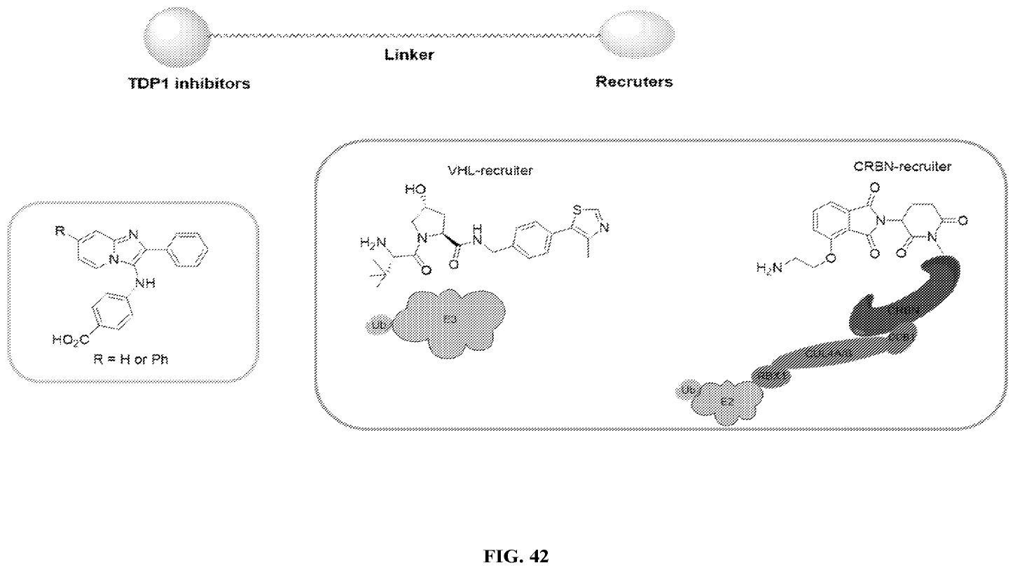

We designed a series of Proteolysis Targeting Chimeras (PROTAC) for the ubiquitin proteasome degradation of TDP1 based on the binding mode of XZ634. The overall design is shown in FIG. 42. Conjugates were prepared using VHL recruiters (FIG. 43) and CRBN recruiters (FIG. 44).

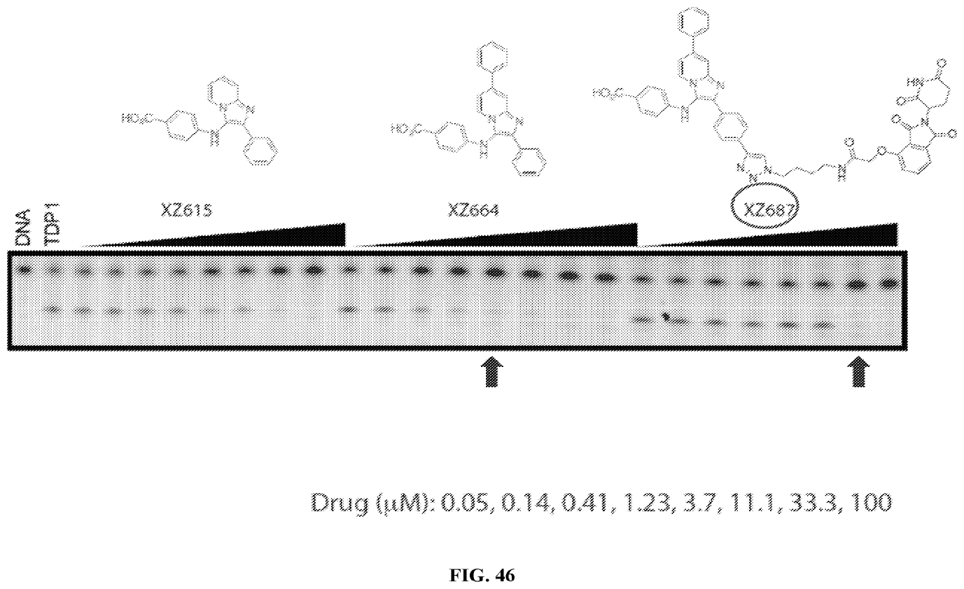

Inhibition studies on various TDP1 PROTAC conjugates were conducted using a gel-based fluorescence assay in vitro as described above; results are presented in Table 10 and FIGS. 45 and 46.

TABLE 10
Structures and inhibitory potencies of PROTACs in gel-
based fluorescence assay with TDP1 and TDP2 in vitro.

| Compound | TDP1 IC₅₀ (μM) | TDP2 IC₅₀ (μM) |
|---|---|---|
| XZ679 | 70.3 ± 2.4 | >100 |
| XZ680 | >100 | >100 |
| XZ681 | >100 | >100 |
| XZ682 | >100 | >100 |
| XZ683 | >100 | >100 |
| XZ684 | >100 | >100 |
| XZ685 | >100 | >100 |
| XZ686 | >100 | >100 |
| XZ687 | 33.1 ± 7.2 | 37.3 ± 4.5 |
| XZ688 | >100 | >100 |
| XZ689 | >100 | >100 |
| XZ742 | >100 | >100 |
| XZ743 | >100 | >100 |
| XZ747 | 7.41 ± 0.19 | 11.2 ± 0.55 |
| XZ748 | 18.5 ± 1.55 | 21.2 ± 0.15 |
| XZ749 | 12.34 ± 1.5 | 16.8 ± 0.3 |

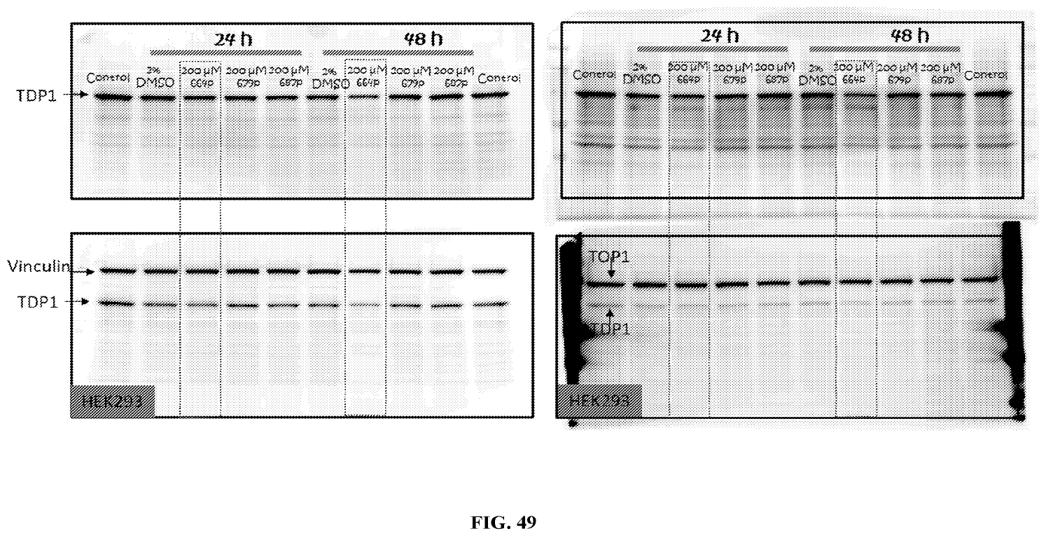

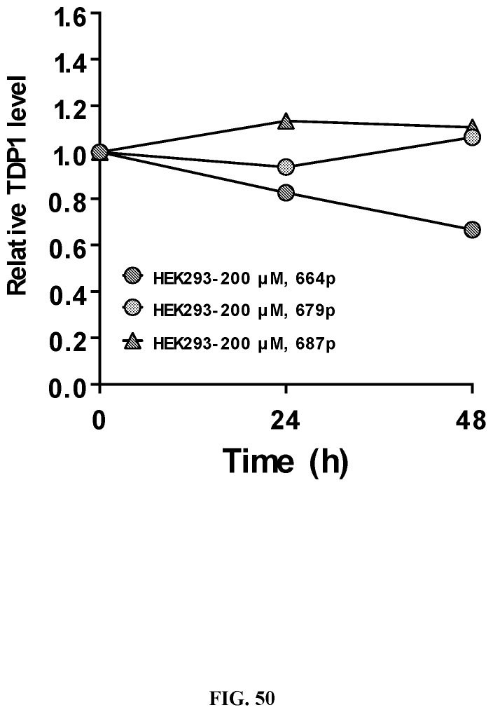

Degradation studies were conducted with PROTAC conjugates XZ679 and XZ687, along with precursor XZ664, in HEK293 and HCT116 cells. Results are shown in FIGS. 47-50.

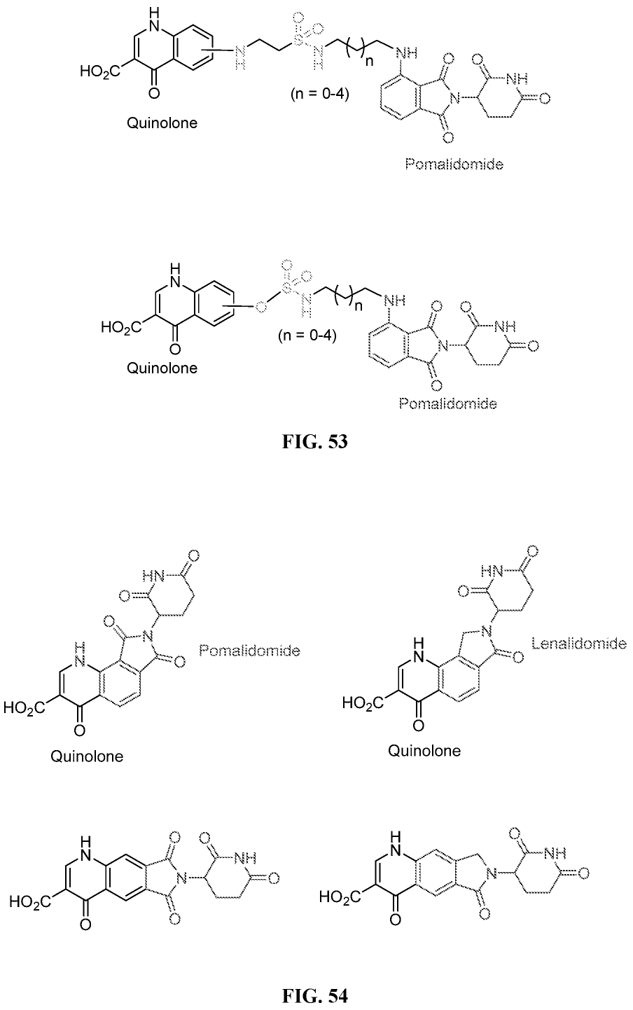

Further PROTAC conjugates were prepared as outlined above with modified linkers (FIGS. 51-52) and with quinolones as the TDP1 binder (FIGS. 53-54).

1. A compound of Formula I 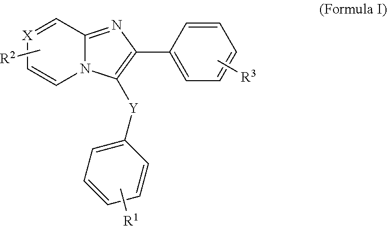
or a pharmaceutically acceptable salt thereof, wherein
R¹ is absent or represents one or more substituents independently selected from H, halogen, hydroxyl, cyano, nitro, amino, —COOH, —CHO, —CONH₂, —SO₃H, —SO₂F, —OSO₂F, —SO₂(C₁-C₂alkyl), —(C₁-C₂alkyl)CO₂H, —PO₃H, —PO₂(C₁-C₂alkyl), —PO₂NH(C₁-C₂alkyl), —(C₁-C₂alkyl)PO₃H, —(C₁-C₂alkyl)PO₂NH(C₁-C₂alkyl), phenyl, phenoxy, benzyl, benzyloxy, C₁-C₆alkyl, C₁-C₆alkoxy, C₁-C₂haloalkyl, and C₁-C₂haloalkoxy, mono- or di(C₁-C₄alkyl)amino, C₃-C₇cycloalkyl, or 5-7-membered heterocycloalkyl, trifluoromethyl, or trifluoromethoxy;
R², and R³ are each independently absent, or represent one or more substituents independently selected from H, halogen, hydroxyl, cyano, nitro, amino, —COOH, —CHO, —CONH₂, —SO₃H, —SO₂F, —OSO₂F, —SO₂(C₁-C₂alkyl), phenyl, phenoxy, benzyl, benzyloxy, C₁-C₆alkyl, C₁-C₆alkoxy, C₁-C₂haloalkyl, and C₁-C₂haloalkoxy, mono- or di(C₁-C₄alkyl)amino, C₃-C₇cycloalkyl, or 5-7-membered heterocycloalkyl, trifluoromethyl, trifluoromethoxy, —(C₁-C₄)ONH₂, an oxime of formula —(C₁-C₄)ON═C—R²⁰, wherein the oxime is formed by the reaction of —(C₁-C₄)ONH₂ and an aldehyde 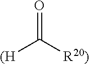
selected from the group consisting of A1-T12 in Table 4; or
-L-Pec, wherein L is a linker selected from the group consisting of 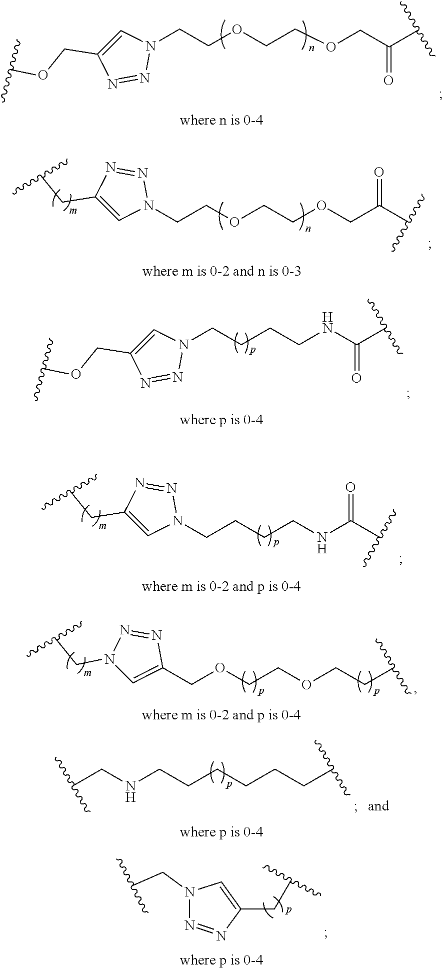
and
Rec is an E3 ubiquitin ligase recruiter;
wherein not all of R¹, R², and R³ are absent and at least one of R¹, R², and R³ is phenyl, —COOH or nitro;
X is C or N; and
Y is NH, O, or NSO₂F.

2. The compound of claim 1, wherein Rec is a von Hippel-Lindau (VHL) E3 ubiquitin ligase recruiter, a cereblon (CRBN) E3 ubiquitin ligase recruiter, a Inhibitors of Apoptosis Protein (IAPs) E3 ubiquitin ligase recruiter, or a mouse double minute 2 homolog (MDM2) E3 ubiquitin ligase recruiter.

3. The compound of claim 2, wherein Rec is a VHL recruiter according to 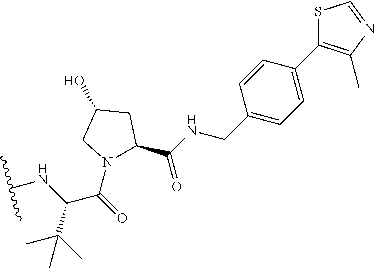 [SMILES: *N[C@H](C(=O)N1C[C@H](O)C[C@H]1C(=O)NCc1ccc(-c2scnc2C)cc1)C(C)(C)C]
or
Rec is a CRBN recruiter according to 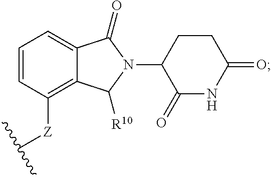
where R¹⁰ is H or carbonyl; and
Z is O, NH, or N(C₁-C₃alkyl).

4. The compound of claim 1, wherein
R¹ is absent or one or more substituents independently chosen from hydroxyl, nitro, and —COOH;
R² is absent or one or more substituents independently chosen from bromo, —COOH, —SO₂CH₃, and phenyl; and
R³ is absent or one or more substituents independently chosen from —COOH, —SO₃H, hydroxyl, nitro, methyl, methoxy, butoxy, phenyl, morpholinyl, trifluoromethyl, and trifluoromethoxy.

5. The compound of claim 1, wherein the compound is a compound of Formula I.5 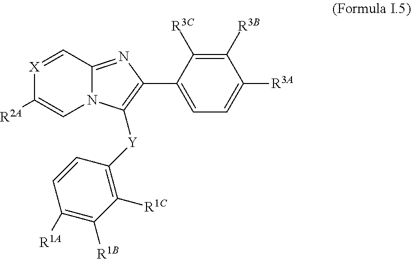
or a pharmaceutically acceptable salt thereof, wherein
R^{1A} is absent or represents H, nitro, —COOH;
R^{1B} is absent or represents H, —COOH;
R^{1C} is absent or represents H or hydroxyl;
R^{2A} is absent or represents H, halogen, —COOH, —SO₂Me, —CH₂ONH₂, an oxime of formula —CH₂ON═C—R²⁰, wherein the oxime is formed by the reaction of —CH₂ONH₂ and an aldehyde 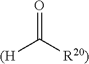
selected from the group consisting of B7, D1, E6, F₅, G6, G7, G8, I7, M10, M11, N3, N4, O11, P1, P3, P4, P8, R¹⁰, R¹¹, S4, S12, and T12 from Table 4;
R^{2B} is absent or represents H, —COOH, phenyl;
R^{3A} is absent or represents H, —COOH, hydroxyl, C₁-C₃alkyl, phenyl, benzyloxy, trifluoromethyl, nitro, —SO₃H, —SO₂(C₁-C₂alkyl), morpholinyl, —CH₂ONH₂, 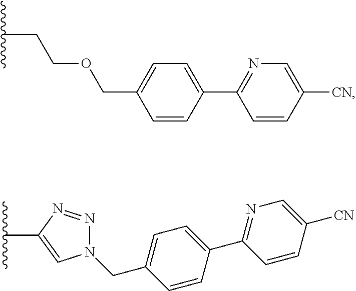 [SMILES: *CCOCc1ccc(-c2ccc(C#N)cn2)cc1 ; *c1cn(Cc2ccc(-c3ccc(C#N)cn3)cc2)nn1]
or an oxime of formula —CH₂ON═C—R²⁰, wherein the oxime is formed by the reaction of —CH₂ONH₂ and an aldehyde 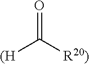
selected from the group consisting of A6, B7, B9, B11, D1, D10, E6, F1, F3, F9, F10, F11, G6, G8, H1, H7, H11, I1, I3, I7, I8, J9, K1, K9, L8, L11, M10, M1, N2, N3, N4, N8, O11, P2, P3, P4, P8, Q9, Q12, R7, R10, R11, S2, S11, T1, T3, and T12 from Table 4;
R^{3B} is absent or represents H, hydroxyl;
R^{3C} is absent or represents H, hydroxyl;
X is C—R^{2B} or N; and
Y is NH.

6. The compound of claim 5, wherein
X is C—R^{2B}, R^{1A} is —COOH, R^{3C} is hydroxyl, C— and R^{1B}, R^{1C}, R^{2A}, R^{2B}, and R^{3A} are all H (8a);
X is C—R^{2B}, R^{1A} is —COOH, R^{3A} is methyl, and R^{1B}, R^{1C}, R^{2A}, R^{2B}, and R^{3C} are all H (8b);
X is C—R^{2B}, R^{1A} is —COOH, R^{3A} is benzyloxy, and R^{1B}, R^{1C}, R^{2A}, R^{2B}, and R^{3C} are all H (8c);
X is C—R^{2B}, R^{1A} is —COOH, R^{3A} is trifluoromethyl, and R^{1B}, R^{1C}, R^{2A}, R^{2B}, and R^{3C} are all H (8d);
X is C—R^{2B}, R^{1A} is —COOH, R^{3A} is nitro, and R^{1B}, R^{1C}, R^{2A}, R^{2B}, and R^{3C} are all H (8e);
X is C—R^{2B}, R^{3A} is —COOH, and R^{1A}, R^{1B}, R^{1C}, R^{2A}, R^{2B}, and R^{3C} are all H (8i);
X is C—R^{2B}, R^{2A} is —COOH, and R^{1A}, R^{1B}, R^{1C}, R^{2B}, R^{3A}, and R^{3C} are all H (8k);
X is C—R^{2B}; R^{1A} is nitro, R^{2A} is —COOH, and R^{1B}, R^{1C}, R^{2B}, R^{3A}, and R^{3C} are all H (8l);
X is C—R^{2B}, R^{1A} is —COOH, R^{2A} is —COOH, and R^{1B}, R^{1C}, R^{2B}, R^{3A}, and R^{3C} are all H (8m);
X is C—R^{2B}, R^{1A} is —COOH, R^{2A} is —COOH, R^{3A} is phenyl, and R^{1B}, R^{1C}, R^{2B}, and R^{3C} are all H (8n);
X is C—R^{2B}, R^{1A} is —COOH, R^{2A} is Br, and R^{1B}, R^{1C}, R^{2B}, R^{3A}, and R^{3C} are all H (8o);
X is C—R^{2B}, R^{1A} is —COOH, R^{2B} is phenyl, and R^{1B}, R^{1C}, R^{2A}, R^{3A}, and R^{3C} are all H (8p);
X is C—R^{2B}, R^{1A} is —COOH, R^{3A} is —SO₂Me, and R^{1B}, R^{1C}, R^{2A}, R^{2B}, and R^{3C} are all H (8q);
X is C—R^{2B}, R^{1A} is —COOH, R^{2A} is —SO₂Me, R^{3A} is phenyl, and R^{1B}, R^{1C}, R^{2B}, and R^{3C} are all H (8r);
X is C—R^{2B}; R^{1A} is nitro, R^{2A} is —SO₂Me, R^{1B}, R^{1C}, R^{2B}, R^{3A}, and R^{3C} are all H (6u);
X is C—R^{2B}, R^{1A} is —COOH, R^{2B} is —COOH, and R^{1B}, R^{1C}, R^{2A}, R^{3A}, and R^{3C} are all H (8s);
X is C—R^{2B}, R^{1A} is —COOH, R^{1B} is —COOH, and R^{1C}, R^{2A}, R^{2B}, R^{3A}, and R^{3C} are all H (10b);
X is N, R^{1A} is —COOH, R^{3C} is hydroxyl, and R^{1B}, R^{1C}, R^{2A}, R^{3A}, R^{3B}, R^{3D} are all H (M7);
X is N, R^{1B} is —COOH, R^{3A} is methyl, and R^{1A}, R^{1C}, R^{2A}, R^{3A}, R^{3B}, R^{3D} are all H (M8);
X is N, R^{1A} is —COOH, and R^{1B}, R^{1C}, R^{2A}, R^{3A}, R^{3B}, R^{3C}, R^{3D} are all H (7b);
X is N, R^{1A} is —COOH, R^{3B} is hydroxyl, and R^{1B}, R^{1C}, R^{2A}, R^{3A}, R^{3C}, R^{3D} are all H (7d);
X is N, R^{1A} is —COOH, R^{3A} is hydroxyl, and R^{1B}, R^{1C}, R^{2A}, R^{3B}, R^{3C}, R^{3D} are all H (7e);
X is N, R^{1A} is —COOH, R^{1C} is hydroxyl, and R^{1B}, R^{2A}, R^{3A}, R^{3B}, R^{3C}, R^{3D} are all H (7f);
X is N, R^{1B} is —COOH, R^{3C} is hydroxyl, and R^{1A}, R^{1C}, R^{2A}, R^{3A}, R^{3B}, R^{3D} are all H (7m);
X is N, R^{1B} is —COOH, R^{3A} is hydroxyl, and R^{1A}, R^{1C}, R^{2A}, R^{3B}, R^{3C}, R^{3D} are all H (7o);
X is N, R^{1B} is —COOH, R^{3A} is morpholinyl, and R^{1A}, R^{1C}, R^{2A}, R^{3B}, R^{3C}, R^{3D} are all H (7p);
X is N, R^{1A} and R^{1B} are —COOH, and R^{1C}, R^{2A}, R^{3A}, R^{3B}, R^{3C}, R^{3D} are all H (10a); and
X is C—R^{2B}, R^{1A} is —COOH; R^{2B} is phenyl; R^{3A} is —(C₁-C₄)ONH₂, R^{1B}, R^{1C}, R^{2A}, R^{3B}, and R^{3C} are H;
X is C—R^{2B}; R^{1A} is —COOH; R^{2B} is phenyl; R^{3A}, R^{1B}, R^{1C}, R^{2A}, R^{3B}, and R^{3C} are H (664);
X is C—R^{2B}; R^{1A} is —COOH; R^{2B} is phenyl; R^{3A} is —CH₂ONH₂, R^{1B}, R^{1C}, R^{2A}, R^{3B}, and R^{3C} are H (699);
X is C—R^{2B}, R^{1A} is —COOH; R^{2B} is phenyl; R^{1B}, R^{1C}, R^{2A}, R^{3B}, and R^{3C} are H: R^{3A} is an oxime of formula —CH₂ON═C—R²⁰, wherein the oxime is formed by the reaction of —CH₂ONH₂ and an aldehyde 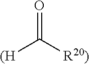
selected from the group consisting of A6, B7 (xz699-B7; 6-B7; or XZ710 (E) isomer; XZ703 (Z) isomer), B9, B11, D1 (XZ699-D1; 6-D1; XZ701 (E) isomer; XZ708 (Z) isomer)), D10, E6 (XZ699-E6; 6-E7; XZ702 (E) isomer; XZ709 (Z) isomer), F₁, F₃, F₉, F₁₀, F₁₁, G6, G8, H1, H7, H11, I1, I3, I7, I8, J9, K1, K9, L8, L11, M10 (XZ699-M10; 6-M10; XZ705 (E) isomer: XZ712 (Z) isomer), M11, N2, N3, N4, N8, O11, P2, P3 (XZ699-P3; 6-P3; XZ704 (E) isomer: XZ711 (Z) isomer), P4, P8, Q9, Q12, R7, R10, R11, S2, S11, T1, T3, or T12 from Table 4;
X is C—R^{2B}, R^{1A} is —COOH; R^{2B} is H or —(C₁-C₄)ONH₂, R^{1B}, R^{1C}, R^{2A}, R^{3A}, R^{3B}, and R^{3C} are H;
X is C—R^{2B}, R^{1A} is —COOH; R^{1B}, R^{1C}, R^{2A}, R^{2B}, R^{3A}, R^{3B}, and R^{3C} are H (XZ615);
X is C—R^{2B}, R^{1A} is —COOH; R^{2A} is —CH₂ONH₂, R^{1B}, R^{1C}, R^{2B}, R^{3A}, R^{3B}, and R^{3C} are H (XZ700); or
X is C—R^{2B}, R^{1A} is —COOH; R^{1B}, R^{1C}, R^{2B}, R^{3A}, R^{3B}, and R^{3C} are H: R^{2A} is an oxime of formula —CH₂ON═C—R²⁰, wherein the oxime is formed by the reaction of —CH₂ONH₂ and an aldehyde 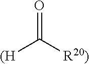
selected from the group consisting of B7 (xz700-B7; 5-B7), D1 (XZ700-D1; 5-D1; XZ706 (E) isomer; XZ717 (Z) isomer)), E6 (XZ700-E6; 5-E6), F₅, G6, G7, G8, I7, M10 (XZ700-M10; 5-M10), M11, N3, N4, O11, P1, P3 (XZ700-P3; 5-P3; XZ707 (E) isomer; XZ713 (Z) isomer), P4, P8, R¹⁰, R¹¹, S4, S12, and T12 from Table 4; and
wherein for each compound above, Y is NH.

7. (canceled)

8. The compound of claim 5, wherein
Y is NH; X is C—R^{2B}; R^{1A} is —COOH; R^{1B}, R^{1C}, R^{2A}, R^{3A}, R^{3B}, and R^{3C} are H; and
R^{2B} is 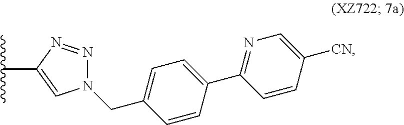
wherein
Y is NH; X is C—R^{2B}; R^{1A} is —COOH; R^{1B}, R^{1C}, R^{2B}, R^{3A}, R^{3B}, and R^{3C} are H; and
R^{2A} is 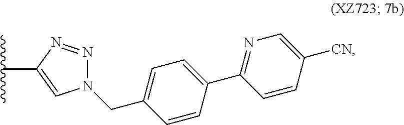
wherein
Y is NH; X is C—R^{2B}, R^{1A} is —COOH, R^{1B}, R^{1C}, R^{2A}, R^{2B}, R^{3B}, and R^{3C} are H;
R^{3A} is —OCH₂—X where X is 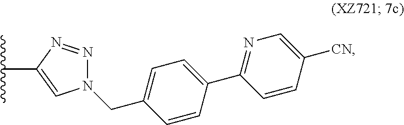
wherein Y is NH; X is C—R^{2B}; R^{1A} is —COOH: R^{2B} is phenyl: R^{1B}, R^{1C}, R^{2A}, R^{3B}, and R^{3C} are H;
R^{3A} is —OCH₂—X where X is 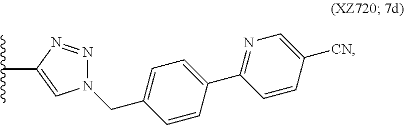
wherein
Y is NH; X is C—R^{2B}; R^{1A} is —COOH: R^{1B}, R^{1C}, R^{2A}, R^{2B}, R^{3B}, and R^{3C} are H;
R^{3A} is 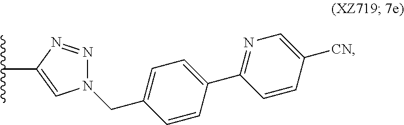
wherein
Y is NH; X is C—R^{2B}; R^{1A} is —COOH: R^{2B} is phenyl; R^{1B}, R^{1C}, R^{2A}, R^{3B}, and R^{3C} are H; R^{3A} 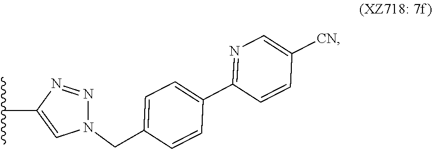
wherein
Y is NH; X is C—R^{2B}; R^{1A} is —COOH; R^{1B}, R^{1C}, R^{2A}, R^{2B}, R^{3B}, and R^{3C} are H;
R^{3A} is 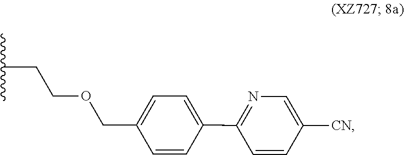 [SMILES: *CCOCc1ccc(-c2ccc(C#N)cn2)cc1]
or wherein
Y is NH; X is C—R^{2B}; R^{1A} is —COOH: R^{2B} is phenyl; R^{1B}, R^{1C}, R^{2A}, R^{3B}, and R^{3C} are H;
R^{3A} is

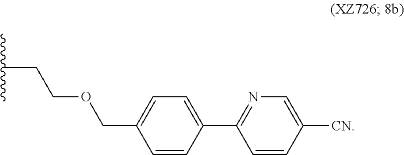 [SMILES: *CCOCc1ccc(-c2ccc(C#N)cn2)cc1]

9-15. (canceled)

16. The compound of claim 5 that is

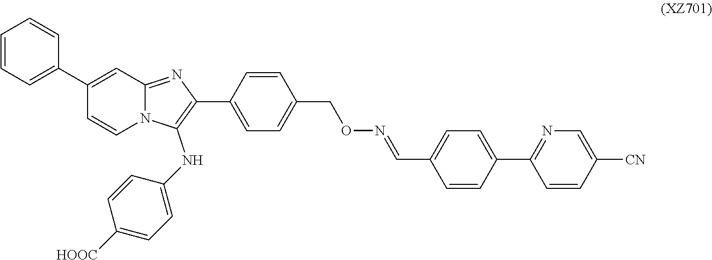 [SMILES: N#Cc1ccc(-c2ccc(/C=N/OCc3ccc(-c4nc5cc(-c6ccccc6)ccn5c4Nc4ccc(C(=O)O)cc4)cc3)cc2)nc1]

17. The compound of claim 1, wherein the compound is a compound of Formula I-A 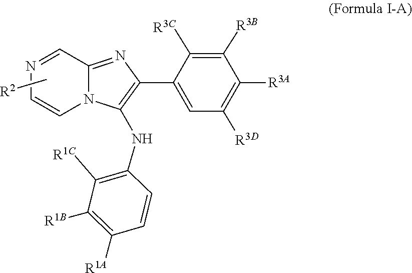
or a pharmaceutically acceptable salt thereof, wherein
one of R^{1A} and R^{1B} is H and the other is —COOH;
R^{1C} is H or hydroxyl;
R² is 0 to 3 substituents independently chosen from hydroxyl, halogen, C₁-C₂alkyl, C₁-C₂alkoxy;
R^{3A} is H, hydroxyl, C₁-C₂alkyl, C₁-C₂alkoxy, C₃-C₇cycloalkyl, or 5-7-membered heterocycloalkyl;
R^{3B}, R^{3C}, and R^{3D} are independently chosen from H, halogen, and hydroxyl.

18-19. (canceled)

20. The compound of claim 1, wherein the compound is a compound of Formula I-B 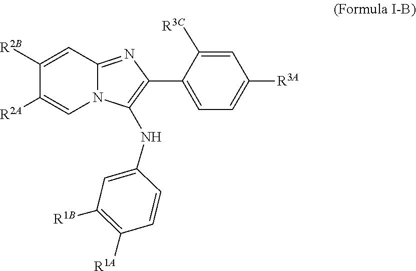
or a pharmaceutically acceptable salt thereof, wherein
R^{1A} and R^{1B} are independently chosen from H, halogen, hydroxyl, cyano, nitro, amino, —COOH, —CHO, —CONH₂, —SO₃H, —PO(OH)₂, —SO₂F, —OSO₂F, C₁-C₆alkyl, C₁-C₆alkoxy, C₁-C₂haloalkyl, and C₁-C₂haloalkoxy;
R^{2A} is H, halogen, hydroxyl, cyano, nitro, amino, —COOH, —CHO, —CONH₂, —SO₃H, —SO₂F, 2-SO₂C₁—C C₁-C₆alkyl, C₁-C₆alkoxy, C₁-C₂haloalkyl, or C₁-C₂haloalkoxy, ethynyl, ethynylbenzene;
R^{2B} is H, halogen, hydroxyl, cyano, C₁-C₆alkyl, C₁-C₆alkoxy, moon- or di(C₁-C₄alkyl)amino, phenyl, benzyl, C₁-C₂haloalkyl, or C₁-C₂haloalkoxy, ethynyl, ethynylbenzene;
R^{3A} is H, halogen, hydroxyl, cyano, nitro, amino, —COOH, —CHO, —CONH₂, —SO₃H, —SO₂F, phenyl, phenoxy, benzyl, benzyloxy, C₁-C₆alkyl, C₁-C₆alkoxy, moon- or di(C₁-C₄alkyl)amino, C₁-C₂haloalkyl, or C₁-C₂haloalkoxy; and
R^{3C} is H, —COOH, —SO₂F, —OSO₂F, halogen, hydroxyl, cyano, C₁-C₆alkyl, C₁-C₆alkoxy, mono- or di(C₁-C₄alkyl)amino, C₁-C₂haloalkyl, or C₁-C₂haloalkoxy;
wherein one of R^{1A}, R^{2A}, and R^{3A} is —COOH.

21-27. (canceled)

28. The compound of claim 1, wherein the compound is a compound of Formula I-C 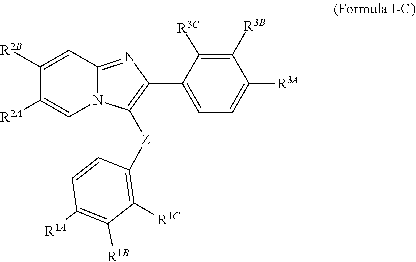
or a pharmaceutically acceptable salt thereof, wherein
R^{1A}, R^{1B} and R^{1C} are each independently H, —COOH, —SO₂F or —OSO₂F;
R^{2A} and R^{2B} are each independently H, phenyl, —SO₂F or —OSO₂F;
R^{3A}, 3B and R^{3C} are each independently H, —COOH, —SO₂F or —OSO₂F;
Z is O, NH, or N—SO₂F;
where at least one of R^{1A}, R^{1B} and R^{1C}, R^{2A} and R^{2B}, R^{3A}, R^{3B}, and R^{3C} is, —SO₂F or —OSO₂F.

29-30. (canceled)

31. The compound of claim 1, wherein the compound is a compound of Formula I-D 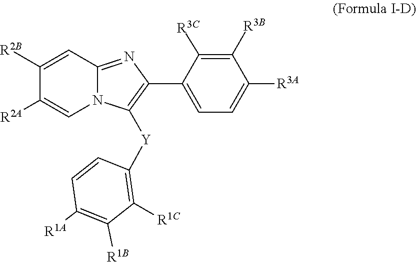
or a pharmaceutically acceptable salt thereof, wherein
R^{1A}, R^{1B} and R^{1C} are each independently H or —COOH;
R^{2A}, R^{B}, R^{3A}, R^{3B} and R^{3C} are each independently H, phenyl or -L-Rec, provided that no more than one of R^{2A}, R^{2B}, R^{3A}, R^{3B} and R^{3C} is -L-Rec; wherein L is a linker selected from the group consisting of 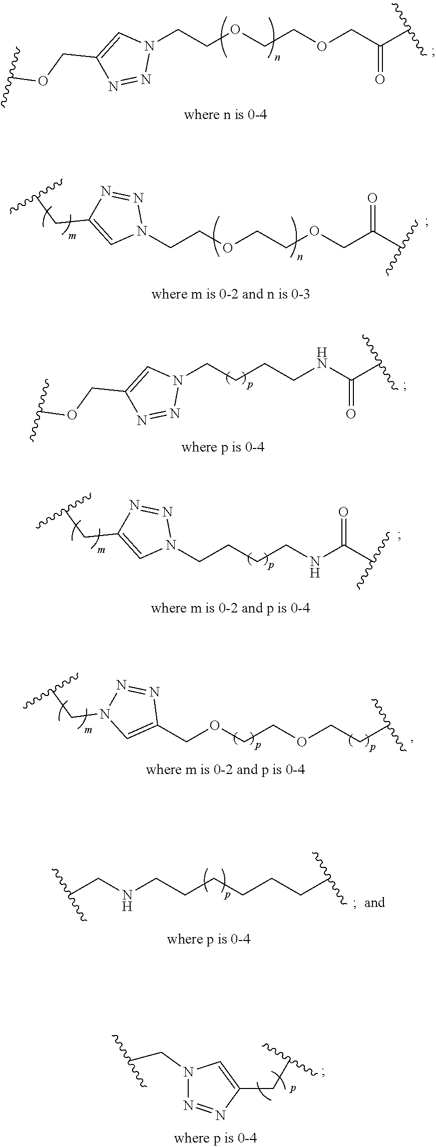
Rec is an E3 ubiquitin ligase recruiter; and
Y is O or NH.

32. The compound of claim 31, wherein Rec is a von Hippel-Lindau (VHL) E3 ubiquitin ligase recruiter, a cereblon (CRBN) E3 ubiquitin ligase recruiter, a Inhibitors of Apoptosis Protein (IAPs) E3 ubiquitin ligase recruiter, or a mouse double minute 2 homolog (MDM2) E3 ubiquitin ligase recruiter.

33. The compound of claim 32, wherein Rec is a VHL recruiter according to 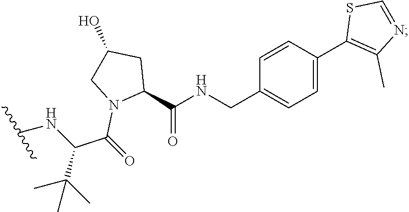 [SMILES: *N[C@H](C(=O)N1C[C@H](O)C[C@H]1C(=O)NCc1ccc(-c2scnc2C)cc1)C(C)(C)C]
or
Rec is a CRBN recruiter according to 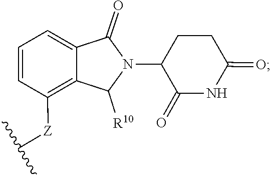
where R¹⁰ is H or carbonyl; and
Z is O, NH, or N(C₁-C₃alkyl).

34. The compound of claim 31, wherein
Y is NH;
R^{1A} is —COOH;
R^{2B} is phenyl;
R^{1B}, R^{1C}, R^{2A}, R^{3B} and R^{3C} are H;
R^{3A} is -L-Rec, wherein L is a linker selected from the group consisting of 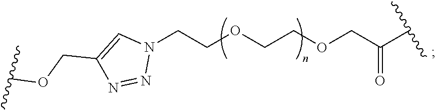
where n is 1-3; and
Rec is a VHL recruiter according to 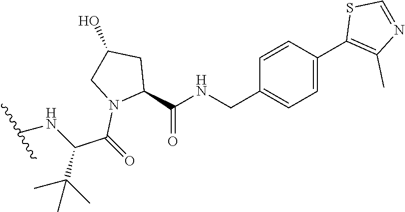 [SMILES: *N[C@H](C(=O)N1C[C@H](O)C[C@H]1C(=O)NCc1ccc(-c2scnc2C)cc1)C(C)(C)C]
or wherein
Y is NH;
R^{1A} is —COOH;
R^{1B}, R^{1C}, R^{2A}, R^{3A}, R^{3B} and R^{3C} are H;
R^{2B} is -L-Rec, wherein L is a linker selected from the group consisting of 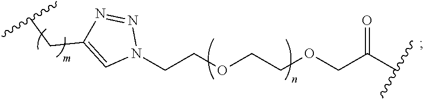
where m is 0-3; and
n is 1-3; and
Rec is a VHL recruiter according to 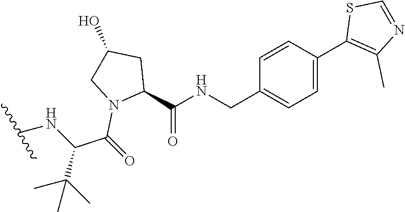 [SMILES: *N[C@H](C(=O)N1C[C@H](O)C[C@H]1C(=O)NCc1ccc(-c2scnc2C)cc1)C(C)(C)C]
or wherein
Y is NH;
R^{1A} is —COOH;
R^{1B}, R^{1C}, R^{2A}, R^{3A}, R^{3B} and R^{3C} are H;
R^{2B} is -L-Rec, wherein L is a linker selected from the group consisting of 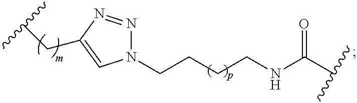
where m is 0-3, and p is 1-3; and
Rec is a CRBN recruiter selected from the group consisting of 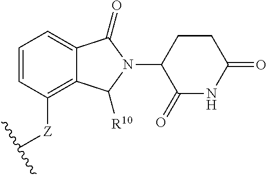
where R¹⁰ is H or carbonyl; and
Z is O or NH, or N(C₁-C₃alkyl)
or wherein
Y is NH;
R^{1A} is —COOH;
R^{2B} is H or phenyl;
R^{1B}, R^{1C}, R^{2A}, R^{3B} and R^{3C} are H;
R^{3A} is -L-Rec, wherein L is a linker selected from the group consisting of 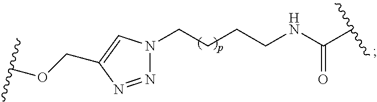
where p is 1-3; and
Rec is a CRBN recruiter selected from the group consisting of 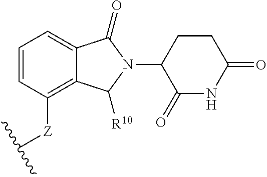
where R¹⁰ is H or carbonyl; and
Z is O or NH, or N(C₁-C₃alkyl)
or wherein
Y is NH;
R^{1A} is —COOH;
R^{2B} is H or phenyl;
R^{1B}, R^{1C}, R^{2A}, R^{3B} and R^{3C} are H;
R^{3A} is -L-Rec, wherein L is a linker selected from the group consisting of 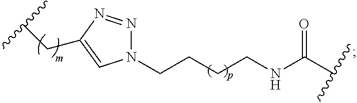
where m is 0-3;
p is 1-3; and
Rec is a CRBN recruiter selected from the group consisting of 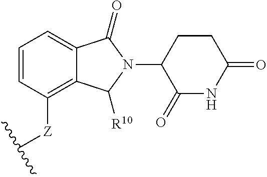
where R¹⁰ is H or carbonyl; and
Z is O or NH, or N(C₁-C₃alkyl)
or wherein
Y is NH;
R^{1A} is —COOH;
R^{2B} is H or phenyl;
R^{1B}, R^{1C}, R^{2A}, R^{3B} and R^{3C} are H;
R^{3A} is -L-Rec, wherein L is a linker selected from the group consisting of 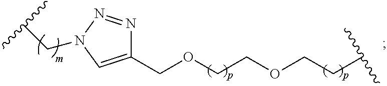
where m is 0-3,
p is 1-3; and
Rec is a CRBN recruiter selected from the group consisting of 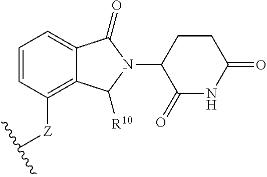
where R¹⁰ is H or carbonyl; and
Z is O or NH, or N(C₁-C₃alkyl)
or wherein
Y is NH;
R^{1A} is —COOH;
R^{2B} is H or phenyl;
R^{1B}, R^{1C}, R^{2A}, R^{3B} and R^{3C} are H;
R^{3A} is -L-Rec, wherein L is a linker selected from the group consisting of 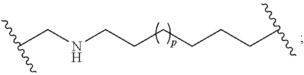
where p is 1-3; and
Rec is a CRBN recruiter selected from the group consisting of 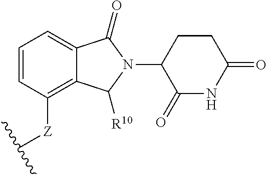
where R¹⁰ is H or carbonyl; and
Z is O or NH, or N(C₁-C₃alkyl)
or wherein
Y is NH;
R^{1A} is —COOH;
R^{2B} is H or phenyl;
R^{1B}, R^{1C}, R^{2A}, R^{3B} and R^{3C} are H;
R^{3A} is -L-Rec, wherein L is a linker selected from the group consisting of 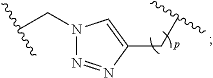
where p is 1-3; and
Rec is a CRBN recruiter selected from the group consisting of 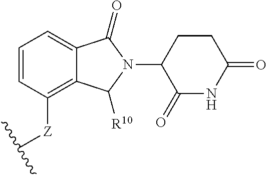
where R¹⁰ is H or carbonyl; and
Z is O or NH, or N(C₁-C₃alkyl).

35-50. (canceled)

51. A compound of Formula X, XI, XII, XIII, or XIV 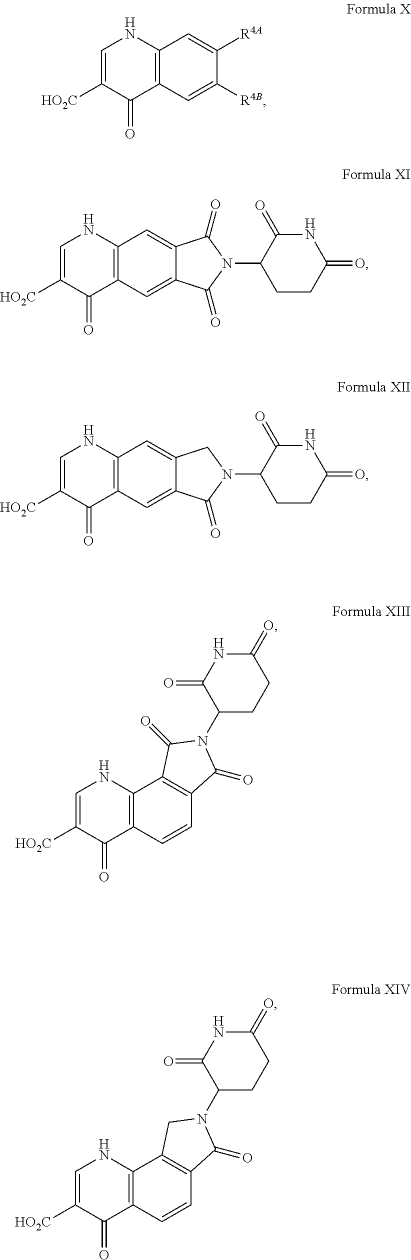
or a pharmaceutically acceptable salt thereof, wherein:
for Formula X, Z is O, NH, or N(C₁-C₃alkyl);
R¹⁰ is H or carbonyl; and
one of R^{4A} or R^{4B} is 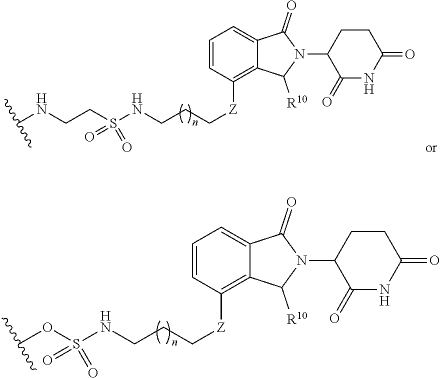
where n is 0-4.

52. (canceled)

53. The compound of claim 1, wherein the compound is a compound of Formula I-E 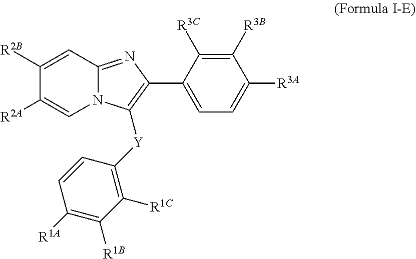
or a pharmaceutically acceptable salt thereof, wherein
R^{1A}, R^{1B} and R^{1C} are H or —COOH;
R^{2B} is H or phenyl;
R^{3B} and R^{3C} are H;
Y is O or NH; and
either R^{2A} or R^{3A} is —(C₁-C₄)ONH₂, an oxime of formula —(C₁-C₄)ON═C—R²⁰, wherein the oxime is formed by the reaction of —(C₁-C₄)ONH₂ and an aldehyde 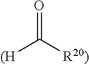
selected from the group consisting of A1-T12 in Table 4.

54. A pharmaceutical composition comprising the compound or salt of claim 1, together with a pharmaceutically acceptable carrier.

55. A method of treating cancer in a patient comprising administering a therapeutically effective amount of a compound of claim 1, pharmaceutically acceptable salt thereof, or a pharmaceutical composition comprising the compound to the patient.

56-62. (canceled)

63. The method of claim 55, wherein the compound or salt is administered together with a topoisomerase I or topoisomerase II inhibitor.

64. The method of claim 55, wherein the cancer is a cancer expressing TDP1.

65. (canceled)

66. A method of degrading TDP1 in a patient, of inhibiting the repair of a TOP1-DNA covalent complex in a patient, of stabilizing a TOP1-DNA complex in a patient, of degrading TOP1 in a patient, or of providing a molecular glue to a patient comprising administering a therapeutically effective amount of a compound claim 1, a pharmaceutically acceptable salt thereof, or a pharmaceutical composition comprising the compound to the patient.

67-105. (canceled)
